SEQUENCE LISTING (225 sequences)

SEQ ID NO: 1 - protein, 22 aa, Artificial Sequence
  CD4 TM domain
MALIVLGGVAGLLLFIGLGIFF

SEQ ID NO: 2 - protein, 21 aa, Artificial sequence
  CD8 TM domain
IYIWAPLAGTCGVLLLSLVIT

SEQ ID NO: 3 - protein, 21 aa, Artificial sequence
  a neurexin3b TM domain
GMVVGIVAAAALCILILLYAM

SEQ ID NO: 4 - protein, 21 aa, Artificial sequence
  Notch receptor polypeptide TM domain
FMYVAAAAFVLLFFVGCGVLL

SEQ ID NO: 5 - protein, 10 aa, Artificial sequence
  NES polypeptide
MVKELQEIRL

SEQ ID NO: 6 - protein, 11 aa, Artificial sequence
  NES polypeptide
MTASALARMEV

SEQ ID NO: 7 - protein, 10 aa, Artificial sequence
  NES polypeptide
LALKLAGLDI

SEQ ID NO: 8 - protein, 10 aa, Artificial sequence
  NES polypeptide
LQKKLEELEL

SEQ ID NO: 9 - protein, 10 aa, Artificial sequence
  NES polypeptide
LESNLRELQI

SEQ ID NO: 10 - protein, 10 aa, Artificial sequence
  NES polypeptide
LCQAFSDVLI

SEQ ID NO: 11 - protein, 12 aa, Artificial sequence
  NES polypeptide
MVKELQEIRLEP

SEQ ID NO: 12 - protein, 11 aa, Artificial sequence
  NES polypeptide
LQKKLEELELA

SEQ ID NO: 13 - protein, 11 aa, Artificial sequence
  NES polypeptide
LALKLAGLDIN

SEQ ID NO: 14 - protein, 12 aa, Artificial sequence
  NES polypeptide
LQLPPLERLTLD

SEQ ID NO: 15 - protein, 11 aa, Artificial sequence
  NES polypeptide
LQKKLEELELE

SEQ ID NO: 16 - protein, 10 aa, Artificial sequence
  NES polypeptide
MTKKFGTLTI

SEQ ID NO: 17 - protein, 10 aa, Artificial sequence
  NES polypeptide
LAEMLEDLHI

SEQ ID NO: 18 - protein, 10 aa, Artificial sequence
  NES polypeptide
LDQQFAGLDL

SEQ ID NO: 19 - protein, 10 aa, Artificial sequence
  NES polypeptide
LCQAFSDVIL

SEQ ID NO: 20 - protein, 9 aa, Artificial sequence
  NES polypeptide
LPVLENLTL

SEQ ID NO: 21 - protein, 16 aa, Artificial sequence
  NES polypeptide
IQQQLGQLTLENLQML

SEQ ID NO: 22 - protein, 26 aa, Artificial sequence
  calmodulin-binding polypeptide
KRRWKKNFIAVSAANRFKKISSSGAL

SEQ ID NO: 23 - protein, 26 aa, Artificial sequence
  calmodulin-binding polypeptide with A14F substitution
KRRWKKNFIAVSAFNRFKKISSSGAL

SEQ ID NO: 24 - protein, 22 aa, Artificial sequence
  calmodulin-binding polypeptide
FNARRKLKGAILTTMLFTRNFS

SEQ ID NO: 25 - protein, 22 aa, Artificial sequence
  calmodulin-binding polypeptide
FNARRKLAGAILFTMLFTRNFS

SEQ ID NO: 26 - protein, 46 aa, Artificial sequence
  calmodulin binding peptide
FNARRKLAGAILFTMLATRNFSGSFNARRKLAGAILFTMLATRNFS

SEQ ID NO: 27 - protein, 22 aa, Artificial sequence
  calmodulin binding peptide
FNARRKLAGAILFTMLATRNFS

SEQ ID NO: 28 - protein, 148 aa, Astyanax mexicanus
MDQLTEEQIAEFKEAFSLFDKDGDGTITTKELGTVMRSLGQNPTEAELQDMINEVDADGDGTIDFPEFLTMMARKMKYTDSEEEIREAFRVFDKDGNGYISAAELRHVMTNLGEKLTDEEVDEMIREADIDGDGQVNYEEFVQMMTAK

SEQ ID NO: 29 - protein, 148 aa, Astyanax mexicanus
MDQLTEEQIAEFKEAFSLLDKDGDGTITTKELGTGMRSLGQNPTEAELQDMINEVDADGDGTIDFPEFLTMMARKMKYTDSEEEIREAFRVFDKDGNGYISAAELRHVMTNLGEKLTDEEVDEMIREADIDGDGQVNYEEFVQMMTAK

SEQ ID NO: 30 - protein, 187 aa, Homo sapiens
MPEVERKPKITASRKLLLKSLMLAKAKECWEQEHEEREAEKVRYLAERIPTLQTRGLSLSALQDLCRELHAKVEVVDEERYDIEAKCLHNTREIKDLKLKVMDLRGKFKRPPLRRVRVSADAMLRALLGSKHKVSMDLRANLKSVKKEDTEKERPVEVGDWRKNVEAMSGMEGRKKMFDAAKSPTSQ

SEQ ID NO: 31 - protein, 20 aa, Artificial sequence
  troponin I polypeptide
KDLKLKVMDLRGKFKRPPLR

SEQ ID NO: 32 - protein, 25 aa, Artificial sequence
  troponin I polypeptide
RMSADAMLKALLGSKHKVAMDLRAN

SEQ ID NO: 33 - protein, 44 aa, Artificial sequence
  troponin I polypeptide
NQKLFDLRGKFKRPPLRRVRMSADAMLKALLGSKHKVAMDLRAN

SEQ ID NO: 34 - protein, 160 aa, Homo sapiens
MTDQQAEARSYLSEEMIAEFKAAFDMFDADGGGDISVKELGTVMRMLGQTPTKEELDAIIEEVDEDGSGTIDFEEFLVMMVRQMKEDAKGKSEEELAECFRIFDRNADGYIDPGELAEIFRASGEHVTDEEIESLMKDGDKNNDGRIDFDEFLKMMEGVQ

SEQ ID NO: 35 - protein, 160 aa, Rattus norvegicus
MTDQQAEARSYLSEEMIAEFKAAFDMFDADGGGDISVKELGTVMRMLGQTPTKEELDAIIEEVDEDGSGTIDFEEFLVMMVRQMKEDAKGKSEEELAECFRIFDRDANGYIDAEELAEIFRASGEHVTDEEIESLMKDGDKNNDGRIDFDEFLKMMEGVQ

SEQ ID NO: 36 - protein, 142 aa, Artificial sequence
  LOV polypeptide
DLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAK

SEQ ID NO: 37 - protein, 142 aa, Artificial sequence
  LOV polypeptide
SLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREAVMLIKKTAEEIDEAAK

SEQ ID NO: 38 - protein, 142 aa, Artificial sequence
  LOV polypeptide
SRATTLERIEKSFVITDPRLPDNPIIFVSDSFLQLTEYSREEILGRNCRFLQGPETDRATVRKIRDAIDNQTEVTVQLINYTKSGKKFWNLFHLQPMRDQKGDVQYFIGVQLDGTERVRDAAEREAVMLVKKTAEEIDEAAK

SEQ ID NO: 39 - protein, 138 aa, Artificial sequence
  LOV light-activated polypeptide
FRATTLERIEKSFVITDPRLPDNPIIFVSDSFLQLTEYSREEILGRNCRFLQGPETDRATVRKIRDAIDNQTEVTVQLINYTKSGKKFWNVFHLQPMRDYKGDVQYFIGVQLDGTERLHGAAEREAVCLVKKTAFQIA

SEQ ID NO: 40 - protein, 138 aa, Artificial sequence
  LOV light-activated polypeptide
SRATTLERIEKSFVITDPRLPDNPIIFVSDSFLQLTEYSREEILGRNCRFLQGPETDRATVRKIRDAIDNQTEVTVQLINYTKSGKKFWNLFHLQPMRDQKGDVQYFIGVQLDGTERVRDAAEREAVMLVKKTAEEID

SEQ ID NO: 41 - protein, 142 aa, Artificial sequence
  LOV light-activated polypeptide
SRATTLERIEKSFVITDPRLPDNPIIFVSDSFLQLTEYSREEILGRNCRFLQGPETDRATVRKIRDAIDNQTEVTVQLINYTKSGKKFWNVFHLQPMRDYKGDVQYFIGVQLDGTERLHGAAEREAVCLVKKTAFEIDEAAK

SEQ ID NO: 42 - protein, 149 aa, Artificial sequence
  LOV light-activated polypeptide
SRATTLERIEKSFVITDPRLPDNPIIFVSDSFLQLTEYSREEILGRNCRFLQGPETDRATVRKIRDAIDNQTEVTVQLINYTKSGKKFWNLFHLQPMRDQKGDVQYFIGVQLDGTERVRDAAEREAVMLVKKTAEEIDEAAKENLYFQM

SEQ ID NO: 43 - protein, 149 aa, Artificial sequence
  LOV light-activated polypeptide
SRATTLERIEKSFVITDPRLPDNPIIFVSDSFLQLTEYSREEILGRNCRFLQGPETDRATVRKIRDAIDNQTEVTVQLINYTKSGKKFWNVFHLQPMRDYKGDVQYFIGVQLDGTERLHGAAEREAVCLVKKTAFEIDEAAKENLYFQM

SEQ ID NO: 44 - protein, 145 aa, Artificial sequence
  LOV light-activated polypeptide
FRATTLERIEKSFVITDPRLPDNPIIFVSDSFLQLTEYSREEILGRNCRFLQGPETDRATVRKIRDAIDNQTEVTVQLINYTKSGKKFWNVFHLQPMRDYKGDVQYFIGVQLDGTERLHGAAEREAVCLVKKTAFQIAENLYFQM

SEQ ID NO: 45 - protein, 145 aa, Artificial sequence
  LOV light-activated polypeptide
SRATTLERIEKSFVITDPRLPDNPIIFVSDSFLQLTEYSREEILGRNCRFLQGPETDRATVRKIRDAIDNQTEVTVQLINYTKSGKKFWNLFHLQPMRDQKGDVQYFIGVQLDGTERVRDAAEREAVMLVKKTAEEIDENLYFQG

SEQ ID NO: 46 - protein, 145 aa, Artificial sequence
  LOV light-activated polypeptide
FRATTLERIEKSFVITDPRLPDNPIIFVSDSFLQLTEYSREEILGRNCRFLQGPETDRATVRKIRDAIDNQTEVTVQLINYTKSGKKFWNVFHLQPMRDYKGDVQYFIGVQLDGTERLHGAAEREAVCLVKKTAFQIAENLYFQG

SEQ ID NO: 47 - protein, 7 aa, Artificial sequence
  proteolytically cleavable linker
PLQGMTS

SEQ ID NO: 48 - protein, 6 aa, Artificial sequence
  proteolytically cleavable linker
PLQGMT

SEQ ID NO: 49 - protein, 7 aa, Artificial sequence
  proteolytically cleavable linker
ENLYFQS

SEQ ID NO: 50 - protein, 7 aa, Artificial sequence
  proteolytically cleavable linker
ENLYFQY

SEQ ID NO: 51 - protein, 5 aa, Artificial sequence
  ER export sequence
  misc_feature (2)..(3): Xaa can be any naturally occurring amino acid
VXXSL

SEQ ID NO: 52 - protein, 5 aa, Artificial sequence
  ER export sequence
VKESL

SEQ ID NO: 53 - protein, 5 aa, Artificial sequence
  ER export sequence
VLGSL

SEQ ID NO: 54 - protein, 16 aa, Artificial sequence
  ER export sequence
NANSFCYENEVALTSK

SEQ ID NO: 55 - protein, 20 aa, Artificial sequence
  ER export sequence
KSRITSEGEYIPLDQIDINV

SEQ ID NO: 56 - protein, 6 aa, Artificial sequence
  ER export sequence
  misc_feature (2)..(2): Xaa can be any naturally occurring amino acid
FXYENE

SEQ ID NO: 57 - protein, 7 aa, Artificial sequence
  ER export sequence
FCYENEV

SEQ ID NO: 58 - protein, 12 aa, Artificial sequence
  cathepsin B cleavage site
SLLIARRMPNFN

SEQ ID NO: 59 - protein, 12 aa, Artificial sequence
  Epstein-Barr virus protease cleavage site
SKLVQASASGVN

SEQ ID NO: 60 - protein, 12 aa, Artificial sequence
  Epstein-Barr virus protease cleavage site
SSYLKASDAPDN

SEQ ID NO: 61 - protein, 12 aa, Artificial sequence
  MMP-3 cleavage site
RPKPQQFFGLMN

SEQ ID NO: 62 - protein, 12 aa, Artificial sequence
  MMP-7 cleavage site
SLRPLALWRSFN

SEQ ID NO: 63 - protein, 12 aa, Artificial sequence
  MMP-9 cleavage site
SPQGIAGQRNFN

SEQ ID NO: 64 - protein, 14 aa, Artificial sequence
  thermolysin-like MMP cleavage site
DVDERDVRGFASFL

SEQ ID NO: 65 - protein, 12 aa, Artificial sequence
  MMP-2 cleavage sitet
SLPLGLWAPNFN

SEQ ID NO: 66 - protein, 12 aa, Artificial sequence
  cathespin L
SLLIFRSWANFN

SEQ ID NO: 67 - protein, 12 aa, Artificial sequence
  cathepsin D cleavage site
SGVVIATVIVIT

SEQ ID NO: 68 - protein, 12 aa, Artificial sequence
  (MMP-1 cleavage site
SLGPQGIWGQFN

SEQ ID NO: 69 - protein, 12 aa, Artificial sequence
  urokinase-type plasminogen activator cleavage site
KKSPGRVVGGSV

SEQ ID NO: 70 - protein, 12 aa, Artificial sequence
  MT-MMP cleavage site
PQGLLGAPGILG

SEQ ID NO: 71 - protein, 31 aa, Artificial sequence
  stromelysin 3 cleavage site
HGPEGLRVGFYESDVMGRGHARLVHVEEPHT

SEQ ID NO: 72 - protein, 12 aa, Artificial sequence
  matrix metalloproteinase 13 cleavage site
GPQGLAGQRGIV

SEQ ID NO: 73 - protein, 12 aa, Artificial sequence
  tissue-type plasminogen activator cleavage site
GGSGQRGRKALE

SEQ ID NO: 74 - protein, 12 aa, Artificial sequence
  human prostate-specific antigen cleavage site
SLSALLSSDIFN

SEQ ID NO: 75 - protein, 12 aa, Artificial sequence
  kallikrein cleavage site
SLPRFKIIGGFN

SEQ ID NO: 76 - protein, 12 aa, Artificial sequence
  neutrophil elastase cleavage site
SLLGIAVPGNFN

SEQ ID NO: 77 - protein, 12 aa, Artificial sequence
  calpain cleavage site
FFKNIVTPRTPP

SEQ ID NO: 78 - protein, 7 aa, Artificial sequence
  proteolytically cleavable linker
  misc_feature (7)..(7): Xaa can be any naturally occurring amino acid
ENLYFQX

SEQ ID NO: 79 - protein, 7 aa, Artificial sequence
  proteolytically cleavable linker
ENLYFQG

SEQ ID NO: 80 - protein, 7 aa, Artificial sequence
  proteolytically cleavable linker
ENLYFQW

SEQ ID NO: 81 - protein, 7 aa, Artificial sequence
  proteolytically cleavable linker
ENLYFQM

SEQ ID NO: 82 - protein, 7 aa, Artificial sequence
  proteolytically cleavable linker
ENLYFQH

SEQ ID NO: 83 - protein, 7 aa, Artificial sequence
  proteolytically cleavable linker
ENLYFQN

SEQ ID NO: 84 - protein, 7 aa, Artificial sequence
  proteolytically cleavable linker
ENLYFQA

SEQ ID NO: 85 - protein, 7 aa, Artificial sequence
  proteolytically cleavable linker
ENLYFQQ

SEQ ID NO: 86 - protein, 7 aa, Artificial sequence
  proteolytically cleavable linker
DEVVECS

SEQ ID NO: 87 - protein, 10 aa, Artificial sequence
  proteolytically cleavable linker
DEAEDVVECS

SEQ ID NO: 88 - protein, 11 aa, Artificial sequence
  proteolytically cleavable linker
EDAAEEVVECS

SEQ ID NO: 89 - protein, 6 aa, Artificial sequence
  proteolytically cleavable linker
PLFAAR

SEQ ID NO: 90 - protein, 11 aa, Artificial sequence
  proteolytically cleavable linker
QQEVYGMMPRD

SEQ ID NO: 91 - protein, 219 aa, Artificial sequence
  TEV protease
GESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMV

SEQ ID NO: 92 - protein, 15 aa, Artificial sequence
  BirA biotin-protein ligase polypeptide.
GLNDIFEAQKIEWHE

SEQ ID NO: 93 - protein, 321 aa, Escherichia coli
MKDNTVPLKLIALLANGEFHSGEQLGETLGMSRAAINKHIQTLRDWGVDVFTVPGKGYSLPEPIQLLNAEEILSQLDGGSVAVLPVIDSTNQYLLDRIGELKSGDACVAEYQQAGRGRRGRKWFSPFGANLYLSMFWRLEQGPAAAIGLSLVIGIVMAEVLRKLGADKVRVKWPNDLYLQDRKLAGILVELTGKTGDAAQIVIGAGINMAMRRVEESVVNQGWITLQEAGINLDRNTLAAMLIRELRAALELFEQEGLAPYLSRWEKLDNFINRPVKLIIGDKEIFGISRGIDKQGALLLEQDGIIKPWMGGEISLRSAEK

SEQ ID NO: 94 - protein, 250 aa, Glycine max
MGKSYPTVSADYQKAVEKAKKKLRGFIAEKRCAPLMLRLAWHSAGTFDKGTKTGGPFGTIKHPAELAHSANNGLDIAVRLLEPLKAEFPILSYADFYQLAGVVAVEVTGGPEVPFHPGREDKPEPPPEGRLPDATKGSDHLRDVFGKAMGLTDQDIVALSGGHTIGAAHKERSGFEGPWTSNPLIFDNSYFTELLSGEKEGLLQLPSDKALLSDPVFRPLVDKYAADEDAFFADYAEAHQKLSELGFADA

SEQ ID NO: 95 - protein, 7 aa, Artificial sequence
  tobacco etch virus (TEV) protease cleavage site
ENLYFQL

SEQ ID NO: 96 - protein, 5 aa, Artificial sequence
  enterokinase cleavage site
DDDDK

SEQ ID NO: 97 - protein, 4 aa, Artificial sequence
  thrombin cleavage site
LVPR

SEQ ID NO: 98 - protein, 6 aa, Artificial sequence
  proteolytically cleavable linker
LVPRGS

SEQ ID NO: 99 - protein, 8 aa, Artificial sequence
  proteolytically cleavable linker
LEVLFQGP

SEQ ID NO: 100 - protein, 10 aa, Artificial sequence
  proteolytically cleavable linker
CGLVPAGSGP

SEQ ID NO: 101 - protein, 12 aa, Artificial sequence
  proteolytically cleavable linker
SLLKSRMVPNFN

SEQ ID NO: 102 - protein, 49 aa, Artificial sequence
  anthopleurin B toxin
GVPCLCDSDGPRPRGNTLSGILWFYPSGCPSGWHNCKAHGPNIGWCCKK

SEQ ID NO: 103 - protein, 79 aa, Artificial sequence
  calitoxin
MKTQVLALFVLCVLFCLAESRTTLNKRNDIEKRIECKCEGDAPDLSHMTGTVYFSCKGGDGSWSKCNTYTAVADCCHQA

SEQ ID NO: 104 - DNA, 54 bp, Artificial sequence
  telRL site
tatcagcacacaattgcccattatacgcgcgtataatggactattgtgtgctga

SEQ ID NO: 105 - DNA, 42 bp, Artificial sequence
  pal site
acctatttcagcatactacgcgcgtagtatgctgaaataggt

SEQ ID NO: 106 - DNA, 22 bp, Artificial sequence
  phi K02 telRL site
ccattatacgcgcgtataatgg

SEQ ID NO: 107 - DNA, 33 bp, Artificial sequence
  FRT site
taacttcgtatagcatacattatacgaagttat

SEQ ID NO: 108 - DNA, 34 bp, Artificial sequence
  FRT site
gaagttcctattctctagaaagtataggaacttc

SEQ ID NO: 109 - DNA, 100 bp, Artificial sequence
  phiC31 attP site
cccaggtcagaagcggttttcgggagtagtgccccaactggggtaacctttgagttctctcagttgggggcgtagggtcgccgacaygacacaaggggtt

SEQ ID NO: 110 - DNA, 101 bp, Artificial sequence
  Lambda attP site
tgatagtgacctgttcgtttgcaacacattgatgagcaatgcttttttataatgccaactttgtacaaaaaagctgaacgagaaacgtaaaatgatataaa

SEQ ID NO: 111 - protein, 16 aa, Artificial sequence
  mitochondrial localization sequence
LGRVIPRKIASRASLM

SEQ ID NO: 112 - protein, 30 aa, Artificial sequence
  mitochondrial localization sequence
MSVLTPLLLRGLTGSARRLPVPRAKIHSLL

SEQ ID NO: 113 - protein, 27 aa, Artificial sequence
  Nav1.6 soma localization signal
TVRVPIAVGESDFENLNTEDVSSESDP

SEQ ID NO: 114 - protein, 7 aa, Artificial sequence
  Nuclear localization signal
PKKKRKV

SEQ ID NO: 115 - protein, 16 aa, Artificial sequence
  Nuclear localization signal
KRPAATKKAGQAKKKK

SEQ ID NO: 116 - protein, 9 aa, Artificial sequence
  Nuclear localization signal
PAAKRVKLD

SEQ ID NO: 117 - protein, 11 aa, Artificial sequence
  Nuclear localization signal
RQRRNELKRSP

SEQ ID NO: 118 - protein, 38 aa, Artificial sequence
  Nuclear localization signal
NQSSNFGPMKGGNFGGRSSGPYGGGGQYFAKPRNQGGY

SEQ ID NO: 119 - protein, 42 aa, Artificial sequence
  Nuclear localization signal
RMRIZFKNKGKDTAELRRRRVEVSVELRKAKKDEQILKRRNV

SEQ ID NO: 120 - protein, 8 aa, Artificial sequence
  Nuclear localization signal
VSRKRPRP

SEQ ID NO: 121 - protein, 8 aa, Artificial sequence
  Nuclear localization signal
PPKKARED

SEQ ID NO: 122 - protein, 8 aa, Artificial sequence
  Nuclear localization signal
PQPKKKPL

SEQ ID NO: 123 - protein, 12 aa, Artificial sequence
  Nuclear localization signal
SALIKKKKKMAP

SEQ ID NO: 124 - protein, 5 aa, Artificial sequence
  Nuclear localization signal
DRLRR

SEQ ID NO: 125 - protein, 7 aa, Artificial sequence
  Nuclear localization signal
PKQKKRK

SEQ ID NO: 126 - protein, 10 aa, Artificial sequence
  Nuclear localization signal
RKLKKKIKKL

SEQ ID NO: 127 - protein, 10 aa, Artificial sequence
  Nuclear localization signal
REKKKFLKRR

SEQ ID NO: 128 - protein, 20 aa, Artificial sequence
  Nuclear localization signal
KRKGDEVDGVDEVAKKKSKK

SEQ ID NO: 129 - protein, 17 aa, Artificial sequence
  Nuclear localization signal
RKCLQAGMNLEARKTKK

SEQ ID NO: 130 - protein, 11 aa, Artificial sequence
  Nuclear localization signal
YGRKKRRQRRR

SEQ ID NO: 131 - protein, 12 aa, Artificial sequence
  Nuclear localization signal
RRQRRTSKLMKR

SEQ ID NO: 132 - protein, 27 aa, Artificial sequence
  Transportan
GWTLNSAGYLLGKINLKALAALAKKIL

SEQ ID NO: 133 - protein, 33 aa, Artificial sequence
  protein transduction domain
KALAWEAKLAKALAKALAKHLAKALAKALKCEA

SEQ ID NO: 134 - protein, 16 aa, Artificial sequence
  protein transduction domain
RQIKIWFQNRRMKWKK

SEQ ID NO: 135 - protein, 9 aa, Artificial sequence
  protein transduction domain
RKKRRQRRR

SEQ ID NO: 136 - protein, 11 aa, Artificial sequence
  protein transduction domain
YARAAARQARA

SEQ ID NO: 137 - protein, 11 aa, Artificial sequence
  protein transduction domain
THRLPRRRRRR

SEQ ID NO: 138 - protein, 11 aa, Artificial sequence
  protein transduction domain
GGRRARRRRRR

SEQ ID NO: 139 - protein, 148 aa, Artificial sequence; identical to SEQ ID NO: 29
  calmodulin polypeptide

SEQ ID NO: 140 - protein, 22 aa, Artificial sequence
  calmodulin-binding polypeptide
FNARRKLKGAILFTMLFTRNFS

SEQ ID NO: 141 - protein, 142 aa, Artificial sequence; identical to SEQ ID NO: 38
  LOV polypeptide

SEQ ID NO: 142 - protein, 335 aa, Artificial sequence
  tTA-VP16 transcription factor
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 143 - protein, 27 aa, Artificial sequence
  Nav1.6 soma localization signal
TVRVPIAVGESDFENLNTEDVSSESDP

SEQ ID NO: 144 - protein, 8 aa, Artificial sequence
  protein transduction domain
RKKRRQRR

SEQ ID NO: 145 - protein, 250 aa, Artificial sequence; identical to SEQ ID NO: 94
  APX peroxidase

SEQ ID NO: 146 - DNA, 42 bp, Artificial sequence; identical to SEQ ID NO: 105
  pal site

SEQ ID NO: 147 - DNA, 100 bp, Artificial sequence; identical to SEQ ID NO: 109
  phiC31 target site

SEQ ID NO: 148 - protein, 22 aa, Artificial sequence
  calmodulin-binding polypeptide
FNARRKLKGAILTTMLATRNFS

SEQ ID NO: 149 - protein, 7 aa, Artificial sequence
  proteolytically cleavable linker
ENLYFQL

SEQ ID NO: 150 - protein, 142 aa, Artificial sequence
  LOV domain
SRATTLERIEKSFVITDPRLPDNPVIFVSDSFLQLTEYSREEILGRNCRFLQGPETDRATVRKIRDAIDNQTEVTVQLINYTKSGKKFWNLFHLQPMRDQKGDVQYFIGVQLDGTERVRDAAEREAVMLVKKTAEEIDEAAK

SEQ ID NO: 151 - protein, 142 aa, Artificial sequence
  LOV domain
SRATTLERIEKSFVITDPRLPDNPIIFVSDSFLQLTEYSREEILGRNCRFLQGPETDRATVRKIRDAIDNQTEVTVQLINYTKSGKKFWNVFHLQPMRDYKGDVQYFIGVQLDGTERLHGAAEREAVCLVKKTAFQIAEAAK

SEQ ID NO: 152 - protein, 138 aa, Artificial sequence
  LOV domain
SRATTLERIEKSFVITDPRLPDNPIIFVSDSFLQLTEYSREEILGRNCRFLQGPETDRATVRKIRDAIDNQTEVTVQLINYTKSGKKFWNVFHLQPMRDYKGDVQYFIGVQLDGTERLHGAAEREAVCLVKKTAFQIA

SEQ ID NO: 153 - protein, 238 aa, Tobacco etch virus
GESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMSKPEEPFQPVKEATQLMNEL

SEQ ID NO: 154 - protein, 242 aa, Tobacco etch virus
GESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMSKPEEPFQPVKEATQLMNELVYSQ

SEQ ID NO: 155 - protein, 242 aa, Artificial sequence
  wtTEV
GESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNELVYSQ

SEQ ID NO: 156 - protein, 1368 aa, Staphylococcus pyogenes [sequence omitted]

SEQ ID NO: 157 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 158 - protein, 310 aa, Artificial sequence
  ChR2
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVILIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVP

SEQ ID NO: 159 - protein, 340 aa, Artificial sequence
  ChR2 with ER export and trafficking signal sequences
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVILIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVPAAAKSRITSEGEYIPLDQIDINVFCYENEV

SEQ ID NO: 160 - protein, 310 aa, Artificial sequence
  ChR2 SSFO
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTSPVILIHLSNLTGLSNDYSRRTMGLLVSAIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVP

SEQ ID NO: 161 - protein, 340 aa, Artificial sequence
  ChR2 SSFO with ER export and trafficking signal sequences
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTSPVILIHLSNLTGLSNDYSRRTMGLLVSAIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVPAAAKSRITSEGEYIPLDQIDINVFCYENEV

SEQ ID NO: 162 - protein, 300 aa, Volvox carteri
MDYPVARSLIVRYPTDLGNGTVCMPRGQCYCEGWLRSRGTSIEKTIAITLQWVVFALSVACLGWYAYQAWRATCGWEEVYVALIEMMKSIIEAFHEFDSPATLWLSSGNGVVWMRYGEWLLTCPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEED

SEQ ID NO: 163 - protein, 330 aa, Artificial sequence
  VChR1 with ER export and trafficking signal sequences
MDYPVARSLIVRYPTDLGNGTVCMPRGQCYCEGWLRSRGTSIEKTIAITLQWVVFALSVACLGWYAYQAWRATCGWEEVYVALIEMMKSIIEAFHEFDSPATLWLSSGNGVVWMRYGEWLLTCPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEEDAAAKSRITSEGEYIPLDQIDINVFCYENEV

SEQ ID NO: 164 - protein, 344 aa, Artificial sequence
  VChR1 with ER export and trafficking signal sequences
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWEEIYVATIEMIKFIIEYFHEFDEPAVIYSSNGNKTVWLRYAEWLLTCPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEED

SEQ ID NO: 165 - protein, 374 aa, Artificial sequence
  C1V1 with ER export and trafficking signal sequences
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWEEIYVATIEMIKFIIEYFHEFDEPAVIYSSNGNKTVWLRYAEWLLTCPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEEDAAAKSRITSEGEYIPLDQIDINVFCYENEV

SEQ ID NO: 166 - protein, 348 aa, Artificial sequence
  C1C2
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWEEIYVATIEMIKFIIEYFHEFDEPAVIYSSNGNKTVWLRYAEWLLTCPVILIHLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAV

SEQ ID NO: 167 - protein, 378 aa, Artificial sequence
  C1C2 with ER export and trafficking signal sequences
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWEEIYVATIEMIKFIIEYFHEFDEPAVIYSSNGNKTVWLRYAEWLLTCPVILIHLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVAAAKSRITSEGEYIPLDQIDINVFCYENEV

SEQ ID NO: 168 - protein, 350 aa, Artificial sequence
  ReaChR (red shifted ChR)
MVSRRPWLLALALAVALAAGSAGASTGSDATVPVATQDGPDYVFHRAHERMLFQTSYTLENNGSVICIPNNGQCFCLAWLKSNGTNAEKLAANILQWVTFALSVACLGWYAYQAWRATCGWEEVYVALIEMMKSIIEAFHEFDSPATLWLSSGNGVVWMRYGEWLLTCPVILIHLSNLTGLKDDYSKRTMGLLVSDVGCIVWGATSAMCTGWTKILFFLISLSYGMYTYFHAAKVYIEAFHTVPKGLCRQLVRAMAWLFFVSWGMFPVLFLLGPEGFGHISPYGSAIGHSILDLIAKNMWGVLGNYLRVKIHEHILLYGDIRKKQKITIAGQEMEVETLVAEEEDKYESS

SEQ ID NO: 169 - protein, 380 aa, Artificial sequence
  ReaChR (red shifted ChR) with ER export and trafficking signal sequences
MVSRRPWLLALALAVALAAGSAGASTGSDATVPVATQDGPDYVFHRAHERMLFQTSYTLENNGSVICIPNNGQCFCLAWLKSNGTNAEKLAANILQWVTFALSVACLGWYAYQAWRATCGWEEVYVALIEMMKSIIEAFHEFDSPATLWLSSGNGVVWMRYGEWLLTCPVILIHLSNLTGLKDDYSKRTMGLLVSDVGCIVWGATSAMCTGWTKILFFLISLSYGMYTYFHAAKVYIEAFHTVPKGLCRQLVRAMAWLFFVSWGMFPVLFLLGPEGFGHISPYGSAIGHSILDLIAKNMWGVLGNYLRVKIHEHILLYGDIRKKQKITIAGQEMEVETLVAEEEDKYESSAAAKSRITSEGEYIPLDQIDINVFCYENEV

SEQ ID NO: 170 - protein, 316 aa, Artificial sequence
  SdChR (CheRiff)
MGGAPAPDAHSAPPGNDSAGGSEYHAPAGYQVNPPYHPVHGYEEQCSSIYIYYGALWEQETARGFQWFAVFLSALFLAFYGWHAYKASVGWEEVYVCSVELIKVILEIYFEFTSPAMLFLYGGNITPWLRYAEWLLTCPVILIHLSNITGLSEEYNKRTMALLVSDLGTICMGVTAALATGWVKWLFYCIGLVYGTQTFYNAGIIYVESYYIMPAGGCKKLVLAMTAVYYSSWLMFPGLFIFGPEGMHTLSVAGSTIGHTIADLLSKNIWGLLGHFLRIKIHEHIIMYGDIRRPVSSQFLGRKVDVLAFVTEEDKV

SEQ ID NO: 171 - protein, 346 aa, Artificial sequence
  SdChR (CheRiff) with ER export and trafficking signal sequences
MGGAPAPDAHSAPPGNDSAGGSEYHAPAGYQVNPPYHPVHGYEEQCSSIYIYYGALWEQETARGFQWFAVFLSALFLAFYGWHAYKASVGWEEVYVCSVELIKVILEIYFEFTSPAMLFLYGGNITPWLRYAEWLLTCPVILIHLSNITGLSEEYNKRTMALLVSDLGTICMGVTAALATGWVKWLFYCIGLVYGTQTFYNAGIIYVESYYIMPAGGCKKLVLAMTAVYYSSWLMFPGLFIFGPEGMHTLSVAGSTIGHTIADLLSKNIWGLLGHFLRIKIHEHIIMYGDIRRPVSSQFLGRKVDVLAFVTEEDKVAAAKSRITSEGEYIPLDQIDINVFCYENEV

SEQ ID NO: 172 - protein, 350 aa, Artificial sequence
  CnChR1 (Chrimson)
MAELISSATRSLFAAGGINPWPNPYHHEDMGCGGMTPTGECFSTEWWCDPSYGLSDAGYGYCFVEATGGYLVVGVEKKQAWLHSRGTPGEKIGAQVCQWIAFSIAIALLTFYGFSAWKATCGWEEVYVCCVEVLFVTLEIFKEFSSPATVYLSTGNHAYCLRYFEWLLSCPVILIKLSNLSGLKNDYSKRTMGLIVSCVGMIVFGMAAGLATDWLKWLLYIVSCIYGGYMYFQAAKCYVEANHSVPKGHCRMVVKLMAYAYFASWGSYPILWAVGPEGLLKLSPYANSIGHSICDIIAKEFWTFLAHHLRIKIHEHILIHGDIRKTTKMEIGGEEVEVEEFVEEEDEDTV

SEQ ID NO: 173 - protein, 380 aa, Artificial sequence
  CnChR1 (Chrimson)
MAELISSATRSLFAAGGINPWPNPYHHEDMGCGGMTPTGECFSTEWWCDPSYGLSDAGYGYCFVEATGGYLVVGVEKKQAWLHSRGTPGEKIGAQVCQWIAFSIAIALLTFYGFSAWKATCGWEEVYVCCVEVLFVTLEIFKEFSSPATVYLSTGNHAYCLRYFEWLLSCPVILIKLSNLSGLKNDYSKRTMGLIVSCVGMIVFGMAAGLATDWLKWLLYIVSCIYGGYMYFQAAKCYVEANHSVPKGHCRMVVKLMAYAYFASWGSYPILWAVGPEGLLKLSPYANSIGHSICDIIAKEFWTFLAHHLRIKIHEHILIHGDIRKTTKMEIGGEEVEVEEFVEEEDEDTVAAAKSRITSEGEYIPLDQIDINVFCYENEV

SEQ ID NO: 174 - protein, 345 aa, Artificial sequence
  CsChrimson
MSRLVAASWLLALLLCGITSTTTASSAPAASSTDGTAAAAVSHYAMNGFDELAKGAVVPEDHFVCGPADKCYCSAWLHSRGTPGEKIGAQVCQWIAFSIAIALLTFYGFSAWKATCGWEEVYVCCVEVLFVTLEIFKEFSSPATVYLSTGNHAYCLRYFEWLLSCPVILIKLSNLSGLKNDYSKRTMGLIVSCVGMIVFGMAAGLATDWLKWLLYIVSCIYGGYMYFQAAKCYVEANHSVPKGHCRMVVKLMAYAYFASWGSYPILWAVGPEGLLKLSPYANSIGHSICDIIAKEFWTFLAHHLRIKIHEHILIHGDIRKTTKMEIGGEEVEVEEFVEEEDEDTV

SEQ ID NO: 175 - protein, 375 aa, Artificial sequence
  CsChrimson
MSRLVAASWLLALLLCGITSTTTASSAPAASSTDGTAAAAVSHYAMNGFDELAKGAVVPEDHFVCGPADKCYCSAWLHSRGTPGEKIGAQVCQWIAFSIAIALLTFYGFSAWKATCGWEEVYVCCVEVLFVTLEIFKEFSSPATVYLSTGNHAYCLRYFEWLLSCPVILIKLSNLSGLKNDYSKRTMGLIVSCVGMIVFGMAAGLATDWLKWLLYIVSCIYGGYMYFQAAKCYVEANHSVPKGHCRMVVKLMAYAYFASWGSYPILWAVGPEGLLKLSPYANSIGHSICDIIAKEFWTFLAHHLRIKIHEHILIHGDIRKTTKMEIGGEEVEVEEFVEEEDEDTVAAAKSRITSEGEYIPLDQIDINVFCYENEV

SEQ ID NO: 176 - protein, 325 aa, Artificial sequence
  ShChR1 (Chronos)
METAATMTHAFISAVPSAEATIRGLLSAAAVVTPAADAHGETSNATTAGADHGCFPHINHGTELQHKIAVGLQWFTVIVAIVQLIFYGWHSFKATTGWEEVYVCVIELVKCFIELFHEVDSPATVYQTNGGAVIWLRYSMWLLTCPVILIHLSNLTGLHEEYSKRTMTILVTDIGNIVWGITAAFTKGPLKILFFMIGLFYGVTCFFQIAKVYIESYHTLPKGVCRKICKIMAYVFFCSWLMFPVMFIAGHEGLGLITPYTSGIGHLILDLISKNTWGFLGHHLRVKIHEHILIHGDIRKTTTINVAGENMEIETFVDEEEEGGV

SEQ ID NO: 177 - protein, 355 aa, Artificial sequence
  ShChR1 (Chronos) with ER export and trafficking signal sequences
METAATMTHAFISAVPSAEATIRGLLSAAAVVTPAADAHGETSNATTAGADHGCFPHINHGTELQHKIAVGLQWFTVIVAIVQLIFYGWHSFKATTGWEEVYVCVIELVKCFIELFHEVDSPATVYQTNGGAVIWLRYSMWLLTCPVILIHLSNLTGLHEEYSKRTMTILVTDIGNIVWGITAAFTKGPLKILFFMIGLFYGVTCFFQIAKVYIESYHTLPKGVCRKICKIMAYVFFCSWLMFPVMFIAGHEGLGLITPYTSGIGHLILDLISKNTWGFLGHHLRVKIHEHILIHGDIRKTTTINVAGENMEIETFVDEEEEGGVAAAKSRITSEGEYIPLDQIDINVFCYENEV

SEQ ID NO: 178 - protein, 258 aa, Artificial sequence
  Archaerhodopsin-3
MDPIALQAGYDLLGDGRPETLWLGIGTLLMLIGTFYFLVRGWGVTDKDAREYYAVTILVPGIASAAYLSMFFGIGLTEVTVGGEMLDIYYARYADWLFTTPLLLLDLALLAKVDRVTIGTLVGVDALMIVTGLIGALSHTAIARYSWWLFSTICMIVVLYFLATSLRSAAKERGPEVASTFNTLTALVLVLWTAYPILWIIGTEGAGVVGLGIETLLFMVLDVTAKVGFGFILLRSRAILGDTEAPEPSAGADVSAAD

SEQ ID NO: 179 - protein, 293 aa, Artificial sequence
  eArch3.0
MDPIALQAGYDLLGDGRPETLWLGIGTLLMLIGTFYFLVRGWGVTDKDAREYYAVTILVPGIASAAYLSMFFGIGLTEVTVGGEMLDIYYARYADWLFTTPLLLLDLALLAKVDRVTIGTLVGVDALMIVTGLIGALSHTAIARYSWWLFSTICMIVVLYFLATSLRSAAKERGPEVASTFNTLTALVLVLWTAYPILWIIGTEGAGVVGLGIETLLFMVLDVTAKVGFGFILLRSRAILGDTEAPEPSAGADVSAADRPVVAAAAKSRITSEGEYIPLDQIDINVFCYENEV

SEQ ID NO: 180 - protein, 248 aa, Artificial sequence
  ArchT
MDPIALQAGYDLLGDGRPETLWLGIGTLLMLIGTFYFIVKGWGVTDKEAREYYSITILVPGIASAAYLSMFFGIGLTEVTVAGEVLDIYYARYADWLFTTPLLLLDLALLAKVDRVSIGTLVGVDALMIVTGLIGALSHTPLARYSWWLFSTICMIVVLYFLATSLRAAAKERGPEVASTFNTLTALVLVLWTAYPILWIIGTEGAGVVGLGIETLLFMVLDVTAKVGFGFILLRSRAILGDTEAPEP

SEQ ID NO: 181 - protein, 278 aa, Artificial sequence
  ArchT with ER export and trafficking signal sequences
MDPIALQAGYDLLGDGRPETLWLGIGTLLMLIGTFYFIVKGWGVTDKEAREYYSITILVPGIASAAYLSMFFGIGLTEVTVAGEVLDIYYARYADWLFTTPLLLLDLALLAKVDRVSIGTLVGVDALMIVTGLIGALSHTPLARYSWWLFSTICMIVVLYFLATSLRAAAKERGPEVASTFNTLTALVLVLWTAYPILWIIGTEGAGVVGLGIETLLFMVLDVTAKVGFGFILLRSRAILGDTEAPEPAAAKSRITSEGEYIPLDQIDINVFCYENEV

SEQ ID NO: 182 - protein, 242 aa, Artificial sequence
  GtR3
MLVGEGAKLDVHGCKTVDMASSFGKALLEFVFIVFACITLLLGINAAKSKAASRVLFPATFVTGIASIAYFSMASGGGWVIAPDCRQLFVARYLDWLITTPLLLIDLGLVAGVSRWDIMALCLSDVLMIATGAFGSLTVGNVKWVWWFFGMCWFLHIIFALGKSWAEAAKAKGGDSASVYSKIAGITVITWFCYPVVWVFAEGFGNFSVTFEVLIYGVLDVISKAVFGLILMSGAATGYESI

SEQ ID NO: 183 - protein, 272 aa, Artificial sequence
  GtR3 with ER export and trafficking signal sequences
MLVGEGAKLDVHGCKTVDMASSFGKALLEFVFIVFACITLLLGINAAKSKAASRVLFPATFVTGIASIAYFSMASGGGWVIAPDCRQLFVARYLDWLITTPLLLIDLGLVAGVSRWDIMALCLSDVLMIATGAFGSLTVGNVKWVWWFFGMCWFLHIIFALGKSWAEAAKAKGGDSASVYSKIAGITVITWFCYPVVWVFAEGFGNFSVTFEVLIYGVLDVISKAVFGLILMSGAATGYESIAAAKSRITSEGEYIPLDQIDINVFCYENEV

SEQ ID NO: 184 - protein, 262 aa, Oxyrrhis marina
MAPLAQDWTYAEWSAVYNALSFGIAGMGSATIFFWLQLPNVTKNYRTALTITGIVTLIATYHYFRIFNSWVAAFNVGLGVNGAYEVTVSGTPFNDAYRYVDWLLTVPLLLVELILVMKLPAKETVCLAWTLGIASAVMVALGYPGEIQDDLSVRWFWWACAMVPFVYVVGTLVVGLGAATAKQPEGVVDLVSAARYLTVVSWLTYPFVYIVKNIGLAGSTATMYEQIGYSAADVTAKAVFGVLIWAIANAKSRLEEEGKLRA

SEQ ID NO: 185 - protein, 292 aa, Artificial sequence
  rhodopsin type II proton pump with ER export and trafficking signal sequences
MAPLAQDWTYAEWSAVYNALSFGIAGMGSATIFFWLQLPNVTKNYRTALTITGIVTLIATYHYFRIFNSWVAAFNVGLGVNGAYEVTVSGTPFNDAYRYVDWLLTVPLLLVELILVMKLPAKETVCLAWTLGIASAVMVALGYPGEIQDDLSVRWFWWACAMVPFVYVVGTLVVGLGAATAKQPEGVVDLVSAARYLTVVSWLTYPFVYIVKNIGLAGSTATMYEQIGYSAADVTAKAVFGVLIWAIANAKSRLEEEGKLRAAAAKSRITSEGEYIPLDQIDINVFCYENEV

SEQ ID NO: 186 - protein, 313 aa, Leptosphaeria maculans
MIVDQFEEVLMKTSQLFPLPTATQSAQPTHVAPVPTVLPDTPIYETVGDSGSKTLWVVFVLMLIASAAFTALSWKIPVNRRLYHVITTIITLTAALSYFAMATGHGVALNKIVIRTQHDHVPDTYETVYRQVYYARYIDWAITTPLLLLDLGLLAGMSGAHIFMAIVADLIMVLTGLFAAFGSEGTPQKWGWYTIACIAYIFVVWHLVLNGGANARVKGEKLRSFFVAIGAYTLILWTAYPIVWGLADGARKIGVDGEIIAYAVLDVLAKGVFGAWLLVTHANLRESDVELNGFWANGLNREGAIRIGEDDGA

SEQ ID NO: 187 - protein, 351 aa, Artificial sequence
  Mac 3.0
MIVDQFEEVLMKTSQLFPLPTATQSAQPTHVAPVPTVLPDTPIYETVGDSGSKTLWVVFVLMLIASAAFTALSWKIPVNRRLYHVITTIITLTAALSYFAMATGHGVALNKIVIRTQHDHVPDTYETVYRQVYYARYIDWAITTPLLLLDLGLLAGMSGAHIFMAIVADLIMVLTGLFAAFGSEGTPQKWGWYTIACIAYIFVVWHLVLNGGANARVKGEKLRSFFVAIGAYTLILWTAYPIVWGLADGARKIGVDGEIIAYAVLDVLAKGVFGAWLLVTHANLRESDVELNGFWANGLNREGAIRIGEDDGARPVVAVSKAAAKSRITSEGEYIPLDQIDINVFCYENEV

SEQ ID NO: 188 - protein, 291 aa, Artificial sequence
  NpHR
MTETLPPVTESAVALQAEVTQRELFEFVLNDPLLASSLYINIALAGLSILLFVFMTRGLDDPRAKLIAVSTILVPVVSIASYTGLASGLTISVLEMPAGHFAEGSSVMLGGEEVDGVVTMWGRYLTWALSTPMILLALGLLAGSNATKLFTAITFDIAMCVTGLAAALTTSSHLMRWFWYAISCACFLVVLYILLVEWAQDAKAAGTADMFNTLKLLTVVMWLGYPIVWALGVEGIAVLPVGVTSWGYSFLDIVAKYIFAFLLLNYLTSNESVVSGSILDVPSASGTPADD

SEQ ID NO: 189 - protein, 320 aa, Artificial sequence
  NpHR3.0
MTETLPPVTESAVALQAEVTQRELFEFVLNDPLLASSLYINIALAGLSILLFVFMTRGLDDPRAKLIAVSTILVPVVSIASYTGLASGLTISVLEMPAGHFAEGSSVMLGGEEVDGVVTMWGRYLTWALSTPMILLALGLLAGSNATKLFTAITFDIAMCVTGLAAALTTSSHLMRWFWYAISCACFLVVLYILLVEWAQDAKAAGTADMFNTLKLLTVVMWLGYPIVWALGVEGIAVLPVGVTSWGYSFLDIVAKYIFAFLLLNYLTSNESVVSGSILDVPSASGTPADDAAAKSRITSEGEYIPLDQIDINFCYENEV

SEQ ID NO: 190 - protein, 303 aa, Artificial sequence
  NpHR3.1
MVTQRELFEFVLNDPLLASSLYINIALAGLSILLFVFMTRGLDDPRAKLIAVSTILVPVVSIASYTGLASGLTISVLEMPAGHFAEGSSVMLGGEEVDGVVTMWGRYLTWALSTPMILLALGLLAGSNATKLFTAITFDIAMCVTGLAAALTTSSHLMRWFWYAISCACFLVVLYILLVEWAQDAKAAGTADMFNTLKLLTVVMWLGYPIVWALGVEGIAVLPVGVTSWGYSFLDIVAKYIFAFLLLNYLTSNESVVSGSILDVPSASGTPADDAAAKSRITSEGEYIPLDQIDINFCYENEV

SEQ ID NO: 191 - protein, 365 aa, Dunaliella salina
MRRRESQLAYLCLFVLIAGWAPRLTESAPDLAERRPPSERNTPYANIKKVPNITEPNANVQLDGWALYQDFYYLAGSDKEWVVGPSDQCYCRAWSKSHGTDREGEAAVVWAYIVFAICIVQLVYFMFAAWKATVGWEEVYVNIIELVHIALVIWVEFDKPAMLYLNDGQMVPWLRYSAWLLSCPVILIHLSNLTGLKGDYSKRTMGLLVSDIGTIVFGTSAALAPPNHVKVILFTIGLLYGLFTFFTAAKVYIEAYHTVPKGQCRNLVRAMAWTYFVSWAMFPILFILGREGFGHITYFGSSIGHFILEIFSKNLWSLLGHGLRYRIRQHIIIHGNLTKKNKINIAGDNVEVEEYVDSNDKDSDV

SEQ ID NO: 192 - protein, 395 aa, Artificial sequence
  Dunaliella salina channelrhodopsin with ER export and trafficking signal sequences
MRRRESQLAYLCLFVLIAGWAPRLTESAPDLAERRPPSERNTPYANIKKVPNITEPNANVQLDGWALYQDFYYLAGSDKEWVVGPSDQCYCRAWSKSHGTDREGEAAVVWAYIVFAICIVQLVYFMFAAWKATVGWEEVYVNIIELVHIALVIWVEFDKPAMLYLNDGQMVPWLRYSAWLLSCPVILIHLSNLTGLKGDYSKRTMGLLVSDIGTIVFGTSAALAPPNHVKVILFTIGLLYGLFTFFTAAKVYIEAYHTVPKGQCRNLVRAMAWTYFVSWAMFPILFILGREGFGHITYFGSSIGHFILEIFSKNLWSLLGHGLRYRIRQHIIIHGNLTKKNKINIAGDNVEVEEYVDSNDKDSDVAAAKSRITSEGEYIPLDQIDINVFCYENEV

SEQ ID NO: 193 - protein, 348 aa, Artificial sequence
  iC1C2
MSRRPWLLALALAVALAAGSAGASTGSDATVPVATQDGPDYVFHRAHERMLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTCPVILIRLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSKYGSNVGHTIIDLMSKQCWGLLGHYLRVLIHEHILIHGDIRKTTKLNIGGTEIEVETLVEDEAEAGAV

SEQ ID NO: 194 - protein, 378 aa, Artificial sequence
  iC1C2 with ER export and trafficking signal sequences
MSRRPWLLALALAVALAAGSAGASTGSDATVPVATQDGPDYVFHRAHERMLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTCPVILIRLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSKYGSNVGHTIIDLMSKQCWGLLGHYLRVLIHEHILIHGDIRKTTKLNIGGTEIEVETLVEDEAEAGAVAAAKSRITSEGEYIPLDQIDINVFCYENEV

SEQ ID NO: 195 - protein, 348 aa, Artificial sequence
  SwiChR
  MISC_FEATURE (167)..(167): alanine, serine, or threonine
MSRRPWLLALALAVALAAGSAGASTGSDATVPVATQDGPDYVFHRAHERMLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTXPVILIRLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSKYGSNVGHTIIDLMSKQCWGLLGHYLRVLIHEHILIHGDIRKTTKLNIGGTEIEVETLVEDEAEAGAV

SEQ ID NO: 196 - protein, 378 aa, Artificial sequence
  SwiChR with ER export and trafficking signal sequences
  MISC_FEATURE (167)..(167): alanine, serine, or threonine
MSRRPWLLALALAVALAAGSAGASTGSDATVPVATQDGPDYVFHRAHERMLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTXPVILIRLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSKYGSNVGHTIIDLMSKQCWGLLGHYLRVLIHEHILIHGDIRKTTKLNIGGTEIEVETLVEDEAEAGAVAAAKSRITSEGEYIPLDQIDINVFCYENEV

SEQ ID NO: 197 - protein, 309 aa, Artificial sequence
  ibC1C2
MDYGGALSAVGLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTCPVILIRLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSKYGSNVGHTIIDLMSKQCWGLLGHYLRVLIHEHILIHGDIRKTTKLNIGGTEIEVETLVEDEAEAGAV

SEQ ID NO: 198 - protein, 339 aa, Artificial sequence
  ibC1C2 with ER export and trafficking signal sequences
MDYGGALSAVGLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTCPVILIRLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSKYGSNVGHTIIDLMSKQCWGLLGHYLRVLIHEHILIHGDIRKTTKLNIGGTEIEVETLVEDEAEAGAVAAAKSRITSEGEYIPLDQIDINVFCYENEV

SEQ ID NO: 199 - protein, 310 aa, Artificial sequence
  iChR2
MDYGGALSAVGRELLFVTNPVVVNGSVLVPEDQCYCAGWIESRGTNGAQTASNVLQWLSAGFSILLLMFYAYQTWKSTCGWEEIYVCAISMVKVILEFFFSFKNPSMLYLATGHRVKWLRYASWLLTCPVILIRLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSKYGSNVGHTIIDLMSKQCWGLLGHYLRVLIHEHILIHGDIRKTTKLNIGGTEIEVETLVEDEAEAGAVP

SEQ ID NO: 200 - protein, 340 aa, Artificial sequence
  iChR2 with ER export and trafficking signal sequences
MDYGGALSAVGRELLFVTNPVVVNGSVLVPEDQCYCAGWIESRGTNGAQTASNVLQWLSAGFSILLLMFYAYQTWKSTCGWEEIYVCAISMVKVILEFFFSFKNPSMLYLATGHRVKWLRYASWLLTCPVILIRLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSKYGSNVGHTIIDLMSKQCWGLLGHYLRVLIHEHILIHGDIRKTTKLNIGGTEIEVETLVEDEAEAGAVPAAAKSRITSEGEYIPLDQIDINVFCYENEV

SEQ ID NO: 201 - protein, 344 aa, Artificial sequence
  iC1V1
MSRRPWLLALALAVALAAGSAGASTGSDATVPVATQDGPDYVFHRAHERMLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTCPVLLIRLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISKYGSNIGHSILDLIAKQMWGVLGNYLRVKIHEHILLYGDIRKKQKITIAGQEMEVETLVAEEED

SEQ ID NO: 202 - protein, 374 aa, Artificial sequence
  iC1V1 with ER export and trafficking signal sequences
MSRRPWLLALALAVALAAGSAGASTGSDATVPVATQDGPDYVFHRAHERMLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTCPVLLIRLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISKYGSNIGHSILDLIAKQMWGVLGNYLRVKIHEHILLYGDIRKKQKITIAGQEMEVETLVAEEEDAAAKSRITSEGEYIPLDQIDINVFCYENEV

SEQ ID NO: 203 - protein, 305 aa, Artificial sequence
  ibC1V1
MDYGGALSAVGLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTCPVLLIRLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISKYGSNIGHSILDLIAKQMWGVLGNYLRVKIHEHILLYGDIRKKQKITIAGQEMEVETLVAEEED

SEQ ID NO: 204 - protein, 335 aa, Artificial sequence
  ibC1V1 with ER export and trafficking signal sequences
MDYGGALSAVGLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTCPVLLIRLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISKYGSNIGHSILDLIAKQMWGVLGNYLRVKIHEHILLYGDIRKKQKITIAGQEMEVETLVAEEEDAAAKSRITSEGEYIPLDQIDINVFCYENEV

SEQ ID NO: 205 - protein, 350 aa, Artificial sequence
  iReaChR
MVSRRPWLLALALAVALAAGSAGASTGSDATVPVATQDGPDYVFHRAHERMLFQTSYTLENNGSVICIPNNGQCFCLAWLKSNGTNAEKLAANILQWVSFALSVACLGWYAYQAWRATCGWEEVYVALISMMKSIIEAFHSFDSPATLWLSSGNGVKWMRYGSWLLTCPVILIRLSNLTGLKDDYSKRTMGLLVSDVGCIVWGATSAMCTGWTKILFFLISLSYGMYTYFHAAKVYIEAFHTVPKGLCRQLVRAMAWLFFVSWGMFPVLFLLGPEGFGHISKYGSNIGHSILDLIAKQMWGVLGNYLRVKIHEHILLYGDIRKKQKITIAGQEMEVETLVAEEEDKYESS

SEQ ID NO: 206 - protein, 380 aa, Artificial sequence
  iReaChR with ER export and trafficking signal sequences
MVSRRPWLLALALAVALAAGSAGASTGSDATVPVATQDGPDYVFHRAHERMLFQTSYTLENNGSVICIPNNGQCFCLAWLKSNGTNAEKLAANILQWVSFALSVACLGWYAYQAWRATCGWEEVYVALISMMKSIIEAFHSFDSPATLWLSSGNGVKWMRYGSWLLTCPVILIRLSNLTGLKDDYSKRTMGLLVSDVGCIVWGATSAMCTGWTKILFFLISLSYGMYTYFHAAKVYIEAFHTVPKGLCRQLVRAMAWLFFVSWGMFPVLFLLGPEGFGHISKYGSNIGHSILDLIAKQMWGVLGNYLRVKIHEHILLYGDIRKKQKITIAGQEMEVETLVAEEEDKYESSAAAKSRITSEGEYIPLDQIDINVFCYENEV

SEQ ID NO: 207 - protein, 310 aa, Artificial sequence
  ibReaChR
MDYGGALSAVGLFQTSYTLENNGSVICIPNNGQCFCLAWLKSNGTNAEKLAANILQWVSFALSVACLGWYAYQAWRATCGWEEVYVALISMMKSIIEAFHSFDSPATLWLSSGNGVKWMRYGSWLLTCPVILIRLSNLTGLKDDYSKRTMGLLVSDVGCIVWGATSAMCTGWTKILFFLISLSYGMYTYFHAAKVYIEAFHTVPKGLCRQLVRAMAWLFFVSWGMFPVLFLLGPEGFGHISKYGSNIGHSILDLIAKQMWGVLGNYLRVKIHEHILLYGDIRKKQKITIAGQEMEVETLVAEEEDKYESS

SEQ ID NO: 208 - protein, 340 aa, Artificial sequence
  ibReaChR with ER export and trafficking signal sequences
MDYGGALSAVGLFQTSYTLENNGSVICIPNNGQCFCLAWLKSNGTNAEKLAANILQWVSFALSVACLGWYAYQAWRATCGWEEVYVALISMMKSIIEAFHSFDSPATLWLSSGNGVKWMRYGSWLLTCPVILIRLSNLTGLKDDYSKRTMGLLVSDVGCIVWGATSAMCTGWTKILFFLISLSYGMYTYFHAAKVYIEAFHTVPKGLCRQLVRAMAWLFFVSWGMFPVLFLLGPEGFGHISKYGSNIGHSILDLIAKQMWGVLGNYLRVKIHEHILLYGDIRKKQKITIAGQEMEVETLVAEEEDKYESSAAAKSRITSEGEYIPLDQIDINVFCYENEV

SEQ ID NO: 209 - protein, 315 aa, Artificial sequence
  estrogen receptor protein
PSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATA

SEQ ID NO: 210 - protein, 116 aa, Artificial sequence
  truncated Neurexin with TM domain)
MHLRIHARRSPPRRPAWTLGIWFLFWGCIVSSVWSQLSSNVASSSSTSSSPGSHYPYDVPDYANPTEPGIRRVPGASEVIRESSSTTGMVVGIVAAAALCILILLYAMYKYRNRDE

SEQ ID NO: 211 - protein, 197 aa, Artificial sequence
  component of Exemplary FLARE component 1
GSGSTSGSGSGGSGGSGGSSGGMNGAIGGDLLLNFPDMSVLERQRAHLKYLNPTFDSPLAGFFADSSMITGGEMDSYLSTAGLNLPMMYGETTVEGDSRLSISPETTLGTGNFKAAKFDTETKDCNEAAKKMTMNRDDLVEEGEEEKSKITEQNNGSTKSIKKMKHKAKKEENNFSNDSSKVTKELEKTDYIHSGSG

SEQ ID NO: 212 - protein, 27 aa, Artificial sequence
  Nav1.6
TVRVPIAVGESDFENLNTEDVSSESDP

SEQ ID NO: 213 - protein, 5 aa, Artificial sequence
  linker
GGSGS

SEQ ID NO: 214 - protein, 46 aa, Artificial sequence; identical to SEQ ID NO: 26
  calmodulin binding peptide

SEQ ID NO: 215 - protein, 17 aa, Artificial sequence
  component of Exemplary FLARE component 1
ELAEKLAGLDINGGASG

SEQ ID NO: 216 - protein, 142 aa, Artificial sequence; identical to SEQ ID NO: 38
  eLOV

SEQ ID NO: 217 - protein, 12 aa, Artificial sequence
  GGGS linker and enterokinase cleavage site
GGGSDYKDDDDK

SEQ ID NO: 218 - protein, 335 aa, Artificial sequence; identical to SEQ ID NO: 142
  tTA-VP16 transcription factor

SEQ ID NO: 219 - protein, 399 aa, Artificial sequence
  Exemplary FLARE component 2protease construct
MDQLTEEQIAEFKEAFSLLDKDGDGTITTKELGTGMRSLGQNPTEAELQDMINEVDADGDGTIDFPEFLTMMARKMKYTDSEEEIREAFRVFDKDGNGYISAAELRHVMTNLGEKLTDEEVDEMIREADIDGDGQVNYEEFVQMMTAKGKPIPNPLLGLDSTGGSGSGSGGSYGSHVDYAGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMV

SEQ ID NO: 220 - protein, 148 aa, Artificial sequence; identical to SEQ ID NO: 29
  CaM-F19L,V35G

SEQ ID NO: 221 - protein, 14 aa, Artificial sequence
  V5 epitope tag
GKPIPNPLLGLDST

SEQ ID NO: 222 - protein, 18 aa, Artificial sequence
  linker
GGSGSGSGGSYGSHVDYA

SEQ ID NO: 223 - protein, 219 aa, Artificial sequence; identical to SEQ ID NO: 91
  TEV-220-242 truncated

SEQ ID NO: 224 - DNA, 1054 bp, Artificial sequence
  Exemplary FLARE component 3reporter construct
tctagacgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgcaaagggcgaattcgatccaccggtcgccaccatggctcgggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggattcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 225 - protein, 904 aa, Artificial sequence
  Exemplary FLARE component 1membrane construct
MHLRIHARRSPPRRPAWTLGIWFLFWGCIVSSVWSQLSSNVASSSSTSSSPGSHYPYDVPDYANPTEPGIRRVPGASEVIRESSSTTGMVVGIVAAAALCILILLYAMYKYRNRDEGSGSTSGSGSGGSGGSGGSSGGMNGAIGGDLLLNFPDMSVLERQRAHLKYLNPTFDSPLAGFFADSSMITGGEMDSYLSTAGLNLPMMYGETTVEGDSRLSISPETTLGTGNFKAAKFDTETKDCNEAAKKMTMNRDDLVEEGEEEKSKITEQNNGSTKSIKKMKHKAKKEENNFSNDSSKVTKELEKTDYIHSGSGTVRVPIAVGESDFENLNTEDVSSESDPGGSGSFNARRKLAGAILFTMLATRNFSGSFNARRKLAGAILFTMLATRNFSELAEKLAGLDINGGASGSRATTLERIEKSFVITDPRLPDNPIIFVSDSFLQLTEYSREEILGRNCRFLQGPETDRATVRKIRDAIDNQTEVTVQLINYTKSGKKFWNLFHLQPMRDQKGDVQYFIGVQLDGTERVRDAAEREAVMLVKKTAEEIDEAAKENLYFQMGGGSDYKDDDDKMSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

(19) United States
(12) Patent Application Publication
(10) Pub. No.: US 2018/0201657 A1
(43) Pub. Date: Jul. 19, 2018

(54) LIGHT-ACTIVATED, CALCIUM-GATED POLYPEPTIDE AND METHODS OF USE THEREOF

(71) Applicant: The Board of Trustees of the Leland Stanford Junior University, Stanford, CA (US)

(72) Inventors: Alice Y. Ting, Stanford, CA (US); Wenjing Wang, Stanford, CA (US)

(21) Appl. No.: 15/855,543

(22) Filed: Dec. 27, 2017

Related U.S. Application Data
(60) Provisional application No. 62/440,857, filed on Dec. 30, 2016.
(60) Provisional application No. 62/523,549, filed on Jun. 22, 2017.

(51) Int. Cl. C07K 14/47; G01N 33/50; C07K 14/73; C12N 9/50

(52) U.S. Cl. CPC C07K 14/47; G01N 33/5005; C07K 14/70514; C07K 2319/03; C12N 9/506; C12Y 304/22044; C07K 14/4728

(57) ABSTRACT

The present disclosure provides a light-activated, calcium-gated polypeptide; and a system comprising: a) the light-activated, calcium-gated polypeptide; and b) a fusion protein comprising a calcium responsive polypeptide and a protease that cleaves a proteolytically cleavable linker present in the light-activated, calcium-gated polypeptide. The present disclosure provides nucleic acids encoding the light-activated, calcium-gated polypeptide or the system, and cells comprising the nucleic acids. The present disclosure provides methods of detecting a change in intracellular calcium ion concentration. The present disclosure provides methods of controlling or modulating an activity of a cell.

### CROSS-REFERENCE

This application claims the benefit of U.S. Provisional Patent Application No. 62/440,857, filed Dec. 30, 2016, and U.S. Provisional Patent Application No. 62/523,549, filed Jun. 22, 2017, which applications are incorporated herein by reference in their entirety.

### INTRODUCTION

Calcium indicators that signal a change in intracellular calcium concentration are useful in a variety of applications. For example, neuronal activity is tightly coupled to rises in cytosolic calcium, both in distal dendrites and in the cell body, or soma, of neurons. Consequently, a very important class of tools for studying calcium signaling is real-time fluorescence calcium indicators, including the GCaMP series and small-molecule dyes such as Fura-2 and Fluo-4. However, these tools have two important limitations. First, the real-time imaging required for the use of calcium indicators is both technically demanding and restricted to small fields of view, should one desire single-cell resolution. Second, these indicators allow one to only passively observe calcium patterns, but not to respond to them—for example, to selectively manipulate or further characterize subsets of neurons based on their history of activity.

There is a need in the art for compositions and methods for detecting, and responding to, changes in intracellular calcium levels.

### SUMMARY

The present disclosure provides a light-activated, calcium-gated polypeptide; and a system comprising: a) the light-activated, calcium-gated polypeptide; and b) a fusion protein comprising a calcium responsive polypeptide and a protease that cleaves a proteolytically cleavable linker present in the light-activated, calcium-gated polypeptide. The present disclosure provides nucleic acids encoding the light-activated, calcium-gated polypeptide or the system, and cells comprising the nucleic acids. The present disclosure provides methods of detecting a change in intracellular calcium ion concentration. The present disclosure provides methods of controlling or modulating an activity of a cell.

The present disclosure provides a light-activated, calcium-gated transcriptional control polypeptide; and a system comprising: a) the light-activated, calcium-gated transcriptional control polypeptide; and b) a fusion protein comprising a calcium responsive polypeptide and a protease that cleaves a proteolytically cleavable linker present in the light-activated, calcium-gated transcriptional control polypeptide. The present disclosure provides nucleic acids encoding the light-activated, calcium-gated transcriptional control polypeptide or the system, and cells comprising the nucleic acids. The present disclosure provides methods of detecting a change in intracellular calcium ion concentration. The present disclosure provides methods of controlling or modulating an activity of a cell.

### BRIEF DESCRIPTION OF THE DRAWINGS

FIG. 1A-1C depicts the FLARE design and optimization of calcium response.

FIG. 2 provides a table of published TEV protease catalytic constants.

FIG. 3A-3C depicts light gating upon LOV domain insertion.

FIG. 4A-4D depicts the directed evolution of the LOV domain.

FIG. 5 depicts FACS plots showing library progression during directed evolution of the LOV domain.

FIG. 6 depicts the sequencing analysis of clones derived from the directed evolution of the LOV domain.

FIG. 7A-7C depicts FACS plots showing the analysis of specific LOV mutants.

FIG. 8A-8B depicts immunofluorescence images showing the directed evolution of the LOV domain.

FIG. 9 depicts an immunofluorescence image showing light gating by eLOV in vivo.

FIG. 10A-10G depicts the FLARE design and optimization of calcium response in neurons.

FIG. 11A-11B depicts the screening of alternative TEV cleavage sites.

FIG. 12A-12B depicts the analysis of FLARE sensitivity in neurons.

FIG. 13A-13B depicts the functional reactivation of neurons marked by FLARE.

FIG. 14 depicts immune fluorescence images showing the results of a second FLARE design.

FIG. 15A-15G provide amino acid sequences of LOV domains of light-activated polypeptides.

FIG. 16A-16B provide amino acid sequences of calmodulin.

FIG. 17A-17D provide amino acid sequences of calmodulin-binding polypeptides.

FIG. 18 provides an amino acid sequence of troponin C.

FIG. 19A-19B provide amino acid sequences of troponin I polypeptides.

FIG. 20A-20D provide amino acid sequences of tobacco etch virus (TEV) protease.

FIG. 21 depicts the amino acid sequence of a Streptomyces pyogenes Cas9 polypeptide.

FIG. 22 depicts the amino acid sequence of a Staphylococcus aureus Cas9 polypeptide.

FIG. 23 provides amino acid sequences of various depolarizing opsins.

FIG. 24 provides amino acid sequences of various hyperpolarizing opsins.

FIG. 25A-25B provide an amino acid sequence of a FLARE component 1 of the present disclosure (e.g., a FLARE component comprising calmodulin-binding polypeptide, a LOV domain polypeptide, a proteolytically cleavable crosslinker, and a transcription factor) (FIG. 25A); and amino acid sequences of the FLARE component 1 (FIG. 25B).

FIG. 26A-26B provide an amino acid sequence of a FLARE component 2 of the present disclosure (e.g., a FLARE component comprising a calmodulin polypeptide and a TEV protease) (FIG. 26A); and amino acid sequences of the FLARE component 2 (FIG. 26B).

FIG. 27 provides a nucleotide sequence of a FLARE component 3 of the present disclosure (e.g., a FLARE component comprising a promoter operably linked to a nucleotide sequence encoding a fluorescent protein.

FIG. 28A-28E depict activity of FLARE in vivo.

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

“Operably linked” refers to a juxtaposition wherein the components so described are in a relationship permitting them to function in their intended manner. For instance, a promoter is operably linked to a coding region of a nucleic acid if the promoter affects transcription or expression of the coding region of a nucleic acid.

A “vector” or “expression vector” is a replicon, such as plasmid, phage, virus, or cosmid, to which another DNA segment, i.e. an “insert”, may be attached so as to bring about the replication of the attached segment in a cell.

“Heterologous,” as used herein, refers to a nucleotide or polypeptide sequence that is not found in the native (e.g., naturally-occurring) nucleic acid or protein, respectively.

As used herein, the term “affinity” refers to the equilibrium constant for the reversible binding of two agents (e.g., a protease and a polypeptide comprising a protease cleavage site) and is expressed as Km. Km is the concentration of peptide at which the catalytic rate of proteolytic cleavage is half of Vmax (maximal catalytic rate). Km is often used in the literature as an approximation of affinity when speaking about enzyme-substrate interactions.

The term “binding” refers to a direct association between two molecules, due to, for example, covalent, electrostatic, hydrophobic, and ionic and/or hydrogen-bond interactions, including interactions such as salt bridges and water bridges. “Specific binding” refers to binding with an affinity of at least about 10⁻⁷ M or greater, e.g., 5×10⁻⁷ M, 10⁻⁸ M, 5×10⁻⁸ M, and greater. “Non-specific binding” refers to binding with an affinity of less than about 10⁻⁷ M, e.g., binding with an affinity of 10⁻⁶ M, 10⁻⁵ M, 10⁻⁴ M, etc.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

An “isolated” polypeptide is one that has been identified and separated and/or recovered from a component of its natural environment. Contaminant components of its natural environment are materials that would interfere with diagnostic or therapeutic uses for the polypeptide, and may include enzymes, hormones, and other proteinaceous or nonproteinaceous solutes. In some embodiments, the polypeptide will be purified (1) to greater than 90%, greater than 95%, or greater than 98%, by weight of antibody as determined by the Lowry method, for example, more than 99% by weight, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or nonreducing conditions using Coomassie blue or silver stain. Isolated polypeptide includes the polypeptide in situ within recombinant cells since at least one component of the polypeptide's natural environment will not be present. In some instances, isolated polypeptide will be prepared by at least one purification step.

The term “genetic modification” refers to a permanent or transient genetic change induced in a cell following introduction into the cell of a heterologous nucleic acid (e.g., a nucleic acid exogenous to the cell). Genetic change (“modification”) can be accomplished by incorporation of the heterologous nucleic acid into the genome of the host cell, or by transient or stable maintenance of the heterologous nucleic acid as an extrachromosomal element. Where the cell is a eukaryotic cell, a permanent genetic change can be achieved by introduction of the nucleic acid into the genome of the cell. Suitable methods of genetic modification include viral infection, transfection, conjugation, protoplast fusion, electroporation, particle gun technology, calcium phosphate precipitation, direct microinjection, use of a CRISPR/Cas9 system, and the like.

A “host cell,” as used herein, denotes an in vivo or in vitro eukaryotic cell, or a cell from a multicellular organism (e.g., a cell line) cultured as a unicellular entity, which eukaryotic cells can be, or have been, used as recipients for a nucleic acid (e.g., an expression vector that comprises a nucleotide sequence encoding an eLOV polypeptide; or any other nucleic acid or expression vector described herein), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a genetically modified eukaryotic host cell is genetically modified by virtue of introduction into a suitable eukaryotic host cell of a heterologous nucleic acid, e.g., an exogenous nucleic acid that is foreign to the eukaryotic host cell, or a recombinant nucleic acid that is not normally found in the eukaryotic host cell, where such nucleic acids and expression vectors are described herein.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a transcription factor” includes a plurality of such transcription factors and reference to “the proteolytically cleavable linker” includes reference to one or more proteolytically cleavable linkers and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides a light-activated, calcium-gated polypeptide; and a system comprising: a) the light-activated, calcium-gated polypeptide; and b) a fusion protein comprising a calcium responsive polypeptide and a protease that cleaves a proteolytically cleavable linker present in the light-activated, calcium-gated polypeptide. The present disclosure provides nucleic acids encoding the light-activated, calcium-gated polypeptide or the system, and cells comprising the nucleic acids. The present disclosure provides methods of detecting a change in intracellular calcium ion concentration. The present disclosure provides methods of controlling or modulating an activity of a cell.

The present disclosure provides a light-activated, calcium-gated transcriptional control polypeptide; and a system comprising: a) the light-activated, calcium-gated transcriptional control polypeptide; and b) a fusion protein comprising a calcium responsive polypeptide and a protease that cleaves a proteolytically cleavable linker present in the light-activated, calcium-gated transcriptional control polypeptide. The present disclosure provides nucleic acids encoding the light-activated, calcium-gated transcriptional control polypeptide or the system, and cells comprising the nucleic acids. The present disclosure provides methods of detecting a change in intracellular calcium ion concentration. The present disclosure provides methods of controlling or modulating an activity of a cell.

A system of the present disclosure is a calcium- and light-gated system. Thus, a system of the present disclosure provides an “AND” gate that can be used to detect a change in intracellular calcium ion concentration, e.g., in response of a cell to any of a variety of stimuli. A system of the present disclosure provides a high signal-to-noise (S/N) ratio. A system of the present disclosure can be used to control an activity of a cell. For example, once a change in intracellular calcium ion concentration in the cell is detected, one or more activities of the cell can be modulated in response. An activity of the cell can be activated; or an activity of the cell can be inhibited. Thus, a system of the present disclosure provides a means not only to detect a change in intracellular calcium ion concentration, but to react to the change by modulating an activity of the cell. Furthermore, a change in intracellular calcium ion concentration can be detected in a temporal manner using a system of the present disclosure; i.e., the change can be detected over time. In addition to, or as an alternative to, modulating (e.g., controlling) an activity of a cell in response to an increase in intracellular calcium ion concentration, the cell can be further characterized; for example, a cell can be further characterized by any of a variety of techniques, including, e.g., proteomic analysis, transcriptomic analysis, imaging with a real-time calcium indicator, imaging with a synaptic marker, etc.

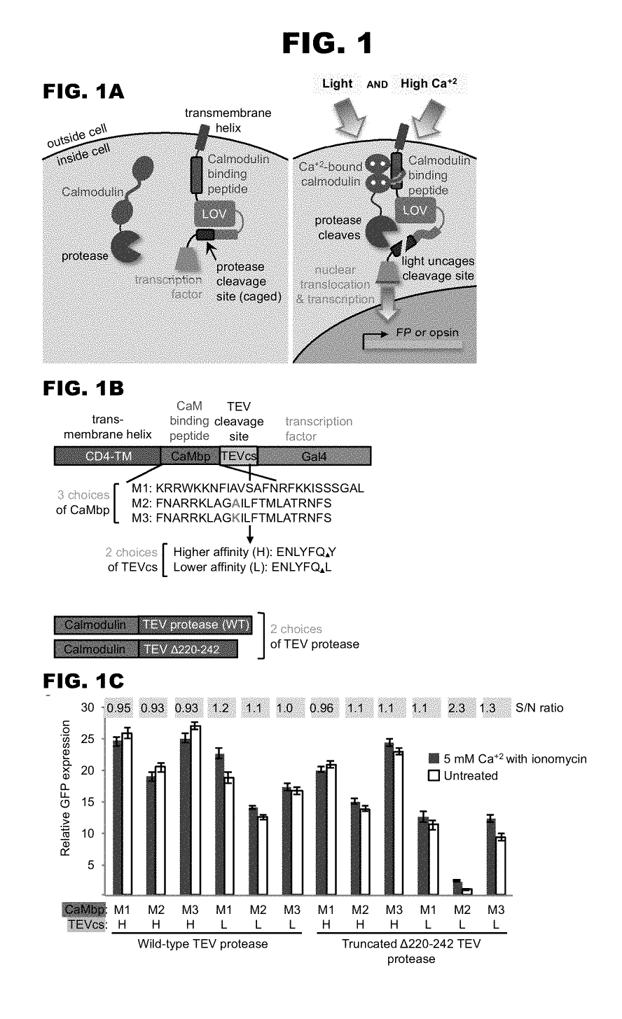

FIG. 1A presents a schematic representation of certain embodiments of a system of the present disclosure. Some embodiments of a system of the present disclosure, e.g., embodiments comprising a transcription factor, are also referred to as “FLARE” for Fast Light and Activity Reporter giving Expression. As depicted schematically in FIG. 1A, a FLARE system of the present disclosure comprises two polypeptides: 1) a first polypeptide comprises: a) a transmembrane domain; b) a polypeptide that binds a calcium-responsive polypeptide; c) a LOV light-activated polypeptide; d) a proteolytically cleavable linker that is caged by the LOV light-activated polypeptide, and that becomes uncaged upon exposure of the LOV light-activated polypeptide to light of an activating wavelength (e.g., blue light); and e) a transcription factor; and 2) a second comprises: a) a calcium-responsive polypeptide; and b) a protease that cleaves the proteolytically cleavable linker.

As depicted in the left panel of FIG. 1A, in the absence of light of an activating wavelength, and under conditions of low intracellular Ca²⁺ concentration, the first polypeptide and the second polypeptide do not substantially bind to one another, as the polypeptide that binds the calcium-responsive polypeptide present in first polypeptide and the calcium-responsive polypeptide present in second polypeptide do not substantially bind to one another under conditions of low intracellular calcium concentration. Furthermore, even if the first polypeptide and the second polypeptide were to bind to one another, since the LOV light-activated polypeptide cages the proteolytically cleavable linker in the absence of light of an activating wavelength, the proteolytically cleavable linker is not accessible to the protease. Thus, two signals are required for: 1) binding of the calcium-responsive polypeptide to the polypeptide that binds the calcium-responsive polypeptide; and 2) cleavage of the proteolytically cleavable linker by the protease.

As shown in the right panel of FIG. 1A, in the presence of a high intracellular Ca²⁺ concentration in the cell, and upon exposure of the cell to light of an activating wavelength, the first polypeptide and the second polypeptide bind to one another. The high intracellular Ca²⁺ concentration in the cell triggers binding of the calcium-responsive polypeptide present in the second polypeptide to the polypeptide that binds the calcium-responsive polypeptide present in the first polypeptide. Exposure of the cell to light of an activating wavelength induces a conformational change in the LOV light-activated polypeptide, exposing the proteolytically cleavable linker in the first polypeptide to the protease present in the second polypeptide. Cleavage of the proteolytically cleavable linker releases the transcription factor, which can enter the nucleus and modulate transcription of a coding region operably linked to a promoter that is recognized by the transcription factor. The coding region can encode any of a variety of gene products, including, e.g., an inhibitory RNA; a guide RNA; a reporter gene product; an opsin; a toxin; a DREADD; an RNA-guided endonuclease; a kinase; a biotin ligase; a transcription factor; a recombinase; an antibiotic resistance factor; a calcium sensor; a peroxidase; a fluorescent protein; a synaptic marker; etc.

A FLARE system of the present disclosure, when present in a cell, provides a signal-to-noise ratio of at least 3:1, at least 4:1, at least 5:1, at least 6:1, at least 7:1, at least 8:1, at least 9:1, at least 10:1, from 10:1 to 15:1, from 15:1 to 20:1, or more than 20:1 (e.g., from 20:1 to 50:1, from 50:1 to 100:1, from 100:1 to 150:1, or more than 150:1); i.e., the signal produced when the cell is exposed to light of an activating wavelength (e.g., blue light) and to a second signal that increases the intracellular calcium concentration in the cell above about 100 nM is at least 2-fold, at lease 3-fold, at least 4-fold, at least 5-fold, at least 6-fold, at least 7-fold, at least 8-fold, at least 9-fold, at least 10-fold, at least 15-fold, at least 20-fold, or more than 20-fold (e.g., more than 25-fold, more than 50-fold, more than 75-fold, more than 100-fold, more than 125-fold, or more than 150-fold), higher than the signal produced by the cell when the cell is: i) not exposed to either light of an activating wavelength or to a second signal that increases the intracellular calcium concentration in the cell above about 100 nM; ii) exposed to light of an activating wavelength, but not to a second signal that increases the intracellular calcium concentration in the cell above about 100 nM; or iii) exposed to a second signal that increases the intracellular calcium concentration in the cell above about 100 nM, but not to light of an activating wavelength.

A FLARE system of the present disclosure, its components, and methods of use are described in detail herein.

### Light- and Calcium-Gated Systems

System 1.

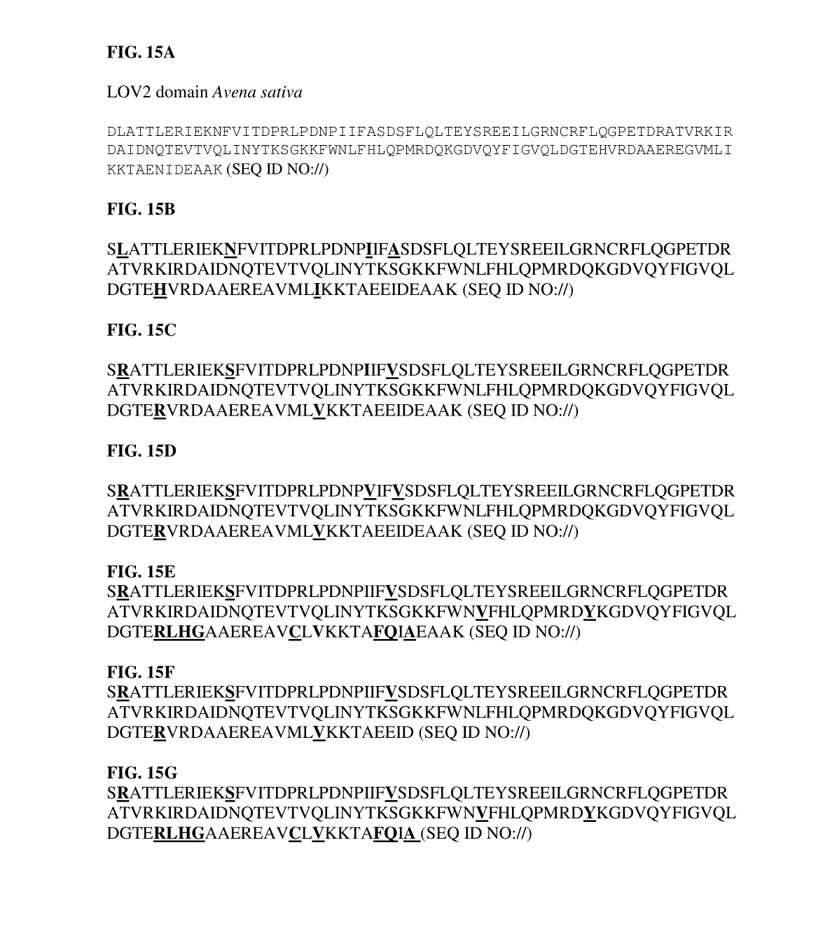

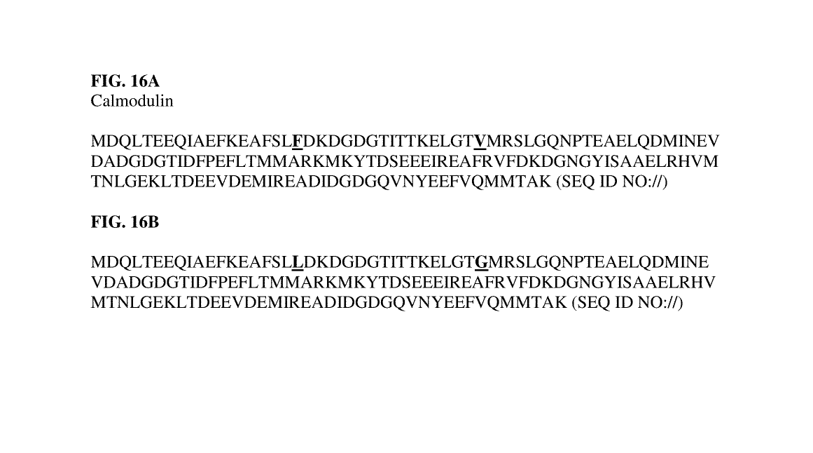

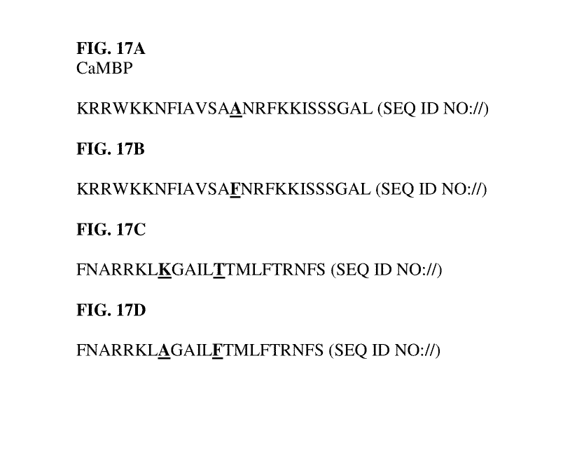

The present disclosure provides a nucleic acid system comprising: A) a first nucleic acid comprising, in order from 5′ to 3′: a) a nucleotide sequence encoding a light-activated, calcium-gated fusion polypeptide comprising, in order from amino terminus to carboxyl terminus: i) a transmembrane domain; ii) a calmodulin-binding polypeptide or a troponin I polypeptide; iii) a LOV-domain light-activated polypeptide comprising an amino acid sequence having at least 80% amino acid sequence identity to the amino acid sequence depicted in any one of FIG. 15A-15D; and iv) a proteolytically cleavable linker; and b) an insertion site for a nucleic acid comprising a nucleotide sequence encoding a polypeptide of interest; and B) a second nucleic acid comprising a nucleotide sequence encoding a second fusion polypeptide comprising: i) a calcium-binding polypeptide selected from a calmodulin polypeptide and troponin C polypeptide; and ii) a protease that cleaves the proteolytically cleavable linker. This nucleic acid system allows the user to insert into the insertion site a nucleic acid comprising a nucleotide sequence encoding a polypeptide of interest.

The present disclosure provides a nucleic acid system comprising: A) a first nucleic acid comprising, in order from 5′ to 3′: a) a nucleotide sequence encoding a light-activated, calcium-gated fusion polypeptide comprising, in order from amino terminus to carboxyl terminus: i) a transmembrane domain; ii) a calmodulin-binding polypeptide or a troponin I polypeptide; iii) a LOV-domain light-activated polypeptide comprising an amino acid sequence having at least 80% amino acid sequence identity to the amino acid sequence depicted in any one of FIG. 15E-15G; and iv) a proteolytically cleavable linker; and b) an insertion site for a nucleic acid comprising a nucleotide sequence encoding a polypeptide of interest; and B) a second nucleic acid comprising a nucleotide sequence encoding a second fusion polypeptide comprising: i) a calcium-binding polypeptide selected from a calmodulin polypeptide and troponin C polypeptide; and ii) a protease that cleaves the proteolytically cleavable linker. This nucleic acid system allows the user to insert into the insertion site a nucleic acid comprising a nucleotide sequence encoding a polypeptide of interest.

In some cases, the insertion site is a multiple cloning site. For example, the insertion site can comprise multiple (e.g., 2, 3, 4, or more) restriction endonuclease cleavage sites. The insertion site can comprise a restriction endonuclease cleavage site; in such a case, a nucleic acid comprising a nucleotide sequence encoding a polypeptide of interest can comprise, at its 5′ and 3′ ends, nucleotide sequences (e.g., complementary overhangs) that anneal with the ends created by restriction endonuclease cleavage.

The insertion site is within 10 nucleotides (nt), within 9 nt, within 8 nt, within 7 nt, within 6 nt, within 5 nt, within 4 nt, within 3 nt, within 2 nt, or 1 nt, of the 3′ end of the nucleotide sequence encoding the light-activated, calcium-gated fusion polypeptide. The insertion site is positioned relative to the nucleotide sequence encoding the light-activated, calcium-gated fusion polypeptide such that, after insertion of a nucleic acid comprising a nucleotide sequence encoding a gene product of interest, and after transcription and translation, a fusion polypeptide comprising: i) a transmembrane domain; ii) a calmodulin-binding polypeptide or a troponin I polypeptide; iii) a LOV-domain light-activated polypeptide comprising an amino acid sequence having at least 80% amino acid sequence identity to the amino acid sequence depicted any one of FIG. 15A-15D; iv) a proteolytically cleavable linker; and v) the gene product of interest, is produced.

The insertion site is within 10 nucleotides (nt), within 9 nt, within 8 nt, within 7 nt, within 6 nt, within 5 nt, within 4 nt, within 3 nt, within 2 nt, or 1 nt, of the 3′ end of the nucleotide sequence encoding the light-activated, calcium-gated fusion polypeptide. The insertion site is positioned relative to the nucleotide sequence encoding the light-activated, calcium-gated fusion polypeptide such that, after insertion of a nucleic acid comprising a nucleotide sequence encoding a gene product of interest, and after transcription and translation, a fusion polypeptide comprising: i) a transmembrane domain; ii) a calmodulin-binding polypeptide or a troponin I polypeptide; iii) a LOV-domain light-activated polypeptide comprising an amino acid sequence having at least 80% amino acid sequence identity to the amino acid sequence depicted any one of FIG. 15E-15G; iv) a proteolytically cleavable linker; and v) the gene product of interest, is produced.

System 2.

The present disclosure provides nucleic acid system comprising: a) a first nucleic acid comprising a nucleotide sequence encoding a light-activated, calcium-gated fusion polypeptide comprising, in order from amino terminus to carboxyl terminus: i) a transmembrane domain; ii) a calmodulin-binding polypeptide or a troponin I polypeptide; iii) a LOV light-activated polypeptide comprising an amino acid sequence having at least 80% amino acid sequence identity to the amino acid sequence depicted in any one of FIG. 15A-15D; iv) a proteolytically cleavable linker; and v) a gene product of interest; and b) a second nucleic acid comprising a nucleotide sequence encoding a second fusion polypeptide comprising: i) a calcium-binding polypeptide selected from a calmodulin polypeptide and troponin C polypeptide; and ii) a protease that cleaves the proteolytically cleavable linker. Thus, in some cases, the present disclosure provides a nucleic acid system in which the first nucleic acid comprises a nucleotide sequence encoding a light-activated, calcium-gated fusion polypeptide that comprises a gene product of interest.

The present disclosure provides nucleic acid system comprising: a) a first nucleic acid comprising a nucleotide sequence encoding a light-activated, calcium-gated fusion polypeptide comprising, in order from amino terminus to carboxyl terminus: i) a transmembrane domain; ii) a calmodulin-binding polypeptide or a troponin I polypeptide; iii) a LOV light-activated polypeptide comprising an amino acid sequence having at least 80% amino acid sequence identity to the amino acid sequence depicted in any one of FIG. 15E-15G; iv) a proteolytically cleavable linker; and v) a gene product of interest; and b) a second nucleic acid comprising a nucleotide sequence encoding a second fusion polypeptide comprising: i) a calcium-binding polypeptide selected from a calmodulin polypeptide and troponin C polypeptide; and ii) a protease that cleaves the proteolytically cleavable linker. Thus, in some cases, the present disclosure provides a nucleic acid system in which the first nucleic acid comprises a nucleotide sequence encoding a light-activated, calcium-gated fusion polypeptide that comprises a gene product of interest.

A transmembrane domain, a calmodulin polypeptide, a calmodulin-binding polypeptide, a troponin C polypeptide, a troponin I polypeptide, a LOV-domain light-activated polypeptide, a proteolytically cleavable linker, and a protease, that can be encoded by a nucleotide sequence included in one or more embodiments of System 1 or System 2 are described below.

### Polypeptides

The present disclosure provides a light-activated, calcium-gated polypeptide comprising, in order from amino terminus to carboxyl terminus: i) a transmembrane domain; ii) a calmodulin-binding polypeptide or a troponin I polypeptide; iii) a LOV-domain light-activated polypeptide comprising an amino acid sequence having at least 80% amino acid sequence identity to the amino acid sequence depicted in any one of FIG. 15A-15D; iv) a proteolytically cleavable linker; and v) a polypeptide of interest. The present disclosure provides a light-activated, calcium-gated polypeptide comprising, in order from amino terminus to carboxyl terminus: i) a transmembrane domain; ii) a calmodulin-binding polypeptide or a troponin I polypeptide; iii) a LOV-domain light-activated polypeptide comprising an amino acid sequence having at least 80% amino acid sequence identity to the amino acid sequence depicted in any one of FIG. 15E-15G; iv) a proteolytically cleavable linker; and v) a polypeptide of interest.

Suitable transmembrane domains, calmodulin-binding polypeptides, troponin I polypeptides, LOV-domain light-activated polypeptides, proteolytically cleavable linkers, and polypeptides of interest are described below.

In some cases, a light-activated, calcium-gated polypeptide of the present disclosure is isolated. In some cases, a light-activated, calcium-gated polypeptide of the present disclosure is present in a cell in vitro. In some cases, a light-activated, calcium-gated polypeptide of the present disclosure is present in a cell in vivo. Suitable cells are described below.

### System Components

The present disclosure provides components of a system of the present disclosure, e.g., components of System 1 and System 2.

For example, the present disclosure provides a nucleic acid comprising: a) a nucleotide sequence encoding a light-activated, calcium-gated fusion polypeptide comprising, in order from amino terminus to carboxyl terminus: i) a transmembrane domain; ii) a calmodulin-binding polypeptide or a troponin I polypeptide; iii) a LOV-domain light-activated polypeptide comprising an amino acid sequence having at least 80% amino acid sequence identity to the amino acid sequence depicted in any one of FIG. 15A-15D; and iv) a proteolytically cleavable linker; and b) an insertion site for a nucleic acid comprising a nucleotide sequence encoding a polypeptide of interest. In some cases, the nucleotide sequence encoding the light-activated, calcium-gated fusion polypeptide is operably linked to a promoter. Suitable promoters are described below. In some cases, the nucleic acid is present in a recombinant expression vector, e.g., a recombinant viral vector. Suitable vectors are described below. The present disclosure provides a genetically modified host cell that is genetically modified with the nucleic acid. The present disclosure provides a genetically modified host cell that is genetically modified with the recombinant expression vector. Suitable host cells are described below.

As another example, the present disclosure provides a nucleic acid comprising: a) a nucleotide sequence encoding a light-activated, calcium-gated fusion polypeptide comprising, in order from amino terminus to carboxyl terminus: i) a transmembrane domain; ii) a calmodulin-binding polypeptide or a troponin I polypeptide; iii) a LOV-domain light-activated polypeptide comprising an amino acid sequence having at least 80% amino acid sequence identity to the amino acid sequence depicted in any one of FIG. 15E-15G; and iv) a proteolytically cleavable linker; and b) an insertion site for a nucleic acid comprising a nucleotide sequence encoding a polypeptide of interest. In some cases, the nucleotide sequence encoding the light-activated, calcium-gated fusion polypeptide is operably linked to a promoter. Suitable promoters are described below. In some cases, the nucleic acid is present in a recombinant expression vector, e.g., a recombinant viral vector. Suitable vectors are described below. The present disclosure provides a genetically modified host cell that is genetically modified with the nucleic acid. The present disclosure provides a genetically modified host cell that is genetically modified with the recombinant expression vector. Suitable host cells are described below.

As another example, the present disclosure provides a nucleic acid comprising a nucleotide sequence encoding a fusion polypeptide comprising: i) a calcium-binding polypeptide selected from a calmodulin polypeptide and troponin C polypeptide; and ii) a protease. In some cases, the nucleotide sequence encoding the fusion polypeptide is operably linked to a promoter. Suitable promoters are described below. In some cases, the nucleic acid is present in a recombinant expression vector, e.g., a recombinant viral vector. Suitable vectors are described below. The present disclosure provides a genetically modified host cell that is genetically modified with the nucleic acid. The present disclosure provides a genetically modified host cell that is genetically modified with the recombinant expression vector. Suitable host cells are described below.

As another example, the present disclosure provides a nucleic acid comprising a nucleotide sequence encoding a light-activated, calcium-gated fusion polypeptide comprising, in order from amino terminus to carboxyl terminus: i) a transmembrane domain; ii) a calmodulin-binding polypeptide or a troponin I polypeptide; iii) a LOV-domain light-activated polypeptide comprising an amino acid sequence having at least 80% amino acid sequence identity to the amino acid sequence depicted in any one of FIG. 15A-15D; iv) a proteolytically cleavable linker; and v) a polypeptide of interest. In some cases, the nucleotide sequence encoding the light-activated, calcium-gated fusion polypeptide is operably linked to a promoter. Suitable promoters are described below. In some cases, the nucleic acid is present in a recombinant expression vector, e.g., a recombinant viral vector. Suitable vectors are described below. The present disclosure provides a genetically modified host cell that is genetically modified with the nucleic acid. The present disclosure provides a genetically modified host cell that is genetically modified with the recombinant expression vector. Suitable host cells are described below.

As another example, the present disclosure provides a nucleic acid comprising a nucleotide sequence encoding a light-activated, calcium-gated fusion polypeptide comprising, in order from amino terminus to carboxyl terminus: i) a transmembrane domain; ii) a calmodulin-binding polypeptide or a troponin I polypeptide; iii) a LOV-domain light-activated polypeptide comprising an amino acid sequence having at least 80% amino acid sequence identity to the amino acid sequence depicted in any one of FIG. 15E-15G; iv) a proteolytically cleavable linker; and v) a polypeptide of interest. In some cases, the nucleotide sequence encoding the light-activated, calcium-gated fusion polypeptide is operably linked to a promoter. Suitable promoters are described below. In some cases, the nucleic acid is present in a recombinant expression vector, e.g., a recombinant viral vector. Suitable vectors are described below. The present disclosure provides a genetically modified host cell that is genetically modified with the nucleic acid. The present disclosure provides a genetically modified host cell that is genetically modified with the recombinant expression vector. Suitable host cells are described below.

### Transmembrane Domain

Any of a variety of transmembrane domains (polypeptides) can be used in a light-activated, calcium-gated transcriptional control polypeptide of the present disclosure. A suitable transmembrane domain is any polypeptide that is thermodynamically stable in a membrane, e.g., a eukaryotic cell membrane such as a mammalian cell membrane. Suitable transmembrane domains include a single alpha helix, a transmembrane beta barrel, or any other structure.

A “mammalian cell membrane” includes the membrane of a membrane-bound organelle (e.g., the nucleus, a mitochondrion, a lysosome, the endoplasmic reticulum, the Golgi apparatus, a vacuole, a chloroplast); and the plasma membrane. Thus, a suitable transmembrane domain is in some cases a transmembrane domain that provides for insertion into the plasma membrane. In some cases, a suitable transmembrane domain provides for insertion into a chloroplast membrane. In some cases, a suitable transmembrane domain provides for insertion into a mitochondrial membrane. In some cases, a suitable transmembrane domain provides for insertion into a lysosome.

A suitable transmembrane domain can have a length of from about 10 to 50 amino acids, e.g., from about 10 amino acids to about 40 amino acids, from about 20 amino acids to about 40 amino acids, from about 15 amino acids to about 25 amino acids, e.g., from about 10 amino acids to about 15 amino acids, from about 15 amino acids to about 20 amino acids, from about 20 amino acids to about 25 amino acids, from about 25 amino acids to about 30 amino acids, from about 30 amino acids to about 35 amino acids, from about 35 amino acids to about 40 amino acids, from about 40 amino acids to about 45 amino acids, or from about 45 amino acids to about 50 amino acids.

Suitable transmembrane (TM) domains include, e.g., a Syne homology nuclear TM domain; a CD4 TM domain; a CD8 TM domain; a KASH protein TM domain; a neurexin3b TM domain; a Notch receptor polypeptide TM domain; etc.

For example, a CD4 TM domain can comprise the amino acid sequence MALIVLGGVAGLLLFIGLGIFF (SEQ ID NO://); a CD8 TM domain can comprise the amino acid sequence IYIWAPLAGTCGVLLLSLVIT (SEQ ID NO://); a neurexin3b TM domain can comprise the amino acid sequence GMVVGIVAAAALCILILLYAM (SEQ ID NO://); a Notch receptor polypeptide TM domain can comprise the amino acid sequence FMYVAAAAFVLLFFVGCGVLL (SEQ ID NO://).

### Alternative Tethers

In some cases, in place of a transmembrane domain, the light-activated, calcium-gated fusion polypeptide comprises a polypeptide that tethers the light-activated, calcium-gated fusion polypeptide to actin. A suitable actin-binding polypeptide includes, e.g., filamin, spectrin, transgelin, fimbrin, villin, fascin, formin, tensin, tropomodulin, gelsolin, and actin-binding fragments thereof.

In some cases, in place of a transmembrane domain, the light-activated, calcium-gated fusion polypeptide comprises a polypeptide that excludes the light-activated, calcium-gated fusion polypeptide from the nucleus. Such a polypeptide can be a nuclear exclusion signal (NES) or nuclear export signal. Suitable NES polypeptides include, e.g., MVKELQEIRL (SEQ ID NO://); MTASALARMEV (SEQ ID NO://); LALKLAGLDI (SEQ ID NO://); LQKKLEELEL (SEQ ID NO://); LESNLRELQI (SEQ ID NO://); LCQAFSDVLI (SEQ ID NO://); MVKELQEIRLEP (SEQ ID NO://); LQKKLEELELA (SEQ ID NO://); LALKLAGLDIN (SEQ ID NO://); LQLPPLERLTLD (SEQ ID NO://); LQKKLEELELE (SEQ ID NO://); MTKKFGTLTI (SEQ ID NO://); LAEMLEDLHI (SEQ ID NO://); LDQQFAGLDL (SEQ ID NO://); LCQAFSDVIL (SEQ ID NO://); LPVLENLTL (SEQ ID NO://); and IQQQLGQLTLENLQML (SEQ ID NO://).

Another suitable protein is an estrogen receptor protein. For example, an estrogen receptor protein can comprise an amino acid sequence having at least 85%, at least 90%, at least 95%, at least 98%, at least 99%, or 100%, amino acid sequence identity to the following amino acid sequence: PSAGDMRAANLWPSPLMIKRSKKNSLALSLTADQMVSALLDAEPPILYSEYDPTRPFSE ASMMGLLTNLADRELVHMINWAKRVPGFVDLTLHDQVHLLECAWLEILMIGLVWRSM EHPVKLLFAPNLLLDRNQGKCVEGMVEIFDMLLATSSRFRMMNLQGEEFVCLKSIILLN SGVYTFLSSTLKSLEEKDHIHRVLDKITDTLIHLMAKAGLTLQQQHQRLAQLLLILSHIRH MSNKGMEHLYSMKCKNVVPLYDLLLEAADAHRLHAPTSRGGASVEETDQSHLATAGS TSSHSLQKYYITGEAEGFPATA; where the amino acid sequence is a MyoD-ERT2 fusion polypeptide, comprising the ligand-binding domain of estrogen receptor (amino acids 203-440), a basic domain in helix-loop-helix proteins of the MYOD family (amino acids 1-114).

### Calmodulin/Calmodulin-Binding Polypeptide

In some cases, the light-activated, calcium-gated fusion polypeptide comprises a calmodulin-binding polypeptide; and the second fusion polypeptide comprises a calmodulin polypeptide.

A suitable calmodulin-binding polypeptide binds a calmodulin polypeptide under conditions of high Ca²⁺ concentration. For example, a suitable calmodulin-binding polypeptide binds a calmodulin polypeptide when the concentration of Ca²⁺ is greater than 100 nM, greater than 150 nM, greater than 200 nM, greater than 250 nM, greater than 300 nM, greater than 350 nM, greater than 400 nM, greater than 500 nM, or greater than 750 nM.

A suitable calmodulin-binding polypeptide does not substantially bind a calmodulin polypeptide under conditions of low Ca²⁺ concentration. For example, a suitable calmodulin-binding polypeptide does not substantially bind a calmodulin polypeptide when the intracellular Ca²⁺ concentration is less than about 300 nM, less than about 250 nM, less than about 200 nM, less than about 110 nM, less than about 105 nM, or less than about 100 nM.

A calmodulin-binding polypeptide can have a length of from about 10 amino acids to about 50 amino acids, e.g., from about 10 amino acids to about 40 amino acids, from about 20 amino acids to about 40 amino acids, from about 15 amino acids to about 25 amino acids, e.g., from about 10 amino acids to about 15 amino acids, from about 15 amino acids to about 20 amino acids, from about 20 amino acids to about 25 amino acids, from about 25 amino acids to about 30 amino acids, from about 30 amino acids to about 35 amino acids, from about 35 amino acids to about 40 amino acids, from about 40 amino acids to about 45 amino acids, or from about 45 amino acids to about 50 amino acids.

A suitable calmodulin-binding polypeptide in some cases comprises an amino acid sequence having at least 90%, at least 95%, at least 98%, at least 99%, or 100%, amino acid sequence identity to the following amino acid sequence: KRRWKKNFIAVSAANRFKKISSSGAL (SEQ ID NO://); and has a length of from about 26 amino acids to about 30 amino acids.

In some cases, a suitable calmodulin-binding polypeptide comprises an amino acid sequence having at least 90%, at least 95%, at least 98%, at least 99%, or 100%, amino acid sequence identity to the following amino acid sequence: KRRWKKNFIAVSAANRFKKISSSGAL (SEQ ID NO://); and has a substitution of A14; and has a length of from about 26 amino acids to about 30 amino acids. In some cases, a suitable calmodulin-binding polypeptide comprises an amino acid sequence having at least 90%, at least 95%, at least 98%, at least 99%, or 100%, amino acid sequence identity to the following amino acid sequence: KRRWKKNFIAVSAANRFKKISSSGAL (SEQ ID NO://); and has an A14F substitution; and has a length of from about 26 amino acids to about 30 amino acids. In some cases, a suitable calmodulin-binding polypeptide comprises the following amino acid sequence: KRRWKKNFIAVSAFNRFKKISSSGAL (SEQ ID NO://); and has a length of 26 amino acids.

In some cases, a suitable calmodulin-binding polypeptide comprises an amino acid sequence having at least 90%, at least 95%, at least 98%, at least 99%, or 100%, amino acid sequence identity to the following amino acid sequence: FNARRKLKGAILTTMLFTRNFS (SEQ ID NO://); and has a length of from 22 amino acids to about 25 amino acids. In some cases, a suitable calmodulin-binding polypeptide comprises an amino acid sequence having at least 90%, at least 95%, at least 98%, at least 99%, or 100%, amino acid sequence identity to the following amino acid sequence: FNARRKLKGAILTTMLFTRNFS (SEQ ID NO://); and has a K8 amino acid substitution; and has a length of from 22 amino acids to about 25 amino acids. In some cases, a suitable calmodulin-binding polypeptide comprises an amino acid sequence having at least 90%, at least 95%, at least 98%, at least 99%, or 100%, amino acid sequence identity to the following amino acid sequence: FNARRKLKGAILTTMLFTRNFS (SEQ ID NO://); and has a K8A amino acid substitution; and has a length of from 22 amino acids to about 25 amino acids. In some cases, a suitable calmodulin-binding polypeptide comprises an amino acid sequence having at least 90%, at least 95%, at least 98%, at least 99%, or 100%, amino acid sequence identity to the following amino acid sequence: FNARRKLKGAILTTMLFTRNFS (SEQ ID NO://); and has a T13 substitution; and has a length of from 22 amino acids to about 25 amino acids. In some cases, a suitable calmodulin-binding polypeptide comprises an amino acid sequence having at least 90%, at least 95%, at least 98%, at least 99%, or 100%, amino acid sequence identity to the following amino acid sequence: FNARRKLKGAILTTMLFTRNFS (SEQ ID NO://); and has a T13F substitution; and has a length of from 22 amino acids to about 25 amino acids. In some cases, a suitable calmodulin-binding polypeptide comprises the following amino acid sequence: FNARRKLKGAILFTMLFTRNFS; and has a length of 22 amino acids. In some cases, a suitable calmodulin-binding polypeptide comprises the following amino acid sequence: FNARRKLAGAILFTMLFTRNFS; and has a length of 22 amino acids.

In some cases, two copies of a calmodulin-binding polypeptide are used. For example, a calmodulin-binding polypeptide can comprise the amino acid sequence FNARRKLAGAILFTMLATRNFSGSFNARRKLAGAILFTMLATRNFS (SEQ ID NO://) which contains two copies of FNARRKLAGAILFTMLATRNFS (SEQ ID NO://) and an intervening Gly-Ser (GS) linker.

A suitable calmodulin polypeptide comprises an amino acid sequence having at least 80%, at least 85%, at least 90%, at least 95%, at least 98%, at least 99%, or 100%, amino acid sequence identity to the amino acid sequence depicted in FIG. 16A or FIG. 16B.

A suitable calmodulin polypeptide comprises an amino acid sequence having at least 80%, at least 85%, at least 90%, at least 95%, at least 98%, at least 99%, or 100%, amino acid sequence identity to the following calmodulin amino acid sequence: MDQLTEEQIAEFKEAFSLFDKDGDGTITTKELGTVMRSLGQNPTEAELQDMINEVDADG DGTIDFPEFLTMMARKMKYTDSEEEIREAFRVFDKDGNGYISAAELRHVMTNLGEKLTD EEVDEMIREADIDGDGQVNYEEFVQMMTAK (SEQ ID NO://); and has a length of from about 148 amino acids to about 160 amino acids. In some cases, the calmodulin polypeptide has a length of 148 amino acids.

In some cases, a suitable calmodulin polypeptide comprises an amino acid sequence having at least 80%, at least 85%, at least 90%, at least 95%, at least 98%, at least 99%, or 100%, amino acid sequence identity to the following calmodulin amino acid sequence: MDQLTEEQIAEFKEAFSLFDKDGDGTITTKELGTVMRSLGQNPTEAELQDMINEVDADG DGTIDFPEFLTMMARKMKYTDSEEEIREAFRVFDKDGNGYISAAELRHVMTNLGEKLTD EEVDEMIREADIDGDGQVNYEEFVQMMTAK (SEQ ID NO://); and has a substitution of F19; and has a length of from about 148 amino acids to about 160 amino acids. In some cases, the calmodulin polypeptide has a length of 148 amino acids. In some cases, the F19 substitution is an F19L substitution, an F19I substitution, an F19V substitution, or an F19A substitution.

In some cases, a suitable calmodulin polypeptide comprises an amino acid sequence having at least 80%, at least 85%, at least 90%, at least 95%, at least 98%, at least 99%, or 100%, amino acid sequence identity to the following calmodulin amino acid sequence: MDQLTEEQIAEFKEAFSLFDKDGDGTITTKELGTVMRSLGQNPTEAELQDMINEVDADG DGTIDFPEFLTMMARKMKYTDSEEEIREAFRVFDKDGNGYISAAELRHVMTNLGEKLTD EEVDEMIREADIDGDGQVNYEEFVQMMTAK (SEQ ID NO://); and has a substitution of V35; and has a length of from about 148 amino acids to about 160 amino acids. In some cases, the calmodulin polypeptide has a length of 148 amino acids. In some cases, the V35 substitution is a V35G substitution, a V35A substitution, a V35L substitution, or a V35I substitution.

In some cases, a suitable calmodulin polypeptide comprises an amino acid sequence having at least 80%, at least 85%, at least 90%, at least 95%, at least 98%, at least 99%, or 100%, amino acid sequence identity to the following calmodulin amino acid sequence: MDQLTEEQIAEFKEAFSLFDKDGDGTITTKELGTVMRSLGQNPTEAELQDMINEVDADG DGTIDFPEFLTMMARKMKYTDSEEEIREAFRVFDKDGNGYISAAELRHVMTNLGEKLTD EEVDEMIREADIDGDGQVNYEEFVQMMTAK (SEQ ID NO://); and has an F19 substitution (e.g., an F19L substitution, an F19I substitution, an F19V substitution, or an F19A substitution) and a V35 substitution (e.g., a V35G substitution, a V35A substitution, a V35L substitution, or a V35I substitution); and has a length of from about 148 amino acids to about 160 amino acids. In some cases, the calmodulin polypeptide has a length of 148 amino acids.

In some cases, a suitable calmodulin polypeptide comprises an amino acid sequence having at least 80%, at least 85%, at least 90%, at least 95%, at least 98%, at least 99%, or 100%, amino acid sequence identity to the following calmodulin amino acid sequence: MDQLTEEQIAEFKEAFSLLDKDGDGTITTKELGTGMRSLGQNPTEAELQDMINEVDADG DGTIDFPEFLTMMARKMKYTDSEEEIREAFRVFDKDGNGYISAAELRHVMTNLGEKLTD EEVDEMIREADIDGDGQVNYEEFVQMMTAK (SEQ ID NO://); and comprises a Leu at amino acid 19 and a Gly at amino acid 35; and has a length of from about 148 amino acids to about 160 amino acids. In some cases, the calmodulin polypeptide has a length of 148 amino acids.

### Troponin C/Troponin I

In some cases, the light-activated, calcium-gated fusion polypeptide comprises a troponin C-binding polypeptide (e.g., a troponin I polypeptide); and the second fusion polypeptide comprises a troponin C polypeptide.

A suitable troponin I polypeptide binds a troponin C polypeptide under conditions of high Ca²⁺ concentration. For example, a suitable troponin I polypeptide binds a troponin C polypeptide when the concentration of Ca²⁺ is greater than 100 nM, greater than 150 nM, greater than 200 nM, greater than 250 nM, greater than 300 nM, greater than 350 nM, greater than 400 nM, greater than 500 nM, or greater than 750 nM.

A suitable troponin I polypeptide does not substantially bind a troponin C polypeptide under conditions of low Ca²⁺ concentration. For example, a suitable troponin I polypeptide does not substantially bind a troponin C polypeptide when the intracellular Ca²⁺ concentration is less than about 300 nM, less than about 250 nM, less than about 200 nM, less than about 110 nM, less than about 105 nM, or less than about 100 nM.

A troponin I polypeptide can have a length of from about 10 amino acids to about 200 amino acids, e.g., from about 10 amino acids to about 40 amino acids, from about 20 amino acids to about 40 amino acids, from about 15 amino acids to about 25 amino acids, e.g., from about 10 amino acids to about 15 amino acids, from about 15 amino acids to about 20 amino acids, from about 20 amino acids to about 25 amino acids, from about 25 amino acids to about 30 amino acids, from about 30 amino acids to about 35 amino acids, from about 35 amino acids to about 40 amino acids, from about 40 amino acids to about 45 amino acids, from about 45 amino acids to about 50 amino acids, from about amino acids to about 75 amino acids, from about 75 amino acids to about 100 amino acids, from about 100 amino acids to about 150 amino acids, or from about 150 amino acids to about 200 amino acids.

In some cases, a suitable troponin I polypeptide comprises an amino acid sequence having at least 80%, at least 85%, at least 90%, at least 95%, at least 98%, at least 99%, or 100%, amino acid sequence identity to the following troponin I amino acid sequence:

(SEQ ID NO: //)
MPEVERKPKI TASRKLLLKS LMLAKAKECW EQEHEEREAE
KVRYLAERIP TLQTRGLSLS ALQDLCRELH AKVEVVDEER
YDIEAKCLHN TREIKDLKLK VMDLRGKFKR PPLRRVRVSA
DAMLRALLGS KHKVSMDLRA NLKSVKKEDT EKERPVEVGD
WRKNVEAMSG MEGRKKMFDA AKSPTSQ.

A fragment of troponin I can be used. See, e.g., Tung et al. (2000) Protein Sci. 9:1312. For example, troponin I (95-114) can be used. Thus, for example, in some cases, the troponin I polypeptide can comprise an amino acid sequence having at least 80%, at least 85%, at least 90%, at least 95%, at least 98%, at least 99%, or 100%, amino acid sequence identity to the following troponin I amino acid sequence: KDLKLK VMDLRGKFKR PPLR (SEQ ID NO://); and has a length of about 20 amino acids to about 50 amino acids (e.g., from about 20 amino acids to about 25 amino acids, from about 25 amino acids to about 30 amino acids, from about 30 amino acids to about 35 amino acids, from about 35 amino acids to about 40 amino acids, from about 40 amino acids to about 45 amino acids, or from about 45 amino acids to about 50 amino acids). In some cases, the troponin I polypeptide has a length of 20 amino acids. In some cases, the troponin I polypeptide has the amino acid sequence: KDLKLK VMDLRGKFKR PPLR (SEQ ID NO://); and has a length of 20 amino acids.

In some cases, a suitable troponin I polypeptide comprises an amino acid sequence having at least 80%, at least 85%, at least 90%, at least 95%, at least 98%, at least 99%, or 100%, amino acid sequence identity to the following troponin I amino acid sequence: RMSADAMLKALLGSKHKVAMDLRAN (SEQ ID NO://); and has a length of from about 25 amino acids to about 50 amino acids (e.g., from about 25 amino acids to about 30 amino acids, from about 30 amino acids to about 35 amino acids, from about 35 amino acids to about 40 amino acids, from about 40 amino acids to about 45 amino acids, or from about 45 amino acids to about 50 amino acids). In some cases, the troponin I polypeptide has the amino acid sequence: RMSADAMLKALLGSKHKVAMDLRAN (SEQ ID NO://); and has a length of 25 amino acids.

In some cases, a suitable troponin I polypeptide comprises an amino acid sequence having at least 80%, at least 85%, at least 90%, at least 95%, at least 98%, at least 99%, or 100%, amino acid sequence identity to the following troponin I amino acid sequence: NQKLFDLRGKFKRPPLRRVRMSADAMLKALLGSKHKVAMDLRAN (SEQ ID NO://); and has a length of from about 44 amino acids to about 50 amino acids (e.g., 44, 45, 46, 47, 4, 49, or 50 amino acids). In some cases, the troponin I polypeptide has the amino acid sequence: NQKLFDLRGKFKRPPLRRVRMSADAMLKALLGSKHKVAMDLRAN (SEQ ID NO://); and has a length of 44 amino acids.

A suitable troponin C polypeptide comprises an amino acid sequence having at least 80%, at least 85%, at least 90%, at least 95%, at least 98%, at least 99%, or 100%, amino acid sequence identity to the following troponin C amino acid sequence: MTDQQAEARS YLSEEMIAEF KAAFDMFDAD GGGDISVKEL GTVMRMLGQT PTKEELDAII EEVDEDGSGT IDFEEFLVMM VRQMKEDAKG KSEEELAECF RIFDRNADGY IDPGELAEIF RASGEHVTDE EIESLMKDGD KNNDGRIDFD EFLKMMEGVQ (SEQ ID NO://).

A suitable troponin C polypeptide can have a length of from about 100 amino acids to about 175 amino acids, e.g., from about 100 amino acids to about 125 amino acids, from about 125 amino acids to about 150 amino acids, or from about 150 amino acids to about 175 amino acids.

A suitable troponin C polypeptide comprises an amino acid sequence having at least 80%, at least 85%, at least 90%, at least 95%, at least 98%, at least 99%, or 100%, amino acid sequence identity to the following troponin C amino acid sequence: MTDQQAEARSYLSEEMIAEFKAAFDMFDADGGGDISVKELGTVMRMLGQTPTKEELD AIIEEVDEDGSGTIDFEEFLVMMVRQMKEDAKGKSEEELAECFRIFDRDANGYIDAEELA EIFRASGEHVTDEEIESLMKDGDKNNDGRIDFDEFLKMMEGVQ (SEQ ID NO://); and has a length of from about 160 amino acids to about 175 amino acids (e.g., from about 160 amino acids to about 165 amino acids, from about 165 amino acids to about 170 amino acids, or from about 170 amino acids to about 175 amino acids. In some cases, a suitable troponin C polypeptide comprises the amino acid sequence: MTDQQAEARSYLSEEMIAEFKAAFDMFDADGGGDISVKELGTVMRMLGQTPTKEELD AIIEEVDEDGSGTIDFEEFLVMMVRQMKEDAKGKSEEELAECFRIFDRDANGYIDAEELA EIFRASGEHVTDEEIESLMKDGDKNNDGRIDFDEFLKMMEGVQ (SEQ ID NO://); and has a length of 160 amino acids.

### LOV-Domain Light-Activated Polypeptide

A LOV domain light-activated polypeptide that can be encoded by a nucleotide sequence present in a nucleic acid of a system (System 1 or System 2) of the present disclosure is activatable by blue light, and can cage a proteolytically cleavable linker attached to the light-activated polypeptide. Thus, in the absence of blue light, the proteolytically cleavable linker is caged, i.e., inaccessible to a protease. In the presence of blue light, the light-activated polypeptide undergoes a conformational change, such that the proteolytically cleavable linker is uncaged and becomes accessible to a protease. A LOV domain light-activated polypeptide comprises a light, oxygen, or voltage (LOV) domain (a “LOV polypeptide”).

A suitable LOV domain light-activated polypeptide can have a length of from about 100 amino acids to about 150 amino acids. For example, a LOV polypeptide can comprise an amino acid sequence having at least 80%, at least 85%, at least 90%, at least 95%, at least 98%, at least 99%, or 100%, amino acid sequence identity to the LOV2 domain of Avena sativa phototropin 1 (AsLOV2).

In some cases, a suitable LOV domain light-activated polypeptide comprises an amino sequence having at least 80%, at least 85%, at least 90%, at least 95%, at least 98%, at least 99%, or 100%, amino acid sequence identity to the following LOV2 amino acid sequence: DLATTLERIEKNFVITDPRLPDNPIIFASDSFLQLTEYSREEILGRNCRFLQGPETDRATVRKI RDAIDNQTEVTVQLINYTKSGKKFWNLFHLQPMRDQKGDVQYFIGVQLDGTEHVRDAAEREGVM LIKKTAENIDEAAK (SEQ ID NO://); GenBank AF033096. In some cases, a suitable LOV polypeptide comprises an amino sequence having at least 80%, at least 85%, at least 90%, at least 95%, at least 98%, at least 99%, or 100%, amino acid sequence identity to the following LOV2 amino acid sequence: DLATTLERIEKNFVITDPRLPDNPIIFASDSFLQLTEYSREEILGRNCRFLQGPETDRATVRKI RDAIDNQTEVTVQLINYTKSGKKFWNLFHLQPMRDQKGDVQYFIGVQLDGTEHVRDAAEREGVM LIKKTAENIDEAAK (SEQ ID NO://); and has a length of from 142 amino acids to 150 amino acids. In some cases, a suitable LOV domain light-activated polypeptide comprises an amino sequence having at least 80%, at least 85%, at least 90%, at least 95%, at least 98%, at least 99%, or 100%, amino acid sequence identity to the following LOV2 amino acid sequence: DLATTLERIEKNFVITDPRLPDNPIIFASDSFLQLTEYSREEILGRNCRFLQGPETDRATVRKI RDAIDNQTEVTVQLINYTKSGKKFWNLFHLQPMRDQKGDVQYFIGVQLDGTEHVRDAAEREGVM LIKKTAENIDEAAK (SEQ ID NO://); and has a length of 142 amino acids.

In some cases, a suitable LOV domain light-activated polypeptide comprises an amino sequence having at least 80%, at least 85%, at least 90%, at least 95%, at least 98%, at least 99%, or 100%, amino acid sequence identity to the following amino acid sequence: SLATTLERIEKNFVITDPRLPDNPIIFASDSFLQLTEYSREEILGRNCRFLQGPETDRATVR KIRDAIDNQTEVTVQLINYTKSGKKFWNLFHLQPMRDQKGDVQYFIGVQLDGTEHVRD AAEREAVMLIKKTAEEIDEAAK (SEQ ID NO://). In some cases, a suitable LOV domain light-activated polypeptide comprises an amino sequence having at least 80%, at least 85%, at least 90%, at least 95%, at least 98%, at least 99%, or 100%, amino acid sequence identity to the following amino acid sequence: SLATTLERIEKNFVITDPRLPDNPIIFASDSFLQLTEYSREEILGRNCRFLQGPETDRATVR KIRDAIDNQTEVTVQLINYTKSGKKFWNLFHLQPMRDQKGDVQYFIGVQLDGTEHVRD AAEREAVMLIKKTAEEIDEAAK (SEQ ID NO://); and has a length of from about 142 amino acids to about 150 amino acids. In some cases, a suitable LOV domain light-activated polypeptide comprises an amino sequence having at least 80%, at least 85%, at least 90%, at least 95%, at least 98%, at least 99%, or 100%, amino acid sequence identity to the following amino acid sequence: SLATTLERIEKNFVITDPRLPDNPIIFASDSFLQLTEYSREEILGRNCRFLQGPETDRATVR KIRDAIDNQTEVTVQLINYTKSGKKFWNLFHLQPMRDQKGDVQYFIGVQLDGTEHVRD AAEREAVMLIKKTAEEIDEAAK (SEQ ID NO://); and has a length of 142 amino acids.

In some cases, a suitable LOV domain light-activated polypeptide comprises an amino sequence having at least 80%, at least 85%, at least 90%, at least 95%, at least 98%, at least 99%, or 100%, amino acid sequence identity to the following amino acid sequence: SLATTLERIEKNFVITDPRLPDNPIIFASDSFLQLTEYSREEILGRNCRFLQGPETDRATVR KIRDAIDNQTEVTVQLINYTKSGKKFWNLFHLQPMRDQKGDVQYFIGVQLDGTEHVRD AAEREAVMLIKKTAEEIDEAAK (SEQ ID NO://); and comprises a substitution at one or more of amino acids L2, N12, A28, H117, and I130, where the numbering is based on the amino acid sequence SLATTLERIEKNFVITDPRLPDNPIIFASDSFLQLTEYSREEILGRNCRFLQGPETDRATVR KIRDAIDNQTEVTVQLINYTKSGKKFWNLFHLQPMRDQKGDVQYFIGVQLDGTEHVRD AAEREAVMLIKKTAEEIDEAAK (SEQ ID NO://).

A suitable LOV domain light-activated polypeptide comprises one or more amino acid substitutions relative to the LOV2 amino acid sequence depicted in FIG. 15A. In some cases, a suitable LOV domain light-activated polypeptide comprises one or more amino acid substitutions at positions selected from 1, 2, 12, 25, 28, 91, 100, 117, 118, 119, 120, 126, 128, 135, 136, and 138, relative to the LOV2 amino acid sequence depicted in FIG. 15A. Suitable substitutions include, Asp→Ser at amino acid 1; Asp→Phe at amino acid 1; Leu→Arg at amino acid 2; Asn→Ser at amino acid 12; Ile→Val at amino acid 12; Ala→Val at amino acid 28; Leu→Val at amino acid 91; Gln→Tyr at amino acid 100; His→Arg at amino acid 117; Val→Leu at amino acid 118; Arg→His at amino acid 119; Asp→Gly at amino acid 120; Gly→Ala at amino acid 126; Met→Cys at amino acid 128; Glu→Phe at amino acid 135; Asn→Gln at amino acid 136; Asn→Glu at amino acid 136; and Asp→Ala at amino acid 138, where the amino acid numbering is based on the number of the LOV2 amino acid sequence depicted in FIG. 15A.

In some cases, a suitable LOV domain light-activated polypeptide comprises an amino acid sequence having at least 80%, at least 85%, at least 90%, at least 95%, at least 98%, at least 99%, or 100%, amino acid sequence identity to the amino acid sequence depicted in FIG. 15B, where amino acid 1 is Ser, amino acid 28 is Ala, amino acid 126 is Ala, and amino acid 136 is Glu. In some case, the suitable LOV domain light-activated polypeptide has a length of 142 amino acids.

In some cases, a suitable LOV domain light-activated polypeptide comprises an amino acid sequence having at least 80%, at least 85%, at least 90%, at least 95%, at least 98%, at least 99%, or 100%, amino acid sequence identity to the amino acid sequence depicted in FIG. 15C, where amino acid 1 is Ser; amino acid 2 is Arg; amino acid 12 is Ser; amino acid 28 is Ala; amino acid 117 is Arg; amino acid 126 is Ala; and amino acid 136 is Glu. In some case, the suitable LOV domain light-activated polypeptide has a length of 142 amino acids.

In some cases, a suitable LOV domain light-activated polypeptide comprises an amino acid sequence having at least 80%, at least 85%, at least 90%, at least 95%, at least 98%, at least 99%, or 100%, amino acid sequence identity to the amino acid sequence depicted in FIG. 15D, where amino acid 1 is Ser; amino acid 2 is Arg; amino acid 12 is Ser; amino acid 25 is Val; amino acid 28 is Val; amino acid 117 is Arg; amino acid 126 is Ala; amino acid 130 is Val; and amino acid 136 is Glu. In some case, the LOV domain light-activated polypeptide has a length of 142 amino acids.

In some cases, a suitable LOV domain light-activated polypeptide comprises an amino acid sequence having at least 80%, at least 85%, at least 90%, at least 95%, at least 98%, at least 99%, or 100%, amino acid sequence identity to the amino acid sequence depicted in FIG. 15E, where amino acid 1 is Ser; amino acid 2 is Arg; amino acid 12 is Ser; amino acid 28 is Ala; amino acid 91 is Val; amino acid 100 is Tyr; amino acid 117 is Arg; amino acid 118 is Leu; amino acid 119 is His; amino acid 120 is Gly; amino acid 126 is Ala; amino acid 128 is Cys; amino acid 130 is Val; amino acid 135 is Phe; amino acid 136 is Gln; and amino acid 138 is Ala. In some case, the LOV domain light-activated polypeptide has a length of 142 amino acids.

In some cases, a suitable LOV domain light-activated polypeptide comprises an amino acid sequence having at least 80%, at least 85%, at least 90%, at least 95%, at least 98%, at least 99%, or 100%, amino acid sequence identity to the amino acid sequence depicted in FIG. 15F, where amino acid 1 is Ser; amino acid 2 is Arg; amino acid 12 is Ser; amino acid 28 is Val; amino acid 117 is Arg; amino acid 126 is Ala; amino acid 130 is Val; and amino acid 136 is Glu. In some case, the LOV domain light-activated polypeptide has a length of 138 amino acids.

In some cases, a suitable LOV domain light-activated polypeptide comprises an amino acid sequence having at least 80%, at least 85%, at least 90%, at least 95%, at least 98%, at least 99%, or 100%, amino acid sequence identity to the amino acid sequence depicted in FIG. 15G, where amino acid 1 is Ser; amino acid 2 is Arg; amino acid 12 is Ser; amino acid 28 is Val; amino acid 91 is Val; amino acid 100 is Tyr; amino acid 117 is Arg; amino acid 118 is Leu; amino acid 119 is His; amino acid 120 is Gly; amino acid 126 is Ala; amino acid 128 is Cys; amino acid 130 is Val; amino acid 135 is Phe; amino acid 136 is Gln; and amino acid 138 is Ala. In some case, the LOV domain light-activated polypeptide has a length of 138 amino acids.

In some cases, the LOV domain light-activated polypeptide comprises a substitution selected from an L2R substitution, an L2H substitution, an L2P substitution, and an L2K substitution. In some cases, the LOV polypeptide comprises a substitution selected from an N12S substitution, an N12T substitution, and an N12Q substitution. In some cases, the LOV polypeptide comprises a substitution selected from an A28V substitution, an A28I substitution, and an A28L substitution. In some cases, the LOV polypeptide comprises a substitution selected from an H117R substitution, and an H117K substitution. In some cases, the LOV polypeptide comprises a substitution selected from an I130V substitution, an I130A substitution, and an I130L substitution. In some cases, the LOV polypeptide comprises substitutions at amino acids L2, N12, and I130. In some cases, the LOV polypeptide comprises substitutions at amino acids L2, N12, H117, and I130. In some cases, the LOV polypeptide comprises substitutions at amino acids A28 and H117. In some cases, the LOV polypeptide comprises substitutions at amino acids N12 and I130. In some cases, the LOV polypeptide comprises an L2R substitution, an N12S substitution, and an I130V substitution. In some cases, the LOV polypeptide comprises an N12S substitution and an I130V substitution. In some cases, the LOV polypeptide comprises an A28V substitution and an H117R substitution. In some cases, the LOV polypeptide comprises an L2P substitution, an N12S substitution, an I130V substitution, and an H117R substitution. In some cases, the LOV polypeptide comprises an L2P substitution, an N12S substitution, an A28V substitution, an H117R substitution, and an I130V substitution. In some cases, the LOV polypeptide comprises an L2P substitution, an N12S substitution, an I130V substitution, and an H117R substitution. In some cases, the LOV polypeptide comprises an L2R substitution, an N12S substitution, an A28V substitution, an H117R substitution, and an I130V substitution. In some cases, the LOV polypeptide has a length of 142 amino acids, 143 amino acids, 144 amino acids, 145 amino acids, 146 amino acids, 147 amino acids, 148 amino acids, 149 amino acids, or 150 amino acids. In some cases, the LOV polypeptide has a length of 142 amino acids.

In some cases, a suitable LOV polypeptide comprises an amino sequence having at least 80%, at least 85%, at least 90%, at least 95%, at least 98%, at least 99%, or 100%, amino acid sequence identity to the following amino acid sequence: SRATTLERIEKSFVITDPRLPDNPIIFVSDSFLQLTEYSREEILGRNCRFLQGPETDRATVR KIRDAIDNQTEVTVQLINYTKSGKKFWNLFHLQPMRDQKGDVQYFIGVQLDGTERVRD AAEREAVMLVKKTAEEIDEAAK (SEQ ID NO://); and has an Arg at amino acid 2, a Ser at amino acid 12, a Val at amino acid 28, an Arg at amino acid 117, and a Val at amino acid 130, as indicated by bold and underlined letters; and has a length of 142 amino acids, 143 amino acids, 144 amino acids, 145 amino acids, 146 amino acids, 147 amino acids, 148 amino acids, 149 amino acids, or 150 amino acids. In some cases, a suitable LOV polypeptide comprises the following amino acid sequence: SRATTLERIEKSFVITDPRLPDNPIIFVSDSFLQLTEYSREEILGRNCRFLQGPETDRATVR KIRDAIDNQTEVTVQLINYTKSGKKFWNLFHLQPMRDQKGDVQYFIGVQLDGTERVRD AAEREAVMLVKKTAEEIDEAAK (SEQ ID NO://); and has a length of 142 amino acids.

In some cases, a suitable LOV polypeptide comprises an amino sequence having at least 80%, at least 85%, at least 90%, at least 95%, at least 98%, at least 99%, or 100%, amino acid sequence identity to the following amino acid sequence: SRATTLERIEKSFVITDPRLPDNPVIFVSDSFLQLTEYSREEILGRNCRFLQGPETDRATVR KIRDAIDNQTEVTVQLINYTKSGKKFWNLFHLQPMRDQKGDVQYFIGVQLDGTERVRD AAEREAVMLVKKTAEEIDEAAK (SEQ ID NO://); and has an Arg at amino acid 2, a Ser at amino acid 12, a Val at amino acid 25, a Val at amino acid 28, an Arg at amino acid 117, and a Val at amino acid 130, as indicated by bold and underlined letters; and has a length of 142 amino acids, 143 amino acids, 144 amino acids, 145 amino acids, 146 amino acids, 147 amino acids, 148 amino acids, 149 amino acids, or 150 amino acids. In some cases, a suitable LOV polypeptide comprises the following amino acid sequence: SRATTLERIEKSFVITDPRLPDNPVIFVSDSFLQLTEYSREEILGRNCRFLQGPETDRATVR KIRDAIDNQTEVTVQLINYTKSGKKFWNLFHLQPMRDQKGDVQYFIGVQLDGTERVRD AAEREAVMLVKKTAEEIDEAAK (SEQ ID NO://); and has a length of 142 amino acids.

In some cases, a LOV light-activated polypeptide comprises the following amino acid sequence:

(SEQ ID NO://)
FRATTLERIEKSFVITDPRLPDNPIIFVSDSFLQLTEYSREEILGRNCRF
LQGPETDRATVRKIRDAIDNQTEVTVQLINYTKSGKKFWNVFHLQPMRDY
KGDVQYFIGVQLDGTERLHGAAEREAVCLVKKTAFQIA.

In some cases, a LOV light-activated polypeptide comprises the following amino acid sequence:

(SEQ ID NO://)
SRATTLERIEKSFVITDPRLPDNPIIFVSDSFLQLTEYSREEILGRNCRF
LQGPETDRATVRKIRDAIDNQTEVTVQLINYTKSGKKFWNLFHLQPMRDQ
KGDVQYFIGVQLDGTERVRDAAEREAVMLVKKTAEEID.

In some cases, a LOV light-activated polypeptide comprises the following amino acid sequence:

(SEQ ID NO://)
FRATTLERIEKSFVITDPRLPDNPIIFVSDSFLQLTEYSREEILGRNCRF
LQGPETDRATVRKIRDAIDNQTEVTVQLINYTKSGKKFWNVFHLQPMRDY
KGDVQYFIGVQLDGTERLHGAAEREAVCLVKKTAFQIA.

In some cases, a LOV light-activated polypeptide comprises the following amino acid sequence:

(SEQ ID NO://)
SRATTLERIEKSFVITDPRLPDNPIIFVSDSFLQLTEYSREEILGRNCRF
LQGPETDRATVRKIRDAIDNQTEVTVQLINYTKSGKKFWNVFHLQPMRDY
KGDVQYFIGVQLDGTERLHGAAEREAVCLVKKTAFEIDEAAK.

In some cases, a LOV light-activated polypeptide comprises the following amino acid sequence:

(SEQ ID NO: //)
SRATTLERIEKSFVITDPRLPDNPIIFVSDSFLQLTEYSREEILGRN
CRFLQGPETDRATVRKIRDAIDNQTEVTVQLINYTKSGKKFWNLFHL
QPMRDQKGDVQYFIGVQLDGTERVRDAAEREAVMLVKKTAEEIDEAA
K.

LOV light-activated polypeptide cages the proteolytically cleavable linker in the absence of light of an activating wavelength, the proteolytically cleavable linker is substantially not accessible to the protease. Thus, e.g., in the absence of light of an activating wavelength (e.g., in the dark; or in the presence of light of a wavelength other than blue light), the proteolytically cleavable linker is cleaved, if at all, to a degree that is more than 50% less, more than 60% less, more than 70% less, more than 80% less, more than 90% less, more than 95% less, more than 98% less, or more than 99% less, than the degree of cleavage of the proteolytically cleavable linker in the presence of light of an activating wavelength (e.g., blue light, e.g., light of a wavelength in the range of from about 450 nm to about 495 nm, from about 460 nm to about 490 nm, from about 470 nm to about 480 nm, e.g., 473 nm).

Non-limiting examples of suitable polypeptides comprising: a) a LOV light-activated polypeptide; and b) a proteolytically cleavable linker include the following (where the proteolytically cleavable linker is underlined, and where the triangle indicates the cleavage site):

1)
(SEQ ID NO: //)
SRATTLERIEKSFVITDPRLPDNPIIFVSDSFLQLTEYSREEILGRN
CRFLQGPETDRATVRKIRDAIDNQTEVTVQLINYTKSGKKFWNLFHL
QPMRDQKGDVQYFIGVQLDGTERVRDAAEREAVMLVKKTAEEIDEAA
KENLYFQ_{▴}M;
2)
(SEQ ID NO: //)
SRATTLERIEKSFVITDPRLPDNPIIFVSDSFLQLTEYSREEILGRN
CRFLQGPETDRATVRKIRDAIDNQTEVTVQLINYTKSGKKFWNVFHL
QPMRDYKGDVQYFIGVQLDGTERLHGAAEREAVCLVKKTAFEIDEAA
KENLYFQ_{▴}M;
3)
(SEQ ID NO: //)
FRATTLERIEKSFVITDPRLPDNPIIFVSDSFLQLTEYSREEILGRN
CRFLQGPETDRATVRKIRDAIDNQTEVTVQLINYTKSGKKFWNVFHL
QPMRDYKGDVQYFIGVQLDGTERLHGAAEREAVCLVKKTAFQIAENL
YFQ_{▴}M;
4)
(SEQ ID NO: //)
SRATTLERIEKSFVITDPRLPDNPIIFVSDSFLQLTEYSREEILGRN
CRFLQGPETDRATVRKIRDAIDNQTEVTVQLINYTKSGKKFWNLFHL
QPMRDQKGDVQYFIGVQLDGTERVRDAAEREAVMLVKKTAEEIDENL
YFQ_{▴}G;
and
5)
(SEQ ID NO: //)
FRATTLERIEKSFVITDPRLPDNPIIFVSDSFLQLTEYSREEILGRN
CRFLQGPETDRATVRKIRDAIDNQTEVTVQLINYTKSGKKFWNVFHL
QPMRDYKGDVQYFIGVQLDGTERLHGAAEREAVCLVKKTAFQIAENL
YFQ_{▴}G.

### Proteolytically Cleavable Linker

The proteolytically cleavable linker can include a protease recognition sequence recognized by a protease selected from the group consisting of alanine carboxypeptidase, Armillaria mellea astacin, bacterial leucyl aminopeptidase, cancer procoagulant, cathepsin B, clostripain, cytosol alanyl aminopeptidase, elastase, endoproteinase Arg-C, enterokinase, gastricsin, gelatinase, Gly-X carboxypeptidase, glycyl endopeptidase, human rhinovirus 3C protease, hypodermin C, IgA-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2A, picornain 3C, proendopeptidase, prolyl aminopeptidase, proprotein convertase I, proprotein convertase II, russellysin, saccharopepsin, semenogelase, T-plasminogen activator, thrombin, tissue kallikrein, tobacco etch virus (TEV), togavirin, tryptophanyl aminopeptidase, U-plasminogen activator, V8, venombin A, venombin AB, and Xaa-pro aminopeptidase.

For example, the proteolytically cleavable linker can comprise a matrix metalloproteinase (MMP) cleavage site, e.g., a cleavage site for a MMP selected from collagenase-1, -2, and -3 (MMP-1, -8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP). For example, the cleavage sequence of MMP-9 is Pro-X-X-Hy (wherein, X represents an arbitrary residue; Hy, a hydrophobic residue), e.g., Pro-X-X-Hy-(Ser/Thr), e.g., Pro-Leu/Gln-Gly-Met-Thr-Ser (SEQ ID NO://) or Pro-Leu/Gln-Gly-Met-Thr (SEQ ID NO://). Another example of a protease cleavage site is a plasminogen activator cleavage site, e.g., a uPA or a tissue plasminogen activator (tPA) cleavage site. Another example of a suitable protease cleavage site is a prolactin cleavage site. Specific examples of cleavage sequences of uPA and tPA include sequences comprising Val-Gly-Arg. Another example of a protease cleavage site that can be included in a proteolytically cleavable linker is a tobacco etch virus (TEV) protease cleavage site, e.g., ENLYFQS (SEQ ID NO://), where the protease cleaves between the glutamine and the serine; or ENLYFQY (SEQ ID NO://), where the protease cleaves between the glutamine and the tyrosine; or ENLYFQL (SEQ ID NO://), where the protease cleaves between the glutamine and the leucine. Another example of a protease cleavage site that can be included in a proteolytically cleavable linker is an enterokinase cleavage site, e.g., DDDDK (SEQ ID NO://), where cleavage occurs after the lysine residue. Another example of a protease cleavage site that can be included in a proteolytically cleavable linker is a thrombin cleavage site, e.g., LVPR (SEQ ID NO://) (e.g., where the proteolytically cleavable linker comprises the sequence LVPRGS (SEQ ID NO://)). Additional suitable linkers comprising protease cleavage sites include linkers comprising one or more of the following amino acid sequences: LEVLFQGP (SEQ ID NO://), cleaved by PreScission protease (a fusion protein comprising human rhinovirus 3C protease and glutathione-S-transferase; Walker et al. (1994) Biotechnol. 12:601); a thrombin cleavage site, e.g., CGLVPAGSGP (SEQ ID NO://); SLLKSRMVPNFN (SEQ ID NO://) or SLLIARRMPNFN (SEQ ID NO://), cleaved by cathepsin B; SKLVQASASGVN (SEQ ID NO://) or SSYLKASDAPDN (SEQ ID NO://), cleaved by an Epstein-Barr virus protease; RPKPQQFFGLMN (SEQ ID NO://) cleaved by MMP-3 (stromelysin); SLRPLALWRSFN (SEQ ID NO://) cleaved by MMP-7 (matrilysin); SPQGIAGQRNFN (SEQ ID NO://) cleaved by MMP-9; DVDERDVRGFASFL SEQ ID NO://) cleaved by a thermolysin-like MMP; SLPLGLWAPNFN (SEQ ID NO://) cleaved by matrix metalloproteinase 2 (MMP-2); SLLIFRSWANFN (SEQ ID NO://) cleaved by cathespin L; SGVVIATVIVIT (SEQ ID NO://) cleaved by cathepsin D; SLGPQGIWGQFN (SEQ ID NO://) cleaved by matrix metalloproteinase 1 (MMP-1); KKSPGRVVGGSV (SEQ ID NO://) cleaved by urokinase-type plasminogen activator; PQGLLGAPGILG (SEQ ID NO://) cleaved by membrane type 1 matrixmetalloproteinase (MT-MMP); HGPEGLRVGFYESDVMGRGHARLVHVEEPHT (SEQ ID NO://) cleaved by stromelysin 3 (or MMP-11), thermolysin, fibroblast collagenase and stromelysin-1; GPQGLAGQRGIV (SEQ ID NO://) cleaved by matrix metalloproteinase 13 (collagenase-3); GGSGQRGRKALE (SEQ ID NO://) cleaved by tissue-type plasminogen activator (tPA); SLSALLSSDIFN (SEQ ID NO://) cleaved by human prostate-specific antigen; SLPRFKIIGGFN (SEQ ID NO://) cleaved by kallikrein (hK3); SLLGIAVPGNFN (SEQ ID NO://) cleaved by neutrophil elastase; and FFKNIVTPRTPP (SEQ ID NO://) cleaved by calpain (calcium activated neutral protease).

Suitable proteolytically cleavable linkers also include ENLYFQX (SEQ ID NO://; where X is any amino acid), ENLYFQS (SEQ ID NO://), ENLYFQG (SEQ ID NO://), ENLYFQY (SEQ ID NO://), ENLYFQL (SEQ ID NO://), ENLYFQW (SEQ ID NO://), ENLYFQM (SEQ ID NO://), ENLYFQH (SEQ ID NO://), ENLYFQN (SEQ ID NO://), ENLYFQA (SEQ ID NO://), and ENLYFQQ (SEQ ID NO://).

Suitable proteolytically cleavable linkers also include NS3 protease cleavage sites such as: DEVVECS (SEQ ID NO://), DEAEDVVECS (SEQ ID NO://), EDAAEEVVECS (SEQ ID NO://).

Suitable proteolytically cleavable linkers also include calpain cleavage site, where suitable calpain cleavage sites include, e.g., PLFAAR (SEQ ID NO://) and QQEVYGMMPRD (SEQ ID NO://).

In some cases, the proteolytically cleavable linker comprises an amino acid sequence that is substantially not cleaved by any endogenous protease in a given cell (e.g., a eukaryotic cell; e.g., a mammalian cell; e.g., a particular type of mammalian cell). In some cases, the proteolytically cleavable linker comprises an amino acid sequence that is cleaved by a viral protease, and that is substantially not cleaved by any endogenous protease in a given cell (e.g., a eukaryotic cell; e.g., a mammalian cell; e.g., a particular type of mammalian cell). In some cases, the proteolytically cleavable linker comprises an amino acid sequence that is cleaved by a non-naturally occurring (e.g., engineered) protease, and that is substantially not cleaved by any endogenous protease in a given cell (e.g., a eukaryotic cell; e.g., a mammalian cell; e.g., a particular type of mammalian cell).

In some cases, the proteolytically cleavable linker comprises an amino acid sequence that is cleaved by a protease that is endogenous to a given cell (e.g., a eukaryotic cell; e.g., a mammalian cell; e.g., a particular type of mammalian cell).

### Proteases

In some cases, the protease is a protease that is not normally produced in a particular cell; e.g., the protease is heterologous to the cell. For example, in some cases, the protease is one that is not normally produced in a mammalian cell. Examples of such proteases include viral proteases, insect-specific proteases, venom proteases, and the like.

In some cases, the protease is a protease that is normally produced in a particular cell; e.g., the protease is an endogenous protease (e.g., a calpain protease; etc.).

Suitable proteases include, but are not limited to, alanine carboxypeptidase, Armillaria mellea astacin, bacterial leucyl aminopeptidase, cancer procoagulant, cathepsin B, clostripain, cytosol alanyl aminopeptidase, elastase, endoproteinase Arg-C, enterokinase, gastricsin, gelatinase, Gly-X carboxypeptidase, glycyl endopeptidase, human rhinovirus 3C protease, hypodermin C, IgA-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2A, picornain 3C, proendopeptidase, prolyl aminopeptidase, proprotein convertase I, proprotein convertase II, russellysin, saccharopepsin, semenogelase, T-plasminogen activator, thrombin, tissue kallikrein, tobacco etch virus (TEV), togavirin, tryptophanyl aminopeptidase, U-plasminogen activator, Factor Xa, V8, venombin A, venombin AB, a calpain protease, and an Xaa-pro aminopeptidase.

Suitable proteases include a matrix metalloproteinase (MMP) (e.g., an MMP selected from collagenase-1, -2, and -3 (MMP-1, -8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP); a plasminogen activator (e.g., a uPA or a tissue plasminogen activator (tPA)). Another example of a suitable protease is prolactin. Another example of a suitable protease is a tobacco etch virus (TEV) protease. Another example of suitable protease is enterokinase. Another example of suitable protease is thrombin. Additional examples of suitable protease are: a PreScission protease (a fusion protein comprising human rhinovirus 3C protease and glutathione-S-transferase; Walker et al. (1994) Biotechnol. 12:601); cathepsin B; an Epstein-Barr virus protease; cathespin L; cathepsin D; thermolysin; kallikrein (hK3); neutrophil elastase; calpain (calcium activated neutral protease); and NS3 protease.

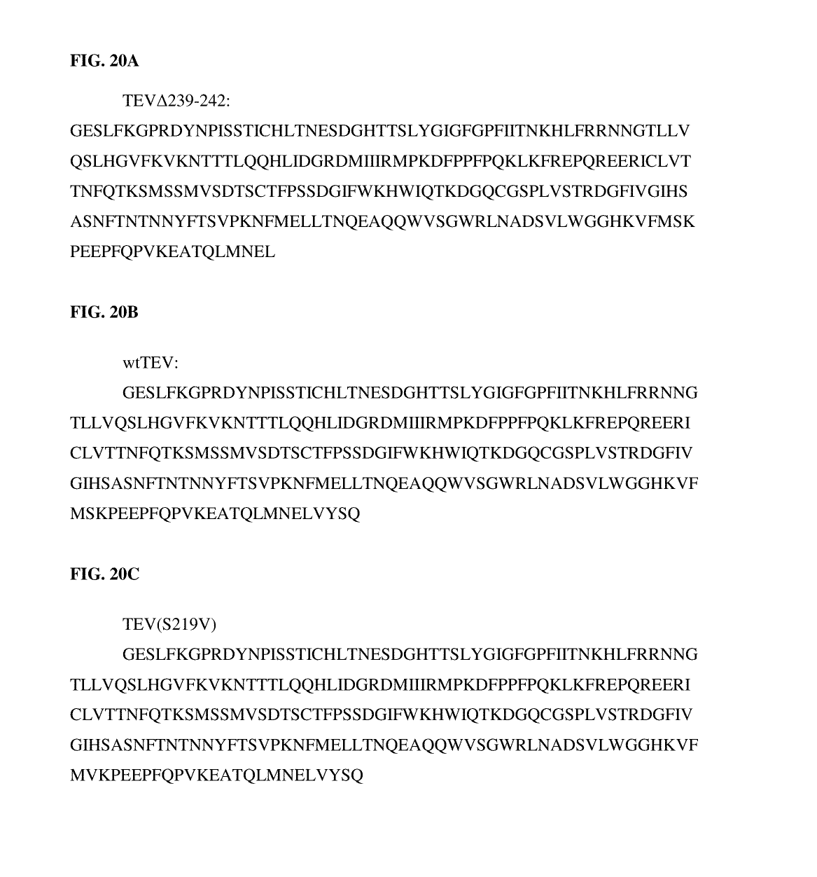

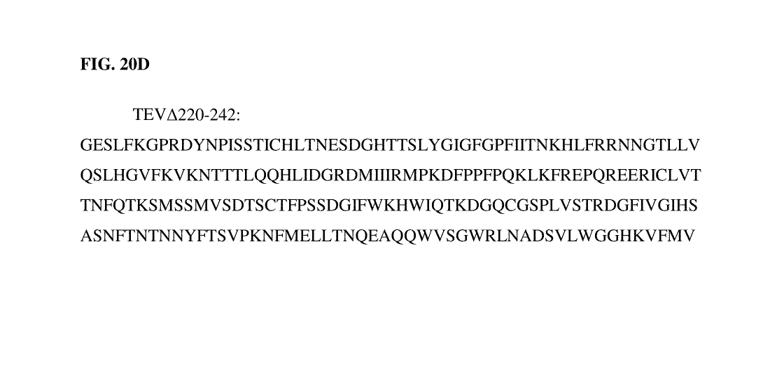

In some cases, a suitable protease is a TEV protease. In some cases, a suitable protease comprises an amino acid sequence having at least 80%, at least 85%, at least 90%, at least 95%, at least 98%, at least 99%, or 100%, amino acid sequence identity to the amino acid sequence depicted in FIG. 20A. In some cases, a suitable protease is a TEV protease. In some cases, a suitable protease comprises an amino acid sequence having at least 80%, at least 85%, at least 90%, at least 95%, at least 98%, at least 99%, or 100%, amino acid sequence identity to the amino acid sequence depicted in FIG. 20B. In some cases, a suitable protease is a TEV protease. In some cases, a suitable protease comprises an amino acid sequence having at least 80%, at least 85%, at least 90%, at least 95%, at least 98%, at least 99%, or 100%, amino acid sequence identity to the amino acid sequence depicted in FIG. 20C. In some cases, a suitable protease is a TEV protease. In some cases, a suitable protease comprises an amino acid sequence having at least 80%, at least 85%, at least 90%, at least 95%, at least 98%, at least 99%, or 100%, amino acid sequence identity to the amino acid sequence depicted in FIG. 20D.

In some cases, a suitable TEV protease comprises the amino acid sequence

(SEQ ID NO: //)
GESLFKGPRDYNPISSTICHLTNESDGHTTSLYGIGFGPFIITNKHL
FRRNNGTLLVQSLHGVFKVKNTTTLQQHLIDGRDMIIIRMPKDFPPF
PQKLKFREPQREERICLVTTNFQTKSMSSMVSDTSCTFPSSDGIFWK
HWIQTKDGQCGSPLVSTRDGFIVGIHSASNFTNTNNYFTSVPKNFME
LLTNQEAQQWVSGWRLNADSVLWGGHKVFMV.

A suitable TEV protease can have a length of from about 200 amino acids to about 250 amino acids. For example, a suitable TEV protease can have a length of from about 200 amino acids to about 220 amino acids, from about 220 amino acids to about 240 amino acids, or from about 240 amino acids to about 250 amino acids. For example, a suitable TEV protease can have a length of 219 amino acids, 242 amino acids, or 238 amino acids.

### System Comprising a Nucleic Acid Comprising a Nucleotide Sequence Encoding a Polypeptide of Interest

As noted above, a system of present disclosure includes a nucleic acid system (“System 2”) comprising: a) a first nucleic acid comprising a nucleotide sequence encoding a light-activated, calcium-gated fusion polypeptide comprising, in order from amino terminus to carboxyl terminus: i) a transmembrane domain; ii) a calmodulin-binding polypeptide or a troponin I polypeptide; iii) a LOV light-activated polypeptide comprising an amino acid sequence having at least 80% amino acid sequence identity to the amino acid sequence depicted in any one of FIG. 15A-15D; iv) a proteolytically cleavable linker; and v) a polypeptide of interest; and b) a second nucleic acid comprising a nucleotide sequence encoding a second fusion polypeptide comprising: i) a calcium-binding polypeptide selected from a calmodulin polypeptide and troponin C polypeptide; and ii) a protease that cleaves the proteolytically cleavable linker. Thus, in some cases, the present disclosure provides a nucleic acid system in which the first nucleic acid comprises a nucleotide sequence encoding a light-activated, calcium-gated fusion polypeptide that comprises a polypeptide of interest.

A system of present disclosure can include a nucleic acid system (“System 2”) comprising: a) a first nucleic acid comprising a nucleotide sequence encoding a light-activated, calcium-gated fusion polypeptide comprising, in order from amino terminus to carboxyl terminus: i) a transmembrane domain; ii) a calmodulin-binding polypeptide or a troponin I polypeptide; iii) a LOV light-activated polypeptide comprising an amino acid sequence having at least 80% amino acid sequence identity to the amino acid sequence depicted in any one of FIG. 15E-15G; iv) a proteolytically cleavable linker; and v) a polypeptide of interest; and b) a second nucleic acid comprising a nucleotide sequence encoding a second fusion polypeptide comprising: i) a calcium-binding polypeptide selected from a calmodulin polypeptide and troponin C polypeptide; and ii) a protease that cleaves the proteolytically cleavable linker. Thus, in some cases, the present disclosure provides a nucleic acid system in which the first nucleic acid comprises a nucleotide sequence encoding a light-activated, calcium-gated fusion polypeptide that comprises a polypeptide of interest.

### Polypeptides of Interest

Suitable polypeptides of interest that can be encoded in a system of the present disclosure include, but are not limited to, a reporter gene product, an opsin, a DREADD, a toxin, an enzyme, a transcription factor, an antibiotic resistance factor, a genome editing endonuclease, an RNA-guided endonuclease, a protease, a kinase, a phosphatase, a phosphorylase, a lipase, a receptor, an antibody, a fluorescent protein, a biotin ligase, a peroxidase such as APEX or APEX2, a base editing enzyme, a recombinase, a synaptic marker, a signaling protein, an effector protein of a receptor, a protein that regulates synaptic vesicle fusion or protein trafficking or organelle trafficking, a portion (e.g., a split half) of any one of the aforementioned polypeptides. In some cases, the gene product is inactive until released from the calcium-gated, light-activated polypeptide. In some cases, the gene product is a nuclear protein. In some cases, the gene product is a cytosolic protein. In some cases, the gene product is a mitochondrial protein. In some cases, the gene product is a transmembrane protein.

### Biotin Ligase

A suitable biotin ligase includes a BirA biotin-protein ligase polypeptide. A BirA biotin-protein ligase activates biotin to form biotinyl 5′ adenylate and transfers the biotin to a biotin-acceptor tag (BAT). A BAT can be present in a fusion protein, where the fusion protein comprises: a) a BAT; and b) a heterologous polypeptide. Suitable BATs include, e.g., GLNDIFEAQKIEWHE (SEQ ID NO://; see, e.g., Fairhead and Howarth (2015) Methods Mol. Biol. 1266:171).

A suitable BirA biotin-protein ligase polypeptide can comprise an amino acid sequence having at least at least 70%, at least 80%, at least 85%, at least 90%, at least 95%, at least 98%, at least 99%, or 100%, amino acid sequence identity to the following amino acid sequence:

(SEQ ID NO: //)
MKDNTVPLKL IALLANGEFH SGEQLGETLG MSRAAINKHI
QTLRDWGVDV FTVPGKGYSL PEPIQLLNAE EILSQLDGGS
VAVLPVIDST NQYLLDRIGE LKSGDACVAE YQQAGRGRRG
RKWFSPFGAN LYLSMFWRLE QGPAAAIGLS LVIGIVMAEV
LRKLGADKVR VKWPNDLYLQ DRKLAGILVE LTGKTGDAAQ
IVIGAGINMA MRRVEESVVN QGWITLQEAG INLDRNTLAA
MLIRELRAAL ELFEQEGLAP YLSRWEKLDN FINRPVKLII
GDKEIFGISR GIDKQGALLL EQDGIIKPWM GGEISLRSAE
K.

### Synaptic Markers

In some cases, a polypeptide of interest is a synaptic marker. Synaptic markers include, but are not limited to, PSD-95, SV2, homer, bassoon, synapsin I, synaptotagmin, synaptophysin, synaptobrevin, SAP102, α-adaptin, GluA1, NMDA receptor, LRRTM1, LRRTM2, SLITRK, neuroligin-1, neuroligin-2, gephyrin, GABA receptor, and the like.

### Nucleic Acid Editing Enzymes

In some cases, a polypeptide of interest is a nucleic acid-editing enzyme. Suitable nucleic acid-editing enzymes include, e.g., a DNA-editing enzyme, a cytidine deaminase, an adenosine deaminase, an apolipoprotein B mRNA-editing complex (APOBEC) family deaminase, an activation-induced cytidine deaminase (AID), an ACF1/ASE deaminase, and an ADAT family deaminase.

### Peroxidases

A suitable polypeptide of interest is in some cases a peroxidase, where suitable peroxidases include, e.g., horse radish peroxidase, yeast cytochrome c peroxidase (CCP), ascorbate peroxidase (APX), bacterial catalase-peroxidase (BCP), APEX, and APEX2. See, e.g., U.S. Patent Publication No. 2014/0206013.

An example of a suitable peroxidase is an APX, which has the following amino acid sequence: MGKSYPTVSA DYQKAVEKAK KKLRGFIAEK RCAPLMLRLA WHSAGTFDKG TKTGGPFGTI KHPAELAHSA NNGLDIAVRL LEPLKAEFPI LSYADFYQLA GVVAVEVTGG PEVPFHPGRE DKPEPPPEGR LPDATKGSDH LRDVFGKAMG LTDQDIVALS GGHTIGAAHK ERSGFEGPWT SNPLIFDNSY FTELLSGEKE GLLQLPSDKA LLSDPVFRPL VDKYAADEDA FFADYAEAHQ KLSELGFADA (SEQ ID NO://). In some cases, the peroxidase comprises a K14D substitution. In some cases, the peroxidase can contain a combination of (a) K14D, E112K, E228K, D229K, K14D/E112K, K14D/E228K, K14D/D229K, E17N/K20A/R21L, or K14D/W41F/E112K, and (b) S69F, G174F, W41F/S69F, D133A/T135F/K136F, W41F/D133A/T135F/K136F, S69F/D133A/T135F/K136F, or W41F/S69F/D133A/T135F/K136F. In some cases, the peroxidase can contain a combination of (a) single mutant K14D, single mutant E112K, single mutant E228K, single mutant D229K, double mutant K14D/E112K, double mutant K14D/E228K, double mutant K14D/D229K, triple mutant E17N/K20A/R21L, or triple mutant K14D/W41F/E112K, and (b) single mutant W41F, single mutant S69F, single mutant G174F, double mutant W41F/S69F, triple mutant D133A/T135F/K136F, quadruple mutant W41F/D133A/T135F/K136F, quadruple mutant S69F/D133A/T135F/K136F, or quintuple mutant W41F/S69F/D133A/T135F/K136F. Examples of such combined mutants include, but are not limited to, K14D/E112K/W41F (APEX), and K 14D/E112K/W41F/D133A/T135F/K136F. The amino acid numbering is based on the above-provided APX amino acid sequence.

### Antibodies

A suitable polypeptide of interest is in some cases an antibody. The terms “antibodies” and “immunoglobulin” include antibodies or immunoglobulins of any isotype, fragments of antibodies that retain specific binding to antigen, including, but not limited to, Fab, Fv, scFv, and Fd fragments, chimeric antibodies, humanized antibodies, single-chain antibodies (scAb), single domain antibodies (dAb), single domain heavy chain antibodies, a single domain light chain antibodies, nanobodies, bi-specific antibodies, multi-specific antibodies, and fusion proteins comprising an antigen-binding (also referred to herein as antigen binding) portion of an antibody and a non-antibody protein. Also encompassed by the term are Fab′, Fv, F(ab′)₂, and or other antibody fragments that retain specific binding to antigen, and monoclonal antibodies.

The term “nanobody” (Nb), as used herein, refers to the smallest antigen binding fragment or single variable domain (V_{HH}) derived from naturally occurring heavy chain antibody and is known to the person skilled in the art. They are derived from heavy chain only antibodies, seen in camelids (Hamers-Casterman et al., 1993; Desmyter et al., 1996). In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a V_{HH} antibody.

“Antibody fragments” comprise a portion of an intact antibody, for example, the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 (1995)); domain antibodies (dAb; Holt et al. (2003) Trends Biotechnol. 21:484); single-chain antibody molecules; and multi-specific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen. Antibody fragments include, e.g., scFv, sdAb, dAb, Fab, Fab′, Fab′₂, F(ab′)₂, Fd, Fv, Feb, and SMIP. An example of an sdAb is a camelid VHH.

“Fv” is the minimum antibody fragment that contains a complete antigen-recognition and -binding site. This region consists of a dimer of one heavy- and one light-chain variable domain in tight, non-covalent association. It is in this configuration that the three complementarity determining regions (CDRs) of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

“Single-chain Fv” or “sFv” or “scFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. (1993) Proc. Natl. Acad. Sci. USA 90:6444-6448.

### DREADDs

A suitable polypeptide of interest is in some cases a Designer Receptors Exclusively Activated by Designer Drugs (DREADD; also known as a “RASSL”). See e.g., Roth (2016) Neuron 89:683; Bang et al. (2016) Exp. Neurobiol. 25:205; Whissell et al. (2016) Front. Genet. 7:70; and U.S. Pat. No. 6,518,480. For example, a modified G protein-coupled receptor (GPCR) is genetically engineered so that it: 1) retains binding affinity for a synthetic small molecule; and 2) has decreased binding affinity for a selected naturally occurring peptide or nonpeptide ligand relative to binding by its corresponding wild-type GPCR (e.g., the GPCR from which the modified GPCR was derived). Synthetic small molecule binding to the modified receptor induces the target cell to respond with a specific physiological response (e.g., cellular proliferation, cellular secretion, cell migration, cell contraction, or pigment production).

Any G protein-coupled receptor having separable domains for: 1) natural ligand (e.g., a natural peptide ligand) binding; 2) synthetic small molecule binding; and 3) G protein interaction can be modified to produce a DREADD.

GPCRs that bind peptide as their natural ligand are in some cases used to generate a DREADD. Such GPCRs, include, but are not limited to: Type-1 Angiotensin II Receptor, Type-1a Angiotensin II Receptor, Type-1B Angiotensin II Receptor, Type-1C Angiotensin II Receptor, Type-2 Angiotensin II Receptor, Neuromedin-B Receptor, Gastrin-releasing Peptide Receptor, Bombesin Subtype-3 Receptor, B1 Bradykinin Receptor, B2 Bradykinin Receptor, Interleukin-8 A Receptor, Interleukin-8 B Receptor, FMet-Leu-Phe Receptor, Monocyte Chemoattractant Protein 1 Receptor, C-C Chemokine Receptor Type 1 Receptor, C5a Anaphylatoxin Receptor, Cholecystokinin Type A Receptor, Gastrin/cholecystokinin Type B Receptor, Endothelin-1 Receptor, Endothelin B Receptor, Follicle Stimulating Hormone (FSH-R) Receptor, Lutropin-choriogonadotropic Hormone (LH/CG-R) Receptor, Adrenocorticotropic Hormone Receptor (ACTH-R), Melanocyte Stimulating Hormone Receptor (MSH-R), Melanocortin-3 Receptor, Melanocortin-4 Receptor, Melanocortin-5 Receptor, Melatonin Type 1A Receptor, Melatonin Type 1B Receptor, Melatonin Type 1C Receptor, Neuropeptide Y Type 1 Receptor, Neuropeptide Y Type 2 Receptor, Neurotensin Receptor, Delta-type Opioid Receptor, Kappa-type Opioid Receptor, Mu-type Opioid, Nociceptin Receptor, Gonadotropin-releasing Hormone Receptor, Somatostatin Type 1 Receptor, Somatostatin Type 2 Receptor, Somatostatin Type 3 Receptor, Somatostatin Type 4 Receptor, Somatostatin Type 5 Receptor, Substance-P Receptor, Substance-K Receptor, Neuromedin K Receptor, Vasopressin Via Receptor, Vasopressin V1B Receptor, Vasopressin V2 Receptor, Oxytocin Receptor, Galanin Receptor, Calcitonin Receptor, Calcitonin A Receptor, Calcitonin B Receptor, Growth Hormone-releasing Hormone Receptor, Parathyroid Hormone/parathyroid Hormone-related Peptide Receptor, Pituitary Adenylate Cyclase Activating Polypeptide Type I Receptor, Secretin Receptor, Vasoactive Intestinal Polypeptide 1 Receptor, and Vasoactive Intestinal Polypeptide 2 Receptor.

A DREADD can interact with a G protein selected from Gi, Gq, and Gs. Thus, a DREADD can be a Gi-coupled DREADD, a Gq-coupled DREADD, or a Gs-coupled DREADD.

DREADDs include, but are not limited to, hM3Dq, a DREADD generated from the human M3 muscarinic receptor; hM4Di, a DREADD generated from the Gi-coupled human M4 muscarinic; a DREADD generated from a kappa opioid receptor (see U.S. Pat. No. 6,518,480); KORD; and the like.

### Transcription Factors

Suitable transcription factors include naturally-occurring transcription factors and recombinant (e.g., non-naturally occurring, engineered, artificial, synthetic) transcription factors. In some cases, the transcription is a transcriptional activator. In some cases, the transcriptional activator is an engineered protein, such as a zinc finger or TALE based DNA binding domain fused to an effector domain such as VP64 (transcriptional activation).

A transcription factor can comprise: i) a DNA binding domain (DBD); and ii) an activation domain (AD). The DBD can be any DBD with a known response element, including synthetic and chimeric DNA binding domains, or analogs, combinations, or modifications thereof. Suitable DNA binding domains include, but are not limited to, a GAL4 DBD, a LexA DBD, a transcription factor DBD, a Group H nuclear receptor member DBD, a steroid/thyroid hormone nuclear receptor superfamily member DBD, a bacterial LacZ DBD, an EcR DBD, a GALA DBD, and a LexA DBD. Suitable ADs include, but are not limited to, a Group H nuclear receptor member AD, a steroid/thyroid hormone nuclear receptor AD, a CJ7 AD, a p65-TA1 AD, a synthetic or chimeric AD, a polyglutamine AD, a basic or acidic amino acid AD, a VP16 AD, a GAL4 AD, an NF-κB AD, a BP64 AD, a B42 acidic activation domain (B42AD), a p65 transactivation domain (p65AD), SAD, NF-1, AP-2, SP1-A, SP1-B, Oct-1, Oct-2, MTF-1, BTEB-2, and LKLF, or an analog, combination, or modification thereof.

Suitable transcription factors include transcriptional activators, where suitable transcriptional activators include, but are not limited to, GAL4-VP16, GAL5-VP64, Tbx21, tTA-VP16, VP16, VP64, GAL4, p65, LexA-VP16, GAL4-NFκB, and the like.

Suitable transcription factors include transcriptional repressors, where suitable transcriptional repressors (e.g., a transcription repressor domain) include, but are not limited to, Krüppel-associated box (KRAB); the Mad mSIN3 interaction domain (SID); the ERF repressor domain (ERD); MDB-2B; v-ErbA; MBD3; and the like.

### Reporter Gene Products

Suitable reporter gene products include polypeptides that generate a detectable signal. Suitable detectable signal-producing proteins include, e.g., fluorescent proteins; enzymes that catalyze a reaction that generates a detectable signal as a product; and the like.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrape1, mRaspberry, mGrape2, mPlum (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, is suitable for use.

Suitable enzymes include, but are not limited to, horse radish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, glucose oxidase (GO), and the like.

### Genome-Editing Endonuclease

A “genome editing endonuclease” is an endonuclease, e.g., sequence-specific endonuclease, which can be used for the editing of a cell's genome (e.g., by cleaving at a targeted location within the cell's genomic DNA). Examples of genome editing endonucleases include but are not limited to: (i) Zinc finger nucleases, (ii) TAL endonucleases, and (iii) CRISPR/Cas endonucleases. Examples of CRISPR/Cas endonucleases include class 2 CRISPR/Cas endonucleases such as: (a) type II CRISPR/Cas proteins, e.g., a Cas9 protein; (b) type V CRISPR/Cas proteins, e.g., a Cpf1 polypeptide, a C2c1 polypeptide, a C2c3 polypeptide, and the like; and (c) type VI CRISPR/Cas proteins, e.g., a C2c2 polypeptide.

Examples of suitable sequence-specific, e.g., genome editing, endonucleases include, but are not limited to, zinc finger nucleases, meganucleases, TAL-effector DNA binding domain-nuclease fusion proteins (transcription activator-like effector nucleases (TALEN®s)), and CRISPR/Cas endonucleases (e.g., class 2 CRISPR/Cas endonucleases such as a type II, type V, or type VI CRISPR/Cas endonucleases). Thus, in some cases, a gene product is a sequence-specific genome editing endonuclease, e.g., genome editing, endonucleases selected from: a zinc finger nuclease, a TAL-effector DNA binding domain-nuclease fusion protein (TALEN), and a CRISPR/Cas endonuclease (e.g., a class 2 CRISPR/Cas endonuclease such as a type II, type V, or type VI CRISPR/Cas endonuclease). In some cases, a sequence-specific genome editing endonuclease includes a zinc finger nuclease or a TALEN. In some cases, a sequence-specific genome editing endonuclease includes a class 2 CRISPR/Cas endonuclease. In some cases, a sequence-specific genome editing endonuclease includes a class 2 type II CRISPR/Cas endonuclease (e.g., a Cas9 protein). In some cases, a sequence-specific genome editing endonuclease includes a class 2 type V CRISPR/Cas endonuclease (e.g., a Cpf1 protein, a C2c1 protein, or a C2c3 protein). In some cases, a sequence-specific genome editing endonuclease includes a class 2 type VI CRISPR/Cas endonuclease (e.g., a C2c2 protein).

RNA-mediated adaptive immune systems in bacteria and archaea rely on Clustered Regularly Interspaced Short Palindromic Repeat (CRISPR) genomic loci and CRISPR-associated (Cas) proteins that function together to provide protection from invading viruses and plasmids. In some cases, an RNA-guided endonuclease is a class 2 CRISPR/Cas endonuclease. In class 2 CRISPR systems, the functions of the effector complex (e.g., the cleavage of target DNA) are carried out by a single endonuclease (e.g., see Zetsche et al, Cell. 2015 Oct. 22; 163(3):759-71; Makarova et al, Nat Rev Microbiol. 2015 November; 13(11):722-36; and Shmakov et al., Mol Cell. 2015 Nov. 5; 60(3):385-97). As such, the term “class 2 CRISPR/Cas protein” is used herein to encompass the endonuclease (the target nucleic acid cleaving protein) from class 2 CRISPR systems. Thus, the term “class 2 CRISPR/Cas endonuclease” as used herein encompasses type II CRISPR/Cas proteins (e.g., Cas9), type V CRISPR/Cas proteins (e.g., Cpf1, C2c1, C2C3), and type VI CRISPR/Cas proteins (e.g., C2c2). To date, class 2 CRISPR/Cas proteins encompass type II, type V, and type VI CRISPR/Cas proteins, but the term is also meant to encompass any class 2 CRISPR/Cas protein suitable for binding to a corresponding guide RNA and forming an RNP complex.

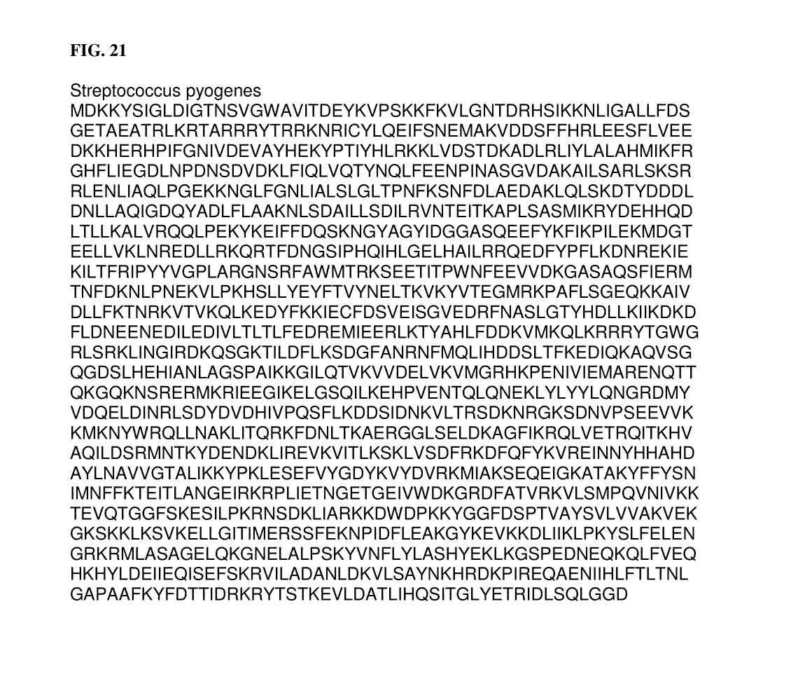

In some cases, a suitable RNA-guided endonuclease comprises an amino acid sequence having at least 30%, at least 40%, at least 50%, at least 60%, at least 70%, at least 80%, at least 85%, at least 90%, at least 95%, at least 98%, at least 99%, or 100%, amino acid sequence identity to the Streptococcus pyogenes Cas9 amino acid sequence depicted in FIG. 21.

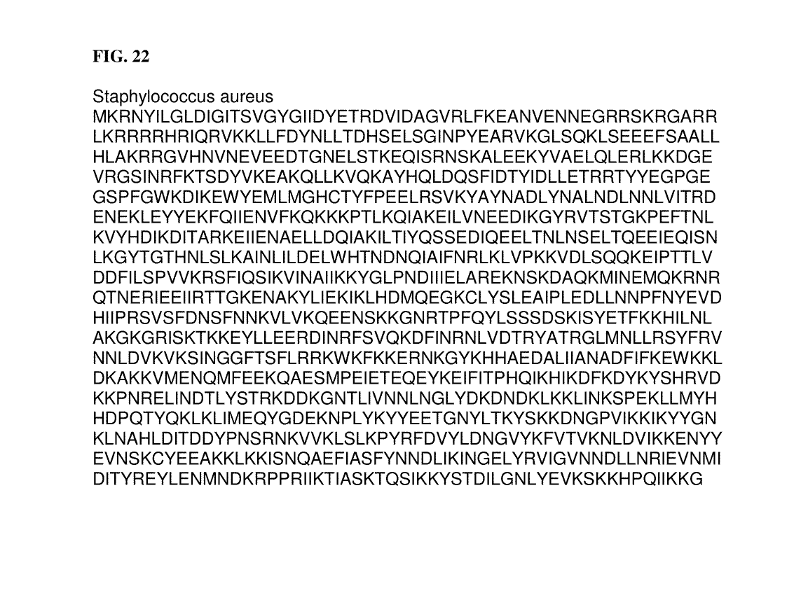

In some cases, a suitable RNA-guided endonuclease comprises an amino acid sequence having at least 30%, at least 40%, at least 50%, at least 60%, at least 70%, at least 80%, at least 85%, at least 90%, at least 95%, at least 98%, at least 99%, or 100%, amino acid sequence identity to the Staphylococcus aureus Cas9 amino acid sequence depicted in FIG. 22.

In some cases, the RNA-guided endonuclease is a nickase. Jinek et al., Science. 2012 Aug. 17; 337(6096):816-21).

In some cases, the RNA-guided endonuclease is a variant Cas9 protein that has reduced catalytic activity (e.g., when a Cas9 protein has a D10, G12, G17, E762, H840, N854, N863, H982, H983, A984, D986, and/or a A987 mutation of the amino acid sequence depicted in FIG. 21, e.g., D10A, G12A, G17A, E762A, H840A, N854A, N863A, H982A, H983A, A984A, and/or D986A); and the variant Cas9 protein retains the ability to bind to target nucleic acid in a site-specific manner (e.g., when complexed with a guide RNA.

In some cases, the RNA-guided endonuclease is a type V CRISPR/Cas protein. In some cases, the RNA-guided endonuclease is a type VI CRISPR/Cas protein. Examples and guidance related to type V and type VI CRISPR/Cas proteins (e.g., Cpf1, C2c1, C2c2, and C2c3 guide RNAs) can be found in the art, for example, see Zetsche et al, Cell. 2015 Oct. 22; 163(3):759-71; Makarova et al, Nat Rev Microbiol. 2015 November; 13(11):722-36; and Shmakov et al., Mol Cell. 2015 Nov. 5; 60(3):385-97.

In some cases, the RNA-guided endonuclease is a chimeric polypeptide (e.g., a fusion polypeptide) comprising: a) an RNA-guided endonuclease; and b) a fusion partner, where the fusion partner provides a functionality or activity other than an endonuclease activity. For example, the fusion partner can be a polypeptide having an enzymatic activity that modifies a polypeptide (e.g., a histone) associated with, or proximal to, a target nucleic acid (e.g., methyltransferase activity, deaminase activity (e.g., cytidine deaminase activity), demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity).

In some cases, the RNA-guided endonuclease is a base editor; for example, in some cases, the RNA-guided endonuclease is a fusion polypeptide comprising: a) an RNA-guided endonuclease; and b) a cytidine deaminase. See, e.g., Komor et al. (2016) Nature 533:420.

### Opsins

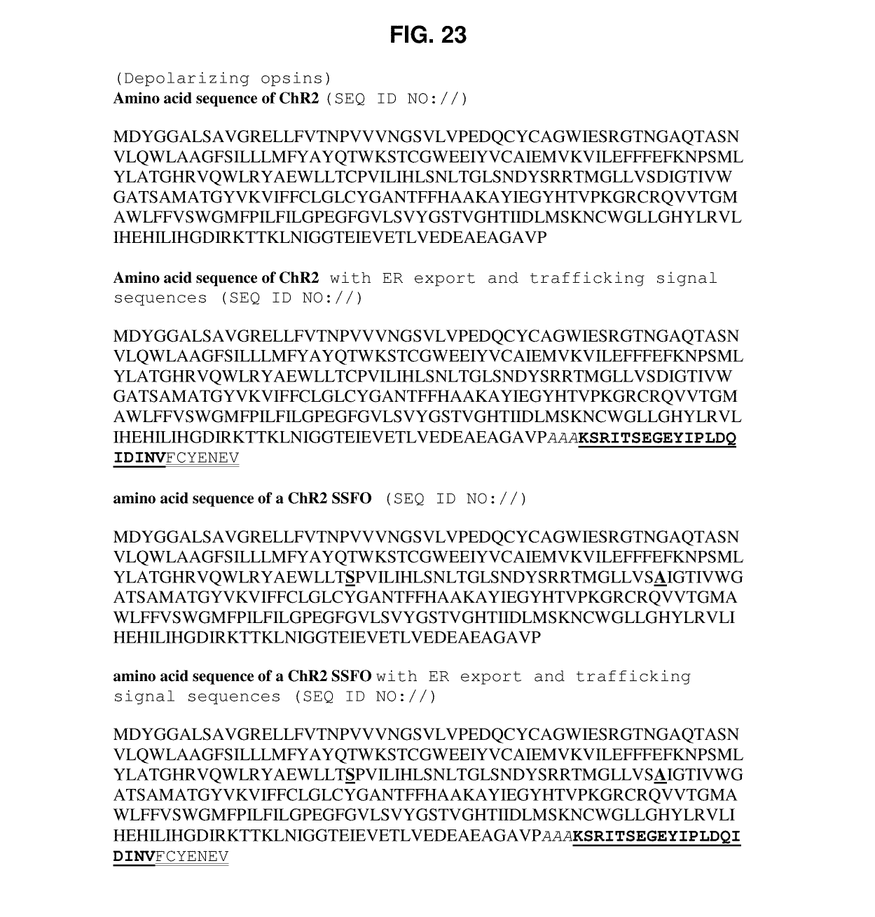

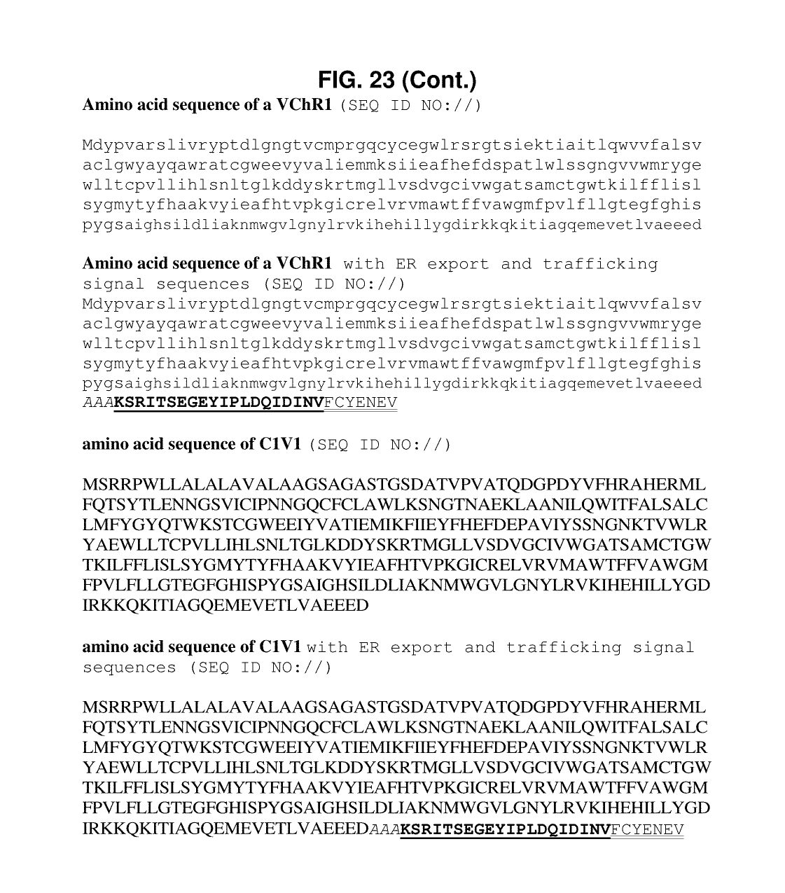

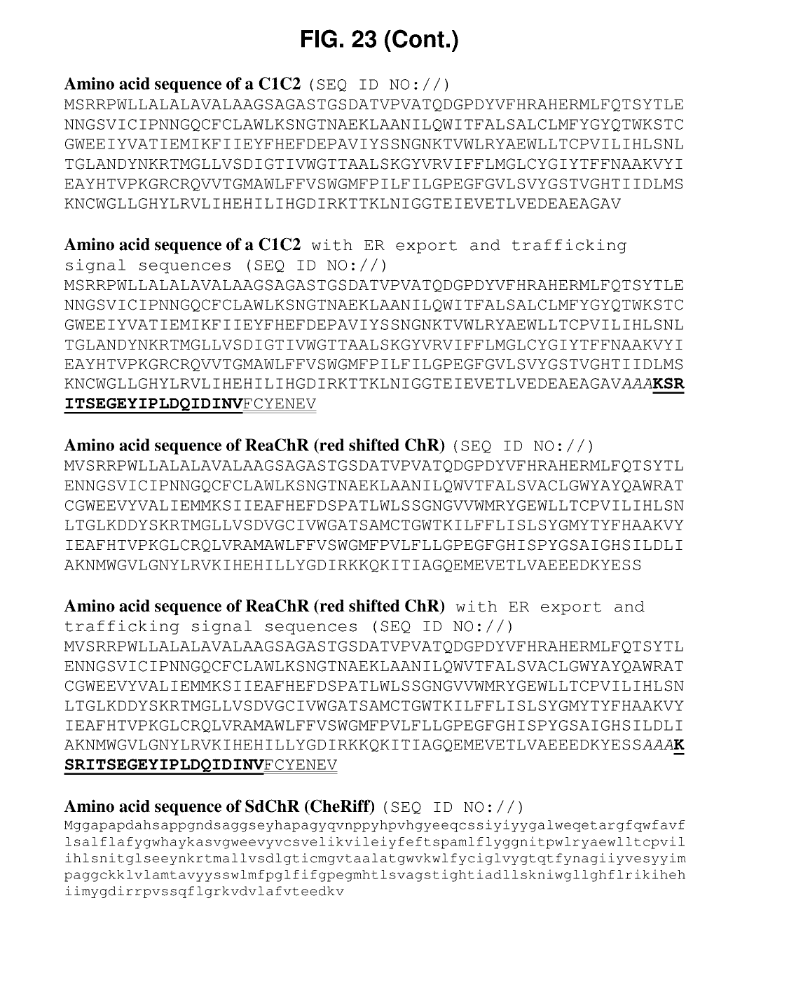

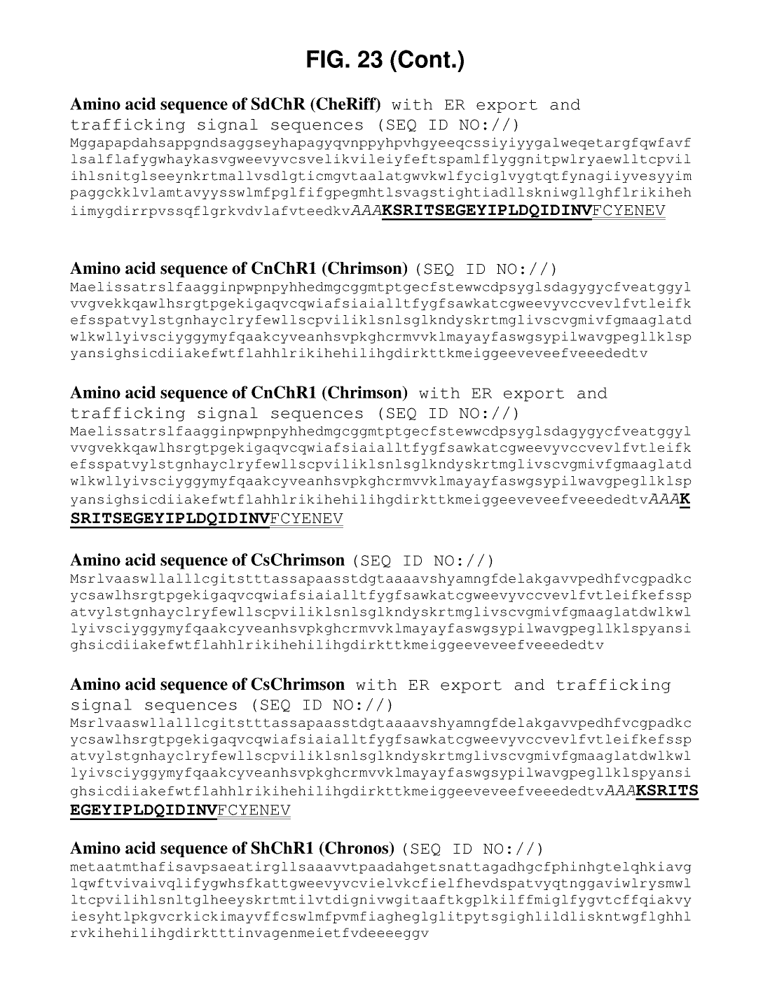

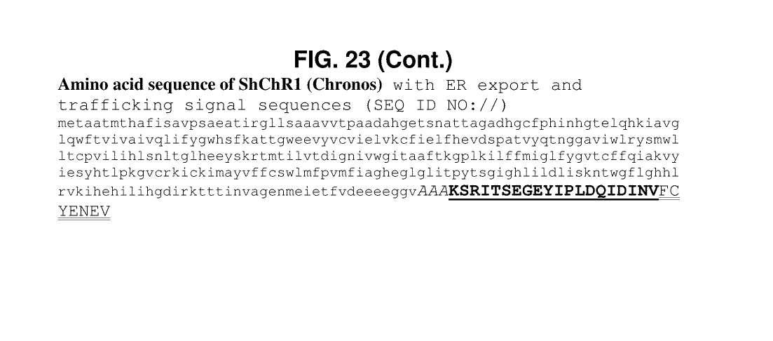

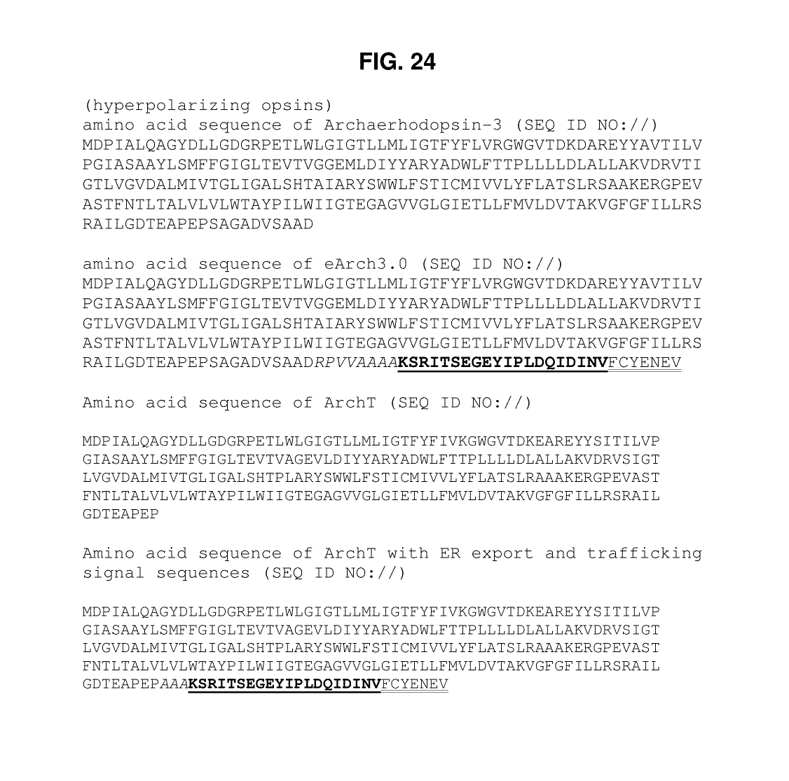

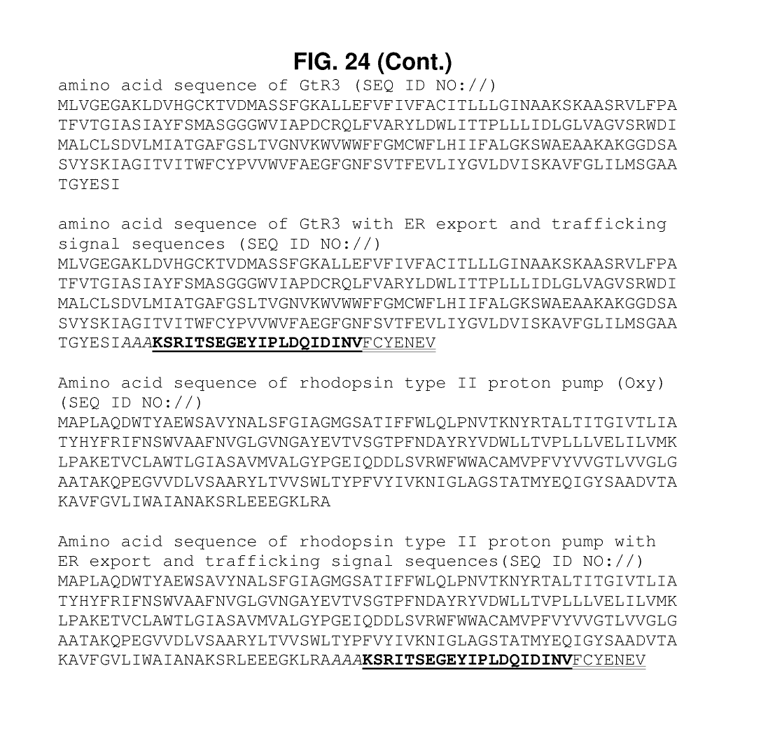

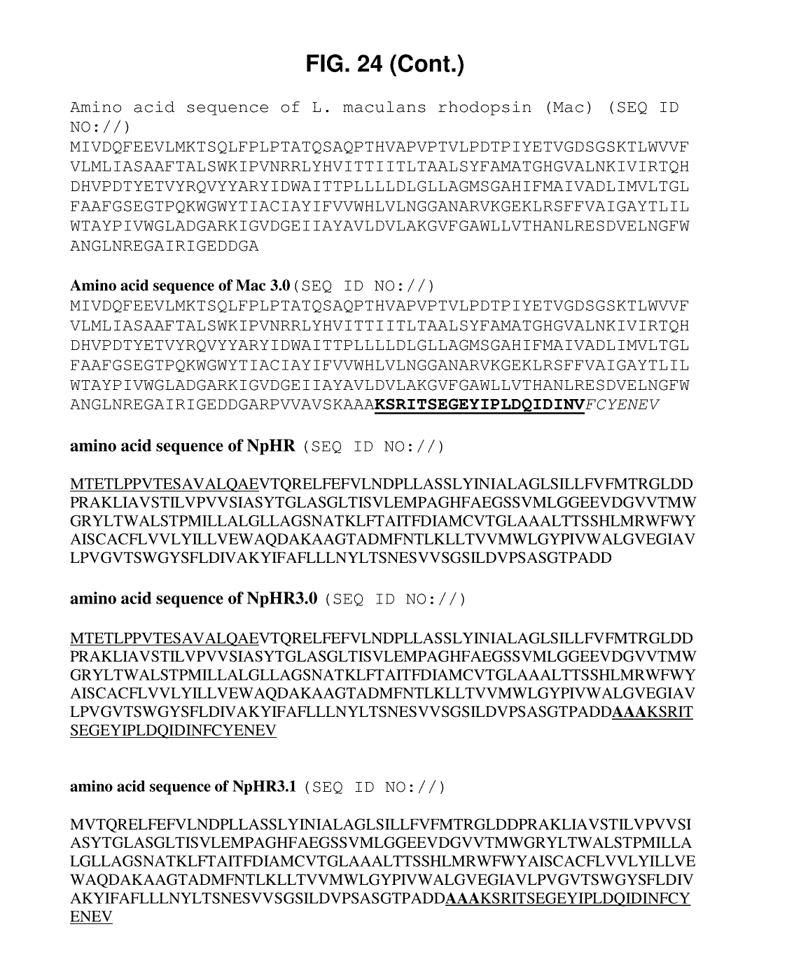

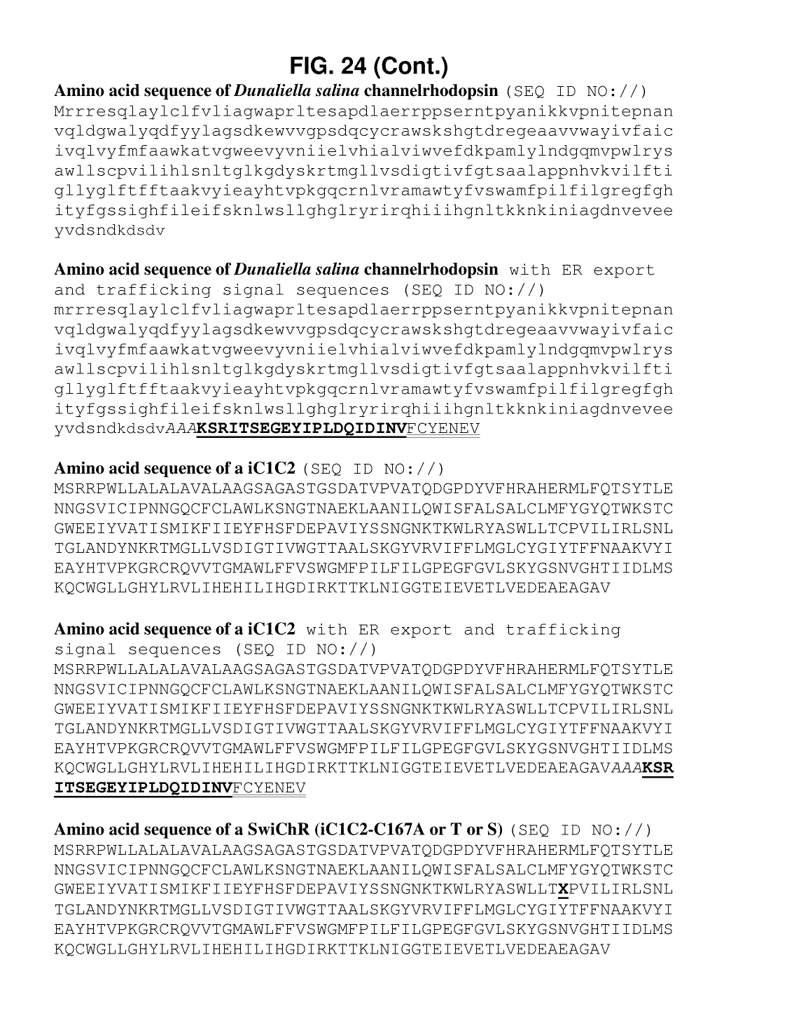

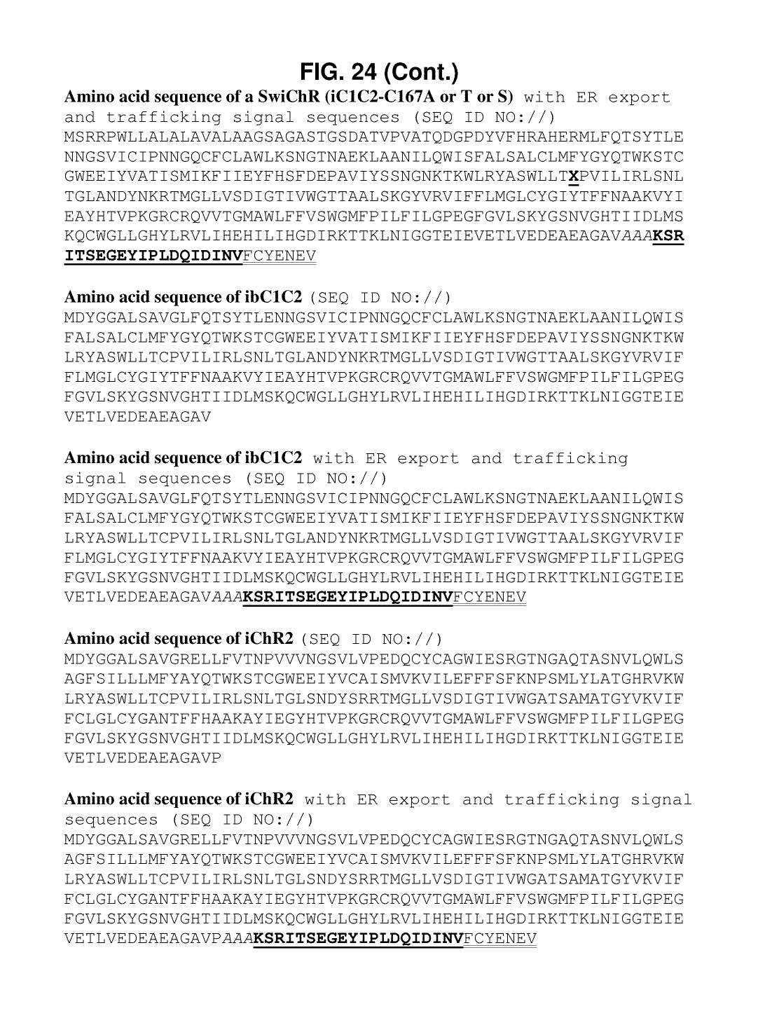

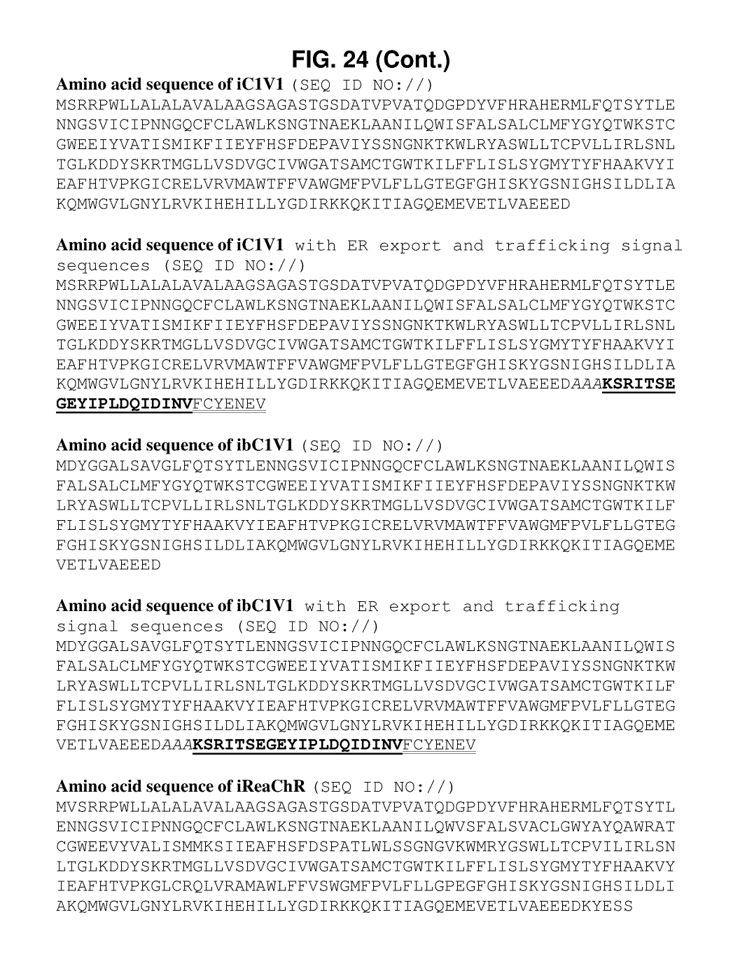

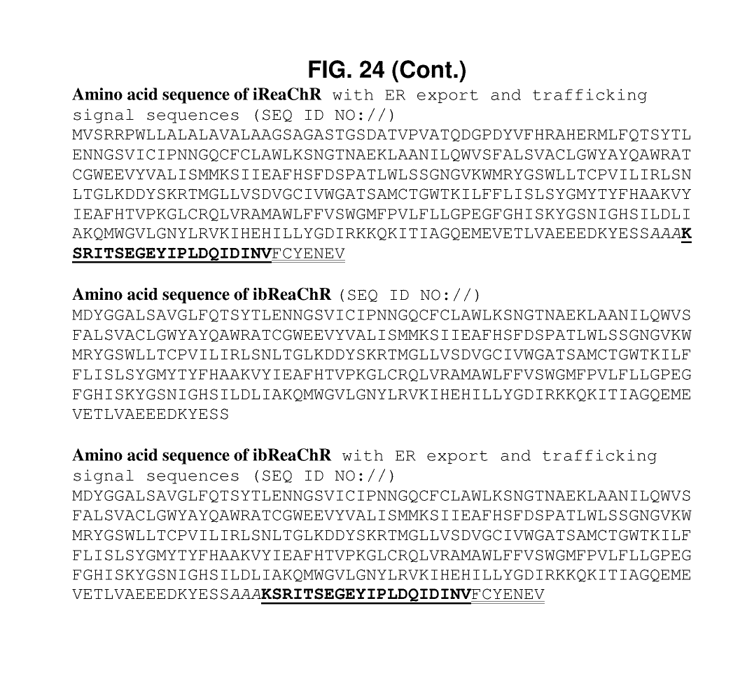

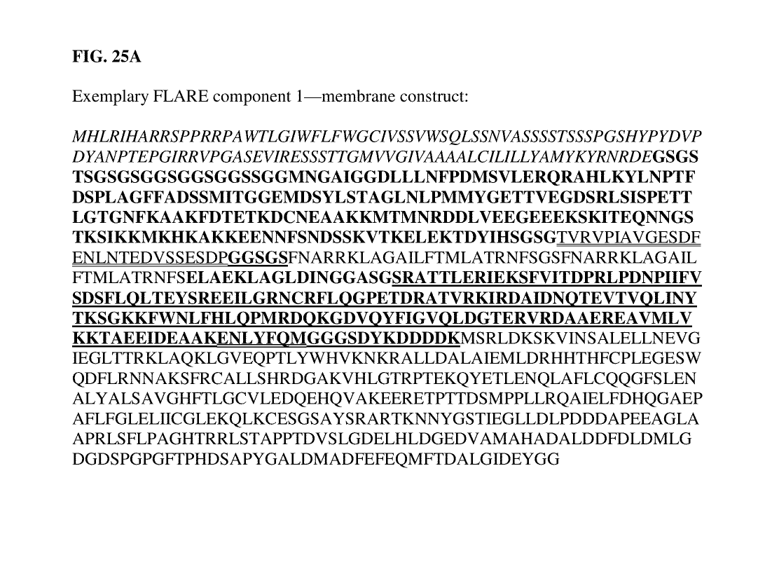

In some cases, a gene product encoded in a system of the present disclosure is a hyperpolarizing or a depolarizing light-activated polypeptide (an “opsin”). The light-activated polypeptide may be a light-activated ion channel or a light-activated ion pump. The light-activated ion channel polypeptides are adapted to allow one or more ions to pass through the plasma membrane of a neuron when the polypeptide is illuminated with light of an activating wavelength. Light-activated proteins may be characterized as ion pump proteins, which facilitate the passage of a small number of ions through the plasma membrane per photon of light, or as ion channel proteins, which allow a stream of ions to freely flow through the plasma membrane when the channel is open. In some embodiments, the light-activated polypeptide depolarizes the neuron when activated by light of an activating wavelength. Suitable depolarizing light-activated polypeptides, without limitation, are shown in FIG. 23. In some embodiments, the light-activated polypeptide hyperpolarizes the neuron when activated by light of an activating wavelength. Suitable hyperpolarizing light-activated polypeptides, without limitation, are shown in FIG. 24.

In some cases, a light-activated polypeptide comprises an amino acid sequence having at least 80%, at least 85%, at least 90%, at least 95%, at least 98%, at least 99%, or 100%, amino acid sequence identity to an opsin amino acid sequence depicted in FIG. 23. In some cases, a light-activated polypeptide comprises an amino acid sequence having at least 80%, at least 85%, at least 90%, at least 95%, at least 98%, at least 99%, or 100%, amino acid sequence identity to an opsin amino acid sequence depicted in FIG. 24.

In some embodiments, the light-activated polypeptides are activated by blue light. In some embodiments, the light-activated polypeptides are activated by green light. In some embodiments, the light-activated polypeptides are activated by yellow light. In some embodiments, the light-activated polypeptides are activated by orange light. In some embodiments, the light-activated polypeptides are activated by red light.

In some embodiments, the light-activated polypeptide expressed in a cell can be fused to one or more amino acid sequence motifs selected from the group consisting of a signal peptide, an endoplasmic reticulum (ER) export signal, a membrane trafficking signal, and/or an N-terminal golgi export signal. The one or more amino acid sequence motifs which enhance light-activated protein transport to the plasma membranes of mammalian cells can be fused to the N-terminus, the C-terminus, or to both the N- and C-terminal ends of the light-activated polypeptide. In some cases, the one or more amino acid sequence motifs which enhance light-activated polypeptide transport to the plasma membranes of mammalian cells is fused internally within a light-activated polypeptide. Optionally, the light-activated polypeptide and the one or more amino acid sequence motifs may be separated by a linker.

In some embodiments, the light-activated polypeptide can be modified by the addition of a trafficking signal (ts) which enhances transport of the protein to the cell plasma membrane. In some embodiments, the trafficking signal can be derived from the amino acid sequence of the human inward rectifier potassium channel Kir2.1. In other embodiments, the trafficking signal can comprise the amino acid sequence KSRITSEGEYIPLDQIDINV (SEQ ID NO:56). Trafficking sequences that are suitable for use can comprise an amino acid sequence having at least 85%, 90%, 91%, 92%, 93%, 94%, 95%, 96%, 97%, 98%, 99%, or 100%, amino acid sequence identity to an amino acid sequence such a trafficking sequence of human inward rectifier potassium channel Kir2.1 (e.g., KSRITSEGEYIPLDQIDINV (SEQ ID NO:56)).

A trafficking sequence can have a length of from about 10 amino acids to about 50 amino acids, e.g., from about 10 amino acids to about 20 amino acids, from about 20 amino acids to about 30 amino acids, from about 30 amino acids to about 40 amino acids, or from about 40 amino acids to about 50 amino acids.

ER export sequences that are suitable for use with a light-activated polypeptide include, e.g., VXXSL (where X is any amino acid; SEQ ID NO:52) (e.g., VKESL (SEQ ID NO:53); VLGSL (SEQ ID NO:54); etc.); NANSFCYENEVALTSK (SEQ ID NO:55); FXYENE (SEQ ID NO:57) (where X is any amino acid), e.g., FCYENEV (SEQ ID NO:58); and the like. An ER export sequence can have a length of from about 5 amino acids to about 25 amino acids, e.g., from about 5 amino acids to about 10 amino acids, from about 10 amino acids to about 15 amino acids, from about 15 amino acids to about 20 amino acids, or from about 20 amino acids to about 25 amino acids.

In some cases, a light-activated polypeptide is a fusion polypeptide that comprises an endoplasmic reticulum (ER) export signal (e.g., FCYENEV). In some cases, a light-activated polypeptide is a fusion polypeptide that comprises a membrane trafficking signal (e.g., KSRITSEGEYIPLDQIDINV). In some cases, a light-activated polypeptide is a fusion polypeptide comprising, in order from N-terminus to C-terminus: a) a light-activated polypeptide comprising an amino acid sequence having at least 80%, at least 85%, at least 90%, at least 95%, at least 98%, at least 99%, or 100%, amino acid sequence identity to an opsin amino acid sequence depicted in FIG. 23 or FIG. 24; b) an ER export signal; and c) a membrane trafficking signal.

### Toxins

Suitable toxins include polypeptide toxins present in a natural source (e.g., naturally-occurring), recombinantly produced toxins, and synthetically produced toxins. Suitable toxins include ribosome inactivating proteins (RIPs); a bacterial toxin; and the like.

Suitable toxins include, e.g., anthopleurin B (GVPCLCDSDG-PRPRGNTLSG-ILWFYPSGCP-SGWHNCKAHG-PNIGWCCKK; SEQ ID NO://), anthopleurin C, anthopleurin Q, calitoxin (MKTQVLALFV LCVLFCLAES RTTLNKRNDI EKRIECKCEG DAPDLSHMTG TVYFSCKGGD GSWSKCNTYT AVADCCHQA; SEQ ID NO://), a conotoxin, ectatomin, HsTx1, omega-atracotoxin, a raventoxin, a scorpion toxin, and the like.

Suitable bacterial toxins include, e.g., cholera toxin, botulinum toxin, diphtheria toxin (produced by Corynebacterium diphtheriae), tetanospasmin, an enterotoxin, hemolysin, shiga toxin, erythrogenic toxin, adenylate cyclase toxin, pertussis toxin, ST toxin, LT toxin, ricin, abrin, tetanus toxin, and the like.

Exemplary Type I RIPS include, but are not limited to, gelonin, dodecandrin, tricosanthin, tricokirin, bryodin, Mirabilis antiviral protein (MAP), barley ribosome-inactivating protein (BRIP), pokeweed antiviral proteins (PAPS), saporins, luffins, and momordins. Exemplary Type II RIPS include, but are not limited to, ricin and abrin.

### Antibiotic Resistance Factors

As noted above, in some cases, the gene product of interest is an antibiotic resistance factor, e.g., a polypeptide that confers antibiotic resistance to a cell that produces the polypeptide.

Suitable antibiotic resistance factors include, but are not limited to, polypeptides that confer resistance to kanamycin, gentamicin, rifampin, trimethoprim, chloramphenicol, tetracycline, penicillin, methicillin, blasticidin, puromycin, hygromycin, or other antimicrobial agent. Suitable antibiotic resistance factors include, but are not limited to, aminoglycoside acetyltransferases, rifampin ADP-ribosyltransferases, dihydrofolate reductases, transporters, β-lactamases, chloramphenicol acetyltransferases, and efflux pumps. See, e.g., McGarvey et al. (2012) Applied Environ. Microbiol. 78:1708. Suitable antibiotic resistance factors include, but are not limited to, aminoglycoside 6′-N-acetyltransferase; gentamycin 3′-N-acetyltransferase; rifampin ADP-ribosyltransferase; dihydrofolate reductase; MFS transporter; ABC transporter; blasticidin-S deaminase; blasticidin acetyltransferase; puromycin N-acetyl-transferease; hygromycin kinase; and the like.

### Recombinases

In some cases, the gene product of interest is a recombinase. The term “recombinase” refers to an enzyme that catalyzes DNA exchange at a specific target site, for example, a palindromic sequence, by excision/insertion, inversion, translocation, and exchange.

Suitable recombinases include, but are not limited to, Cre recombinase; a FLP recombinase; a Tel recombinase; and the like. A suitable recombinase is one that targets (and cleaves) a target site selected from a telRL site, a loxP site, a phi pK02 telRL site, an FRT site, phiC31 attP site, and λattP site.

A suitable recombinase can be selected from the group consisting of: TelN; Tel; Tel (gp26 K02 phage); Cre; Flp; phiC31; Int; and a lambdoid phage integrase (e.g. a phi 80 recombinase, a HK022 recombinase; an HP1 recombinase).

Examples of target sites for such recombinases include, e.g.: a telRL site (targeted by a TelN recombinase): TATCAGCACACAATTGCCCATTATACGCGCGTATAATGGACTAT TGTGTGCTGA (SEQ ID NO://); a pal site: ACCTATTTCAGCATACTACGCGCGTAGTATGCTGAAATAGGT (SEQ ID NO://); a phi K02 telRL site: CCATTATACGCGCGTATAATGG (SEQ ID NO://); a loxP site (targeted by a Cre recombinase): TAACTTCGTATAGCATACATTATACGAAGTTAT (SEQ ID NO://); a FRT site (targeted by a Flp recombinase): GAAGTTCCTATTCTCTAGAAAGTATAGGAACTTC (SEQ ID NO://); a phiC31 attP site (targeted by a phiC31 recombinase): CCCAGGTCAGAAGCGGTTTTCGGGAGTAGTGCCCCAACTGGGGT AACCTTTGAGTTCTCTCAGTTGGGGGCGTAGGGTCGCCGACAYGA CACAAGGGGTT (SEQ ID NO://); a λ attP site: TGATAGTGACCTGTTCGTTTGCAACACATTGATGAGCAATGCTT TTTTATAATGCCAACTTTGTACAAAAAAGCTGAACGAGAAACGTA AAATGATATAAA (SEQ ID NO://).

### Additional Amino Acid Sequences

In some cases, the gene product is a fusion polypeptide comprising a fusion partner, where the fusion partner can be, e.g., a soma localization signal, a nuclear localization signal, a protein transduction domain, a mitochondrial localization signal, a chloroplast localization signal, an endoplasmic reticulum retention signal, an epitope tag, etc. For example, a suitable mitochondrial localization sequence is LGRVIPRKIASRASLM (SEQ ID NO://); or MSVLTPLLLRGLTGSARRLPVPRAKIHSLL (SEQ ID NO://).

### Soma Localization Signal

In some cases, the transcription factor includes a soma localization signal. For example, a 66 amino acid C-terminal sequence of Kv2.1 or a 27 amino acid sequence of Nav1.6 induces localization to the soma of a neuron. For example, the Nav1.6 soma localization signal comprises the amino acid sequence: TVRVPIAVGESDFENLNTEDVSSESDP (SEQ ID NO://).

### Nuclear Localization Signals

Non-limiting examples of NLSs include an NLS sequence derived from: the NLS of the SV40 virus large T-antigen, having the amino acid sequence PKKKRKV (SEQ ID NO://); the NLS from nucleoplasmin (e.g. the nucleoplasmin bipartite NLS with the sequence KRPAATKKAGQAKKKK (SEQ ID NO://)); the c-myc NLS having the amino acid sequence PAAKRVKLD (SEQ ID NO://) or RQRRNELKRSP (SEQ ID NO://); the hRNPA1 M9 NLS having the sequence NQSSNFGPMKGGNFGGRSSGPYGGGGQYFAKPRNQGGY (SEQ ID NO://); the sequence RMRIZFKNKGKDTAELRRRRVEVSVELRKAKKDEQILKRRNV (SEQ ID NO://) of the IBB domain from importin-alpha; the sequences VSRKRPRP (SEQ ID NO://) and PPKKARED (SEQ ID NO://) of the myoma T protein; the sequence PQPKKKPL (SEQ ID NO://) of human p53; the sequence SALIKKKKKMAP (SEQ ID NO://) of mouse c-abl IV; the sequences DRLRR (SEQ ID NO://) and PKQKKRK (SEQ ID NO://) of the influenza virus NS1; the sequence RKLKKKIKKL (SEQ ID NO://) of the Hepatitis virus delta antigen; the sequence REKKKFLKRR (SEQ ID NO://) of the mouse Mx1 protein; the sequence KRKGDEVDGVDEVAKKKSKK (SEQ ID NO://) of the human poly(ADP-ribose) polymerase; and the sequence RKCLQAGMNLEARKTKK (SEQ ID NO://) of the steroid hormone receptors (human) glucocorticoid.

A gene product can include a “Protein Transduction Domain” or PTD (also known as a CPP—cell penetrating peptide), which refers to a polypeptide that facilitates traversing a lipid bilayer, micelle, cell membrane, organelle membrane, or vesicle membrane. A PTD attached to another polypeptide (a polypeptide gene product of interest) facilitates the polypeptide traversing a membrane, for example going from extracellular space to intracellular space, or cytosol to within an organelle. In some cases, a PTD attached to a polypeptide gene product of interest facilitates entry of the polypeptide into the nucleus (e.g., in some cases, a PTD includes a nuclear localization signal). In some cases, a PTD is covalently linked to the amino terminus of a polypeptide gene product of interest. In some cases, a PTD is covalently linked to the carboxyl terminus of a polypeptide gene product of interest. In some cases, a PTD is covalently linked to the amino terminus and to the carboxyl terminus of a polypeptide gene product of interest. Exemplary PTDs include but are not limited to a minimal undecapeptide protein transduction domain (corresponding to residues 47-57 of HIV-1 TAT comprising YGRKKRRQRRR; SEQ ID NO://); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); an Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO://); Transportan GWTLNSAGYLLGKINLKALAALAKKIL (SEQ ID NO://); KALAWEAKLAKALAKALAKHLAKALAKALKCEA (SEQ ID NO://); and RQIKIWFQNRRMKWKK (SEQ ID NO://). Exemplary PTDs include but are not limited to, YGRKKRRQRRR (SEQ ID NO://), RKKRRQRRR (SEQ ID NO://); an arginine homopolymer of from 3 arginine residues to 50 arginine residues; Exemplary PTD domain amino acid sequences include, but are not limited to, any of the following: YGRKKRRQRRR (SEQ ID NO://); RKKRRQRR (SEQ ID NO://); YARAAARQARA (SEQ ID NO://); THRLPRRRRRR (SEQ ID NO://); and GGRRARRRRRR (SEQ ID NO://).

### Nucleic Acids

As noted above, a nucleic acid system of the present disclosure (e.g., System 1; System 2; as described above) comprises two nucleic acids.

In some cases, the nucleotide sequence encoding the light-activated, calcium-gated fusion polypeptide and/or the nucleotide sequence encoding the second fusion polypeptide (the second fusion polypeptide comprising a calmodulin polypeptide or a troponin C polypeptide fused to a protease) is operably linked to a transcriptional control element (e.g., a promoter; an enhancer; etc.). In some cases, the transcriptional control element is inducible. In some cases, the transcriptional control element is constitutive. In some cases, the promoters are functional in eukaryotic cells. In some cases, the promoters are cell type-specific promoters. In some cases, the promoters are tissue-specific promoters. In some cases, the promoter to which the nucleotide sequence encoding the light-activated, calcium-gated fusion polypeptide is operably linked, and the promoter to which the nucleotide sequence encoding the second fusion polypeptide is operably linked, are substantially the same. In other cases, the promoter to which the nucleotide sequence encoding the light-activated, calcium-gated fusion polypeptide is operably linked is different from the promoter to which the nucleotide sequence encoding the second fusion polypeptide is operably linked.

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein.), it may be a spatially restricted promoter (i.e., transcriptional control element, enhancer, etc.)(e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

Suitable promoter and enhancer elements are known in the art. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue-specific promoters. Suitable promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31(17)), a human H1 promoter (H1), and the like.

Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

Inducible promoters suitable for use include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some cases, the promoter is a neuron-specific promoter. Suitable neuron-specific control sequences include, but are not limited to, a neuron-specific enolase (NSE) promoter (see, e.g., EMBL HSENO2, X51956; see also, e.g., U.S. Pat. No. 6,649,811, U.S. Pat. No. 5,387,742); an aromatic amino acid decarboxylase (AADC) promoter; a neurofilament promoter (see, e.g., GenBank HUMNFL, L04147); a synapsin promoter (see, e.g., GenBank HUMSYNIB, M55301); a thy-1 promoter (see, e.g., Chen et al. (1987) Cell 51:7-19; and Llewellyn et al. (2010) Nat. Med. 16:1161); a serotonin receptor promoter (see, e.g., GenBank S62283); a tyrosine hydroxylase promoter (TH) (see, e.g., Nucl. Acids. Res. 15:2363-2384 (1987) and Neuron 6:583-594 (1991)); a GnRH promoter (see, e.g., Radovick et al., Proc. Natl. Acad. Sci. USA 88:3402-3406 (1991)); an L7 promoter (see, e.g., Oberdick et al., Science 248:223-226 (1990)); a DNMT promoter (see, e.g., Bartge et al., Proc. Natl. Acad. Sci. USA 85:3648-3652 (1988)); an enkephalin promoter (see, e.g., Comb et al., EMBO J. 17:3793-3805 (1988)); a myelin basic protein (MBP) promoter; a CMV enhancer/platelet-derived growth factor-β promoter (see, e.g., Liu et al. (2004) Gene Therapy 11:52-60); a motor neuron-specific gene Hb9 promoter (see, e.g., U.S. Pat. No. 7,632,679; and Lee et al. (2004) Development 131:3295-3306); and an alpha subunit of Ca(²⁺)-calmodulin-dependent protein kinase II (CaMKIIα) promoter (see, e.g., Mayford et al. (1996) Proc. Natl. Acad. Sci. USA 93:13250). Other suitable promoters include elongation factor (EF) 1α and dopamine transporter (DAT) promoters.

In some cases, a nucleic acid of a system of the present disclosure is a recombinant expression vector. In some cases, the recombinant expression vector is a viral construct, e.g., a recombinant adeno-associated virus (AAV) construct, a recombinant adenoviral construct, a recombinant lentiviral construct, a recombinant retroviral construct, etc. In some cases, a nucleic acid of a system of the present disclosure is a recombinant lentivirus vector. In some cases, a nucleic acid of a system of the present disclosure is a recombinant AAV vector.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., Hum Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. In some cases, the vector is a lentivirus vector. Also suitable are transposon-mediated vectors, such as piggyback and sleeping beauty vectors.

In some cases, a nucleic acid system of the present disclosure is packaged in a viral particle. For example, in some cases, the nucleic acids of a nucleic acid system of the present disclosure are recombinant AAV vectors, and are packaged in recombinant AAV particles. Thus, the present disclosure provides a recombinant viral particle comprising a nucleic acid system of the present disclosure.

### Genetically Modified Host Cells

The present disclosure provides a genetically modified host cell (e.g., an in vitro genetically modified host cell) comprising a nucleic acid system of the present disclosure. In some cases, one or both of the first and the second nucleic acid of a nucleic acid system of the present disclosure is stably integrated into the genome of the host cell. In some instances, one or both of the first and the second nucleic acid of a nucleic acid system of the present disclosure is present episomally in the genetically modified host cell.

In some cases, the genetically modified host cell is a primary (non-immortalized) cell. In some cases, the genetically modified host cell is an immortalized cell line.

Suitable host cells include mammalian cells, insect cells, reptile cells, amphibian cells, arachnid cells, plant cells, bacterial cells, archaeal cells, yeast cells, algal cells, fungal cells, and the like.

In some cases, the genetically modified host cell is a mammalian cell, e.g., a human cell, a non-human primate cell, a rodent cell, a feline (e.g., a cat) cell, a canine (e.g., a dog) cell, an ungulate cell, an equine (e.g., a horse) cell, an ovine cell, a caprine cell, a bovine cell, etc. In some cases, the genetically modified host cell is a rodent cell (e.g., a rat cell; a mouse cell). In some cases, the genetically modified host cell is a human cell. In some cases, the genetically modified host cell is a non-human primate cell.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

Suitable host cells include cells of, e.g., Bacteria (e.g., Eubacteria); Archaebacteria; Protista; Fungi; Plantae; and Animalia. Suitable host cells include cells of plant-like members of the kingdom Protista, including, but not limited to, algae (e.g., green algae, red algae, glaucophytes, cyanobacteria); fungus-like members of Protista, e.g., slime molds, water molds, etc.; animal-like members of Protista, e.g., flagellates (e.g., Euglena), amoeboids (e.g., amoeba), sporozoans (e.g, Apicomplexa, Myxozoa, Microsporidia), and ciliates (e.g., Paramecium). Suitable host cells include cells of members of the kingdom Fungi, including, but not limited to, members of any of the phyla: Basidiomycota (club fungi; e.g., members of Agaricus, Amanita, Boletus, Cantherellus, etc.); Ascomycota (sac fungi, including, e.g., Saccharomyces); Mycophycophyta (lichens); Zygomycota (conjugation fungi); and Deuteromycota. Suitable host cells include cells of members of the kingdom Plantae, including, but not limited to, members of any of the following divisions: Bryophyta (e.g., mosses), Anthocerotophyta (e.g., hornworts), Hepaticophyta (e.g., liverworts), Lycophyta (e.g., club mosses), Sphenophyta (e.g., horsetails), Psilophyta (e.g., whisk ferns), Ophioglossophyta, Pterophyta (e.g., ferns), Cycadophyta, Gingkophyta, Pinophyta, Gnetophyta, and Magnoliophyta (e.g., flowering plants). Suitable host cells include cells of members of the kingdom Animalia, including, but not limited to, members of any of the following phyla: Porifera (sponges); Placozoa; Orthonectida (parasites of marine invertebrates); Rhombozoa; Cnidaria (corals, anemones, jellyfish, sea pens, sea pansies, sea wasps); Ctenophora (comb jellies); Platyhelminthes (flatworms); Nemertina (ribbon worms); Ngathostomulida (jawed worms)p Gastrotricha; Rotifera; Priapulida; Kinorhyncha; Loricifera; Acanthocephala; Entoprocta; Nemotoda; Nematomorpha; Cycliophora; Mollusca (mollusks); Sipuncula (peanut worms); Annelida (segmented worms); Tardigrada (water bears); Onychophora (velvet worms); Arthropoda (including the subphyla: Chelicerata, Myriapoda, Hexapoda, and Crustacea, where the Chelicerata include, e.g., arachnids, Merostomata, and Pycnogonida, where the Myriapoda include, e.g., Chilopoda (centipedes), Diplopoda (millipedes), Paropoda, and Symphyla, where the Hexapoda include insects, and where the Crustacea include shrimp, krill, barnacles, etc.; Phoronida; Ectoprocta (moss animals); Brachiopoda; Echinodermata (e.g. starfish, sea daisies, feather stars, sea urchins, sea cucumbers, brittle stars, brittle baskets, etc.); Chaetognatha (arrow worms); Hemichordata (acorn worms); and Chordata. Suitable members of Chordata include any member of the following subphyla: Urochordata (sea squirts; including Ascidiacea, Thaliacea, and Larvacea); Cephalochordata (lancelets); Myxini (hagfish); and Vertebrata, where members of Vertebrata include, e.g., members of Petromyzontida (lampreys), Chondrichthyces (cartilaginous fish), Actinopterygii (ray-finned fish), Actinista (coelocanths), Dipnoi (lungfish), Reptilia (reptiles, e.g., snakes, alligators, crocodiles, lizards, etc.), Aves (birds); and Mammalian (mammals). Suitable plant cells include cells of any monocotyledon and cells of any dicotyledon. Plant cells include, e.g., a cell of a leaf, a root, a tuber, a flower, and the like. In some cases, the genetically modified host cell is a plant cell. In some cases, the genetically modified host cell is a bacterial cell. In some cases, the genetically modified host cell is an archaeal cell.

Suitable eukaryotic host cells include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, Chlamydomonas reinhardtii, and the like. In some cases, subject genetically modified host cell is a yeast cell. In some instances, the yeast cell is Saccharomyces cerevisiae.

Suitable prokaryotic cells include any of a variety of bacteria, including laboratory bacterial strains, pathogenic bacteria, etc. Suitable prokaryotic hosts include, but are not limited, to any of a variety of gram-positive, gram-negative, or gram-variable bacteria. Examples include, but are not limited to, cells belonging to the genera: Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Phormidium, Pseudomonas, Rhodobacter, Rhodopseudomonas, Rhodospirillum, Rhodococcus, Salmonella, Scenedesmun, Serratia, Shigella, Staphylococcus, Strepromyces, Synnecoccus, and Zymomonas. Examples of prokaryotic strains include, but are not limited to: Bacillus subtilis, Bacillus amyloliquefacines, Brevibacterium ammoniagenes, Brevibacterium immariophilum, Clostridium beigerinckii, Enterobacter sakazakii, Escherichia coli, Lactococcus lactis, Mesorhizobium loti, Pseudomonas aeruginosa, Pseudomonas mevalonii, Pseudomonas pudica, Rhodobacter capsulatus, Rhodobacter sphaeroides, Rhodospirillum rubrum, Salmonella enterica, Salmonella typhi, Salmonella typhimurium, Shigella dysenteriae, Shigella flexneri, Shigella sonnei, and Staphylococcus aureus. One example of a suitable bacterial host cell is Escherichia coli cell.

Suitable plant cells include cells of a monocotyledon; cells of a dicotyledon; cells of an angiosperm; cells of a gymnosperm; etc.

### System for Light-Activated, Calcium-Gated Transcription Control

The present disclosure provides a system (a “FLARE” system) for light-activated, calcium-gated transcriptional control of expression of a target gene product. A FLARE system of the present disclosure in some cases comprises 3 components: 1) a first fusion polypeptide comprising: a) a calcium-binding polypeptide; and b) a protease; 2) a second fusion polypeptide comprising: a) a transmembrane domain; b) a polypeptide that binds the calcium-binding polypeptide under certain Ca²⁺ concentration conditions (e.g., a Ca²⁺ concentration above about 100 nM); c) a light-activated polypeptide comprising a LOV domain; d) a proteolytically cleavable linker that is caged by the light-activated polypeptide in the absence of blue light; and e) a transcription factor; and 3) a construct that comprises: a) a promoter that is activated by the transcription factor; and b) a nucleotide sequence encoding a gene product of interest, where the nucleotide sequence is operably linked to the promoter. Each of these components is described in detail below. In some cases, a FLARE system of the present disclosure comprises one of the above-mentioned components. In some cases, a FLARE system of the present disclosure comprises two of the above-mentioned components.

The present disclosure provides one or more nucleic acids comprising nucleotide sequences encoding one or more components of a FLARE system of the present disclosure, as well as genetically modified host cells comprising the one or more nucleic acids.

Thus, the present disclosure provides a system comprising: 1) a first fusion polypeptide comprising: a) a calcium-binding polypeptide selected from a calmodulin polypeptide and a troponin C polypeptide; and b) a protease; 2) a second fusion polypeptide comprising: a) a transmembrane domain; b) a polypeptide that binds the calcium-binding polypeptide under certain Ca²⁺ concentration conditions (e.g., a Ca²⁺ concentration above about 100 nM); c) a light-activated polypeptide comprising a LOV domain; d) a proteolytically cleavable linker that is caged by the light-activated polypeptide in the absence of blue light; and e) a transcription factor. The present disclosure provides a nucleic acid system comprising: 1) a first nucleic acid comprising a nucleotide sequence encoding the first fusion polypeptide; and 2) a second nucleic acid comprising a nucleotide sequence encoding the second fusion polypeptide. In some cases, the system comprises a genetically modified host cell, where the host cell is genetically modified with a nucleotide sequence encoding a gene product of interest, where the nucleotide sequence is operably linked to a promoter that is controlled by the transcription factor.

The present disclosure provides a system comprising: a nucleic acid comprising: a) a nucleotide sequence encoding a fusion polypeptide comprising: i) a transmembrane domain; ii) calmodulin-binding polypeptide or a troponin I polypeptide that binds calmodulin or troponin C, respectively, under certain Ca²⁺ concentration conditions (e.g., a Ca²⁺ concentration above about 100 nM); ii) a light-activated polypeptide comprising a LOV domain; and iii) a proteolytically cleavable linker that is caged by the light-activated polypeptide in the absence of blue light; and b) an insertion site for inserting a nucleic acid comprising a nucleotide sequence encoding a transcription factor.

### Fusion Polypeptide Comprising a Calcium-Binding Protein and a Protease

As noted above, a component of a FLARE system of the present disclosure can include a fusion polypeptide comprising: a) a calcium-binding polypeptide selected from a calmodulin polypeptide and a troponin C polypeptide; and b) a protease.

### Calmodulin

A suitable calmodulin polypeptide comprises an amino acid sequence having at least 80%, at least 85%, at least 90%, at least 95%, at least 98%, at least 99%, or 100%, amino acid sequence identity to the following calmodulin amino acid sequence: MDQLTEEQIAEFKEAFSLFDKDGDGTITTKELGTVMRSLGQNPTEAELQDMINEVDADG DGTIDFPEFLTMMARKMKYTDSEEEIREAFRVFDKDGNGYISAAELRHVMTNLGEKLTD EEVDEMIREADIDGDGQVNYEEFVQMMTAK (SEQ ID NO://); and has a length of from about 148 amino acids to about 160 amino acids. In some cases, the calmodulin polypeptide has a length of 148 amino acids.

In some cases, a suitable calmodulin polypeptide comprises an amino acid sequence having at least 80%, at least 85%, at least 90%, at least 95%, at least 98%, at least 99%, or 100%, amino acid sequence identity to the following calmodulin amino acid sequence: MDQLTEEQIAEFKEAFSLFDKDGDGTITTKELGTVMRSLGQNPTEAELQDMINEVDADG DGTIDFPEFLTMMARKMKYTDSEEEIREAFRVFDKDGNGYISAAELRHVMTNLGEKLTD EEVDEMIREADIDGDGQVNYEEFVQMMTAK (SEQ ID NO://); and has a substitution of F19; and has a length of from about 148 amino acids to about 160 amino acids. In some cases, the calmodulin polypeptide has a length of 148 amino acids. In some cases, the F19 substitution is an F19L substitution, an F19I substitution, an F19V substitution, or an F19A substitution.

In some cases, a suitable calmodulin polypeptide comprises an amino acid sequence having at least 80%, at least 85%, at least 90%, at least 95%, at least 98%, at least 99%, or 100%, amino acid sequence identity to the following calmodulin amino acid sequence: MDQLTEEQIAEFKEAFSLFDKDGDGTITTKELGTVMRSLGQNPTEAELQDMINEVDADG DGTIDFPEFLTMMARKMKYTDSEEEIREAFRVFDKDGNGYISAAELRHVMTNLGEKLTD EEVDEMIREADIDGDGQVNYEEFVQMMTAK (SEQ ID NO://); and has a substitution of V35; and has a length of from about 148 amino acids to about 160 amino acids. In some cases, the calmodulin polypeptide has a length of 148 amino acids. In some cases, the V35 substitution is a V35G substitution, a V35A substitution, a V35L substitution, or a V35I substitution.

In some cases, a suitable calmodulin polypeptide comprises an amino acid sequence having at least 80%, at least 85%, at least 90%, at least 95%, at least 98%, at least 99%, or 100%, amino acid sequence identity to the following calmodulin amino acid sequence: MDQLTEEQIAEFKEAFSLFDKDGDGTITTKELGTVMRSLGQNPTEAELQDMINEVDADG DGTIDFPEFLTMMARKMKYTDSEEEIREAFRVFDKDGNGYISAAELRHVMTNLGEKLTD EEVDEMIREADIDGDGQVNYEEFVQMMTAK (SEQ ID NO://); and has an F19 substitution (e.g., an F19L substitution, an F19I substitution, an F19V substitution, or an F19A substitution) and a V35 substitution (e.g., a V35G substitution, a V35A substitution, a V35L substitution, or a V35I substitution); and has a length of from about 148 amino acids to about 160 amino acids. In some cases, the calmodulin polypeptide has a length of 148 amino acids.

In some cases, a suitable calmodulin polypeptide comprises an amino acid sequence having at least 80%, at least 85%, at least 90%, at least 95%, at least 98%, at least 99%, or 100%, amino acid sequence identity to the following calmodulin amino acid sequence: MDQLTEEQIAEFKEAFSLLDKDGDGTITTKELGTGMRSLGQNPTEAELQDMINEVDADG DGTIDFPEFLTMMARKMKYTDSEEEIREAFRVFDKDGNGYISAAELRHVMTNLGEKLTD EEVDEMIREADIDGDGQVNYEEFVQMMTAK (SEQ ID NO://); and comprises a Leu at amino acid 19 and a Gly at amino acid 35; and has a length of from about 148 amino acids to about 160 amino acids. In some cases, the calmodulin polypeptide has a length of 148 amino acids.

### Troponin C

A suitable troponin C polypeptide comprises an amino acid sequence having at least 80%, at least 85%, at least 90%, at least 95%, at least 98%, at least 99%, or 100%, amino acid sequence identity to the following troponin C amino acid sequence: MTDQQAEARS YLSEEMIAEF KAAFDMFDAD GGGDISVKEL GTVMRMLGQT PTKEELDAII EEVDEDGSGT IDFEEFLVMM VRQMKEDAKG KSEEELAECF RIFDRNADGY IDPGELAEIF RASGEHVTDE EIESLMKDGD KNNDGRIDFD EFLKMMEGVQ (SEQ ID NO://).

A suitable troponin C polypeptide can have a length of from about 100 amino acids to about 175 amino acids, e.g., from about 100 amino acids to about 125 amino acids, from about 125 amino acids to about 150 amino acids, or from about 150 amino acids to about 175 amino acids.

A suitable troponin C polypeptide comprises an amino acid sequence having at least 80%, at least 85%, at least 90%, at least 95%, at least 98%, at least 99%, or 100%, amino acid sequence identity to the following troponin C amino acid sequence: MTDQQAEARSYLSEEMIAEFKAAFDMFDADGGGDISVKELGTVMRMLGQTPTKEELD AIIEEVDEDGSGTIDFEEFLVMMVRQMKEDAKGKSEEELAECFRIFDRDANGYIDAEELA EIFRASGEHVTDEEIESLMKDGDKNNDGRIDFDEFLKMMEGVQ (SEQ ID NO://; and has a length of from about 160 amino acids to about 175 amino acids (e.g., from about 160 amino acids to about 165 amino acids, from about 165 amino acids to about 170 amino acids, or from about 170 amino acids to about 175 amino acids. In some cases, a suitable troponin C polypeptide comprises the amino acid sequence: MTDQQAEARSYLSEEMIAEFKAAFDMFDADGGGDISVKELGTVMRMLGQTPTKEELD AIIEEVDEDGSGTIDFEEFLVMMVRQMKEDAKGKSEEELAECFRIFDRDANGYIDAEELA EIFRASGEHVTDEEIESLMKDGDKNNDGRIDFDEFLKMMEGVQ (SEQ ID NO://; and has a length of 160 amino acids.

### Proteases

In some cases, the protease is a protease that is not normally produced in a particular cell; e.g., the protease is heterologous to the cell. For example, in some cases, the protease is one that is not normally produced in a mammalian cell. Examples of such proteases include viral proteases, insect-specific proteases, and the like.

In some cases, the protease is a protease that is normally produced in a particular cell; e.g., the protease is an endogenous protease.

Suitable proteases include, but are not limited to, alanine carboxypeptidase, Armillaria mellea astacin, bacterial leucyl aminopeptidase, cancer procoagulant, cathepsin B, clostripain, cytosol alanyl aminopeptidase, elastase, endoproteinase Arg-C, enterokinase, gastricsin, gelatinase, Gly-X carboxypeptidase, glycyl endopeptidase, human rhinovirus 3C protease, hypodermin C, IgA-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2A, picornain 3C, proendopeptidase, prolyl aminopeptidase, proprotein convertase I, proprotein convertase II, russellysin, saccharopepsin, semenogelase, T-plasminogen activator, thrombin, tissue kallikrein, tobacco etch virus (TEV), togavirin, tryptophanyl aminopeptidase, U-plasminogen activator, V8, venombin A, venombin AB, and Xaa-pro aminopeptidase.

Suitable proteases include a matrix metalloproteinase (MMP) (e.g., an MMP selected from collagenase-1, -2, and -3 (MMP-1, -8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP); a plasminogen activator (e.g., a uPA or a tissue plasminogen activator (tPA)). Another example of a suitable protease is prolactin. Another example of a suitable protease is a tobacco etch virus (TEV) protease. Another example of suitable protease is enterokinase. Another example of suitable protease is thrombin. Additional examples of suitable protease are: a PreScission protease (a fusion protein comprising human rhinovirus 3C protease and glutathione-S-transferase; Walker et al. (1994) Biotechnol. 12:601); cathepsin B; an Epstein-Barr virus protease; cathespin L; cathepsin D; thermolysin; kallikrein (hK3); neutrophil elastase; calpain (calcium activated neutral protease); and NS3 protease.

### Fusion Polypeptide Comprising a Transcription Factor

As noted above, a component of a FLARE system of the present disclosure can include a fusion polypeptide comprising: a) a transmembrane domain; b) a polypeptide that binds a calmodulin polypeptide or a troponin C polypeptide under certain Ca²⁺ concentration conditions (e.g., a Ca²⁺ concentration above about 100 nM); c) a light-activated polypeptide comprising a LOV domain; d) a proteolytically cleavable linker that is caged by the light-activated polypeptide in the absence of blue light; and e) a transcription factor.

The present disclosure provides a light-activated, calcium-gated transcriptional control polypeptide. A light-activated, calcium-gated transcriptional control polypeptide can comprise, in order from amino terminus (N-terminus) to carboxyl terminus (C-terminus): i) a transmembrane domain; ii) a polypeptide that binds a calmodulin polypeptide or a troponin C polypeptide under certain Ca²⁺ concentration conditions (e.g., a Ca²⁺ concentration above about 100 nM); iii) a light-activated polypeptide that comprises a LOV domain; iv) a proteolytically cleavable linker; and v) a transcription factor.

### Transmembrane Domain

Any of a variety of transmembrane domains (transmembrane polypeptides) can be used in a light-activated, calcium-gated transcriptional control polypeptide of the present disclosure. A suitable transmembrane domain is any polypeptide that is thermodynamically stable in a membrane, e.g., a eukaryotic cell membrane such as a mammalian cell membrane. Suitable transmembrane domains include a single alpha helix, a transmembrane beta barrel, or any other structure.

A suitable transmembrane domain can have a length of from about 10 to 50 amino acids, e.g., from about 10 amino acids to about 40 amino acids, from about 20 amino acids to about 40 amino acids, from about 15 amino acids to about 25 amino acids, e.g., from about 10 amino acids to about 15 amino acids, from about 15 amino acids to about 20 amino acids, from about 20 amino acids to about 25 amino acids, from about 25 amino acids to about 30 amino acids, from about 30 amino acids to about 35 amino acids, from about 35 amino acids to about 40 amino acids, from about 40 amino acids to about 45 amino acids, or from about 45 amino acids to about 50 amino acids.

Suitable transmembrane (TM) domains include, e.g., a Syne homology nuclear TM domain; a CD4 TM domain; a CD8 TM domain; a KASH protein TM domain; a neurexin3b TM domain; a Notch receptor polypeptide TM domain; etc.

For example, a CD4 TM domain can comprise the amino acid sequence MALIVLGGVAGLLLFIGLGIFF (SEQ ID NO://); a CD8 TM domain can comprise the amino acid sequence IYIWAPLAGTCGVLLLSLVIT (SEQ ID NO://); a neurexin3b TM domain can comprise the amino acid sequence GMVVGIVAAAALCILILLYAM (SEQ ID NO://); a Notch receptor polypeptide TM domain can comprise the amino acid sequence FMYVAAAAFVLLFFVGCGVLL (SEQ ID NO://).

### Calmodulin-Binding Polypeptides and Troponin I Polypeptides

In some cases, a light-activated, calcium-gated transcriptional control polypeptide comprises a calmodulin-binding polypeptide. In some cases, a light-activated, calcium-gated transcriptional control polypeptide comprises a troponin I polypeptide.

### Calmodulin-Binding Polypeptides

A suitable troponin I polypeptide binds a troponin C polypeptide under conditions of high Ca²⁺ concentration. For example, a suitable troponin I polypeptide binds a troponin C polypeptide when the concentration of Ca²⁺ is greater than 100 nM, greater than 150 nM, greater than 200 nM, greater than 250 nM, greater than 300 nM, greater than 350 nM, greater than 400 nM, greater than 500 nM, or greater than 750 nM.

A suitable troponin I polypeptide does not substantially bind a troponin C polypeptide under conditions of low Ca²⁺ concentration. For example, a suitable troponin I polypeptide does not substantially bind a troponin C polypeptide when the intracellular Ca²⁺ concentration is less than about 300 nM, less than about 250 nM, less than about 200 nM, less than about 110 nM, less than about 105 nM, or less than about 100 nM.

A troponin I polypeptide can have a length of from about 10 amino acids to about 200 amino acids, e.g., from about 10 amino acids to about 40 amino acids, from about 20 amino acids to about 40 amino acids, from about 15 amino acids to about 25 amino acids, e.g., from about 10 amino acids to about 15 amino acids, from about 15 amino acids to about 20 amino acids, from about 20 amino acids to about 25 amino acids, from about 25 amino acids to about 30 amino acids, from about 30 amino acids to about 35 amino acids, from about 35 amino acids to about 40 amino acids, from about 40 amino acids to about 45 amino acids, from about 45 amino acids to about 50 amino acids, from about amino acids to about 75 amino acids, from about 75 amino acids to about 100 amino acids, from about 100 amino acids to about 150 amino acids, or from about 150 amino acids to about 200 amino acids.

In some cases, a suitable troponin I polypeptide comprises an amino acid sequence having at least 80%, at least 85%, at least 90%, at least 95%, at least 98%, at least 99%, or 100%, amino acid sequence identity to the following troponin I amino acid sequence:

(SEQ ID NO: //)
MPEVERKPKI TASRKLLLKS LMLAKAKECW EQEHEEREAE
KVRYLAERIP TLQTRGLSLS ALQDLCRELH AKVEVVDEER
YDIEAKCLHN TREIKDLKLK VMDLRGKFKR PPLRRVRVSA
DAMLRALLGS KHKVSMDLRA NLKSVKKEDT EKERPVEVGD
WRKNVEAMSG MEGRKKMFDA AKSPTSQ.

A fragment of troponin I can be used. See, e.g., Tung et al. (2000) Protein Sci. 9:1312. For example, troponin I (95-114) can be used. Thus, for example, in some cases, the troponin I polypeptide can comprise an amino acid sequence having at least 80%, at least 85%, at least 90%, at least 95%, at least 98%, at least 99%, or 100%, amino acid sequence identity to the following troponin I amino acid sequence: KDLKLK VMDLRGKFKR PPLR (SEQ ID NO://); and has a length of about 20 amino acids to about 50 amino acids (e.g., from about 20 amino acids to about 25 amino acids, from about 25 amino acids to about 30 amino acids, from about 30 amino acids to about 35 amino acids, from about 35 amino acids to about 40 amino acids, from about 40 amino acids to about 45 amino acids, or from about 45 amino acids to about 50 amino acids). In some cases, the troponin I polypeptide has a length of 20 amino acids. In some cases, the troponin I polypeptide has the amino acid sequence: KDLKLK VMDLRGKFKR PPLR (SEQ ID NO://); and has a length of 20 amino acids.

In some cases, a suitable troponin I polypeptide comprises an amino acid sequence having at least 80%, at least 85%, at least 90%, at least 95%, at least 98%, at least 99%, or 100%, amino acid sequence identity to the following troponin I amino acid sequence: RMSADAMLKALLGSKHKVAMDLRAN (SEQ ID NO://); and has a length of from about 25 amino acids to about 50 amino acids (e.g., from about 25 amino acids to about 30 amino acids, from about 30 amino acids to about 35 amino acids, from about 35 amino acids to about 40 amino acids, from about 40 amino acids to about 45 amino acids, or from about 45 amino acids to about 50 amino acids). In some cases, the troponin I polypeptide has the amino acid sequence: RMSADAMLKALLGSKHKVAMDLRAN (SEQ ID NO://); and has a length of 25 amino acids.

In some cases, a suitable troponin I polypeptide comprises an amino acid sequence having at least 80%, at least 85%, at least 90%, at least 95%, at least 98%, at least 99%, or 100%, amino acid sequence identity to the following troponin I amino acid sequence: NQKLFDLRGKFKRPPLRRVRMSADAMLKALLGSKHKVAMDLRAN (SEQ ID NO://); and has a length of from about 44 amino acids to about 50 amino acids (e.g., 44, 45, 46, 47, 4, 49, or 50 amino acids). In some cases, the troponin I polypeptide has the amino acid sequence: NQKLFDLRGKFKRPPLRRVRMSADAMLKALLGSKHKVAMDLRAN (SEQ ID NO://); and has a length of 44 amino acids.

A suitable calmodulin-binding polypeptide binds a calmodulin polypeptide under conditions of high Ca²⁺ concentration. For example, a suitable calmodulin-binding polypeptide binds a calmodulin polypeptide when the concentration of Ca²⁺ is greater than 100 nM, greater than 150 nM, greater than 200 nM, greater than 250 nM, greater than 300 nM, greater than 350 nM, greater than 400 nM, greater than 500 nM, or greater than 750 nM.

### Calmodulin-Binding Polypeptides

A suitable calmodulin-binding polypeptide does not substantially bind a calmodulin polypeptide under conditions of low Ca²⁺ concentration. For example, a suitable calmodulin-binding polypeptide does not substantially bind a calmodulin polypeptide when the intracellular Ca²⁺ concentration is less than about 300 nM, less than about 250 nM, less than about 200 nM, less than about 110 nM, less than about 105 nM, or less than about 100 nM.

A calmodulin-binding polypeptide can have a length of from about 10 amino acids to about 50 amino acids, e.g., from about 10 amino acids to about 40 amino acids, from about 20 amino acids to about 40 amino acids, from about 15 amino acids to about 25 amino acids, e.g., from about 10 amino acids to about 15 amino acids, from about 15 amino acids to about 20 amino acids, from about 20 amino acids to about 25 amino acids, from about 25 amino acids to about 30 amino acids, from about 30 amino acids to about 35 amino acids, from about 35 amino acids to about 40 amino acids, from about 40 amino acids to about 45 amino acids, or from about 45 amino acids to about 50 amino acids.

A suitable calmodulin-binding polypeptide in some cases comprises an amino acid sequence having at least 90%, at least 95%, at least 98%, at least 99%, or 100%, amino acid sequence identity to the following amino acid sequence: KRRWKKNFIAVSAANRFKKISSSGAL (SEQ ID NO://); and has a length of from about 26 amino acids to about 30 amino acids.

In some cases, a suitable calmodulin-binding polypeptide comprises an amino acid sequence having at least 90%, at least 95%, at least 98%, at least 99%, or 100%, amino acid sequence identity to the following amino acid sequence: KRRWKKNFIAVSAANRFKKISSSGAL (SEQ ID NO://); and has a substitution of A14; and has a length of from about 26 amino acids to about 30 amino acids. In some cases, a suitable calmodulin-binding polypeptide comprises an amino acid sequence having at least 90%, at least 95%, at least 98%, at least 99%, or 100%, amino acid sequence identity to the following amino acid sequence: KRRWKKNFIAVSAANRFKKISSSGAL (SEQ ID NO://); and has an A14F substitution; and has a length of from about 26 amino acids to about 30 amino acids. In some cases, a suitable calmodulin-binding polypeptide comprises the following amino acid sequence: KRRWKKNFIAVSAFNRFKKISSSGAL (SEQ ID NO://); and has a length of 26 amino acids.

In some cases, a suitable calmodulin-binding polypeptide comprises an amino acid sequence having at least 90%, at least 95%, at least 98%, at least 99%, or 100%, amino acid sequence identity to the following amino acid sequence: FNARRKLKGAILTTMLFTRNFS (SEQ ID NO://); and has a length of from 22 amino acids to about 25 amino acids. In some cases, a suitable calmodulin-binding polypeptide comprises an amino acid sequence having at least 90%, at least 95%, at least 98%, at least 99%, or 100%, amino acid sequence identity to the following amino acid sequence: FNARRKLKGAILTTMLFTRNFS (SEQ ID NO://); and has a K8 amino acid substitution; and has a length of from 22 amino acids to about 25 amino acids. In some cases, a suitable calmodulin-binding polypeptide comprises an amino acid sequence having at least 90%, at least 95%, at least 98%, at least 99%, or 100%, amino acid sequence identity to the following amino acid sequence: FNARRKLKGAILTTMLFTRNFS (SEQ ID NO://); and has a K8A amino acid substitution; and has a length of from 22 amino acids to about 25 amino acids. In some cases, a suitable calmodulin-binding polypeptide comprises an amino acid sequence having at least 90%, at least 95%, at least 98%, at least 99%, or 100%, amino acid sequence identity to the following amino acid sequence: FNARRKLKGAILTTMLFTRNFS (SEQ ID NO://); and has a T13 substitution; and has a length of from 22 amino acids to about 25 amino acids. In some cases, a suitable calmodulin-binding polypeptide comprises an amino acid sequence having at least 90%, at least 95%, at least 98%, at least 99%, or 100%, amino acid sequence identity to the following amino acid sequence: FNARRKLKGAILTTMLFTRNFS (SEQ ID NO://); and has a T13F substitution; and has a length of from 22 amino acids to about 25 amino acids. In some cases, a suitable calmodulin-binding polypeptide comprises the following amino acid sequence: FNARRKLKGAILFTMLFTRNFS; and has a length of 22 amino acids. In some cases, a suitable calmodulin-binding polypeptide comprises the following amino acid sequence: FNARRKLAGAILFTMLFTRNFS; and has a length of 22 amino acids.

A suitable calmodulin polypeptide comprises an amino acid sequence having at least 80%, at least 85%, at least 90%, at least 95%, at least 98%, at least 99%, or 100%, amino acid sequence identity to the amino acid sequence depicted in FIG. 16A or FIG. 16B.

A suitable calmodulin polypeptide comprises an amino acid sequence having at least 80%, at least 85%, at least 90%, at least 95%, at least 98%, at least 99%, or 100%, amino acid sequence identity to the following calmodulin amino acid sequence: MDQLTEEQIAEFKEAFSLFDKDGDGTITTKELGTVMRSLGQNPTEAELQDMINEVDADG DGTIDFPEFLTMMARKMKYTDSEEEIREAFRVFDKDGNGYISAAELRHVMTNLGEKLTD EEVDEMIREADIDGDGQVNYEEFVQMMTAK (SEQ ID NO://); and has a length of from about 148 amino acids to about 160 amino acids. In some cases, the calmodulin polypeptide has a length of 148 amino acids.

In some cases, a suitable calmodulin polypeptide comprises an amino acid sequence having at least 80%, at least 85%, at least 90%, at least 95%, at least 98%, at least 99%, or 100%, amino acid sequence identity to the following calmodulin amino acid sequence: MDQLTEEQIAEFKEAFSLFDKDGDGTITTKELGTVMRSLGQNPTEAELQDMINEVDADG DGTIDFPEFLTMMARKMKYTDSEEEIREAFRVFDKDGNGYISAAELRHVMTNLGEKLTD EEVDEMIREADIDGDGQVNYEEFVQMMTAK (SEQ ID NO://); and has a substitution of F19; and has a length of from about 148 amino acids to about 160 amino acids. In some cases, the calmodulin polypeptide has a length of 148 amino acids. In some cases, the F19 substitution is an F19L substitution, an F19I substitution, an F19V substitution, or an F19A substitution.

In some cases, a suitable calmodulin polypeptide comprises an amino acid sequence having at least 80%, at least 85%, at least 90%, at least 95%, at least 98%, at least 99%, or 100%, amino acid sequence identity to the following calmodulin amino acid sequence: MDQLTEEQIAEFKEAFSLFDKDGDGTITTKELGTVMRSLGQNPTEAELQDMINEVDADG DGTIDFPEFLTMMARKMKYTDSEEEIREAFRVFDKDGNGYISAAELRHVMTNLGEKLTD EEVDEMIREADIDGDGQVNYEEFVQMMTAK (SEQ ID NO://); and has a substitution of V35; and has a length of from about 148 amino acids to about 160 amino acids. In some cases, the calmodulin polypeptide has a length of 148 amino acids. In some cases, the V35 substitution is a V35G substitution, a V35A substitution, a V35L substitution, or a V35I substitution.

In some cases, a suitable calmodulin polypeptide comprises an amino acid sequence having at least 80%, at least 85%, at least 90%, at least 95%, at least 98%, at least 99%, or 100%, amino acid sequence identity to the following calmodulin amino acid sequence: MDQLTEEQIAEFKEAFSLFDKDGDGTITTKELGTVMRSLGQNPTEAELQDMINEVDADG DGTIDFPEFLTMMARKMKYTDSEEEIREAFRVFDKDGNGYISAAELRHVMTNLGEKLTD EEVDEMIREADIDGDGQVNYEEFVQMMTAK (SEQ ID NO://); and has an F19 substitution (e.g., an F19L substitution, an F19I substitution, an F19V substitution, or an F19A substitution) and a V35 substitution (e.g., a V35G substitution, a V35A substitution, a V35L substitution, or a V35I substitution); and has a length of from about 148 amino acids to about 160 amino acids. In some cases, the calmodulin polypeptide has a length of 148 amino acids.

In some cases, a suitable calmodulin polypeptide comprises an amino acid sequence having at least 80%, at least 85%, at least 90%, at least 95%, at least 98%, at least 99%, or 100%, amino acid sequence identity to the following calmodulin amino acid sequence: MDQLTEEQIAEFKEAFSLLDKDGDGTITTKELGTGMRSLGQNPTEAELQDMINEVDADG DGTIDFPEFLTMMARKMKYTDSEEEIREAFRVFDKDGNGYISAAELRHVMTNLGEKLTD EEVDEMIREADIDGDGQVNYEEFVQMMTAK (SEQ ID NO://); and comprises a Leu at amino acid 19 and a Gly at amino acid 35; and has a length of from about 148 amino acids to about 160 amino acids. In some cases, the calmodulin polypeptide has a length of 148 amino acids.

### LOV Domain Light-Responsive Polypeptide

A LOV domain light-activated polypeptide suitable for inclusion in a light-activated, calcium-gated transcriptional control polypeptide of the present disclosure is activatable by blue light, and can cage a proteolytically cleavable linker attached to the light-activated polypeptide. Thus, in the absence of blue light, the proteolytically cleavable linker is caged, i.e., inaccessible to a protease. In the presence of blue light, the light-activated polypeptide undergoes a conformational change, such that the proteolytically cleavable linker is uncaged and becomes accessible to a protease. A light-activated polypeptide suitable for inclusion in a light-activated, calcium-gated transcriptional control polypeptide of the present disclosure is a light, oxygen, or voltage (LOV) polypeptide.

A LOV polypeptide suitable for inclusion in a light-activated, calcium-gated transcriptional control polypeptide of the present disclosure can have a length of from about 100 amino acids to about 150 amino acids. For example, a LOV polypeptide can comprise an amino acid sequence having at least 80%, at least 85%, at least 90%, at least 95%, at least 98%, at least 99%, or 100%, amino acid sequence identity to the LOV2 domain of Avena sativa phototropin 1 (AsLOV2).

In some cases, a suitable LOV polypeptide comprises an amino sequence having at least 80%, at least 85%, at least 90%, at least 95%, at least 98%, at least 99%, or 100%, amino acid sequence identity to the following LOV2 amino acid sequence: DLATTLERIEKNFVITDPRLPDNPIIFASDSFLQLTEYSREEILGRNCRFLQGPETDRATVRKI RDAIDNQTEVTVQLINYTKSGKKFWNLFHLQPMRDQKGDVQYFIGVQLDGTEHVRDAAEREGVM LIKKTAENIDEAAK (SEQ ID NO://); GenBank AF033096. In some cases, a suitable LOV polypeptide comprises an amino sequence having at least 80%, at least 85%, at least 90%, at least 95%, at least 98%, at least 99%, or 100%, amino acid sequence identity to the following LOV2 amino acid sequence: DLATTLERIEKNFVITDPRLPDNPIIFASDSFLQLTEYSREEILGRNCRFLQGPETDRATVRKI RDAIDNQTEVTVQLINYTKSGKKFWNLFHLQPMRDQKGDVQYFIGVQLDGTEHVRDAAEREGVM LIKKTAENIDEAAK (SEQ ID NO://); and has a length of from 142 amino acids to 150 amino acids. In some cases, a suitable LOV polypeptide comprises an amino sequence having at least 80%, at least 85%, at least 90%, at least 95%, at least 98%, at least 99%, or 100%, amino acid sequence identity to the following LOV2 amino acid sequence: DLATTLERIEKNFVITDPRLPDNPIIFASDSFLQLTEYSREEILGRNCRFLQGPETDRATVRKI RDAIDNQTEVTVQLINYTKSGKKFWNLFHLQPMRDQKGDVQYFIGVQLDGTEHVRDAAEREGVM LIKKTAENIDEAAK (SEQ ID NO://); and has a length of 142 amino acids.

In some cases, a suitable LOV polypeptide comprises an amino sequence having at least 80%, at least 85%, at least 90%, at least 95%, at least 98%, at least 99%, or 100%, amino acid sequence identity to the following amino acid sequence: SLATTLERIEKNFVITDPRLPDNPIIFASDSFLQLTEYSREEILGRNCRFLQGPETDRATVR KIRDAIDNQTEVTVQLINYTKSGKKFWNLFHLQPMRDQKGDVQYFIGVQLDGTEHVRD AAEREAVMLIKKTAEEIDEAAK (SEQ ID NO://). In some cases, a suitable LOV polypeptide comprises an amino sequence having at least 80%, at least 85%, at least 90%, at least 95%, at least 98%, at least 99%, or 100%, amino acid sequence identity to the following amino acid sequence: SLATTLERIEKNFVITDPRLPDNPIIFASDSFLQLTEYSREEILGRNCRFLQGPETDRATVR KIRDAIDNQTEVTVQLINYTKSGKKFWNLFHLQPMRDQKGDVQYFIGVQLDGTEHVRD AAEREAVMLIKKTAEEIDEAAK (SEQ ID NO://); and has a length of from about 142 amino acids to about 150 amino acids. In some cases, a suitable LOV polypeptide comprises an amino sequence having at least 80%, at least 85%, at least 90%, at least 95%, at least 98%, at least 99%, or 100%, amino acid sequence identity to the following amino acid sequence: SLATTLERIEKNFVITDPRLPDNPIIFASDSFLQLTEYSREEILGRNCRFLQGPETDRATVR KIRDAIDNQTEVTVQLINYTKSGKKFWNLFHLQPMRDQKGDVQYFIGVQLDGTEHVRD AAEREAVMLIKKTAEEIDEAAK (SEQ ID NO://); and has a length of 142 amino acids.

In some cases, a suitable LOV polypeptide comprises an amino sequence having at least 80%, at least 85%, at least 90%, at least 95%, at least 98%, at least 99%, or 100%, amino acid sequence identity to the following amino acid sequence: SLATTLERIEKNFVITDPRLPDNPIIFASDSFLQLTEYSREEILGRNCRFLQGPETDRATVR KIRDAIDNQTEVTVQLINYTKSGKKFWNLFHLQPMRDQKGDVQYFIGVQLDGTEHVRD AAEREAVMLIKKTAEEIDEAAK (SEQ ID NO://); and comprises a substitution at one or more of amino acids L2, N12, A28, H117, and I130, where the numbering is based on the amino acid sequence SLATTLERIEKNFVITDPRLPDNPIIFASDSFLQLTEYSREEILGRNCRFLQGPETDRATVR KIRDAIDNQTEVTVQLINYTKSGKKFWNLFHLQPMRDQKGDVQYFIGVQLDGTEHVRD AAEREAVMLIKKTAEEIDEAAK (SEQ ID NO://). In some cases, the LOV polypeptide comprises a substitution selected from an L2R substitution, an L2H substitution, an L2P substitution, and an L2K substitution. In some cases, the LOV polypeptide comprises a substitution selected from an N12S substitution, an N12T substitution, and an N12Q substitution. In some cases, the LOV polypeptide comprises a substitution selected from an A28V substitution, an A28I substitution, and an A28L substitution. In some cases, the LOV polypeptide comprises a substitution selected from an H117R substitution, and an H117K substitution. In some cases, the LOV polypeptide comprises a substitution selected from an I130V substitution, an I130A substitution, and an I130L substitution. In some cases, the LOV polypeptide comprises substitutions at amino acids L2, N12, and I130. In some cases, the LOV polypeptide comprises substitutions at amino acids L2, N12, H117, and I130. In some cases, the LOV polypeptide comprises substitutions at amino acids A28 and H117. In some cases, the LOV polypeptide comprises substitutions at amino acids N12 and I130. In some cases, the LOV polypeptide comprises an L2R substitution, an N12S substitution, and an I130V substitution. In some cases, the LOV polypeptide comprises an N12S substitution and an I130V substitution. In some cases, the LOV polypeptide comprises an A28V substitution and an H117R substitution. In some cases, the LOV polypeptide comprises an L2P substitution, an N12S substitution, an I130V substitution, and an H117R substitution. In some cases, the LOV polypeptide comprises an L2P substitution, an N12S substitution, an A28V substitution, an H117R substitution, and an I130V substitution. In some cases, the LOV polypeptide comprises an L2P substitution, an N12S substitution, an I130V substitution, and an H117R substitution. In some cases, the LOV polypeptide comprises an L2R substitution, an N12S substitution, an A28V substitution, an H117R substitution, and an I130V substitution. In some cases, the LOV polypeptide has a length of 142 amino acids, 143 amino acids, 144 amino acids, 145 amino acids, 146 amino acids, 147 amino acids, 148 amino acids, 149 amino acids, or 150 amino acids. In some cases, the LOV polypeptide has a length of 142 amino acids.

In some cases, a suitable LOV polypeptide comprises an amino sequence having at least 80%, at least 85%, at least 90%, at least 95%, at least 98%, at least 99%, or 100%, amino acid sequence identity to the following amino acid sequence: SRATTLERIEKSFVITDPRLPDNPIIFVSDSFLQLTEYSREEILGRNCRFLQGPETDRATVR KIRDAIDNQTEVTVQLINYTKSGKKFWNLFHLQPMRDQKGDVQYFIGVQLDGTERVRD AAEREAVMLVKKTAEEIDEAAK (SEQ ID NO://); and has an Arg at amino acid 2, a Ser at amino acid 12, a Val at amino acid 28, an Arg at amino acid 117, and a Val at amino acid 130, as indicated by bold and underlined letters; and has a length of 142 amino acids, 143 amino acids, 144 amino acids, 145 amino acids, 146 amino acids, 147 amino acids, 148 amino acids, 149 amino acids, or 150 amino acids. In some cases, a suitable LOV polypeptide comprises the following amino acid sequence: SRATTLERIEKSFVITDPRLPDNPIIFVSDSFLQLTEYSREEILGRNCRFLQGPETDRATVR KIRDAIDNQTEVTVQLINYTKSGKKFWNLFHLQPMRDQKGDVQYFIGVQLDGTERVRD AAEREAVMLVKKTAEEIDEAAK (SEQ ID NO://); and has a length of 142 amino acids.

In some cases, a suitable LOV polypeptide comprises an amino sequence having at least 80%, at least 85%, at least 90%, at least 95%, at least 98%, at least 99%, or 100%, amino acid sequence identity to the following amino acid sequence: SRATTLERIEKSFVITDPRLPDNPVIFVSDSFLQLTEYSREEILGRNCRFLQGPETDRATVR KIRDAIDNQTEVTVQLINYTKSGKKFWNLFHLQPMRDQKGDVQYFIGVQLDGTERVRD AAEREAVMLVKKTAEEIDEAAK (SEQ ID NO://); and has an Arg at amino acid 2, a Ser at amino acid 12, a Val at amino acid 25, a Val at amino acid 28, an Arg at amino acid 117, and a Val at amino acid 130, as indicated by bold and underlined letters; and has a length of 142 amino acids, 143 amino acids, 144 amino acids, 145 amino acids, 146 amino acids, 147 amino acids, 148 amino acids, 149 amino acids, or 150 amino acids. In some cases, a suitable LOV polypeptide comprises the following amino acid sequence: SRATTLERIEKSFVITDPRLPDNPVIFVSDSFLQLTEYSREEILGRNCRFLQGPETDRATVR KIRDAIDNQTEVTVQLINYTKSGKKFWNLFHLQPMRDQKGDVQYFIGVQLDGTERVRD AAEREAVMLVKKTAEEIDEAAK (SEQ ID NO://); and has a length of 142 amino acids.

A suitable LOV domain light-activated polypeptide comprises one or more amino acid substitutions relative to the LOV2 amino acid sequence depicted in FIG. 15A. In some cases, a suitable LOV domain light-activated polypeptide comprises one or more amino acid substitutions at positions selected from 1, 2, 12, 25, 28, 91, 100, 117, 118, 119, 120, 126, 128, 135, 136, and 138, relative to the LOV2 amino acid sequence depicted in FIG. 15A. Suitable substitutions include, Asp→Ser at amino acid 1; Asp→Phe at amino acid 1; Leu→Arg at amino acid 2; Asn→Ser at amino acid 12; Ile→Val at amino acid 12; Ala→Val at amino acid 28; Leu→Val at amino acid 91; Gln→Tyr at amino acid 100; His→Arg at amino acid 117; Val→Leu at amino acid 118; Arg→His at amino acid 119; Asp→Gly at amino acid 120; Gly→Ala at amino acid 126; Met→Cys at amino acid 128; Glu→Phe at amino acid 135; Asn→Gln at amino acid 136; Asn→Glu at amino acid 136; and Asp→Ala at amino acid 138, where the amino acid numbering is based on the number of the LOV2 amino acid sequence depicted in FIG. 15A.

In some cases, a suitable LOV domain light-activated polypeptide comprises an amino acid sequence having at least 80%, at least 85%, at least 90%, at least 95%, at least 98%, at least 99%, or 100%, amino acid sequence identity to the amino acid sequence depicted in FIG. 15B, where amino acid 1 is Ser, amino acid 28 is Ala, amino acid 126 is Ala, and amino acid 136 is Glu. In some case, the suitable LOV domain light-activated polypeptide has a length of 142 amino acids.

In some cases, a suitable LOV domain light-activated polypeptide comprises an amino acid sequence having at least 80%, at least 85%, at least 90%, at least 95%, at least 98%, at least 99%, or 100%, amino acid sequence identity to the amino acid sequence depicted in FIG. 15C, where amino acid 1 is Ser; amino acid 2 is Arg; amino acid 12 is Ser; amino acid 28 is Ala; amino acid 117 is Arg; amino acid 126 is Ala; and amino acid 136 is Glu. In some case, the suitable LOV domain light-activated polypeptide has a length of 142 amino acids.

In some cases, a suitable LOV domain light-activated polypeptide comprises an amino acid sequence having at least 80%, at least 85%, at least 90%, at least 95%, at least 98%, at least 99%, or 100%, amino acid sequence identity to the amino acid sequence depicted in FIG. 15D, where amino acid 1 is Ser; amino acid 2 is Arg; amino acid 12 is Ser; amino acid 25 is Val; amino acid 28 is Val; amino acid 117 is Arg; amino acid 126 is Ala; amino acid 130 is Val; and amino acid 136 is Glu. In some case, the LOV domain light-activated polypeptide has a length of 142 amino acids.

In some cases, a suitable LOV domain light-activated polypeptide comprises an amino acid sequence having at least 80%, at least 85%, at least 90%, at least 95%, at least 98%, at least 99%, or 100%, amino acid sequence identity to the amino acid sequence depicted in FIG. 15E, where amino acid 1 is Ser; amino acid 2 is Arg; amino acid 12 is Ser; amino acid 28 is Ala; amino acid 91 is Val; amino acid 100 is Tyr; amino acid 117 is Arg; amino acid 118 is Leu; amino acid 119 is His; amino acid 120 is Gly; amino acid 126 is Ala; amino acid 128 is Cys; amino acid 130 is Val; amino acid 135 is Phe; amino acid 136 is Gln; and amino acid 138 is Ala. In some case, the LOV domain light-activated polypeptide has a length of 142 amino acids.

In some cases, a suitable LOV domain light-activated polypeptide comprises an amino acid sequence having at least 80%, at least 85%, at least 90%, at least 95%, at least 98%, at least 99%, or 100%, amino acid sequence identity to the amino acid sequence depicted in FIG. 15F, where amino acid 1 is Ser; amino acid 2 is Arg; amino acid 12 is Ser; amino acid 28 is Val; amino acid 117 is Arg; amino acid 126 is Ala; amino acid 130 is Val; and amino acid 136 is Glu. In some case, the LOV domain light-activated polypeptide has a length of 138 amino acids.

In some cases, a suitable LOV domain light-activated polypeptide comprises an amino acid sequence having at least 80%, at least 85%, at least 90%, at least 95%, at least 98%, at least 99%, or 100%, amino acid sequence identity to the amino acid sequence depicted in FIG. 15G, where amino acid 1 is Ser; amino acid 2 is Arg; amino acid 12 is Ser; amino acid 28 is Val; amino acid 91 is Val; amino acid 100 is Tyr; amino acid 117 is Arg; amino acid 118 is Leu; amino acid 119 is His; amino acid 120 is Gly; amino acid 126 is Ala; amino acid 128 is Cys; amino acid 130 is Val; amino acid 135 is Phe; amino acid 136 is Gln; and amino acid 138 is Ala. In some case, the LOV domain light-activated polypeptide has a length of 138 amino acids.

In some cases, a LOV light-activated polypeptide comprises the following amino acid sequence:

(SEQ ID NO: //)
FRATTLERIEKSFVITDPRLPDNPIIFVSDSFLQLTEYSREEILGRN
CRFLQGPETDRATVRKIRDAIDNQTEVTVQLINYTKSGKKFWNVFHL
QPMRDYKGDVQYFIGVQLDGTERLHGAAEREAVCLVKKTAFQIA.

In some cases, a LOV light-activated polypeptide comprises the following amino acid sequence:

(SEQ ID NO: //)
SRATTLERIEKSFVITDPRLPDNPIIFVSDSFLQLTEYSREEILGRN
CRFLQGPETDRATVRKIRDAIDNQTEVTVQLINYTKSGKKFWNLFHL
QPMRDQKGDVQYFIGVQLDGTERVRDAAEREAVMLVKKTAEEID.

In some cases, a LOV light-activated polypeptide comprises the following amino acid sequence:

(SEQ ID NO: //)
FRATTLERIEKSFVITDPRLPDNPIIFVSDSFLQLTEYSREEILGRN
CRFLQGPETDRATVRKIRDAIDNQTEVTVQLINYTKSGKKFWNVFHL
QPMRDYKGDVQYFIGVQLDGTERLHGAAEREAVCLVKKTAFQIA.

In some cases, a LOV light-activated polypeptide comprises the following amino acid sequence:

(SEQ ID NO: //)
SRATTLERIEKSFVITDPRLPDNPIIFVSDSFLQLTEYSREEILGRN
CRFLQGPETDRATVRKIRDAIDNQTEVTVQLINYTKSGKKFWNVFHL
QPMRDYKGDVQYFIGVQLDGTERLHGAAEREAVCLVKKTAFEIDEAA
K.

In some cases, a LOV light-activated polypeptide comprises the following amino acid sequence:

(SEQ ID NO: //)
SRATTLERIEKSFVITDPRLPDNPIIFVSDSFLQLTEYSREEILGRN
CRFLQGPETDRATVRKIRDAIDNQTEVTVQLINYTKSGKKFWNLFHL
QPMRDQKGDVQYFIGVQLDGTERVRDAAEREAVMLVKKTAEEIDEAA
K.

LOV light-activated polypeptide cages the proteolytically cleavable linker in the absence of light of an activating wavelength, the proteolytically cleavable linker is substantially not accessible to the protease. Thus, e.g., in the absence of light of an activating wavelength (e.g., in the dark; or in the presence of light of a wavelength other than blue light), the proteolytically cleavable linker is cleaved, if at all, to a degree that is more than 50% less, more than 60% less, more than 70% less, more than 80% less, more than 90% less, more than 95% less, more than 98% less, or more than 99% less, than the degree of cleavage of the proteolytically cleavable linker in the presence of light of an activating wavelength (e.g., blue light, e.g., light of a wavelength in the range of from about 450 nm to about 495 nm, from about 460 nm to about 490 nm, from about 470 nm to about 480 nm, e.g., 473 nm).

### Proteolytically Cleavable Linker

The proteolytically cleavable linker can include a protease recognition sequence recognized by a protease selected from the group consisting of alanine carboxypeptidase, Armillaria mellea astacin, bacterial leucyl aminopeptidase, cancer procoagulant, cathepsin B, clostripain, cytosol alanyl aminopeptidase, elastase, endoproteinase Arg-C, enterokinase, gastricsin, gelatinase, Gly-X carboxypeptidase, glycyl endopeptidase, human rhinovirus 3C protease, hypodermin C, IgA-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2A, picornain 3C, proendopeptidase, prolyl aminopeptidase, proprotein convertase I, proprotein convertase II, russellysin, saccharopepsin, semenogelase, T-plasminogen activator, thrombin, tissue kallikrein, tobacco etch virus (TEV), togavirin, tryptophanyl aminopeptidase, U-plasminogen activator, V8, venombin A, venombin AB, and Xaa-pro aminopeptidase.

For example, the proteolytically cleavable linker can comprise a matrix metalloproteinase (MMP) cleavage site, e.g., a cleavage site for a MMP selected from collagenase-1, -2, and -3 (MMP-1, -8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP). For example, the cleavage sequence of MMP-9 is Pro-X-X-Hy (wherein, X represents an arbitrary residue; Hy, a hydrophobic residue), e.g., Pro-X-X-Hy-(Ser/Thr), e.g., Pro-Leu/Gln-Gly-Met-Thr-Ser (SEQ ID NO://) or Pro-Leu/Gln-Gly-Met-Thr (SEQ ID NO://). Another example of a protease cleavage site is a plasminogen activator cleavage site, e.g., a uPA or a tissue plasminogen activator (tPA) cleavage site. Another example of a suitable protease cleavage site is a prolactin cleavage site. Specific examples of cleavage sequences of uPA and tPA include sequences comprising Val-Gly-Arg. Another example of a protease cleavage site that can be included in a proteolytically cleavable linker is a tobacco etch virus (TEV) protease cleavage site, e.g., ENLYFQS (SEQ ID NO://), where the protease cleaves between the glutamine and the serine; or ENLYFQY (SEQ ID NO://), where the protease cleaves between the glutamine and the tyrosine; or ENLYFQL (SEQ ID NO://), where the protease cleaves between the glutamine and the leucine. Another example of a protease cleavage site that can be included in a proteolytically cleavable linker is an enterokinase cleavage site, e.g., DDDDK (SEQ ID NO://), where cleavage occurs after the lysine residue. Another example of a protease cleavage site that can be included in a proteolytically cleavable linker is a thrombin cleavage site, e.g., LVPR (SEQ ID NO://) (e.g., where the proteolytically cleavable linker comprises the sequence LVPRGS (SEQ ID NO://)). Additional suitable linkers comprising protease cleavage sites include linkers comprising one or more of the following amino acid sequences: LEVLFQGP (SEQ ID NO://), cleaved by PreScission protease (a fusion protein comprising human rhinovirus 3C protease and glutathione-S-transferase; Walker et al. (1994) Biotechnol. 12:601); a thrombin cleavage site, e.g., CGLVPAGSGP (SEQ ID NO://); SLLKSRMVPNFN (SEQ ID NO://) or SLLIARRMPNFN (SEQ ID NO://), cleaved by cathepsin B; SKLVQASASGVN (SEQ ID NO://) or SSYLKASDAPDN (SEQ ID NO://), cleaved by an Epstein-Barr virus protease; RPKPQQFFGLMN (SEQ ID NO://) cleaved by MMP-3 (stromelysin); SLRPLALWRSFN (SEQ ID NO://) cleaved by MMP-7 (matrilysin); SPQGIAGQRNFN (SEQ ID NO://) cleaved by MMP-9; DVDERDVRGFASFL SEQ ID NO://) cleaved by a thermolysin-like MMP; SLPLGLWAPNFN (SEQ ID NO://) cleaved by matrix metalloproteinase 2 (MMP-2); SLLIFRSWANFN (SEQ ID NO://) cleaved by cathespin L; SGVVIATVIVIT (SEQ ID NO://) cleaved by cathepsin D; SLGPQGIWGQFN (SEQ ID NO://) cleaved by matrix metalloproteinase 1 (MMP-1); KKSPGRVVGGSV (SEQ ID NO://) cleaved by urokinase-type plasminogen activator; PQGLLGAPGILG (SEQ ID NO://) cleaved by membrane type 1 matrixmetalloproteinase (MT-MMP); HGPEGLRVGFYESDVMGRGHARLVHVEEPHT (SEQ ID NO://) cleaved by stromelysin 3 (or MMP-11), thermolysin, fibroblast collagenase and stromelysin-1; GPQGLAGQRGIV (SEQ ID NO://) cleaved by matrix metalloproteinase 13 (collagenase-3); GGSGQRGRKALE (SEQ ID NO://) cleaved by tissue-type plasminogen activator (tPA); SLSALLSSDIFN (SEQ ID NO://) cleaved by human prostate-specific antigen; SLPRFKIIGGFN (SEQ ID NO://) cleaved by kallikrein (hK3); SLLGIAVPGNFN (SEQ ID NO://) cleaved by neutrophil elastase; and FFKNIVTPRTPP (SEQ ID NO://) cleaved by calpain (calcium activated neutral protease).

Suitable proteolytically cleavable linkers also include ENLYFQS (SEQ ID NO://), ENLYFQY (SEQ ID NO://), ENLYFQL (SEQ ID NO://), ENLYFQW (SEQ ID NO://), ENLYFQM (SEQ ID NO://), ENLYFQH (SEQ ID NO://), ENLYFQN (SEQ ID NO://), ENLYFQA (SEQ ID NO://), and ENLYFQQ (SEQ ID NO://).

Suitable proteolytically cleavable linkers also include NS3 protease cleavage sites such as: DEVVECS (SEQ ID NO://), DEAEDVVECS (SEQ ID NO://), EDAAEEVVECS (SEQ ID NO://).

Suitable proteolytically cleavable linkers also include ENLYFQX (SEQ ID NO://; where X is any amino acid), ENLYFQS (SEQ ID NO://), ENLYFQG (SEQ ID NO://), ENLYFQY (SEQ ID NO://), ENLYFQL (SEQ ID NO://), ENLYFQW (SEQ ID NO://), ENLYFQM (SEQ ID NO://), ENLYFQH (SEQ ID NO://), ENLYFQN (SEQ ID NO://), ENLYFQA (SEQ ID NO://), and ENLYFQQ (SEQ ID NO://).

Suitable proteolytically cleavable linkers also include calpain cleavage site, where suitable calpain cleavage sites include, e.g., PLFAAR (SEQ ID NO://) and QQEVYGMMPRD (SEQ ID NO://).

In some cases, the proteolytically cleavable linker comprises an amino acid sequence that is substantially not cleaved by any endogenous protease in a given cell (e.g., a eukaryotic cell; e.g., a mammalian cell; e.g., a particular type of mammalian cell). In some cases, the proteolytically cleavable linker comprises an amino acid sequence that is cleaved by a viral protease, and that is substantially not cleaved by any endogenous protease in a given cell (e.g., a eukaryotic cell; e.g., a mammalian cell; e.g., a particular type of mammalian cell). In some cases, the proteolytically cleavable linker comprises an amino acid sequence that is cleaved by a non-naturally occurring (e.g., engineered) protease, and that is substantially not cleaved by any endogenous protease in a given cell (e.g., a eukaryotic cell; e.g., a mammalian cell; e.g., a particular type of mammalian cell).

### Transcription Factor

Suitable transcription factors include naturally-occurring transcription factors and recombinant (e.g., non-naturally occurring, engineered, artificial, synthetic) transcription factors. In some cases the transcriptional activator is an engineered protein, such as a zinc finger or TALE based DNA binding domain fused to an effector domain such as VP64 (transcriptional activation).

A transcription factor can comprise: i) a DNA binding domain (DBD); and ii) an activation domain (AD). The DBD can be any DBD with a known response element, including synthetic and chimeric DNA binding domains, or analogs, combinations, or modifications thereof. Suitable DNA binding domains include, but are not limited to, a GAL4 DBD, a LexA DBD, a transcription factor DBD, a Group H nuclear receptor member DBD, a steroid/thyroid hormone nuclear receptor superfamily member DBD, a bacterial LacZ DBD, an EcR DBD, a GALA DBD, and a LexA DBD. Suitable ADs include, but are not limited to, a Group H nuclear receptor member AD, a steroid/thyroid hormone nuclear receptor AD, a CJ7 AD, a p65-TA1 AD, a synthetic or chimeric AD, a polyglutamine AD, a basic or acidic amino acid AD, a VP16 AD, a GAL4 AD, an NF-κB AD, a BP64 AD, a B42 acidic activation domain (B42AD), a p65 transactivation domain (p65AD), SAD, NF-1, AP-2, SP1-A, SP1-B, Oct-1, Oct-2, MTF-1, BTEB-2, and LKLF, or an analog, combination, or modification thereof.

Suitable transcription factors include transcriptional activators, where suitable transcriptional activators include, but are not limited to, GAL4-VP16, GAL5-VP64, Tbx21, tTA-VP16, VP16, VP64, GAL4, p65, LexA-VP16, GAL4-NFκB, and the like. Amino acid sequences of suitable transcriptional activators are known in the art. For example, a tTA-VP16 transcription factor can comprise an amino acid sequence having at least 85%, at least 90%, at least 95%, at least 98%, at least 99%, or 100%, to the following amino acid sequence:

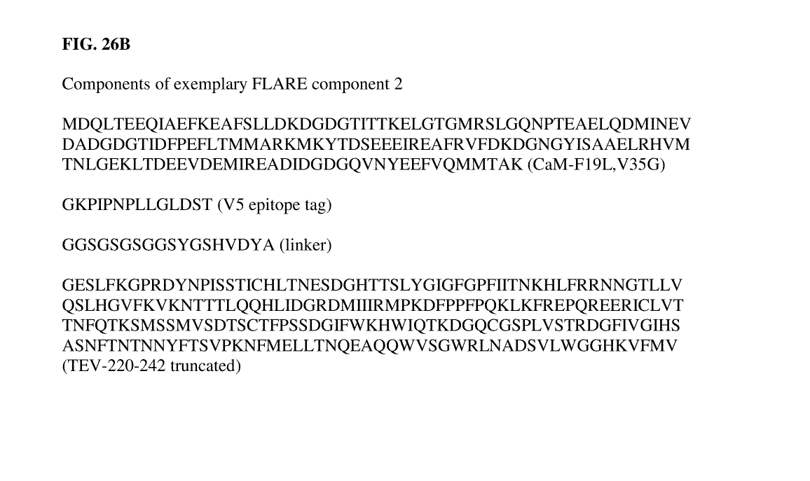

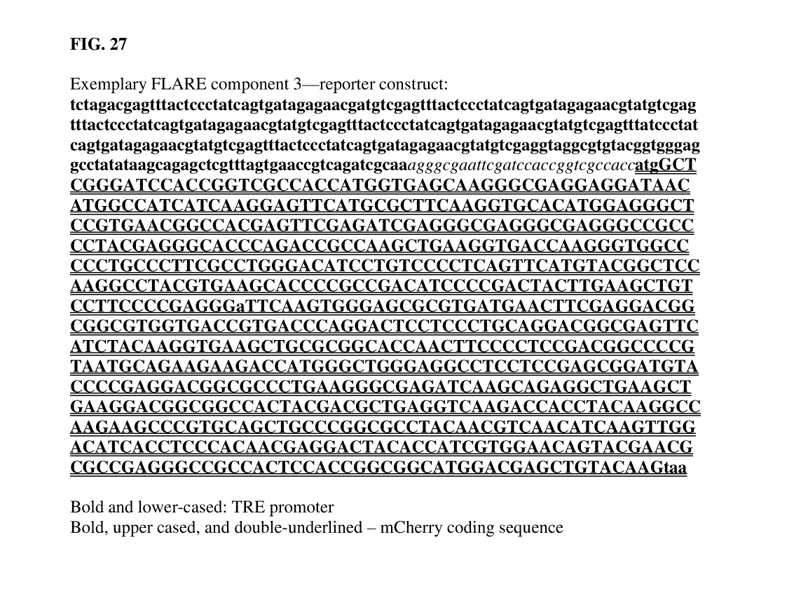

MSRLDKSKVINSALELLNEVGIEGLTTRKLAQKLGVEQPTLYWHVKNKRALLD ALAIEMLDRHHTHFCPLEGESWQDFLRNNAKSFRCALLSHRDGAKVHLGTRPTEKQYE TLENQLAFLCQQGFSLENALYALSAVGHFTLGCVLEDQEHQVAKEERETPTTDSMPPLL RQAIELFDHQGAEPAFLFGLELIICGLEKQLKCESGSAYSRARTKNNYGSTIEGLLDLPDD DAPEEAGLAAPRLSFLPAGHTRRLSTAPPTDVSLGDELHLDGEDVAMAHADALDDFDL DMLGDGDSPGPGFTPHDSAPYGALDMADFEFEQMFTDALGIDEYGG (SEQ ID NO://). A tTA-VP16 transcription activator binds to, e.g., a TRE promoter (see, e.g., FIGS. 27A and 27B).

Suitable transcription factors include transcriptional repressors, where suitable transcriptional repressors (e.g., a transcription repressor domain) include, but are not limited to, Krüppel-associated box (KRAB); the Mad mSIN3 interaction domain (SID); the ERF repressor domain (ERD); MDB-2B; v-ErbA; MBD3; and the like.

### Additional Amino Acid Sequences

A fusion polypeptide comprising: a) a TM domain; b) a polypeptide that binds a calcium-binding polypeptide; c) a light-activated polypeptide comprising a LOV domain; d) a proteolytically cleavable linker; and e) a transcription factor can include one or more additional polypeptides. The one or more additional polypeptides can be, e.g., a soma localization signal; a nuclear localization signal; etc.

### Soma Localization Signal

In some cases, the transcription factor includes a soma localization signal. For example, a 66 amino acid C-terminal sequence of Kv2.1 or a 27 amino acid sequence of Nav1.6 induces localization to the soma of a neuron. For example, the Nav1.6 soma localization signal comprises the amino acid sequence: TVRVPIAVGESDFENLNTEDVSSESDP (SEQ ID NO://).

### Nuclear Localization Signals

Non-limiting examples of NLSs include an NLS sequence derived from: the NLS of the SV40 virus large T-antigen, having the amino acid sequence PKKKRKV (SEQ ID NO://); the NLS from nucleoplasmin (e.g. the nucleoplasmin bipartite NLS with the sequence KRPAATKKAGQAKKKK (SEQ ID NO://)); the c-myc NLS having the amino acid sequence PAAKRVKLD (SEQ ID NO://) or RQRRNELKRSP (SEQ ID NO://); the hRNPA1 M9 NLS having the sequence NQSSNFGPMKGGNFGGRSSGPYGGGGQYFAKPRNQGGY (SEQ ID NO://); the sequence RMRIZFKNKGKDTAELRRRRVEVSVELRKAKKDEQILKRRNV (SEQ ID NO://) of the IBB domain from importin-alpha; the sequences VSRKRPRP (SEQ ID NO://) and PPKKARED (SEQ ID NO://) of the myoma T protein; the sequence PQPKKKPL (SEQ ID NO://) of human p53; the sequence SALIKKKKKMAP (SEQ ID NO://) of mouse c-abl IV; the sequences DRLRR (SEQ ID NO://) and PKQKKRK (SEQ ID NO://) of the influenza virus NS1; the sequence RKLKKKIKKL (SEQ ID NO://) of the Hepatitis virus delta antigen; the sequence REKKKFLKRR (SEQ ID NO://) of the mouse Mx1 protein; the sequence KRKGDEVDGVDEVAKKKSKK (SEQ ID NO://) of the human poly(ADP-ribose) polymerase; and the sequence RKCLQAGMNLEARKTKK (SEQ ID NO://) of the steroid hormone receptors (human) glucocorticoid.

A transcription factor can include a “Protein Transduction Domain” or PTD (also known as a CPP—cell penetrating peptide), which refers to a polypeptide that facilitates traversing a lipid bilayer, micelle, cell membrane, organelle membrane, or vesicle membrane. A PTD attached to another polypeptide (a polypeptide gene product of interest) facilitates the polypeptide traversing a membrane, for example going from extracellular space to intracellular space, or cytosol to within an organelle. In some cases, a PTD attached to a polypeptide gene product of interest facilitates entry of the polypeptide into the nucleus (e.g., in some cases, a PTD includes a nuclear localization signal). In some cases, a PTD is covalently linked to the amino terminus of a polypeptide gene product of interest. In some cases, a PTD is covalently linked to the carboxyl terminus of a polypeptide gene product of interest. In some cases, a PTD is covalently linked to the amino terminus and to the carboxyl terminus of a polypeptide gene product of interest. Exemplary PTDs include but are not limited to a minimal undecapeptide protein transduction domain (corresponding to residues 47-57 of HIV-1 TAT comprising YGRKKRRQRRR; SEQ ID NO://); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); an Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO://); Transportan GWTLNSAGYLLGKINLKALAALAKKIL (SEQ ID NO://); KALAWEAKLAKALAKALAKHLAKALAKALKCEA (SEQ ID NO://); and RQIKIWFQNRRMKWKK (SEQ ID NO://). Exemplary PTDs include but are not limited to, YGRKKRRQRRR (SEQ ID NO://), RKKRRQRRR (SEQ ID NO://); an arginine homopolymer of from 3 arginine residues to 50 arginine residues; Exemplary PTD domain amino acid sequences include, but are not limited to, any of the following: YGRKKRRQRRR (SEQ ID NO://); RKKRRQRR (SEQ ID NO://); YARAAARQARA (SEQ ID NO://); THRLPRRRRRR (SEQ ID NO://); and GGRRARRRRRR (SEQ ID NO://).

### Target Genes

The transcription factor can control expression of any of a variety of gene products. “Gene products” as used herein, include polypeptide gene products and nucleic acid gene products.

Suitable nucleic acid gene products include, but are not limited to, an inhibitory nucleic acid, a ribozyme, a guide RNA that binds a target nucleic acid and an RNA-guided endonuclease, a microRNA, and the like.

### Polypeptide Gene Products

In some cases, a transcription factor present in a light-activated, calcium-gated transcription control polypeptide of the present disclosure, when released from the light-activated, calcium-gated transcription control polypeptide by cleavage of the proteolytically cleavable linker, controls transcription of a nucleotide sequence encoding a polypeptide.

Suitable polypeptide gene products include, but are not limited to, a reporter gene product, an opsin, a DREADD, a toxin, an enzyme, a transcription factor, an antibiotic resistance factor, a genome editing endonuclease, an RNA-guided endonuclease, a protease, a kinase, a phosphatase, a phosphorylase, a lipase, a receptor, an antibody, a fluorescent protein, a peroxidase such as APEX or APEX2, a base editing enzyme, a recombinase, a synaptic marker, a signaling protein, an effector protein of a receptor, a protein that regulates synaptic vesicle fusion or protein trafficking or organelle trafficking, a portion (e.g., a split half) of any one of the aforementioned polypeptides.

### Synaptic Markers

In some cases, a polypeptide of interest is a synaptic marker. Synaptic markers include, but are not limited to, PSD-95, SV2, homer, bassoon, synapsin I, synaptotagmin, synaptophysin, synaptobrevin, SAP102, α-adaptin, GluA1, NMDA receptor, LRRTM1, LRRTM2, SLITRK, neuroligin-1, neuroligin-2, gephyrin, GABA receptor, and the like.

### Nucleic Acid Editing Enzymes

In some cases, a polypeptide of interest is a nucleic acid-editing enzyme. Suitable nucleic acid-editing enzymes include, e.g., a DNA-editing enzyme, a cytidine deaminase, an adenosine deaminase, an apolipoprotein B mRNA-editing complex (APOBEC) family deaminase, an activation-induced cytidine deaminase (AID), an ACF1/ASE deaminase, and an ADAT family deaminase.

### Peroxidases

A suitable polypeptide of interest is in some cases a peroxidase, where suitable peroxidases include, e.g., horse radish peroxidase, yeast cytochrome c peroxidase (CCP), ascorbate peroxidase (APX), bacterial catalase-peroxidase (BCP), APEX, and APEX2. See, e.g., U.S. Patent Publication No. 2014/0206013.

An example of a suitable peroxidase is an APX, which has the following amino acid sequence: MGKSYPTVSA DYQKAVEKAK KKLRGFIAEK RCAPLMLRLA WHSAGTFDKG TKTGGPFGTI KHPAELAHSA NNGLDIAVRL LEPLKAEFPI LSYADFYQLA GVVAVEVTGG PEVPFHPGRE DKPEPPPEGR LPDATKGSDH LRDVFGKAMG LTDQDIVALS GGHTIGAAHK ERSGFEGPWT SNPLIFDNSY FTELLSGEKE GLLQLPSDKA LLSDPVFRPL VDKYAADEDA FFADYAEAHQ KLSELGFADA (SEQ ID NO://). In some cases, the peroxidase comprises a K14D substitution. In some cases, the peroxidase can contain a combination of (a) K14D, E112K, E228K, D229K, K14D/E112K, K14D/E228K, K14D/D229K, E17N/K20A/R21L, or K14D/W41F/E112K, and (b) S69F, G174F, W41F/S69F, D133A/T135F/K136F, W41F/D133A/T135F/K136F, S69F/D133A/T135F/K136F, or W41F/S69F/D133A/T135F/K136F. In some cases, the peroxidase can contain a combination of (a) single mutant K14D, single mutant E112K, single mutant E228K, single mutant D229K, double mutant K14D/E112K, double mutant K14D/E228K, double mutant K14D/D229K, triple mutant E17N/K20A/R21L, or triple mutant K14D/W41F/E112K, and (b) single mutant W41F, single mutant S69F, single mutant G174F, double mutant W41F/S69F, triple mutant D133A/T135F/K136F, quadruple mutant W41F/D133A/T135F/K136F, quadruple mutant S69F/D133A/T135F/K136F, or quintuple mutant W41F/S69F/D133A/T135F/K136F. Examples of such combined mutants include, but are not limited to, K14D/E112K/W41F (APEX), and K 14D/E112K/W41F/D133A/T135F/K136F. The amino acid numbering is based on the above-provided APX amino acid sequence.

### Antibodies

A suitable polypeptide of interest is in some cases an antibody. The terms “antibodies” and “immunoglobulin” include antibodies or immunoglobulins of any isotype, fragments of antibodies that retain specific binding to antigen, including, but not limited to, Fab, Fv, scFv, and Fd fragments, chimeric antibodies, humanized antibodies, single-chain antibodies (scAb), single domain antibodies (dAb), single domain heavy chain antibodies, a single domain light chain antibodies, nanobodies, bi-specific antibodies, multi-specific antibodies, and fusion proteins comprising an antigen-binding (also referred to herein as antigen binding) portion of an antibody and a non-antibody protein. Also encompassed by the term are Fab′, Fv, F(ab′)₂, and or other antibody fragments that retain specific binding to antigen, and monoclonal antibodies.

The term “nanobody” (Nb), as used herein, refers to the smallest antigen binding fragment or single variable domain (V_{HH}) derived from naturally occurring heavy chain antibody and is known to the person skilled in the art. They are derived from heavy chain only antibodies, seen in camelids (Hamers-Casterman et al., 1993; Desmyter et al., 1996). In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a V_{HH} antibody.

“Antibody fragments” comprise a portion of an intact antibody, for example, the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 (1995)); domain antibodies (dAb; Holt et al. (2003) Trends Biotechnol. 21:484); single-chain antibody molecules; and multi-specific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen. Antibody fragments include, e.g., scFv, sdAb, dAb, Fab, Fab′, Fab′₂, F(ab′)₂, Fd, Fv, Feb, and SMIP. An example of an sdAb is a camelid VHH.

“Fv” is the minimum antibody fragment that contains a complete antigen-recognition and -binding site. This region consists of a dimer of one heavy- and one light-chain variable domain in tight, non-covalent association. It is in this configuration that the three complementarity determining regions (CDRs) of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

“Single-chain Fv” or “sFv” or “scFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. (1993) Proc. Natl. Acad. Sci. USA 90:6444-6448.

### Reporter Gene Products

Suitable reporter gene products include polypeptides that generate a detectable signal. Suitable detectable signal-producing proteins include, e.g., fluorescent proteins; enzymes that catalyze a reaction that generates a detectable signal as a product; and the like.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrape1, mRaspberry, mGrape2, mPlum (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, is suitable for use.

Suitable enzymes include, but are not limited to, horse radish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, glucose oxidase (GO), and the like.

### Genome-Editing Endonuclease

A “genome editing endonuclease” is an endonuclease, e.g., sequence-specific endonuclease, which can be used for the editing of a cell's genome (e.g., by cleaving at a targeted location within the cell's genomic DNA). Examples of genome editing endonucleases include but are not limited to: (i) Zinc finger nucleases, (ii) TAL endonucleases, and (iii) CRISPR/Cas endonucleases. Examples of CRISPR/Cas endonucleases include class 2 CRISPR/Cas endonucleases such as: (a) type II CRISPR/Cas proteins, e.g., a Cas9 protein; (b) type V CRISPR/Cas proteins, e.g., a Cpf1 polypeptide, a C2c1 polypeptide, a C2c3 polypeptide, and the like; and (c) type VI CRISPR/Cas proteins, e.g., a C2c2 polypeptide.

Examples of suitable sequence-specific, e.g., genome editing, endonucleases include, but are not limited to, zinc finger nucleases, meganucleases, TAL-effector DNA binding domain-nuclease fusion proteins (transcription activator-like effector nucleases (TALEN®s)), and CRISPR/Cas endonucleases (e.g., class 2 CRISPR/Cas endonucleases such as a type II, type V, or type VI CRISPR/Cas endonucleases). Thus, in some cases, a gene product is a sequence-specific genome editing endonuclease, e.g., genome editing, endonucleases selected from: a zinc finger nuclease, a TAL-effector DNA binding domain-nuclease fusion protein (TALEN), and a CRISPR/Cas endonuclease (e.g., a class 2 CRISPR/Cas endonuclease such as a type II, type V, or type VI CRISPR/Cas endonuclease). In some cases, a sequence-specific genome editing endonuclease includes a zinc finger nuclease or a TALEN. In some cases, a sequence-specific genome editing endonuclease includes a class 2 CRISPR/Cas endonuclease. In some cases, a sequence-specific genome editing endonuclease includes a class 2 type II CRISPR/Cas endonuclease (e.g., a Cas9 protein). In some cases, a sequence-specific genome editing endonuclease includes a class 2 type V CRISPR/Cas endonuclease (e.g., a Cpf1 protein, a C2c1 protein, or a C2c3 protein). In some cases, a sequence-specific genome editing endonuclease includes a class 2 type VI CRISPR/Cas endonuclease (e.g., a C2c2 protein).

RNA-mediated adaptive immune systems in bacteria and archaea rely on Clustered Regularly Interspaced Short Palindromic Repeat (CRISPR) genomic loci and CRISPR-associated (Cas) proteins that function together to provide protection from invading viruses and plasmids. In some cases, an RNA-guided endonuclease is a class 2 CRISPR/Cas endonuclease. In class 2 CRISPR systems, the functions of the effector complex (e.g., the cleavage of target DNA) are carried out by a single endonuclease (e.g., see Zetsche et al, Cell. 2015 Oct. 22; 163(3):759-71; Makarova et al, Nat Rev Microbiol. 2015 November; 13(11):722-36; and Shmakov et al., Mol Cell. 2015 Nov. 5; 60(3):385-97). As such, the term “class 2 CRISPR/Cas protein” is used herein to encompass the endonuclease (the target nucleic acid cleaving protein) from class 2 CRISPR systems. Thus, the term “class 2 CRISPR/Cas endonuclease” as used herein encompasses type II CRISPR/Cas proteins (e.g., Cas9), type V CRISPR/Cas proteins (e.g., Cpf1, C2c1, C2C3), and type VI CRISPR/Cas proteins (e.g., C2c2). To date, class 2 CRISPR/Cas proteins encompass type II, type V, and type VI CRISPR/Cas proteins, but the term is also meant to encompass any class 2 CRISPR/Cas protein suitable for binding to a corresponding guide RNA and forming an RNP complex.

In some cases, a suitable RNA-guided endonuclease comprises an amino acid sequence having at least 30%, at least 40%, at least 50%, at least 60%, at least 70%, at least 80%, at least 85%, at least 90%, at least 95%, at least 98%, at least 99%, or 100%, amino acid sequence identity to the Streptococcus pyogenes Cas9 amino acid sequence depicted in FIG. 21.

In some cases, a suitable RNA-guided endonuclease comprises an amino acid sequence having at least 30%, at least 40%, at least 50%, at least 60%, at least 70%, at least 80%, at least 85%, at least 90%, at least 95%, at least 98%, at least 99%, or 100%, amino acid sequence identity to the Staphylococcus aureus Cas9 amino acid sequence depicted in FIG. 22.

In some cases, the RNA-guided endonuclease is a nickase. Jinek et al., Science. 2012 Aug. 17; 337(6096):816-21).

In some cases, the RNA-guided endonuclease is a variant Cas9 protein that has reduced catalytic activity (e.g., when a Cas9 protein has a D10, G12, G17, E762, H840, N854, N863, H982, H983, A984, D986, and/or a A987 mutation of the amino acid sequence depicted in FIG. 21, e.g., D10A, G12A, G17A, E762A, H840A, N854A, N863A, H982A, H983A, A984A, and/or D986A); and the variant Cas9 protein retains the ability to bind to target nucleic acid in a site-specific manner (e.g., when complexed with a guide RNA.

In some cases, the RNA-guided endonuclease is a type V CRISPR/Cas protein. In some cases, the RNA-guided endonuclease is a type VI CRISPR/Cas protein. Examples and guidance related to type V and type VI CRISPR/Cas proteins (e.g., Cpf1, C2c1, C2c2, and C2c3 guide RNAs) can be found in the art, for example, see Zetsche et al, Cell. 2015 Oct. 22; 163(3):759-71; Makarova et al, Nat Rev Microbiol. 2015 November; 13(11):722-36; and Shmakov et al., Mol Cell. 2015 Nov. 5; 60(3):385-97.

In some cases, the RNA-guided endonuclease is a chimeric polypeptide (e.g., a fusion polypeptide) comprising: a) an RNA-guided endonuclease; and b) a fusion partner, where the fusion partner provides a functionality or activity other than an endonuclease activity. For example, the fusion partner can be a polypeptide having an enzymatic activity that modifies a polypeptide (e.g., a histone) associated with, or proximal to, a target nucleic acid (e.g., methyltransferase activity, deaminase activity (e.g., cytidine deaminase activity), demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity).

In some cases, the RNA-guided endonuclease is a base editor; for example, in some cases, the RNA-guided endonuclease is a fusion polypeptide comprising: a) an RNA-guided endonuclease; and b) a cytidine deaminase. See, e.g., Komor et al. (2016) Nature 533:420.

### Opsins

In some cases, a gene product encoded in a system of the present disclosure is a hyperpolarizing or a depolarizing light-activated polypeptide (an “opsin”). The light-activated polypeptide may be a light-activated ion channel or a light-activated ion pump. The light-activated ion channel polypeptides are adapted to allow one or more ions to pass through the plasma membrane of a neuron when the polypeptide is illuminated with light of an activating wavelength. Light-activated proteins may be characterized as ion pump proteins, which facilitate the passage of a small number of ions through the plasma membrane per photon of light, or as ion channel proteins, which allow a stream of ions to freely flow through the plasma membrane when the channel is open. In some embodiments, the light-activated polypeptide depolarizes the neuron when activated by light of an activating wavelength. Suitable depolarizing light-activated polypeptides, without limitation, are shown in FIG. 23. In some embodiments, the light-activated polypeptide hyperpolarizes the neuron when activated by light of an activating wavelength. Suitable hyperpolarizing light-activated polypeptides, without limitation, are shown in FIG. 24.

In some cases, a light-activated polypeptide comprises an amino acid sequence having at least 80%, at least 85%, at least 90%, at least 95%, at least 98%, at least 99%, or 100%, amino acid sequence identity to an opsin amino acid sequence depicted in FIG. 23. In some cases, a light-activated polypeptide comprises an amino acid sequence having at least 80%, at least 85%, at least 90%, at least 95%, at least 98%, at least 99%, or 100%, amino acid sequence identity to an opsin amino acid sequence depicted in FIG. 24.

In some embodiments, the light-activated polypeptides are activated by blue light. In some embodiments, the light-activated polypeptides are activated by green light. In some embodiments, the light-activated polypeptides are activated by yellow light. In some embodiments, the light-activated polypeptides are activated by orange light. In some embodiments, the light-activated polypeptides are activated by red light.

In some embodiments, the light-activated polypeptide expressed in a cell can be fused to one or more amino acid sequence motifs selected from the group consisting of a signal peptide, an endoplasmic reticulum (ER) export signal, a membrane trafficking signal, and/or an N-terminal golgi export signal. The one or more amino acid sequence motifs which enhance light-activated protein transport to the plasma membranes of mammalian cells can be fused to the N-terminus, the C-terminus, or to both the N- and C-terminal ends of the light-activated polypeptide. In some cases, the one or more amino acid sequence motifs which enhance light-activated polypeptide transport to the plasma membranes of mammalian cells is fused internally within a light-activated polypeptide. Optionally, the light-activated polypeptide and the one or more amino acid sequence motifs may be separated by a linker.

In some embodiments, the light-activated polypeptide can be modified by the addition of a trafficking signal (ts) which enhances transport of the protein to the cell plasma membrane. In some embodiments, the trafficking signal can be derived from the amino acid sequence of the human inward rectifier potassium channel Kir2.1. In other embodiments, the trafficking signal can comprise the amino acid sequence KSRITSEGEYIPLDQIDINV (SEQ ID NO:56). Trafficking sequences that are suitable for use can comprise an amino acid sequence having at least 85%, 90%, 91%, 92%, 93%, 94%, 95%, 96%, 97%, 98%, 99%, or 100%, amino acid sequence identity to an amino acid sequence such a trafficking sequence of human inward rectifier potassium channel Kir2.1 (e.g., KSRITSEGEYIPLDQIDINV (SEQ ID NO:56)).

A trafficking sequence can have a length of from about 10 amino acids to about 50 amino acids, e.g., from about 10 amino acids to about 20 amino acids, from about 20 amino acids to about 30 amino acids, from about 30 amino acids to about 40 amino acids, or from about 40 amino acids to about 50 amino acids.

ER export sequences that are suitable for use with a light-activated polypeptide include, e.g., VXXSL (where X is any amino acid; SEQ ID NO:52) (e.g., VKESL (SEQ ID NO:53); VLGSL (SEQ ID NO:54); etc.); NANSFCYENEVALTSK (SEQ ID NO:55); FXYENE (SEQ ID NO:57) (where X is any amino acid), e.g., FCYENEV (SEQ ID NO:58); and the like. An ER export sequence can have a length of from about 5 amino acids to about 25 amino acids, e.g., from about 5 amino acids to about 10 amino acids, from about 10 amino acids to about 15 amino acids, from about 15 amino acids to about 20 amino acids, or from about 20 amino acids to about 25 amino acids.

In some cases, a light-activated polypeptide is a fusion polypeptide that comprises an endoplasmic reticulum (ER) export signal (e.g., FCYENEV). In some cases, a light-activated polypeptide is a fusion polypeptide that comprises a membrane trafficking signal (e.g., KSRITSEGEYIPLDQIDINV). In some cases, a light-activated polypeptide is a fusion polypeptide comprising, in order from N-terminus to C-terminus: a) a light-activated polypeptide comprising an amino acid sequence having at least 80%, at least 85%, at least 90%, at least 95%, at least 98%, at least 99%, or 100%, amino acid sequence identity to an opsin amino acid sequence depicted in FIG. 23 or FIG. 24; b) an ER export signal; and c) a membrane trafficking signal.

### Transcription Factors

Suitable transcription factors include naturally-occurring transcription factors and recombinant (e.g., non-naturally occurring, engineered, artificial, synthetic) transcription factors. In some cases, the transcription is a transcriptional activator. In some cases, the transcriptional activator is an engineered protein, such as a zinc finger or TALE based DNA binding domain fused to an effector domain such as VP64 (transcriptional activation).

A transcription factor can comprise: i) a DNA binding domain (DBD); and ii) an activation domain (AD). The DBD can be any DBD with a known response element, including synthetic and chimeric DNA binding domains, or analogs, combinations, or modifications thereof. Suitable DNA binding domains include, but are not limited to, a GAL4 DBD, a LexA DBD, a transcription factor DBD, a Group H nuclear receptor member DBD, a steroid/thyroid hormone nuclear receptor superfamily member DBD, a bacterial LacZ DBD, an EcR DBD, a GALA DBD, and a LexA DBD. Suitable ADs include, but are not limited to, a Group H nuclear receptor member AD, a steroid/thyroid hormone nuclear receptor AD, a CJ7 AD, a p65-TA1 AD, a synthetic or chimeric AD, a polyglutamine AD, a basic or acidic amino acid AD, a VP16 AD, a GAL4 AD, an NF-κB AD, a BP64 AD, a B42 acidic activation domain (B42AD), a p65 transactivation domain (p65AD), SAD, NF-1, AP-2, SP1-A, SP1-B, Oct-1, Oct-2, MTF-1, BTEB-2, and LKLF, or an analog, combination, or modification thereof.

Suitable transcription factors include transcriptional activators, where suitable transcriptional activators include, but are not limited to, GAL4-VP16, GAL5-VP64, Tbx21, tTA-VP16, VP16, VP64, GAL4, p65, LexA-VP16, GAL4-NFκB, and the like.

Suitable transcription factors include transcriptional repressors, where suitable transcriptional repressors (e.g., a transcription repressor domain) include, but are not limited to, Krüppel-associated box (KRAB); the Mad mSIN3 interaction domain (SID); the ERF repressor domain (ERD); MDB-2B; v-ErbA; MBD3; and the like.

### Toxins

Suitable toxins include polypeptide toxins present in a natural source (e.g., naturally-occurring), recombinantly produced toxins, and synthetically produced toxins. Suitable toxins include ribosome inactivating proteins (RIPs); a bacterial toxin; and the like.

Suitable toxins include, e.g., anthopleurin B (GVPCLCDSDG-PRPRGNTLSG-ILWFYPSGCP-SGWHNCKAHG-PNIGWCCKK; SEQ ID NO://), anthopleurin C, anthopleurin Q, calitoxin (MKTQVLALFV LCVLFCLAES RTTLNKRNDI EKRIECKCEG DAPDLSHMTG TVYFSCKGGD GSWSKCNTYT AVADCCHQA; SEQ ID NO://), a conotoxin, ectatomin, HsTx1, omega-atracotoxin, a raventoxin, a scorpion toxin, and the like.

Suitable bacterial toxins include, e.g., cholera toxin, botulinum toxin, diphtheria toxin (produced by Corynebacterium diphtheriae), tetanospasmin, an enterotoxin, hemolysin, shiga toxin, erythrogenic toxin, adenylate cyclase toxin, pertussis toxin, ST toxin, LT toxin, ricin, abrin, tetanus toxin, and the like.

Exemplary Type I RIPS include, but are not limited to, gelonin, dodecandrin, tricosanthin, tricokirin, bryodin, Mirabilis antiviral protein (MAP), barley ribosome-inactivating protein (BRIP), pokeweed antiviral proteins (PAPS), saporins, luffins, and momordins. Exemplary Type II RIPS include, but are not limited to, ricin and abrin.

### Antibiotic Resistance Factors

As noted above, in some cases, the gene product of interest is an antibiotic resistance factor, e.g., a polypeptide that confers antibiotic resistance to a cell that produces the polypeptide.

Suitable antibiotic resistance factors include, but are not limited to, polypeptides that confer resistance to kanamycin, gentamicin, rifampin, trimethoprim, chloramphenicol, tetracycline, penicillin, methicillin, blasticidin, puromycin, hygromycin, or other antimicrobial agent. Suitable antibiotic resistance factors include, but are not limited to, aminoglycoside acetyltransferases, rifampin ADP-ribosyltransferases, dihydrofolate reductases, transporters, β-lactamases, chloramphenicol acetyltransferases, and efflux pumps. See, e.g., McGarvey et al. (2012) Applied Environ. Microbiol. 78:1708. Suitable antibiotic resistance factors include, but are not limited to, aminoglycoside 6′-N-acetyltransferase; gentamycin 3′-N-acetyltransferase; rifampin ADP-ribosyltransferase; dihydrofolate reductase; MFS transporter; ABC transporter; blasticidin-S deaminase; blasticidin acetyltransferase; puromycin N-acetyl-transferease; hygromycin kinase; and the like.

### Recombinases

In some cases, the gene product of interest is a recombinase. The term “recombinase” refers to an enzyme that catalyzes DNA exchange at a specific target site, for example, a palindromic sequence, by excision/insertion, inversion, translocation, and exchange.

Suitable recombinases include, but are not limited to, Cre recombinase; a FLP recombinase; a Tel recombinase; and the like. A suitable recombinase is one that targets (and cleaves) a target site selected from a telRL site, a loxP site, a phi pK02 telRL site, an FRT site, phiC31 attP site, and λattP site.

A suitable recombinase can be selected from the group consisting of: TelN; Tel; Tel (gp26 K02 phage); Cre; Flp; phiC31; Int; and a lambdoid phage integrase (e.g. a phi 80 recombinase, a HK022 recombinase; an HP1 recombinase).

Examples of target sites for such recombinases include, e.g.: a telRL site (targeted by a TelN recombinase): TATCAGCACACAATTGCCCATTATACGCGCGTATAATGGACTAT TGTGTGCTGA (SEQ ID NO://); a pal site: ACCTATTTCAGCATACTACGCGCGTAGTATGCTGAAATAGGT (SEQ ID NO://); a phi K02 telRL site: CCATTATACGCGCGTATAATGG (SEQ ID NO://); a loxP site (targeted by a Cre recombinase): TAACTTCGTATAGCATACATTATACGAAGTTAT (SEQ ID NO://); a FRT site (targeted by a Flp recombinase): GAAGTTCCTATTCTCTAGAAAGTATAGGAACTTC (SEQ ID NO://); a phiC31 attP site (targeted by a phiC31 recombinase): CCCAGGTCAGAAGCGGTTTTCGGGAGTAGTGCCCCAACTGGGGT AACCTTTGAGTTCTCTCAGTTGGGGGCGTAGGGTCGCCGACAYGA CACAAGGGGTT (SEQ ID NO://); a λ attP site: TGATAGTGACCTGTTCGTTTGCAACACATTGATGAGCAATGCTT TTTTATAATGCCAACTTTGTACAAAAAAGCTGAACGAGAAACGTA AAATGATATAAA (SEQ ID NO://).

### DREADDs

A suitable polypeptide of interest is in some cases a Designer Receptors Exclusively Activated by Designer Drugs (DREADD; also known as a “RASSL”). See e.g., Roth (2016) Neuron 89:683; Bang et al. (2016) Exp. Neurobiol. 25:205; Whissell et al. (2016) Front. Genet. 7:70; and U.S. Pat. No. 6,518,480. For example, a modified G protein-coupled receptor (GPCR) is genetically engineered so that it: 1) retains binding affinity for a synthetic small molecule; and 2) has decreased binding affinity for a selected naturally occurring peptide or nonpeptide ligand relative to binding by its corresponding wild-type GPCR (e.g., the GPCR from which the modified GPCR was derived). Synthetic small molecule binding to the modified receptor induces the target cell to respond with a specific physiological response (e.g., cellular proliferation, cellular secretion, cell migration, cell contraction, or pigment production).

Any G protein-coupled receptor having separable domains for: 1) natural ligand (e.g., a natural peptide ligand) binding; 2) synthetic small molecule binding; and 3) G protein interaction can be modified to produce a DREADD.

GPCRs that bind peptide as their natural ligand are in some cases used to generate a DREADD. Such GPCRs, include, but are not limited to: Type-1 Angiotensin II Receptor, Type-1a Angiotensin II Receptor, Type-1B Angiotensin II Receptor, Type-1C Angiotensin II Receptor, Type-2 Angiotensin II Receptor, Neuromedin-B Receptor, Gastrin-releasing Peptide Receptor, Bombesin Subtype-3 Receptor, B1 Bradykinin Receptor, B2 Bradykinin Receptor, Interleukin-8 A Receptor, Interleukin-8 B Receptor, FMet-Leu-Phe Receptor, Monocyte Chemoattractant Protein 1 Receptor, C-C Chemokine Receptor Type 1 Receptor, C5a Anaphylatoxin Receptor, Cholecystokinin Type A Receptor, Gastrin/cholecystokinin Type B Receptor, Endothelin-1 Receptor, Endothelin B Receptor, Follicle Stimulating Hormone (FSH-R) Receptor, Lutropin-choriogonadotropic Hormone (LH/CG-R) Receptor, Adrenocorticotropic Hormone Receptor (ACTH-R), Melanocyte Stimulating Hormone Receptor (MSH-R), Melanocortin-3 Receptor, Melanocortin-4 Receptor, Melanocortin-5 Receptor, Melatonin Type 1A Receptor, Melatonin Type 1B Receptor, Melatonin Type 1C Receptor, Neuropeptide Y Type 1 Receptor, Neuropeptide Y Type 2 Receptor, Neurotensin Receptor, Delta-type Opioid Receptor, Kappa-type Opioid Receptor, Mu-type Opioid, Nociceptin Receptor, Gonadotropin-releasing Hormone Receptor, Somatostatin Type 1 Receptor, Somatostatin Type 2 Receptor, Somatostatin Type 3 Receptor, Somatostatin Type 4 Receptor, Somatostatin Type 5 Receptor, Substance-P Receptor, Substance-K Receptor, Neuromedin K Receptor, Vasopressin Via Receptor, Vasopressin V1B Receptor, Vasopressin V2 Receptor, Oxytocin Receptor, Galanin Receptor, Calcitonin Receptor, Calcitonin A Receptor, Calcitonin B Receptor, Growth Hormone-releasing Hormone Receptor, Parathyroid Hormone/parathyroid Hormone-related Peptide Receptor, Pituitary Adenylate Cyclase Activating Polypeptide Type I Receptor, Secretin Receptor, Vasoactive Intestinal Polypeptide 1 Receptor, and Vasoactive Intestinal Polypeptide 2 Receptor.

A DREADD can interact with a G protein selected from Gi, Gq, and Gs. Thus, a DREADD can be a Gi-coupled DREADD, a Gq-coupled DREADD, or a Gs-coupled DREADD.

DREADDs include, but are not limited to, hM3Dq, a DREADD generated from the human M3 muscarinic receptor; hM4Di, a DREADD generated from the Gi-coupled human M4 muscarinic; a DREADD generated from a kappa opioid receptor (see U.S. Pat. No. 6,518,480); KORD; and the like.

### Nucleic Acid Gene Products

In some cases, a transcription factor present in a light-activated, calcium-gated transcription control polypeptide of the present disclosure, when released from the light-activated, calcium-gated transcription control polypeptide by cleavage of the proteolytically cleavable linker, controls transcription of a nucleotide sequence encoding a nucleic acid gene product.

Suitable nucleic acid gene products include, but are not limited to, an inhibitory nucleic acid, a ribozyme, a guide RNA that binds a target nucleic acid and an RNA-guided endonuclease, a microRNA (miRNA), an antisense RNA, a ribozyme, a decoy RNA, an anti-mir RNA, a long non-coding RNA, and the like. Typically, the nucleic acid gene product is not translated.

### Guide RNAs

Guide RNAs include RNAs (where a guide RNA can be a single RNA molecule or two RNA molecules) that comprise a first segment that comprises a nucleotide sequence that is complementary to (and hybridizes with) a target nucleotide sequence (e.g., a target nucleotide sequence present in genomic DNA), and a second segment that comprises a nucleotide sequence that binds to an RNA-guided endonuclease (e.g., a Cas9 polypeptide, a Cpf1 polypeptide, a C2c2 polypeptide, as described above).

In some cases, the guide RNA(s) bind to a Cas9 polypeptide. The first segment (targeting segment) of a Cas9 guide RNA includes a nucleotide sequence (a guide sequence) that is complementary to (and therefore hybridizes with) a specific sequence (a target site) within a target nucleic acid (e.g., a target ssRNA, a target ssDNA, the complementary strand of a double stranded target DNA, etc.). The protein-binding segment (or “protein-binding sequence”) interacts with (binds to) a Cas9 polypeptide. The protein-binding segment of a Cas9 guide RNA includes two complementary stretches of nucleotides that hybridize to one another to form a double stranded RNA duplex (dsRNA duplex). Site-specific binding and/or cleavage of a target nucleic acid (e.g., genomic DNA) can occur at locations (e.g., target sequence of a target locus) determined by base-pairing complementarity between the Cas9 guide RNA (the guide sequence of the Cas9 guide RNA) and the target nucleic acid.

In some cases, a guide RNA includes two separate nucleic acid molecules: an “activator” and a “targeter” and is referred to herein as a “dual guide RNA”, a “double-molecule guide RNA”, a “two-molecule guide RNA”, or a “dgRNA.” In some cases, the guide RNA is one molecule (e.g., for some class 2 CRISPR/Cas proteins, the corresponding guide RNA is a single molecule; and in some cases, an activator and targeter are covalently linked to one another, e.g., via intervening nucleotides), and the guide RNA is referred to as a “single guide RNA”, a “single-molecule guide RNA,” a “one-molecule guide RNA”, or simply “sgRNA.”

A “target nucleic acid” as used herein is a polynucleotide (e.g. a chromosomal DNA sequence; or an extrachromosomal sequence, e.g., an episomal sequence, a minicircle sequence, a mitochondrial sequence, a chloroplast sequence, etc.) that includes a site (“target site” “target sequence” or “endonuclease-recognized sequence”) targeted by a sequence-specific endonuclease, e.g., genome-editing endonuclease. When the sequence-specific endonuclease, e.g., genome editing endonuclease, is a CRISPR/Cas endonuclease, the target sequence is the sequence to which the guide sequence of a CRISPR/Cas guide RNA (e.g., a Cas9 guide RNA) will hybridize. For example, the target site (or target sequence) 5′-GAGCAUAUC-3′ within a target nucleic acid is targeted by (or is bound by, or hybridizes with, or is complementary to) the sequence 5′-GAUAUGCUC-3′. Suitable hybridization conditions include physiological conditions normally present in a cell. For a double stranded target nucleic acid, the strand of the target nucleic acid that is complementary to and hybridizes with the guide RNA is referred to as the “complementary strand” or “target strand”; while the strand of the target nucleic acid that is complementary to the “target strand” (and is therefore not complementary to the guide RNA) is referred to as the “non-target strand” or “non-complementary strand”.

Guide RNAs are well known in the art. Nucleotide sequences of the portion of the guide RNA that binds to a particular RNA-guided endonuclease (e.g., Cas9, Cpf1, C2c2, etc.) are known in the art. The portion of the guide RNA that hybridizes to a target nucleic acid can be designed based on the sequence of the target nucleic acid.

### Inhibitory RNAs

Inhibitory RNAs are well known in the art. RNAi is the sequence-specific, post-transcriptional silencing of a gene's expression by double-stranded RNA. RNAi is mediated by 21- to 25-nucleotide, double-stranded RNA molecules referred to as small interfering RNAs (siRNAs). siRNAs can be derived by enzymatic cleavage of double-stranded precursor short interfering RNAs (shRNA) expressed from genetic constructs or micro RNA precursors in cells.

### Cells Comprising a Polypeptide System

The present disclosure provides a cell comprising a FLARE system of the present disclosure. In some cases, the cell is in vitro. In some cases, the cell is in vivo.

The present disclosure provides a cell comprising a fusion polypeptide comprising: a) a transmembrane domain; b) a polypeptide that binds a calmodulin polypeptide or a troponin C polypeptide under certain Ca²⁺ concentration conditions (e.g., a Ca²⁺ concentration above about 100 nM); c) a light-activated polypeptide comprising a LOV domain; d) a proteolytically cleavable linker that is caged by the light-activated polypeptide in the absence of blue light; and e) a transcription factor.

The present disclosure provides a cell comprising a fusion polypeptide comprising: a) a calmodulin polypeptide; and b) a protease. The present disclosure provides a cell comprising a fusion polypeptide comprising: a) a troponin C polypeptide; and b) a protease.

The present disclosure provides a cell comprising: a first fusion polypeptide comprising: a) a transmembrane domain; b) a calmodulin-binding polypeptide that binds a calmodulin polypeptide under certain Ca²⁺ concentration conditions (e.g., a Ca²⁺ concentration above about 100 nM); c) a light-activated polypeptide comprising a LOV domain; d) a proteolytically cleavable linker that is caged by the light-activated polypeptide in the absence of blue light; and e) a transcription factor; and a second fusion polypeptide comprising: a) a calmodulin polypeptide; and b) a protease that cleaves the proteolytically cleavable linker under certain conditions.

The present disclosure provides a cell comprising: a first fusion polypeptide comprising: a) a transmembrane domain; b) a troponin I polypeptide that binds a troponin C polypeptide under certain Ca²⁺ concentration conditions (e.g., a Ca²⁺ concentration above about 100 nM); c) a light-activated polypeptide comprising a LOV domain; d) a proteolytically cleavable linker that is caged by the light-activated polypeptide in the absence of blue light; and e) a transcription factor; and a second fusion polypeptide comprising: a) a troponin C polypeptide; and b) a protease that cleaves the proteolytically cleavable linker under certain conditions.

Suitable cells include mammalian cells, amphibian cells, avian cells, insect cells, reptile cells, arachnid cells, and the like. In some cases, the cell is a primary (non-immortalized) cell. In some cases, the cell is an immortalized cell line.

In some cases, the cell is a mammalian cell, e.g., a human cell, a non-human primate cell, a rodent cell, a feline (e.g., a cat) cell, a canine (e.g., a dog) cell, an ungulate cell, an equine (e.g., a horse) cell, an ovine cell, a caprine cell, a bovine cell, etc. In some cases, the genetically modified host cell is a rodent cell (e.g., a rat cell; a mouse cell). In some cases, the genetically modified host cell is a human cell. In some cases, the genetically modified host cell is a non-human primate cell.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

Suitable host cells include cells of, e.g., Bacteria (e.g., Eubacteria); Archaebacteria; Protista; Fungi; Plantae; and Animalia. Suitable host cells include cells of plant-like members of the kingdom Protista, including, but not limited to, algae (e.g., green algae, red algae, glaucophytes, cyanobacteria); fungus-like members of Protista, e.g., slime molds, water molds, etc.; animal-like members of Protista, e.g., flagellates (e.g., Euglena), amoeboids (e.g., amoeba), sporozoans (e.g., Apicomplexa, Myxozoa, Microsporidia), and ciliates (e.g., Paramecium). Suitable host cells include cells of members of the kingdom Fungi, including, but not limited to, members of any of the phyla: Basidiomycota (club fungi; e.g., members of Agaricus, Amanita, Boletus, Cantherellus, etc.); Ascomycota (sac fungi, including, e.g., Saccharomyces); Mycophycophyta (lichens); Zygomycota (conjugation fungi); and Deuteromycota. Suitable host cells include cells of members of the kingdom Plantae, including, but not limited to, members of any of the following divisions: Bryophyta (e.g., mosses), Anthocerotophyta (e.g., hornworts), Hepaticophyta (e.g., liverworts), Lycophyta (e.g., club mosses), Sphenophyta (e.g., horsetails), Psilophyta (e.g., whisk ferns), Ophioglossophyta, Pterophyta (e.g., ferns), Cycadophyta, Gingkophyta, Pinophyta, Gnetophyta, and Magnoliophyta (e.g., flowering plants). Suitable host cells include cells of members of the kingdom Animalia, including, but not limited to, members of any of the following phyla: Porifera (sponges); Placozoa; Orthonectida (parasites of marine invertebrates); Rhombozoa; Cnidaria (corals, anemones, jellyfish, sea pens, sea pansies, sea wasps); Ctenophora (comb jellies); Platyhelminthes (flatworms); Nemertina (ribbon worms); Ngathostomulida (jawed worms)p Gastrotricha; Rotifera; Priapulida; Kinorhyncha; Loricifera; Acanthocephala; Entoprocta; Nemotoda; Nematomorpha; Cycliophora; Mollusca (mollusks); Sipuncula (peanut worms); Annelida (segmented worms); Tardigrada (water bears); Onychophora (velvet worms); Arthropoda (including the subphyla: Chelicerata, Myriapoda, Hexapoda, and Crustacea, where the Chelicerata include, e.g., arachnids, Merostomata, and Pycnogonida, where the Myriapoda include, e.g., Chilopoda (centipedes), Diplopoda (millipedes), Paropoda, and Symphyla, where the Hexapoda include insects, and where the Crustacea include shrimp, krill, barnacles, etc.; Phoronida; Ectoprocta (moss animals); Brachiopoda; Echinodermata (e.g. starfish, sea daisies, feather stars, sea urchins, sea cucumbers, brittle stars, brittle baskets, etc.); Chaetognatha (arrow worms); Hemichordata (acorn worms); and Chordata. Suitable members of Chordata include any member of the following subphyla: Urochordata (sea squirts; including Ascidiacea, Thaliacea, and Larvacea); Cephalochordata (lancelets); Myxini (hagfish); and Vertebrata, where members of Vertebrata include, e.g., members of Petromyzontida (lampreys), Chondrichthyces (cartilaginous fish), Actinopterygii (ray-finned fish), Actinista (coelocanths), Dipnoi (lungfish), Reptilia (reptiles, e.g., snakes, alligators, crocodiles, lizards, etc.), Aves (birds); and Mammalian (mammals). Suitable plant cells include cells of any monocotyledon and cells of any dicotyledon. Plant cells include, e.g., a cell of a leaf, a root, a tuber, a flower, and the like. In some cases, the genetically modified host cell is a plant cell. In some cases, the genetically modified host cell is a bacterial cell. In some cases, the genetically modified host cell is an archaeal cell.

Suitable eukaryotic host cells include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, Chlamydomonas reinhardtii, and the like. In some cases, subject genetically modified host cell is a yeast cell. In some instances, the yeast cell is Saccharomyces cerevisiae.

Suitable prokaryotic cells include any of a variety of bacteria, including laboratory bacterial strains, pathogenic bacteria, etc. Suitable prokaryotic hosts include, but are not limited, to any of a variety of gram-positive, gram-negative, or gram-variable bacteria. Examples include, but are not limited to, cells belonging to the genera: Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Phormidium, Pseudomonas, Rhodobacter, Rhodopseudomonas, Rhodospirillum, Rhodococcus, Salmonella, Scenedesmun, Serratia, Shigella, Staphylococcus, Strepromyces, Synnecoccus, and Zymomonas. Examples of prokaryotic strains include, but are not limited to: Bacillus subtilis, Bacillus amyloliquefacines, Brevibacterium ammoniagenes, Brevibacterium immariophilum, Clostridium beigerinckii, Enterobacter sakazakii, Escherichia coli, Lactococcus lactis, Mesorhizobium loti, Pseudomonas aeruginosa, Pseudomonas mevalonii, Pseudomonas pudica, Rhodobacter capsulatus, Rhodobacter sphaeroides, Rhodospirillum rubrum, Salmonella enterica, Salmonella typhi, Salmonella typhimurium, Shigella dysenteriae, Shigella flexneri, Shigella sonnei, and Staphylococcus aureus. One example of a suitable bacterial host cell is Escherichia coli cell.

Suitable plant cells include cells of a monocotyledon; cells of a dicotyledon; cells of an angiosperm; cells of a gymnosperm; etc.

### Nucleic Acids, Expression Vectors, and Host Cells

The present disclosure provides nucleic acid(s) comprising nucleotide sequences encoding one or more components of a FLARE system of the present disclosure. The present disclosure provides host cells genetically modified with the one or more nucleic acid(s).

The present disclosure provides a nucleic acid system comprising: a) a first nucleic acid comprising a nucleotide sequence encoding a first fusion polypeptide comprising: i) a transmembrane domain; ii) a calmodulin-binding polypeptide or a troponin I polypeptide that binds calmodulin or troponin C, respectively, under certain Ca²⁺ concentration conditions (e.g., a Ca²⁺ concentration above about 100 nM); ii) a light-activated polypeptide comprising a LOV domain; iii) a proteolytically cleavable linker that is caged by the light-activated polypeptide in the absence of blue light; and iv) a transcription factor; and b) a second nucleic acid comprising a nucleotide sequence encoding a second fusion polypeptide comprising: a) a calmodulin polypeptide or a troponin C polypeptide; and b) a protease that cleaves the proteolytically cleavable linker under certain conditions.

The present disclosure provides a nucleic acid system comprising: a) a first nucleic acid comprising a nucleotide sequence encoding a first fusion polypeptide comprising: i) a transmembrane domain; ii) a calmodulin-binding polypeptide that binds calmodulin under certain Ca²⁺ concentration conditions (e.g., a Ca²⁺ concentration above about 100 nM); ii) a light-activated polypeptide comprising a LOV domain; iii) a proteolytically cleavable linker that is caged by the light-activated polypeptide in the absence of blue light; and iv) a transcription factor; and b) a second nucleic acid comprising a nucleotide sequence encoding a second fusion polypeptide comprising: a) a calmodulin polypeptide; and b) a protease that cleaves the proteolytically cleavable linker under certain conditions.

The present disclosure provides a nucleic acid system comprising: a) a first nucleic acid comprising a nucleotide sequence encoding a first fusion polypeptide comprising: i) a transmembrane domain; ii) a troponin I polypeptide that binds a troponin C polypeptide under certain Ca²⁺ concentration conditions (e.g., a Ca²⁺ concentration above about 100 nM); ii) a light-activated polypeptide comprising a LOV domain; iii) a proteolytically cleavable linker that is caged by the light-activated polypeptide in the absence of blue light; and iv) a transcription factor; and b) a second nucleic acid comprising a nucleotide sequence encoding a second fusion polypeptide comprising: a) a troponin C polypeptide; and b) a protease that cleaves the proteolytically cleavable linker under certain conditions.

The present disclosure provides a nucleic acid comprising: a nucleic acid comprising: a) a nucleotide sequence encoding a fusion polypeptide comprising: i) a transmembrane domain; ii) calmodulin-binding polypeptide or a troponin I polypeptide that binds calmodulin or troponin C, respectively, under certain Ca²⁺ concentration conditions (e.g., a Ca²⁺ concentration above about 100 nM); ii) a light-activated polypeptide comprising a LOV domain; and iii) a proteolytically cleavable linker that is caged by the light-activated polypeptide in the absence of blue light; and b) an insertion site for inserting a nucleic acid comprising a nucleotide sequence encoding a transcription factor. The insertion site is within 10 nucleotides (nt), within 9 nt, within 8 nt, within 7 nt, within 6 nt, within 5 nt, within 4 nt, within 3 nt, within 2 nt, or 1 nt, of the 3′ end of the nucleotide sequence encoding the light-activated, calcium-gated fusion polypeptide. The insertion site is positioned relative to the nucleotide sequence encoding the light-activated, calcium-gated fusion polypeptide such that, after insertion of a nucleic acid comprising a nucleotide sequence encoding a transcription factor, and after transcription and translation, a fusion polypeptide comprising: i) a transmembrane domain; ii) a calmodulin-binding polypeptide or a troponin I polypeptide; iii) a LOV-domain light-activated polypeptide comprising an amino acid sequence having at least 80% amino acid sequence identity to the amino acid sequence depicted in any one of FIG. 15A-15G; iv) a proteolytically cleavable linker; and v) the transcription factor, is produced. In some cases, the insertion site is a multiple cloning site.

In any of the above embodiments, the nucleic acid(s) can be present in a recombinant expression vector. In some cases, the recombinant expression vector is a viral construct, e.g., a recombinant adeno-associated virus (AAV) construct, a recombinant adenoviral construct, a recombinant lentiviral construct, a recombinant retroviral construct, etc. In some cases, a nucleic acid of a system of the present disclosure is a recombinant lentivirus vector. In some cases, a nucleic acid of a system of the present disclosure is a recombinant AAV vector.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., Hum Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. In some cases, the vector is a lentivirus vector. Also suitable are transposon-mediated vectors, such as piggyback and sleeping beauty vectors.

In some cases, a nucleic acid or a nucleic acid system of the present disclosure is packaged in a viral particle. For example, in some cases, one or more of the nucleic acids of a nucleic acid system of the present disclosure are recombinant AAV vectors, and are packaged in recombinant AAV particles. Thus, the present disclosure provides a recombinant viral particle comprising a nucleic acid or a nucleic acid system of the present disclosure.

The present disclosure provides genetically modified host cells, where a host cell is genetically modified with a nucleic acid(s) comprising nucleotide sequences encoding one or more FLARE components, as described above. In some cases, a nucleic acid(s) comprising nucleotide sequences encoding one or more FLARE components, as described above, is stably integrated into the genome of the host cell. In some cases, a nucleic acid(s) comprising nucleotide sequences encoding one or more FLARE components, as described above, is present in the host cell episomally. The genetically modified cell can be in vitro or in vivo.

In some cases, the genetically modified host cell is a primary (non-immortalized) cell. In some cases, the genetically modified host cell is an immortalized cell line.

A genetically modified host cell of the present disclosure is a eukaryotic cell. Suitable host cells include mammalian cells, insect cells, reptile cells, amphibian cells, arachnid cells, and the like.

In some cases, the genetically modified host cell is a mammalian cell, e.g., a human cell, a non-human primate cell, a rodent cell, a feline (e.g., a cat) cell, a canine (e.g., a dog) cell, an ungulate cell, an equine (e.g., a horse) cell, an ovine cell, a caprine cell, a bovine cell, etc. In some cases, the genetically modified host cell is a rodent cell (e.g., a rat cell; a mouse cell). In some cases, the genetically modified host cell is a human cell. In some cases, the genetically modified host cell is a non-human primate cell.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

Suitable host cells include cells of, e.g., Bacteria (e.g., Eubacteria); Archaebacteria; Protista; Fungi; Plantae; and Animalia. Suitable host cells include cells of plant-like members of the kingdom Protista, including, but not limited to, algae (e.g., green algae, red algae, glaucophytes, cyanobacteria); fungus-like members of Protista, e.g., slime molds, water molds, etc.; animal-like members of Protista, e.g., flagellates (e.g., Euglena), amoeboids (e.g., amoeba), sporozoans (e.g, Apicomplexa, Myxozoa, Microsporidia), and ciliates (e.g., Paramecium). Suitable host cells include cells of members of the kingdom Fungi, including, but not limited to, members of any of the phyla: Basidiomycota (club fungi; e.g., members of Agaricus, Amanita, Boletus, Cantherellus, etc.); Ascomycota (sac fungi, including, e.g., Saccharomyces); Mycophycophyta (lichens); Zygomycota (conjugation fungi); and Deuteromycota. Suitable host cells include cells of members of the kingdom Plantae, including, but not limited to, members of any of the following divisions: Bryophyta (e.g., mosses), Anthocerotophyta (e.g., hornworts), Hepaticophyta (e.g., liverworts), Lycophyta (e.g., club mosses), Sphenophyta (e.g., horsetails), Psilophyta (e.g., whisk ferns), Ophioglossophyta, Pterophyta (e.g., ferns), Cycadophyta, Gingkophyta, Pinophyta, Gnetophyta, and Magnoliophyta (e.g., flowering plants). Suitable host cells include cells of members of the kingdom Animalia, including, but not limited to, members of any of the following phyla: Porifera (sponges); Placozoa; Orthonectida (parasites of marine invertebrates); Rhombozoa; Cnidaria (corals, anemones, jellyfish, sea pens, sea pansies, sea wasps); Ctenophora (comb jellies); Platyhelminthes (flatworms); Nemertina (ribbon worms); Ngathostomulida (jawed worms)p Gastrotricha; Rotifera; Priapulida; Kinorhyncha; Loricifera; Acanthocephala; Entoprocta; Nemotoda; Nematomorpha; Cycliophora; Mollusca (mollusks); Sipuncula (peanut worms); Annelida (segmented worms); Tardigrada (water bears); Onychophora (velvet worms); Arthropoda (including the subphyla: Chelicerata, Myriapoda, Hexapoda, and Crustacea, where the Chelicerata include, e.g., arachnids, Merostomata, and Pycnogonida, where the Myriapoda include, e.g., Chilopoda (centipedes), Diplopoda (millipedes), Paropoda, and Symphyla, where the Hexapoda include insects, and where the Crustacea include shrimp, krill, barnacles, etc.; Phoronida; Ectoprocta (moss animals); Brachiopoda; Echinodermata (e.g. starfish, sea daisies, feather stars, sea urchins, sea cucumbers, brittle stars, brittle baskets, etc.); Chaetognatha (arrow worms); Hemichordata (acorn worms); and Chordata. Suitable members of Chordata include any member of the following subphyla: Urochordata (sea squirts; including Ascidiacea, Thaliacea, and Larvacea); Cephalochordata (lancelets); Myxini (hagfish); and Vertebrata, where members of Vertebrata include, e.g., members of Petromyzontida (lampreys), Chondrichthyces (cartilaginous fish), Actinopterygii (ray-finned fish), Actinista (coelocanths), Dipnoi (lungfish), Reptilia (reptiles, e.g., snakes, alligators, crocodiles, lizards, etc.), Aves (birds); and Mammalian (mammals). Suitable plant cells include cells of any monocotyledon and cells of any dicotyledon. Plant cells include, e.g., a cell of a leaf, a root, a tuber, a flower, and the like. In some cases, the genetically modified host cell is a plant cell. In some cases, the genetically modified host cell is a bacterial cell. In some cases, the genetically modified host cell is an archaeal cell.

Suitable eukaryotic host cells include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, Chlamydomonas reinhardtii, and the like. In some cases, subject genetically modified host cell is a yeast cell. In some instances, the yeast cell is Saccharomyces cerevisiae.

Suitable prokaryotic cells include any of a variety of bacteria, including laboratory bacterial strains, pathogenic bacteria, etc. Suitable prokaryotic hosts include, but are not limited, to any of a variety of gram-positive, gram-negative, or gram-variable bacteria. Examples include, but are not limited to, cells belonging to the genera: Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Phormidium, Pseudomonas, Rhodobacter, Rhodopseudomonas, Rhodospirillum, Rhodococcus, Salmonella, Scenedesmun, Serratia, Shigella, Staphylococcus, Strepromyces, Synnecoccus, and Zymomonas. Examples of prokaryotic strains include, but are not limited to: Bacillus subtilis, Bacillus amyloliquefacines, Brevibacterium ammoniagenes, Brevibacterium immariophilum, Clostridium beigerinckii, Enterobacter sakazakii, Escherichia coli, Lactococcus lactis, Mesorhizobium loti, Pseudomonas aeruginosa, Pseudomonas mevalonii, Pseudomonas pudica, Rhodobacter capsulatus, Rhodobacter sphaeroides, Rhodospirillum rubrum, Salmonella enterica, Salmonella typhi, Salmonella typhimurium, Shigella dysenteriae, Shigella flexneri, Shigella sonnei, and Staphylococcus aureus. One example of a suitable bacterial host cell is Escherichia coli cell.

Suitable plant cells include cells of a monocotyledon; cells of a dicotyledon; cells of an angiosperm; cells of a gymnosperm; etc.

### Enhanced LOV Polypeptide

The present disclosure provides an enhanced LOV-domain light-activated polypeptide (also referred to herein as an “enhanced LOV polypeptide” or an “eLOV polypeptide”). The present disclosure provides a nucleic acid comprising a nucleotide sequence encoding eLOV polypeptide of the present disclosure, and a recombinant expression vector comprising the nucleic acid. The present disclosure provides a genetically modified host cell comprising a nucleic acid comprising a nucleotide sequence encoding eLOV polypeptide of the present disclosure, or a recombinant expression vector comprising the nucleic acid.

In some cases, an eLOV polypeptide comprises an amino sequence having at least 80%, at least 85%, at least 90%, at least 95%, at least 98%, at least 99%, or 100%, amino acid sequence identity to the following amino acid sequence: SLATTLERIEKNFVITDPRLPDNPIIFASDSFLQLTEYSREEILGRNCRFLQGPETDRATVR KIRDAIDNQTEVTVQLINYTKSGKKFWNLFHLQPMRDQKGDVQYFIGVQLDGTEHVRD AAEREAVMLIKKTAEEIDEAAK (SEQ ID NO://); and comprises a substitution at one or more of amino acids L2, N12, A28, H117, and I130, where the numbering is based on the amino acid sequence SLATTLERIEKNFVITDPRLPDNPIIFASDSFLQLTEYSREEILGRNCRFLQGPETDRATVR KIRDAIDNQTEVTVQLINYTKSGKKFWNLFHLQPMRDQKGDVQYFIGVQLDGTEHVRD AAEREAVMLIKKTAEEIDEAAK (SEQ ID NO://). In some cases, the eLOV polypeptide comprises a substitution selected from an L2R substitution, an L2H substitution, an L2P substitution, and an L2K substitution. In some cases, the eLOV polypeptide comprises a substitution selected from an N12S substitution, an N12T substitution, and an N12Q substitution. In some cases, the eLOV polypeptide comprises a substitution selected from an A28V substitution, an A28I substitution, and an A28L substitution. In some cases, the eLOV polypeptide comprises a substitution selected from an H117R substitution, and an H117K substitution. In some cases, the eLOV polypeptide comprises a substitution selected from an I130V substitution, an I130A substitution, and an I130L substitution. In some cases, the eLOV polypeptide comprises substitutions at amino acids L2, N12, and I130. In some cases, the eLOV polypeptide comprises substitutions at amino acids L2, N12, H117, and I130. In some cases, the eLOV polypeptide comprises substitutions at amino acids A28 and H117. In some cases, the eLOV polypeptide comprises substitutions at amino acids N12 and I130. In some cases, the eLOV polypeptide comprises an L2R substitution, an N12S substitution, and an I130V substitution. In some cases, the eLOV polypeptide comprises an N12S substitution and an I130V substitution. In some cases, the eLOV polypeptide comprises an A28V substitution and an H117R substitution. In some cases, the eLOV polypeptide comprises an L2P substitution, an N12S substitution, an I130V substitution, and an H117R substitution. In some cases, the eLOV polypeptide comprises an L2P substitution, an N12S substitution, an A28V substitution, an H117R substitution, and an I130V substitution. In some cases, the eLOV polypeptide comprises an L2P substitution, an N12S substitution, an I130V substitution, and an H117R substitution. In some cases, the eLOV polypeptide comprises an L2R substitution, an N12S substitution, an A28V substitution, an H117R substitution, and an I130V substitution. In some cases, the eLOV polypeptide has a length of 142 amino acids, 143 amino acids, 144 amino acids, 145 amino acids, 146 amino acids, 147 amino acids, 148 amino acids, 149 amino acids, or 150 amino acids. In some cases, the LOV polypeptide has a length of 142 amino acids.

In some cases, an eLOV polypeptide comprises an amino sequence having at least 80%, at least 85%, at least 90%, at least 95%, at least 98%, at least 99%, or 100%, amino acid sequence identity to the following amino acid sequence: SRATTLERIEKSFVITDPRLPDNPIIFVSDSFLQLTEYSREEILGRNCRFLQGPETDRATVR KIRDAIDNQTEVTVQLINYTKSGKKFWNLFHLQPMRDQKGDVQYFIGVQLDGTERVRD AAEREAVMLVKKTAEEIDEAAK (SEQ ID NO://); and has an Arg at amino acid 2, a Ser at amino acid 12, a Val at amino acid 28, an Arg at amino acid 117, and a Val at amino acid 130, as indicated by bold and underlined letters; and has a length of 142 amino acids, 143 amino acids, 144 amino acids, 145 amino acids, 146 amino acids, 147 amino acids, 148 amino acids, 149 amino acids, or 150 amino acids. In some cases, an eLOV polypeptide comprises the following amino acid sequence: SRATTLERIEKSFVITDPRLPDNPIIFVSDSFLQLTEYSREEILGRNCRFLQGPETDRATVR KIRDAIDNQTEVTVQLINYTKSGKKFWNLFHLQPMRDQKGDVQYFIGVQLDGTERVRD AAEREAVMLVKKTAEEIDEAAK (SEQ ID NO://); and has a length of 142 amino acids.

In some cases, an eLOV polypeptide comprises an amino sequence having at least 80%, at least 85%, at least 90%, at least 95%, at least 98%, at least 99%, or 100%, amino acid sequence identity to the following amino acid sequence: SRATTLERIEKSFVITDPRLPDNPVIFVSDSFLQLTEYSREEILGRNCRFLQGPETDRATVR KIRDAIDNQTEVTVQLINYTKSGKKFWNLFHLQPMRDQKGDVQYFIGVQLDGTERVRD AAEREAVMLVKKTAEEIDEAAK (SEQ ID NO://); and has an Arg at amino acid 2, a Ser at amino acid 12, a Val at amino acid 25, a Val at amino acid 28, an Arg at amino acid 117, and a Val at amino acid 130, as indicated by bold and underlined letters; and has a length of 142 amino acids, 143 amino acids, 144 amino acids, 145 amino acids, 146 amino acids, 147 amino acids, 148 amino acids, 149 amino acids, or 150 amino acids. In some cases, an eLOV polypeptide comprises the following amino acid sequence: SRATTLERIEKSFVITDPRLPDNPVIFVSDSFLQLTEYSREEILGRNCRFLQGPETDRATVR KIRDAIDNQTEVTVQLINYTKSGKKFWNLFHLQPMRDQKGDVQYFIGVQLDGTERVRD AAEREAVMLVKKTAEEIDEAAK (SEQ ID NO://); and has a length of 142 amino acids.

In some cases, an eLOV polypeptide of the present disclosure comprises one or more amino acid substitutions relative to the LOV2 amino acid sequence depicted in FIG. 15A. In some cases, an eLOV polypeptide of the present disclosure comprises one or more amino acid substitutions at positions selected from 1, 2, 12, 25, 28, 91, 100, 117, 118, 119, 120, 126, 128, 135, 136, and 138, relative to the LOV2 amino acid sequence depicted in FIG. 15A. Suitable substitutions include, Asp→Ser at amino acid 1; Asp→Phe at amino acid 1; Leu→Arg at amino acid 2; Asn→Ser at amino acid 12; Ile→Val at amino acid 12; Ala→Val at amino acid 28; Leu→Val at amino acid 91; Gln→Tyr at amino acid 100; His→Arg at amino acid 117; Val→Leu at amino acid 118; Arg→His at amino acid 119; Asp→Gly at amino acid 120; Gly→Ala at amino acid 126; Met→Cys at amino acid 128; Glu→Phe at amino acid 135; Asn→Gln at amino acid 136; Asn→Glu at amino acid 136; and Asp→Ala at amino acid 138, where the amino acid numbering is based on the number of the LOV2 amino acid sequence depicted in FIG. 15A.

In some cases, an eLOV polypeptide of the present disclosure comprises an amino acid sequence having at least 80%, at least 85%, at least 90%, at least 95%, at least 98%, at least 99%, or 100%, amino acid sequence identity to the amino acid sequence depicted in FIG. 15B, where amino acid 1 is Ser, amino acid 28 is Ala, amino acid 126 is Ala, and amino acid 136 is Glu. In some case, an eLOV polypeptide of the present disclosure has a length of 142 amino acids.

In some cases, an eLOV polypeptide of the present disclosure comprises an amino acid sequence having at least 80%, at least 85%, at least 90%, at least 95%, at least 98%, at least 99%, or 100%, amino acid sequence identity to the amino acid sequence depicted in FIG. 15C, where amino acid 1 is Ser; amino acid 2 is Arg; amino acid 12 is Ser; amino acid 28 is Ala; amino acid 117 is Arg; amino acid 126 is Ala; and amino acid 136 is Glu. In some case, an eLOV polypeptide of the present disclosure has a length of 142 amino acids.

In some cases, an eLOV polypeptide of the present disclosure comprises an amino acid sequence having at least 80%, at least 85%, at least 90%, at least 95%, at least 98%, at least 99%, or 100%, amino acid sequence identity to the amino acid sequence depicted in FIG. 15D, where amino acid 1 is Ser; amino acid 2 is Arg; amino acid 12 is Ser; amino acid 25 is Val; amino acid 28 is Val; amino acid 117 is Arg; amino acid 126 is Ala; amino acid 130 is Val; and amino acid 136 is Glu. In some case, an eLOV polypeptide of the present disclosure has a length of 142 amino acids.

In some cases, an eLOV polypeptide of the present disclosure comprises an amino acid sequence having at least 80%, at least 85%, at least 90%, at least 95%, at least 98%, at least 99%, or 100%, amino acid sequence identity to the amino acid sequence depicted in FIG. 15E, where amino acid 1 is Ser; amino acid 2 is Arg; amino acid 12 is Ser; amino acid 28 is Ala; amino acid 91 is Val; amino acid 100 is Tyr; amino acid 117 is Arg; amino acid 118 is Leu; amino acid 119 is His; amino acid 120 is Gly; amino acid 126 is Ala; amino acid 128 is Cys; amino acid 130 is Val; amino acid 135 is Phe; amino acid 136 is Gln; and amino acid 138 is Ala. In some case, an eLOV polypeptide of the present disclosure has a length of 142 amino acids.

In some cases, an eLOV polypeptide of the present disclosure comprises an amino acid sequence having at least 80%, at least 85%, at least 90%, at least 95%, at least 98%, at least 99%, or 100%, amino acid sequence identity to the amino acid sequence depicted in FIG. 15F, where amino acid 1 is Ser; amino acid 2 is Arg; amino acid 12 is Ser; amino acid 28 is Val; amino acid 117 is Arg; amino acid 126 is Ala; amino acid 130 is Val; and amino acid 136 is Glu. In some case, an eLOV polypeptide of the present disclosure has a length of 138 amino acids.

In some cases, an eLOV polypeptide of the present disclosure comprises an amino acid sequence having at least 80%, at least 85%, at least 90%, at least 95%, at least 98%, at least 99%, or 100%, amino acid sequence identity to the amino acid sequence depicted in FIG. 15G, where amino acid 1 is Ser; amino acid 2 is Arg; amino acid 12 is Ser; amino acid 28 is Val; amino acid 91 is Val; amino acid 100 is Tyr; amino acid 117 is Arg; amino acid 118 is Leu; amino acid 119 is His; amino acid 120 is Gly; amino acid 126 is Ala; amino acid 128 is Cys; amino acid 130 is Val; amino acid 135 is Phe; amino acid 136 is Gln; and amino acid 138 is Ala. In some case, an eLOV polypeptide of the present disclosure has a length of 138 amino acids.

In some cases, a LOV light-activated polypeptide comprises the following amino acid sequence:

(SEQ ID NO: //)
FRATTLERIEKSFVITDPRLPDNPIIFVSDSFLQLTEYSREEILGRN
CRFLQGPETDRATVRKIRDAIDNQTEVTVQLINYTKSGKKFWNVFHL
QPMRDYKGDVQYFIGVQLDGTERLHGAAEREAVCLVKKTAFQIA.

In some cases, a LOV light-activated polypeptide comprises the following amino acid sequence:

(SEQ ID NO: //)
SRATTLERIEKSFVITDPRLPDNPIIFVSDSFLQLTEYSREEILGRN
CRFLQGPETDRATVRKIRDAIDNQTEVTVQLINYTKSGKKFWNLFHL
QPMRDQKGDVQYFIGVQLDGTERVRDAAEREAVMLVKKTAEEID.

In some cases, a LOV light-activated polypeptide comprises the following amino acid sequence:

(SEQ ID NO: //)
FRATTLERIEKSFVITDPRLPDNPIIFVSDSFLQLTEYSREEILGRN
CRFLQGPETDRATVRKIRDAIDNQTEVTVQLINYTKSGKKFWNVFHL
QPMRDYKGDVQYFIGVQLDGTERLHGAAEREAVCLVKKTAFQIA.

In some cases, a LOV light-activated polypeptide comprises the following amino acid sequence:

(SEQ ID NO: //)
SRATTLERIEKSFVITDPRLPDNPIIFVSDSFLQLTEYSREEILGRN
CRFLQGPETDRATVRKIRDAIDNQTEVTVQLINYTKSGKKFWNVFHL
QPMRDYKGDVQYFIGVQLDGTERLHGAAEREAVCLVKKTAFEIDEAA
K.

In some cases, a LOV light-activated polypeptide comprises the following amino acid sequence:

(SEQ ID NO: //)
SRATTLERIEKSFVITDPRLPDNPIIFVSDSFLQLTEYSREEILGRN
CRFLQGPETDRATVRKIRDAIDNQTEVTVQLINYTKSGKKFWNLFHL
QPMRDQKGDVQYFIGVQLDGTERVRDAAEREAVMLVKKTAEEIDEAA
K.

When an eLOV polypeptide is present in a fusion polypeptide, e.g., where the fusion polypeptide comprises an eLOV polypeptide and a proteolytically cleavable linker, the eLOV polypeptide cages the proteolytically cleavable linker in the absence of light of an activating wavelength, the proteolytically cleavable linker is substantially not accessible to the protease. Thus, e.g., in the absence of light of an activating wavelength (e.g., in the dark; or in the presence of light of a wavelength other than blue light), the proteolytically cleavable linker is cleaved, if at all, to a degree that is more than 50% less, more than 60% less, more than 70% less, more than 80% less, more than 90% less, more than 95% less, more than 98% less, or more than 99% less, than the degree of cleavage of the proteolytically cleavable linker in the presence of light of an activating wavelength (e.g., blue light, e.g., light of a wavelength in the range of from about 450 nm to about 495 nm, from about 460 nm to about 490 nm, from about 470 nm to about 480 nm, e.g., 473 nm).

The present disclosure provides a nucleic acid comprising a nucleotide sequence encoding an eLOV polypeptide of the present disclosure. In some cases, the nucleotide sequence is operably linked to a transcriptional control element, e.g., a promoter.

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein.), it may be a spatially restricted promoter (i.e., transcriptional control element, enhancer, etc.)(e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

Suitable promoter and enhancer elements are known in the art. For expression in a bacterial cell, suitable promoters include, but are not limited to, lacI, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue-specific promoters and cell type-specific promoters.

Suitable promoters for use in plant cells include, e.g., various ubiquitin gene promoters, cauliflower mosaic virus 35S promoter (CaMV35S), the nopaline synthetase gene promoter, the PR1a gene promoter in tobacco, ribulose 1 in tomato, the 5-diphosphate carboxylase/oxidase small subunit gene promoter, the napin gene promoter, the oleosin gene promoter, etc.

Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

Inducible promoters suitable for use include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some cases, a nucleic acid comprising a nucleotide sequence encoding an eLOV polypeptide of the present disclosure is present in a recombinant expression vector. In some cases, the recombinant expression vector is a viral construct, e.g., a recombinant adeno-associated virus (AAV) construct, a recombinant adenoviral construct, a recombinant lentiviral construct, a recombinant retroviral construct, etc. In some cases, a nucleic acid comprising a nucleotide sequence encoding an eLOV polypeptide of the present disclosure is present in a recombinant lentivirus vector. In some cases, a nucleic acid comprising a nucleotide sequence encoding an eLOV polypeptide of the present disclosure is present in a recombinant AAV vector.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., Hum Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. In some cases, the vector is a lentivirus vector. Also suitable are transposon-mediated vectors, such as piggyback and sleeping beauty vectors.

The present disclosure provides a genetically modified host cell, where the cell is genetically modified with a nucleic acid comprising a nucleotide sequence encoding an eLOV polypeptide of the present disclosure. The present disclosure provides a genetically modified host cell, where the cell is genetically modified with a recombinant expression vector comprising a nucleic acid comprising a nucleotide sequence encoding an eLOV polypeptide of the present disclosure.

In some cases, the genetically modified host cell is a primary (non-immortalized) cell. In some cases, the genetically modified host cell is an immortalized cell line.

Suitable host cells include mammalian cells, insect cells, reptile cells, amphibian cells, arachnid cells, bacterial cells, archael cells, plant cells, fungal cells, yeast cells, algal cells, and the like.

In some cases, the genetically modified host cell is a mammalian cell, e.g., a human cell, a non-human primate cell, a rodent cell, a feline (e.g., a cat) cell, a canine (e.g., a dog) cell, an ungulate cell, an equine (e.g., a horse) cell, an ovine cell, a caprine cell, a bovine cell, etc. In some cases, the genetically modified host cell is a rodent cell (e.g., a rat cell; a mouse cell). In some cases, the genetically modified host cell is a human cell. In some cases, the genetically modified host cell is a non-human primate cell.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

Suitable host cells include cells of, e.g., Bacteria (e.g., Eubacteria); Archaebacteria; Protista; Fungi; Plantae; and Animalia. Suitable host cells include cells of plant-like members of the kingdom Protista, including, but not limited to, algae (e.g., green algae, red algae, glaucophytes, cyanobacteria); fungus-like members of Protista, e.g., slime molds, water molds, etc.; animal-like members of Protista, e.g., flagellates (e.g., Euglena), amoeboids (e.g., amoeba), sporozoans (e.g, Apicomplexa, Myxozoa, Microsporidia), and ciliates (e.g., Paramecium). Suitable host cells include cells of members of the kingdom Fungi, including, but not limited to, members of any of the phyla: Basidiomycota (club fungi; e.g., members of Agaricus, Amanita, Boletus, Cantherellus, etc.); Ascomycota (sac fungi, including, e.g., Saccharomyces); Mycophycophyta (lichens); Zygomycota (conjugation fungi); and Deuteromycota. Suitable host cells include cells of members of the kingdom Plantae, including, but not limited to, members of any of the following divisions: Bryophyta (e.g., mosses), Anthocerotophyta (e.g., hornworts), Hepaticophyta (e.g., liverworts), Lycophyta (e.g., club mosses), Sphenophyta (e.g., horsetails), Psilophyta (e.g., whisk ferns), Ophioglossophyta, Pterophyta (e.g., ferns), Cycadophyta, Gingkophyta, Pinophyta, Gnetophyta, and Magnoliophyta (e.g., flowering plants). Suitable host cells include cells of members of the kingdom Animalia, including, but not limited to, members of any of the following phyla: Porifera (sponges); Placozoa; Orthonectida (parasites of marine invertebrates); Rhombozoa; Cnidaria (corals, anemones, jellyfish, sea pens, sea pansies, sea wasps); Ctenophora (comb jellies); Platyhelminthes (flatworms); Nemertina (ribbon worms); Ngathostomulida (jawed worms)p Gastrotricha; Rotifera; Priapulida; Kinorhyncha; Loricifera; Acanthocephala; Entoprocta; Nemotoda; Nematomorpha; Cycliophora; Mollusca (mollusks); Sipuncula (peanut worms); Annelida (segmented worms); Tardigrada (water bears); Onychophora (velvet worms); Arthropoda (including the subphyla: Chelicerata, Myriapoda, Hexapoda, and Crustacea, where the Chelicerata include, e.g., arachnids, Merostomata, and Pycnogonida, where the Myriapoda include, e.g., Chilopoda (centipedes), Diplopoda (millipedes), Paropoda, and Symphyla, where the Hexapoda include insects, and where the Crustacea include shrimp, krill, barnacles, etc.; Phoronida; Ectoprocta (moss animals); Brachiopoda; Echinodermata (e.g. starfish, sea daisies, feather stars, sea urchins, sea cucumbers, brittle stars, brittle baskets, etc.); Chaetognatha (arrow worms); Hemichordata (acorn worms); and Chordata. Suitable members of Chordata include any member of the following subphyla: Urochordata (sea squirts; including Ascidiacea, Thaliacea, and Larvacea); Cephalochordata (lancelets); Myxini (hagfish); and Vertebrata, where members of Vertebrata include, e.g., members of Petromyzontida (lampreys), Chondrichthyces (cartilaginous fish), Actinopterygii (ray-finned fish), Actinista (coelocanths), Dipnoi (lungfish), Reptilia (reptiles, e.g., snakes, alligators, crocodiles, lizards, etc.), Aves (birds); and Mammalian (mammals). Suitable plant cells include cells of any monocotyledon and cells of any dicotyledon. Plant cells include, e.g., a cell of a leaf, a root, a tuber, a flower, and the like. In some cases, the genetically modified host cell is a plant cell. In some cases, the genetically modified host cell is a bacterial cell. In some cases, the genetically modified host cell is an archaeal cell.

Suitable eukaryotic host cells include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, Chlamydomonas reinhardtii, and the like. In some cases, subject genetically modified host cell is a yeast cell. In some instances, the yeast cell is Saccharomyces cerevisiae.

Suitable prokaryotic cells include any of a variety of bacteria, including laboratory bacterial strains, pathogenic bacteria, etc. Suitable prokaryotic hosts include, but are not limited, to any of a variety of gram-positive, gram-negative, or gram-variable bacteria. Examples include, but are not limited to, cells belonging to the genera: Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Phormidium, Pseudomonas, Rhodobacter, Rhodopseudomonas, Rhodospirillum, Rhodococcus, Salmonella, Scenedesmun, Serratia, Shigella, Staphylococcus, Strepromyces, Synnecoccus, and Zymomonas. Examples of prokaryotic strains include, but are not limited to: Bacillus subtilis, Bacillus amyloliquefacines, Brevibacterium ammoniagenes, Brevibacterium immariophilum, Clostridium beigerinckii, Enterobacter sakazakii, Escherichia coli, Lactococcus lactis, Mesorhizobium loti, Pseudomonas aeruginosa, Pseudomonas mevalonii, Pseudomonas pudica, Rhodobacter capsulatus, Rhodobacter sphaeroides, Rhodospirillum rubrum, Salmonella enterica, Salmonella typhi, Salmonella typhimurium, Shigella dysenteriae, Shigella flexneri, Shigella sonnei, and Staphylococcus aureus. One example of a suitable bacterial host cell is Escherichia coli cell.

Suitable plant cells include cells of a monocotyledon; cells of a dicotyledon; cells of an angiosperm; cells of a gymnosperm; etc.

### Genetically Modified Non-Human Organisms

The present disclosure provides genetically modified non-human organism, where the non-human organism is genetically modified with one or more nucleic acids of the present disclosure. The genetically modified non-human organism can be a vertebrate or an invertebrate animal. The genetically modified non-human organism can be a plant.

The genetically modified non-human organism can be an animal, e.g., a vertebrate animal. In some cases, the genetically modified non-human organism is a mammal. In some cases, the genetically modified non-human organism is an amphibian. In some cases, the genetically modified non-human organism is a reptile. In some cases, the genetically modified non-human organism is an insect. In some cases, the genetically modified non-human organism is an arachnid.

A nucleic acid of the present disclosure can be integrated into the genome of the genetically modified non-human organism. In some cases, the genetically modified non-human organism is heterozygous for the integration of the nucleic acid. In some cases, the genetically modified non-human organism is homozygous for the integration of the nucleic acid.

In some embodiments, a subject genetically modified non-human host cell can generate a subject genetically modified non-human organism (e.g., a mouse, a fish, a frog, a fly, a worm, etc.). For example, if the genetically modified host cell is a pluripotent stem cell (i.e., PSC) or a germ cell (e.g., sperm, oocyte, etc.), an entire genetically modified organism can be derived from the genetically modified host cell. In some embodiments, the genetically modified host cell is a pluripotent stem cell (e.g., embryonic stem cell (ESC), induced PSC (iPSC), pluripotent plant stem cell, etc.) or a germ cell (e.g., sperm cell, oocyte, etc.), either in vivo or in vitro, that can give rise to a genetically modified organism. In some embodiments the genetically modified host cell is a vertebrate PSC (e.g., ESC, iPSC, etc.) and is used to generate a genetically modified organism (e.g. by injecting a PSC into a blastocyst to produce a chimeric/mosaic animal, which could then be mated to generate non-chimeric/non-mosaic genetically modified organisms; grafting in the case of plants; etc.). Any convenient method/protocol for producing a genetically modified organism is suitable for producing a genetically modified host cell comprising a nucleic acid(s) of the present disclosure.

Methods of producing genetically modified organisms are known in the art. For example, see Cho et al., Curr Protoc Cell Biol. 2009 March; Chapter 19:Unit 19.11: Generation of transgenic mice; Gama et al., Brain Struct Funct. 2010 March; 214(2-3):91-109. Epub 2009 Nov. 25: Animal transgenesis: an overview; Husaini et al., GM Crops. 2011 June-December; 2(3): 150-62. Epub 2011 Jun. 1: Approaches for gene targeting and targeted gene expression in plants. A CRISPR/Cas9 system can be used to generate a transgenic organism. See, e.g., U.S. Patent Publication Nos. 2014/0068797 and 2015/0232882.

In some cases, a genetically modified organism comprises a target cell, and thus can be considered a source for target cells. For example, if a genetically modified cell comprising one or more nucleic acids of the present disclosure is used to generate a genetically modified organism, then the cells of the genetically modified organism comprise the one or more exogenous nucleic acids comprising nucleotide sequences encoding a polypeptide of the present disclosure (e.g., a light-activated, calcium-gated polypeptide; a light-activated, calcium-gated transcription factor; an eLOV polypeptide; etc.). In some such embodiments, the DNA of a cell or cells of the genetically modified organism can be targeted for modification by introducing into the cell or cells a nucleic acid(s) of the present disclosure.

A subject genetically modified non-human organism can be any organism other than a human, including for example, a plant; algae; an invertebrate (e.g., a cnidarian, an echinoderm, a worm, a fly, etc.); a vertebrate (e.g., a fish (e.g., zebrafish, puffer fish, gold fish, etc.), an amphibian (e.g., salamander, frog, etc.), a reptile, a bird, a mammal, etc.); an ungulate (e.g., a goat, a pig, a sheep, a cow, etc.); a rodent (e.g., a mouse, a rat, a hamster, a guinea pig); a lagomorpha (e.g., a rabbit); etc.

### Methods

The present disclosure provides methods of detecting a change in the intracellular calcium concentration in a cell in response to a stimulus. The present disclosure provides methods of modulating an activity of a cell. The methods generally involve exposing the cell to two stimuli substantially simultaneously: the first stimulus is blue light; and the second stimulus is any condition, agent, or other stimulus that effects an increase in the intracellular calcium concentration in the cell, such that the intracellular calcium concentration increases to above about 100 nM.

The cell is exposed to the first and the second stimulus substantially simultaneously, e.g., the cell is exposed to the first stimulus within about 1 second to about 60 seconds of the second stimulus, e.g., within about 1 second to about 5 seconds, within about 5 seconds to about 10 seconds, within about 10 seconds to about 15 seconds, within about 15 seconds to about 20 seconds, within about 20 seconds to about 30 seconds, within about 30 seconds to about 45 seconds, or within about 45 seconds to about 60 seconds, of the exposure to the cell of the second stimulus. In some cases, the cell is exposed to the first stimulus within less than 1 second of the exposure of the cell to the second stimulus, e.g., within 900 milliseconds, within 800 milliseconds, within 700 milliseconds, within 600 milliseconds, within 500 milliseconds, within 250 milliseconds, within 100 milliseconds, within 50 milliseconds, within 25 milliseconds, or within 10 milliseconds.

A system of the present disclosure, when present in a cell, can provide for temporal information. Thus, a method of the present disclosure can be carried out over time. For example, a signal generated by a system of the present disclosure can be detected for a continuous period of time following exposure to a first and second stimulus; e.g., for a continuous period of time of from 1 minute to several hours or days (e.g., from 1 minute to 15 minutes, from 15 minutes to 30 minutes, from 30 minutes to 1 hour, from 1 hour to 4 hours, from 4 hours to 8 hours, etc.) following exposure to a first and second stimulus. A signal generated by a system of the present disclosure can be detected periodically over a period of time following exposure to a first and second stimulus; e.g., periodically (e.g., once every 0.5 seconds, once every second, once every 15 seconds, once every 30 seconds, once every 60 seconds, once every 15 minutes, once every 30 minutes, once every hour, etc.) over a period of time of from 1 minute to several hours or days (e.g., from 1 minute to 15 minutes, from 15 minutes to 30 minutes, from 30 minutes to 1 hour, from 1 hour to 4 hours, from 4 hours to 8 hours, etc.) following exposure to a first and second stimulus.

### Detecting a Change in the Intracellular Calcium Concentration Using a FLARE System

The present disclosure provides methods of detecting a change in the intracellular calcium concentration in a cell in response to a stimulus. In some cases, the method comprises: a) exposing the cell to the stimulus; and substantially simultaneously exposing the cell to blue light, where the cell comprises a FLARE system of the present disclosure. An increase in a product of the reporter gene of the FLARE system, compared to a control level of the reporter gene product, indicates that exposure to the stimulus increases the intracellular calcium concentration in the cell.

In some cases, the cell (also referred to as a “target cell”) comprising a FLARE system of the present disclosure is in vitro. In some cases, the cell (also referred to as a “target cell”) comprising a FLARE system of the present disclosure is in vivo. The target cell is generally a eukaryotic cell. The target cell can be a mammalian cell, e.g., a human cell, a non-human primate cell, a rodent cell (e.g., a mouse cell; a rat cell), a lagomorph (e.g., rabbit) cell, etc.; a reptile cell; an amphibian cell; an insect cell; an arachnid cell; etc.

Where the cell is in vitro, a change in the intracellular calcium concentration can be detected by detecting a signal produced by a reporter gene product, e.g., using standard instrumentation (e.g., a colorimeter; a fluorimeter; a luminometer) for detecting such signals.

Where the cell is in vivo, a change in the intracellular calcium concentration can be detected by detecting a signal produced by a reporter gene product (e.g., such as any fluorescent protein (BFP, GFP, RFP, Venus, Neptune, Citrine, mCherry, dsRed, Tomato), an polypeptide with an epitope tag, luciferase, APEX, beta-galactosidase, beta-lactamase, HRP, peroxidase, chloramphenicol transferase, etc., and other reporter gene products listed elsewhere herein). Suitable reporter genes include those that complement a defect in an auxotroph (e.g., uracil, histidine, or leucine biosynthetic enzymes). Suitable reporter genes include drug resistance, antibiotic resistance, and the like.

Suitable target cells include, but are not limited to, neurons, endothelial cells, epithelial cells, astrocytes, glial cells, muscle cells, cardiomyocytes, keratinocytes, hepatocytes, retinal cells, adipocytes, chondrocytes, mesenchymal cells, osteoclasts, osteoblasts, stem cells, adult stem cells, and the like.

In some case, the target cell is in a particular tissue, e.g., brain tissue, kidney, liver, skin, blood, bone, skeletal muscle, cardiac muscle, breast tissue, lung, eye, or other tissue.

In some cases, the tissue is a brain tissue selected from the thalamus (including the central thalamus), sensory cortex (including the somatosensory cortex), zona incerta (ZI), ventral tegmental area (VTA), prefontal cortex (PFC), nucleus accumbens (NAc), amygdala (BLA), substantia nigra, ventral pallidum, globus pallidus, dorsal striatum, ventral striatum, subthalamic nucleus, hippocampus, dentate gyrus, cingulate gyrus, entorhinal cortex, olfactory cortex, primary motor cortex, and cerebellum.

Suitable target cells include stem cells, including iPS cells, ES cells, adult stem cells (e.g., cardiac stem cells; mesenchymal stem cells; etc.), etc.

Suitable target cells include cells of, e.g., Bacteria (e.g., Eubacteria); Archaebacteria; Protista; Fungi; Plantae; and Animalia. Suitable host cells include cells of plant-like members of the kingdom Protista, including, but not limited to, algae (e.g., green algae, red algae, glaucophytes, cyanobacteria); fungus-like members of Protista, e.g., slime molds, water molds, etc.; animal-like members of Protista, e.g., flagellates (e.g., Euglena), amoeboids (e.g., amoeba), sporozoans (e.g, Apicomplexa, Myxozoa, Microsporidia), and ciliates (e.g., Paramecium). Suitable host cells include cells of members of the kingdom Fungi, including, but not limited to, members of any of the phyla: Basidiomycota (club fungi; e.g., members of Agaricus, Amanita, Boletus, Cantherellus, etc.); Ascomycota (sac fungi, including, e.g., Saccharomyces); Mycophycophyta (lichens); Zygomycota (conjugation fungi); and Deuteromycota. Suitable host cells include cells of members of the kingdom Plantae, including, but not limited to, members of any of the following divisions: Bryophyta (e.g., mosses), Anthocerotophyta (e.g., hornworts), Hepaticophyta (e.g., liverworts), Lycophyta (e.g., club mosses), Sphenophyta (e.g., horsetails), Psilophyta (e.g., whisk ferns), Ophioglossophyta, Pterophyta (e.g., ferns), Cycadophyta, Gingkophyta, Pinophyta, Gnetophyta, and Magnoliophyta (e.g., flowering plants). Suitable host cells include cells of members of the kingdom Animalia, including, but not limited to, members of any of the following phyla: Porifera (sponges); Placozoa; Orthonectida (parasites of marine invertebrates); Rhombozoa; Cnidaria (corals, anemones, jellyfish, sea pens, sea pansies, sea wasps); Ctenophora (comb jellies); Platyhelminthes (flatworms); Nemertina (ribbon worms); Ngathostomulida (jawed worms)p Gastrotricha; Rotifera; Priapulida; Kinorhyncha; Loricifera; Acanthocephala; Entoprocta; Nemotoda; Nematomorpha; Cycliophora; Mollusca (mollusks); Sipuncula (peanut worms); Annelida (segmented worms); Tardigrada (water bears); Onychophora (velvet worms); Arthropoda (including the subphyla: Chelicerata, Myriapoda, Hexapoda, and Crustacea, where the Chelicerata include, e.g., arachnids, Merostomata, and Pycnogonida, where the Myriapoda include, e.g., Chilopoda (centipedes), Diplopoda (millipedes), Paropoda, and Symphyla, where the Hexapoda include insects, and where the Crustacea include shrimp, krill, barnacles, etc.; Phoronida; Ectoprocta (moss animals); Brachiopoda; Echinodermata (e.g. starfish, sea daisies, feather stars, sea urchins, sea cucumbers, brittle stars, brittle baskets, etc.); Chaetognatha (arrow worms); Hemichordata (acorn worms); and Chordata. Suitable members of Chordata include any member of the following subphyla: Urochordata (sea squirts; including Ascidiacea, Thaliacea, and Larvacea); Cephalochordata (lancelets); Myxini (hagfish); and Vertebrata, where members of Vertebrata include, e.g., members of Petromyzontida (lampreys), Chondrichthyces (cartilaginous fish), Actinopterygii (ray-finned fish), Actinista (coelocanths), Dipnoi (lungfish), Reptilia (reptiles, e.g., snakes, alligators, crocodiles, lizards, etc.), Aves (birds); and Mammalian (mammals). Suitable plant cells include cells of any monocotyledon and cells of any dicotyledon. Plant cells include, e.g., a cell of a leaf, a root, a tuber, a flower, and the like. In some cases, the genetically modified host cell is a plant cell. In some cases, the genetically modified host cell is a bacterial cell. In some cases, the genetically modified host cell is an archaeal cell.

Suitable eukaryotic host cells include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, Chlamydomonas reinhardtii, and the like. In some cases, subject genetically modified host cell is a yeast cell. In some instances, the yeast cell is Saccharomyces cerevisiae.

Suitable prokaryotic cells include any of a variety of bacteria, including laboratory bacterial strains, pathogenic bacteria, etc. Suitable prokaryotic hosts include, but are not limited, to any of a variety of gram-positive, gram-negative, or gram-variable bacteria. Examples include, but are not limited to, cells belonging to the genera: Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Phormidium, Pseudomonas, Rhodobacter, Rhodopseudomonas, Rhodospirillum, Rhodococcus, Salmonella, Scenedesmun, Serratia, Shigella, Staphylococcus, Strepromyces, Synnecoccus, and Zymomonas. Examples of prokaryotic strains include, but are not limited to: Bacillus subtilis, Bacillus amyloliquefacines, Brevibacterium ammoniagenes, Brevibacterium immariophilum, Clostridium beigerinckii, Enterobacter sakazakii, Escherichia coli, Lactococcus lactis, Mesorhizobium loti, Pseudomonas aeruginosa, Pseudomonas mevalonii, Pseudomonas pudica, Rhodobacter capsulatus, Rhodobacter sphaeroides, Rhodospirillum rubrum, Salmonella enterica, Salmonella typhi, Salmonella typhimurium, Shigella dysenteriae, Shigella flexneri, Shigella sonnei, and Staphylococcus aureus. One example of a suitable bacterial host cell is Escherichia coli cell.

Suitable plant cells include cells of a monocotyledon; cells of a dicotyledon; cells of an angiosperm; cells of a gymnosperm; etc.

In some cases, a FLARE system of the present disclosure provides a high signal-to-noise (S/N) ratio. For example, as described above, in some cases, a cell comprising a FLARE system of the present disclosure comprises: a) a first fusion polypeptide comprising: i) a TM domain; ii) a calmodulin-binding polypeptide or a troponin I polypeptide; iii) a LOV domain light-activated polypeptide; iv) a proteolytically cleavable linker; and v) a transcription factor; and b) a second fusion polypeptide comprising: i) a calmodulin polypeptide or a troponin C polypeptide; and where the cell is genetically modified with a heterologous nucleic acid comprising nucleotide sequence encoding a reporter, where the nucleotide sequence is operably linked to a promoter, and where the promoter is activated by the transcription factor when the transcription factor is released from the light-activated, calcium-gated transcription control polypeptide. For example, following exposure (substantially simultaneously) of such a cell comprising a FLARE system of the present disclosure to blue light and a second stimulus (such that the intracellular calcium concentration of the cell increases to above about 100 nM), the transcription factor is released from the light-activated, calcium-gated transcription control polypeptide (by cleavage of the proteolytically cleavable linker by the protease), and induces transcription of the heterologous nucleic acid, such that the reporter polypeptide is produced in the cell. The signal produced by the reporter polypeptide in a cell exposed substantially simultaneously to blue light and the second stimulus is at least 3-fold, at least 4-fold, at least 5-fold, at least 6-fold, at least 7-fold, at least 8-fold, at least 9-fold, at least 10-fold, or more than 10-fold, higher than the signal produced by the reporter polypeptide in a control cell not exposed substantially simultaneously to blue light and the second stimulus (e.g., in a control cell exposed to blue light and not to the second stimulus; in a control cell exposed to the second stimulus but not the blue light; or in a control cell exposed to both blue light and the second stimulus, but where the exposure is not substantially simultaneous).

### Stimuli

As noted above, a FLARE system of the present disclosure is activated in a target cell (e.g., a first fusion polypeptide (comprising: i) a TM domain; ii) a calmodulin-binding polypeptide or a troponin I polypeptide; iii) a LOV domain light-activated polypeptide; iv) a proteolytically cleavable linker; and v) a transcription factor) and a second fusion polypeptide (comprising: i) a calmodulin polypeptide or a troponin C polypeptide; and ii) a protease that cleaves the proteolytically cleavable linker) are brought into proximity to one another such that: i) the calmodulin polypeptide of the second fusion polypeptide and the calmodulin-binding polypeptide of the first fusion polypeptide bind to one another; and ii) the protease of the second fusion polypeptide cleaves the proteolytically cleavable linker of the first fusion polypeptide) only when the target cell comprising the FLARE system (the target cell comprises the first fusion polypeptide and the second fusion polypeptide, and is genetically modified with a heterologous nucleic acid comprising nucleotide sequence encoding a reporter polypeptide, where the nucleotide sequence is operably linked to a promoter that can be activated by the transcription factor upon release from the first polypeptide) is substantially simultaneously exposed to: a) a first stimulus, where the first stimulus is blue light (e.g., light of a wavelength in the range of from about 450 nm to about 495 nm, from about 460 nm to about 490 nm, from about 470 nm to about 480 nm, e.g., 473 nm); and b) a second stimulus, where the second stimulus induces an increase in the intracellular Ca²⁺ concentration of the cell to above about 100 nM (e.g., an increase of the intracellular Ca²⁺ concentration of the cell to greater than 100 nM, greater than 150 nM, greater than 200 nM, greater than 250 nM, greater than 300 nM, greater than 350 nM, greater than 400 nM, greater than 500 nM, or greater than 750 nM).

The second stimulus (the stimulus that induces an increase in the intracellular Ca²⁺ concentration of the target cell to above about 100 nM) can be any of a variety of stimuli. For example, the second stimulus can be: 1) binding of a ligand to a cell surface receptor present on the surface of the cell; 2) binding of a neurotransmitter to the cell (e.g., to a cell surface receptor for the neurotransmitter); 3) a change in temperature; 4) interaction of the target cell with a second cell (e.g., an effector cell); 5) binding of a hormone to the cell; 6) binding of a cytokine to the cell; 7) binding of a chemokine to the cell; 8) binding of a drug (e.g., a pharmaceutical agent) to the cell; 9) binding of an antibody to the cell (e.g., an antibody specific for an epitope present on the surface of the cell); 10) a change in oxygen concentration in the external environment of the cell (e.g., hypoxic conditions); 11) a change in the ion concentration in the liquid environment of the cell; 12) an electrical charge (e.g., producing a voltage change in the membrane of the cell); 13) a nutrient (e.g., a nutrient present in the external environment of the cell); 14) an adhesion polypeptide; 15) an extracellular matrix; 16) a pathogen (e.g., a virus, a protozoan, a bacterium); 17) a toxin; 18) a mitogen; 19) a drug, such as histamine, that triggers release of calcium from intracellular stores; 20) an ionophore (e.g., ionomycin, etc.); 21) external electrode stimulation; etc.

### Reporter Polypeptides

Suitable reporter polypeptides include polypeptides that generate a detectable signal. Suitable detectable signal-producing proteins include, e.g., fluorescent proteins; enzymes that catalyze a reaction that generates a detectable signal as a product; and the like.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrape1, mRaspberry, mGrape2, mPlum (Shaner et al. (2005) Nat. Methods 2:905-909), Neptune, and the like. Any of a variety of fluorescent and colored proteins from Anthozoan species, as described in, e.g., Matz et al. (1999) Nature Biotechnol. 17:969-973, or Rodriguez et al. (2016) Trends Biochem. Sci. is suitable for use.

Suitable enzymes include, but are not limited to, horse radish peroxidase (HRP), alkaline phosphatase (AP), beta-galactosidase (GAL), β-lactamase, glucose-6-phosphate dehydrogenase, beta-N-acetylglucosaminidase, J-glucuronidase, invertase, Xanthine Oxidase, luciferase, glucose oxidase (GO), engineered ascorbate peroxidase (e.g., APEX; APEX2); and the like. In some cases, the enzyme acts on a substrate to produce a colored product (e.g., a product that can be detected colorimetrically). In some cases, the enzyme acts on a substrate to produce a fluorescent product. In some cases, the enzyme acts on a substrate to produce a luminescent product.

### Detecting the Change in Intracellular Calcium Concentration Over Time

A method for detecting a change in the intracellular calcium concentration according to a method of the present disclosure can be carried out over time, providing information about dynamic changes to the intracellular calcium concentration in response to a given stimulus. For example, the change in the intracellular calcium concentration of a target cell can be detected over a period of time of from 5 seconds to 5 hours, e.g., from about 5 seconds to about 15 seconds, from about 15 seconds to about 30 seconds, from about 30 seconds to about 60 seconds, from about 1 minute to about 5 minutes, from about 5 minutes to about 15 minutes, from about 15 minutes to about 30 minutes, from about 30 minutes to about 60 minutes, from about 60 minutes to about 1 hour, from about 1 hour to about 2 hours, from about 2 hours to about 3 hours, from about 3 hours to about 4 hours, or from about 4 hours to about 5 hours. In some cases, the change in the intracellular calcium concentration of a target cell can be detected over a period of time of more than 5 hours.

### Modulating an Activity of Target Cell in Response to a Change in Intracellular Calcium Concentration

In some cases, a method of detecting a change in the intracellular calcium concentration of a target cell comprises: a) detecting a change in the intracellular calcium concentration; and b) where the detecting step indicates that the intracellular calcium concentration is greater than about 100 nM, modulating an activity of the target cell.

For example, in some cases, the target cell is further genetically modified with a heterologous nucleic acid comprising a nucleotide sequence encoding an “effector polypeptide” where the nucleotide sequence is operably linked to the same promoter to which the nucleotide sequence encoding the reporter gene product is operably linked, e.g., is operably linked to a promoter that is activated by the transcription factor that is released from the first fusion polypeptide.

In other instances, the target cell is further genetically modified with a heterologous nucleic acid comprising a nucleotide sequence encoding an “effector gene product” where the nucleotide sequence encoding the effector gene product is operably linked to a different promoter than the promoter to which the nucleotide sequence encoding the reporter gene product is operably linked, e.g., is operably linked to a promoter that is not activated by the transcription factor that is released from the first fusion polypeptide. An effector gene product can be an effector polypeptide or an effector nucleic acid.

Suitable effector polypeptides include, but are not limited to: 1) an opsin, e.g., a hyperpolarizing opsin or a depolarizing opsin, where suitable opsins are known in the art and are described above; in some cases, the opsin is one that is activated by light of a wavelength that is different from the wavelength of light that activates a LOV-domain light-activated polypeptide; 2) a toxin; 3) an apoptosis-inducing polypeptide; 4) a receptor; 5) a cytokine; 6) a chemokine; 7) an RNA-guided endonuclease (e.g., a Cas9 polypeptide, a Cpf1 polypeptide, a C2c2 polypeptide, etc.); 8) a recombinase (e.g., a Cre recombinase that acts on Lox sites); 9) a kinase; 10) a phosphatase; 11) a DREADD; 12) an antibody; etc.

Suitable effector nucleic acids include, but are not limited to: 1) a guide RNA (e.g., a guide RNA that binds an RNA-guided endonuclease (e.g., a Cas9 polypeptide, a Cpf1 polypeptide, a C2c2 polypeptide, etc.); 2) a ribozyme; 3) an inhibitory RNA; and 4) a microRNA.

Activities of a target cell that can be modulated using a method of the present disclosure include, but are not limited to: 1) proliferation; 2) secretion of a cytokine; 3) secretion of a chemokine; 4) secretion of a neurotransmitter; 4) cell behavior; 5) cell death; 6) cellular differentiation; 7) cell killing of another cell; 8) interaction with another cell; 9) transcription; 10) translation; 11) biosynthesis; 12) metabolism; etc.

### Methods of Modulating an Activity of a Cell Using a Light-Activated, Calcium-Gated Polypeptide

The present disclosure provides a method of modulating the activity of a cell using a light-activated, calcium-gated polypeptide of the present disclosure. The method generally involves exposing the cell to two stimuli substantially simultaneously: the first stimulus is blue light; and the second stimulus is any condition, agent, or other stimulus that effects an increase in the intracellular calcium concentration in the cell, such that the intracellular calcium concentration increases to above about 100 nM.

For example, a target cell comprises: a) a first fusion polypeptide comprising: i) a TM domain; ii) a calmodulin-binding polypeptide or a troponin I polypeptide; iii) a LOV domain light-activated polypeptide; iv) a proteolytically cleavable linker; and v) an effector polypeptide; and b) a second fusion polypeptide comprising: i) a calmodulin polypeptide; and b) a protease that cleaves the proteolytically cleavable linker. The first fusion polypeptide and the second fusion polypeptide are brought into proximity with one another only when the target is exposed, substantially simultaneously to two stimuli: a) blue light; and b) a second stimulus that effects an increase in the intracellular calcium concentration in the cell, such that the intracellular calcium concentration increases to above about 100 nM, e.g., above about 105 nM, above about 110 nM, above about 115 nM, above about 120 nM, above about 125 nM, above about 130 nM, above about 140 nM, above about 150 nM, above about 200 nM, above about 250 nM, above about 300 nM, above about 350 nM, above about 400 nM, above about 450 nM, or above about 500 nM.

The cell is exposed to the first and the second stimulus substantially simultaneously, e.g., the cell is exposed to the first stimulus within about 1 second to about 60 seconds of the second stimulus, e.g., within about 1 second to about 5 seconds, within about 5 seconds to about 10 seconds, within about 10 seconds to about 15 seconds, within about 15 seconds to about 20 seconds, within about 20 seconds to about 30 seconds, within about 30 seconds to about 45 seconds, or within about 45 seconds to about 60 seconds, of the exposure to the cell of the second stimulus. In some cases, the cell is exposed to the first stimulus within less than 1 second of the exposure of the cell to the second stimulus, e.g., within 900 milliseconds, within 800 milliseconds, within 700 milliseconds, within 600 milliseconds, within 500 milliseconds, within 250 milliseconds, within 100 milliseconds, within 50 milliseconds, within 25 milliseconds, or within 10 milliseconds.

In some cases, the cell (also referred to as a “target cell”) comprising a light-activated, calcium-gated system (where the light-activated, calcium-gated system comprises: a) a first fusion polypeptide comprising: i) a TM domain; ii) a calmodulin-binding polypeptide or a troponin I polypeptide; iii) a LOV domain light-activated polypeptide; iv) a proteolytically cleavable linker; and v) an effector polypeptide; and b) a second fusion polypeptide comprising: i) a calmodulin polypeptide; and b) a protease that cleaves the proteolytically cleavable linker) of the present disclosure is in vitro. In some cases, the cell (also referred to as a “target cell”) comprising a light-activated, calcium-gated system of the present disclosure is in vivo. The target cell is generally a eukaryotic cell. The target cell can be a mammalian cell, e.g., a human cell, a non-human primate cell, a rodent cell (e.g., a mouse cell; a rat cell), a lagomorph (e.g., rabbit) cell, etc.; a reptile cell; an amphibian cell; an insect cell; an arachnid cell; etc.

Suitable target cells include, but are not limited to, neurons, endothelial cells, epithelial cells, astrocytes, glial cells, muscle cells, cardiomyocytes, keratinocytes, hepatocytes, retinal cells, adipocytes, chondrocytes, mesenchymal cells, osteoclasts, osteoblasts, stem cells, adult stem cells, and the like.

In some case, the target cell is in a particular tissue, e.g., brain tissue, kidney, liver, skin, blood, bone, skeletal muscle, cardiac muscle, breast tissue, lung, eye, or other tissue.

In some cases, the tissue is a brain tissue selected from the thalamus (including the central thalamus), sensory cortex (including the somatosensory cortex), zona incerta (ZI), ventral tegmental area (VTA), prefontal cortex (PFC), nucleus accumbens (NAc), amygdala (BLA), substantia nigra, ventral pallidum, globus pallidus, dorsal striatum, ventral striatum, subthalamic nucleus, hippocampus, dentate gyrus, cingulate gyrus, entorhinal cortex, olfactory cortex, primary motor cortex, and cerebellum.

Suitable target cells include stem cells, including iPS cells, ES cells, adult stem cells (e.g., cardiac stem cells; mesenchymal stem cells; etc.), etc.

Suitable target cells include cells of, e.g., Bacteria (e.g., Eubacteria); Archaebacteria; Protista; Fungi; Plantae; and Animalia. Suitable host cells include cells of plant-like members of the kingdom Protista, including, but not limited to, algae (e.g., green algae, red algae, glaucophytes, cyanobacteria); fungus-like members of Protista, e.g., slime molds, water molds, etc.; animal-like members of Protista, e.g., flagellates (e.g., Euglena), amoeboids (e.g., amoeba), sporozoans (e.g, Apicomplexa, Myxozoa, Microsporidia), and ciliates (e.g., Paramecium). Suitable host cells include cells of members of the kingdom Fungi, including, but not limited to, members of any of the phyla: Basidiomycota (club fungi; e.g., members of Agaricus, Amanita, Boletus, Cantherellus, etc.); Ascomycota (sac fungi, including, e.g., Saccharomyces); Mycophycophyta (lichens); Zygomycota (conjugation fungi); and Deuteromycota. Suitable host cells include cells of members of the kingdom Plantae, including, but not limited to, members of any of the following divisions: Bryophyta (e.g., mosses), Anthocerotophyta (e.g., hornworts), Hepaticophyta (e.g., liverworts), Lycophyta (e.g., club mosses), Sphenophyta (e.g., horsetails), Psilophyta (e.g., whisk ferns), Ophioglossophyta, Pterophyta (e.g., ferns), Cycadophyta, Gingkophyta, Pinophyta, Gnetophyta, and Magnoliophyta (e.g., flowering plants). Suitable host cells include cells of members of the kingdom Animalia, including, but not limited to, members of any of the following phyla: Porifera (sponges); Placozoa; Orthonectida (parasites of marine invertebrates); Rhombozoa; Cnidaria (corals, anemones, jellyfish, sea pens, sea pansies, sea wasps); Ctenophora (comb jellies); Platyhelminthes (flatworms); Nemertina (ribbon worms); Ngathostomulida (jawed worms)p Gastrotricha; Rotifera; Priapulida; Kinorhyncha; Loricifera; Acanthocephala; Entoprocta; Nemotoda; Nematomorpha; Cycliophora; Mollusca (mollusks); Sipuncula (peanut worms); Annelida (segmented worms); Tardigrada (water bears); Onychophora (velvet worms); Arthropoda (including the subphyla: Chelicerata, Myriapoda, Hexapoda, and Crustacea, where the Chelicerata include, e.g., arachnids, Merostomata, and Pycnogonida, where the Myriapoda include, e.g., Chilopoda (centipedes), Diplopoda (millipedes), Paropoda, and Symphyla, where the Hexapoda include insects, and where the Crustacea include shrimp, krill, barnacles, etc.; Phoronida; Ectoprocta (moss animals); Brachiopoda; Echinodermata (e.g. starfish, sea daisies, feather stars, sea urchins, sea cucumbers, brittle stars, brittle baskets, etc.); Chaetognatha (arrow worms); Hemichordata (acorn worms); and Chordata. Suitable members of Chordata include any member of the following subphyla: Urochordata (sea squirts; including Ascidiacea, Thaliacea, and Larvacea); Cephalochordata (lancelets); Myxini (hagfish); and Vertebrata, where members of Vertebrata include, e.g., members of Petromyzontida (lampreys), Chondrichthyces (cartilaginous fish), Actinopterygii (ray-finned fish), Actinista (coelocanths), Dipnoi (lungfish), Reptilia (reptiles, e.g., snakes, alligators, crocodiles, lizards, etc.), Aves (birds); and Mammalian (mammals). Suitable plant cells include cells of any monocotyledon and cells of any dicotyledon. Plant cells include, e.g., a cell of a leaf, a root, a tuber, a flower, and the like. In some cases, the genetically modified host cell is a plant cell. In some cases, the genetically modified host cell is a bacterial cell. In some cases, the genetically modified host cell is an archaeal cell.

Suitable eukaryotic host cells include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, Chlamydomonas reinhardtii, and the like. In some cases, subject genetically modified host cell is a yeast cell. In some instances, the yeast cell is Saccharomyces cerevisiae.

Suitable prokaryotic cells include any of a variety of bacteria, including laboratory bacterial strains, pathogenic bacteria, etc. Suitable prokaryotic hosts include, but are not limited, to any of a variety of gram-positive, gram-negative, or gram-variable bacteria. Examples include, but are not limited to, cells belonging to the genera: Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Phormidium, Pseudomonas, Rhodobacter, Rhodopseudomonas, Rhodospirillum, Rhodococcus, Salmonella, Scenedesmun, Serratia, Shigella, Staphylococcus, Strepromyces, Synnecoccus, and Zymomonas. Examples of prokaryotic strains include, but are not limited to: Bacillus subtilis, Bacillus amyloliquefacines, Brevibacterium ammoniagenes, Brevibacterium immariophilum, Clostridium beigerinckii, Enterobacter sakazakii, Escherichia coli, Lactococcus lactis, Mesorhizobium loti, Pseudomonas aeruginosa, Pseudomonas mevalonii, Pseudomonas pudica, Rhodobacter capsulatus, Rhodobacter sphaeroides, Rhodospirillum rubrum, Salmonella enterica, Salmonella typhi, Salmonella typhimurium, Shigella dysenteriae, Shigella flexneri, Shigella sonnei, and Staphylococcus aureus. One example of a suitable bacterial host cell is Escherichia coli cell.

Suitable plant cells include cells of a monocotyledon; cells of a dicotyledon; cells of an angiosperm; cells of a gymnosperm; etc.

Activities of a target cell that can be modulated using a method of the present disclosure include, but are not limited to: 1) proliferation; 2) secretion of a cytokine; 3) secretion of a chemokine; 4) secretion of a neurotransmitter; 4) cell behavior; 5) cell death; 6) cellular differentiation; 7) cell killing of another cell; 8) interaction with another cell; 9) transcription; 10) translation; 11) ATP synthesis; 12) protein localization; 13) organelle localization; 14) metabolism; 15) biosynthesis; etc.

Suitable effector polypeptides are described in detail above. Suitable effector polypeptides include, but are not limited to, an opsin, a DREADD, a toxin, an enzyme, a transcription factor, an antibiotic resistance factor, a genome editing endonuclease, an RNA-guided endonuclease, a protease, a kinase, a phosphatase, a phosphorylase, a lipase, a receptor, and the like.

### Kits

The present disclosure provides a kit for using a FLARE system of the present disclosure, e.g., for carrying out a method of the present disclosure. A kit of the present disclosure provides one or more components of a FLARE system of the present disclosure and/or one or more nucleic acids comprising a nucleotide sequence(s) encoding one or more components of a FLARE system of the present disclosure.

In some cases, a kit of the present disclose comprises nucleic acid system comprising: A) a first nucleic acid comprising, in order from 5′ to 3′: a) a nucleotide sequence encoding a light-activated, calcium-gated fusion polypeptide of the present disclosure, e.g., a light-activated, calcium-gated fusion polypeptide comprising, in order from amino terminus to carboxyl terminus: i) a transmembrane domain (or other tethering polypeptide); ii) a calmodulin-binding polypeptide or a troponin I polypeptide; iii) a LOV-domain light-activated polypeptide comprising an amino acid sequence having at least 80%, at least 85%, at least 90%, at least 95%, at least 98%, at least 99%, or 100% amino acid sequence identity to the amino acid sequence depicted in any one of FIG. 15A-15D; and iv) a proteolytically cleavable linker; and b) an insertion site for a nucleic acid comprising a nucleotide sequence encoding a polypeptide of interest; and B) a second nucleic acid comprising a nucleotide sequence encoding a second fusion polypeptide comprising: i) a calcium-binding polypeptide selected from a calmodulin polypeptide and troponin C polypeptide; and ii) a protease that cleaves the proteolytically cleavable linker. In some cases, one or both of the first and the second nucleic acids are stably integrated into the genome of a cell; and the kit provides the cell (e.g., an in vitro cell; e.g., an in vitro mammalian cell) with one or both of the first and the second nucleic acids stably integrated into its genome. In some cases, one or both of the first and the second nucleic acids are present in a recombinant expression vector, e.g., a recombinant viral vector such as a recombinant AAV vector, a recombinant lentiviral vector, etc. In some cases, the polypeptide of interest is a transcription factor, and the kit further comprises a cell that is genetically modified with a nucleic acid comprising: a) a nucleotide sequence encoding a polypeptide; and b) a promoter that is responsive to the transcription factor, where the nucleotide sequence encoding the polypeptide is operably linked to the promoter; in some of these embodiments, the polypeptide is a fluorescent protein or other polypeptide that can be detected. Components of the kit can be provided in one or more containers, e.g., tubes, vials, etc.

In some cases, a kit of the present disclose comprises nucleic acid system comprising: A) a first nucleic acid comprising, in order from 5′ to 3′: a) a nucleotide sequence encoding a light-activated, calcium-gated fusion polypeptide of the present disclosure, e.g., a light-activated, calcium-gated fusion polypeptide comprising, in order from amino terminus to carboxyl terminus: i) a transmembrane domain (or other tethering polypeptide); ii) a calmodulin-binding polypeptide or a troponin I polypeptide; iii) a LOV-domain light-activated polypeptide comprising an amino acid sequence having at least 80%, at least 85%, at least 90%, at least 95%, at least 98%, at least 99%, or 100% amino acid sequence identity to the amino acid sequence depicted in any one of FIG. 15E-15G; and iv) a proteolytically cleavable linker; and b) an insertion site for a nucleic acid comprising a nucleotide sequence encoding a polypeptide of interest; and B) a second nucleic acid comprising a nucleotide sequence encoding a second fusion polypeptide comprising: i) a calcium-binding polypeptide selected from a calmodulin polypeptide and troponin C polypeptide; and ii) a protease that cleaves the proteolytically cleavable linker. In some cases, one or both of the first and the second nucleic acids are stably integrated into the genome of a cell; and the kit provides the cell (e.g., an in vitro cell; e.g., an in vitro mammalian cell) with one or both of the first and the second nucleic acids stably integrated into its genome. In some cases, one or both of the first and the second nucleic acids are present in a recombinant expression vector, e.g., a recombinant viral vector such as a recombinant AAV vector, a recombinant lentiviral vector, etc. In some cases, the polypeptide of interest is a transcription factor, and the kit further comprises a cell that is genetically modified with a nucleic acid comprising: a) a nucleotide sequence encoding a polypeptide; and b) a promoter that is responsive to the transcription factor, where the nucleotide sequence encoding the polypeptide is operably linked to the promoter; in some of these embodiments, the polypeptide is a fluorescent protein or other polypeptide that can be detected. Components of the kit can be provided in one or more containers, e.g., tubes, vials, etc.

In some cases, a kit of the present disclosure comprises a nucleic acid system comprising: a) a first nucleic acid comprising a nucleotide sequence encoding a light-activated, calcium-gated transcription control polypeptide comprising, in order from amino terminus to carboxyl terminus: i) a transmembrane domain; ii) a calmodulin-binding polypeptide or a troponin I polypeptide; iii) a LOV light-activated polypeptide comprising an amino acid sequence having at least 80%, at least 85%, at least 90%, at least 95%, at least 98%, at least 99%, or 100% amino acid sequence identity to the amino acid sequence depicted in one of FIG. 15A-15D; iv) a proteolytically cleavable linker; and v) a transcription factor; and b) a second nucleic acid comprising a nucleotide sequence encoding a fusion polypeptide comprising: i) a calcium-binding polypeptide selected from a calmodulin polypeptide and troponin C polypeptide; and ii) a protease that cleaves the proteolytically cleavable linker. In some cases, one or both of the first and the second nucleic acids are stably integrated into the genome of a cell; and the kit provides the cell (e.g., an in vitro cell; e.g., an in vitro mammalian cell)) with one or both of the first and the second nucleic acids stably integrated into its genome. In some cases, one or both of the first and the second nucleic acids are present in a recombinant expression vector, e.g., a recombinant viral vector such as a recombinant AAV vector, a recombinant lentiviral vector, etc. In some cases, the kit further comprises a cell that is genetically modified with a nucleic acid comprising: a) a nucleotide sequence encoding a polypeptide; and b) a promoter that is responsive to the transcription factor, where the nucleotide sequence encoding the polypeptide is operably linked to the promoter; in some of these embodiments, the polypeptide is a fluorescent protein or other polypeptide that can be detected. Components of the kit can be provided in one or more containers, e.g., tubes, vials, etc.

In some cases, a kit of the present disclosure comprises a nucleic acid system comprising: a) a first nucleic acid comprising a nucleotide sequence encoding a light-activated, calcium-gated transcription control polypeptide comprising, in order from amino terminus to carboxyl terminus: i) a transmembrane domain; ii) a calmodulin-binding polypeptide or a troponin I polypeptide; iii) a LOV light-activated polypeptide comprising an amino acid sequence having at least 80%, at least 85%, at least 90%, at least 95%, at least 98%, at least 99%, or 100% amino acid sequence identity to the amino acid sequence depicted in one of FIG. 15E-15G; iv) a proteolytically cleavable linker; and v) a transcription factor; and b) a second nucleic acid comprising a nucleotide sequence encoding a fusion polypeptide comprising: i) a calcium-binding polypeptide selected from a calmodulin polypeptide and troponin C polypeptide; and ii) a protease that cleaves the proteolytically cleavable linker. In some cases, one or both of the first and the second nucleic acids are stably integrated into the genome of a cell; and the kit provides the cell (e.g., an in vitro cell; e.g., an in vitro mammalian cell)) with one or both of the first and the second nucleic acids stably integrated into its genome. In some cases, one or both of the first and the second nucleic acids are present in a recombinant expression vector, e.g., a recombinant viral vector such as a recombinant AAV vector, a recombinant lentiviral vector, etc. In some cases, the kit further comprises a cell that is genetically modified with a nucleic acid comprising: a) a nucleotide sequence encoding a polypeptide; and b) a promoter that is responsive to the transcription factor, where the nucleotide sequence encoding the polypeptide is operably linked to the promoter; in some of these embodiments, the polypeptide is a fluorescent protein or other polypeptide that can be detected. Components of the kit can be provided in one or more containers, e.g., tubes, vials, etc.

The present disclosure provides a kit comprising a nucleic acid comprising: a) a nucleotide sequence encoding a light-activated, calcium-gated fusion polypeptide comprising, in order from amino terminus to carboxyl terminus: i) a transmembrane domain; ii) a calmodulin-binding polypeptide or a troponin I polypeptide; iii) a LOV-domain light-activated polypeptide comprising an amino acid sequence having at least 80%, at least 85%, at least 90%, at least 95%, at least 98%, at least 99%, or 100% amino acid sequence identity to the amino acid sequence depicted in any one of FIG. 15A-15D; and iv) a proteolytically cleavable linker; and b) an insertion site for a nucleic acid comprising a nucleotide sequence encoding a polypeptide of interest. In some cases, the kit further comprises a second nucleic acid comprising a nucleotide sequence encoding a fusion polypeptide comprising: i) a calcium-binding polypeptide selected from a calmodulin polypeptide and troponin C polypeptide; and ii) a protease that cleaves the proteolytically cleavable linker. One or both of the nucleic acids can be present in a recombinant expression vector, e.g., a recombinant viral vector such as a recombinant AAV vector, a recombinant lentiviral vector, etc. In some cases, one or both of the nucleic acids is stably integrated into the genome of a cell; and the kit provides the cell (e.g., an in vitro cell; e.g., an in vitro mammalian cell)) with one or both of the nucleic acids stably integrated into its genome.

The present disclosure provides a kit comprising a nucleic acid comprising: a) a nucleotide sequence encoding a light-activated, calcium-gated fusion polypeptide comprising, in order from amino terminus to carboxyl terminus: i) a transmembrane domain; ii) a calmodulin-binding polypeptide or a troponin I polypeptide; iii) a LOV-domain light-activated polypeptide comprising an amino acid sequence having at least 80%, at least 85%, at least 90%, at least 95%, at least 98%, at least 99%, or 100% amino acid sequence identity to the amino acid sequence depicted in any one of FIG. 15E-15G; and iv) a proteolytically cleavable linker; and b) an insertion site for a nucleic acid comprising a nucleotide sequence encoding a polypeptide of interest. In some cases, the kit further comprises a second nucleic acid comprising a nucleotide sequence encoding a fusion polypeptide comprising: i) a calcium-binding polypeptide selected from a calmodulin polypeptide and troponin C polypeptide; and ii) a protease that cleaves the proteolytically cleavable linker. One or both of the nucleic acids can be present in a recombinant expression vector, e.g., a recombinant viral vector such as a recombinant AAV vector, a recombinant lentiviral vector, etc. In some cases, one or both of the nucleic acids is stably integrated into the genome of a cell; and the kit provides the cell (e.g., an in vitro cell; e.g., an in vitro mammalian cell)) with one or both of the nucleic acids stably integrated into its genome.

A kit of the present disclosure can further include one or more additional reagents, where such additional reagents can be selected from: a buffer; a wash buffer; a control reagent; a positive control; a negative control; a reagent(s) for detecting production of a cleavage product of enzymatic cleavage of a substrate; and the like.

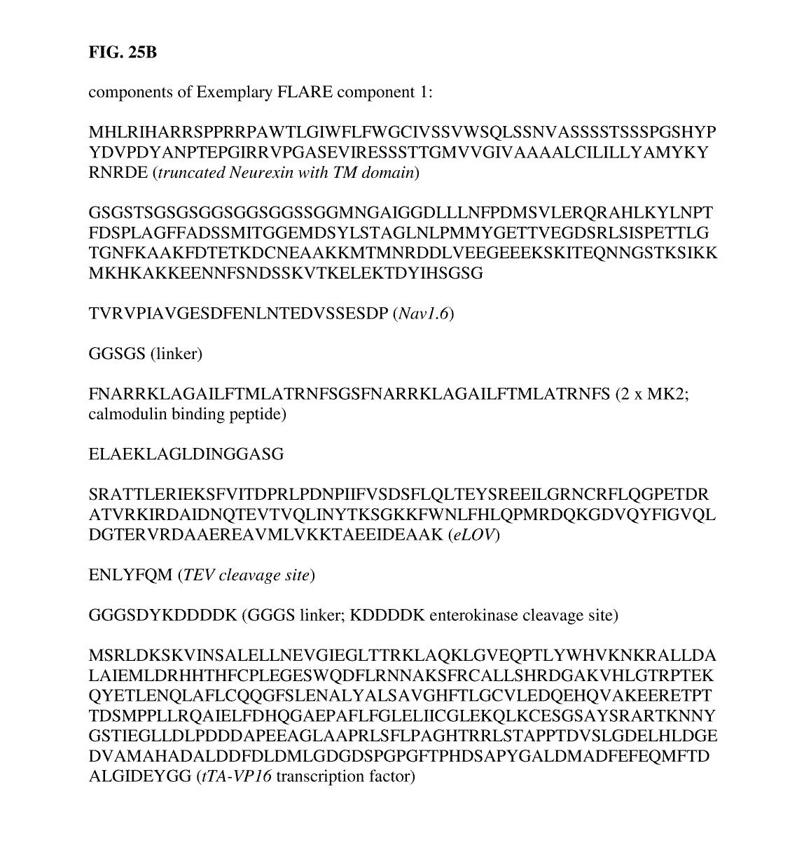

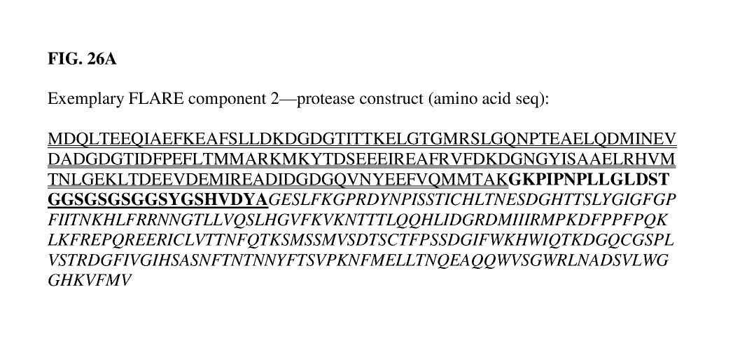

A suitable positive control can comprise: a) one or more nucleic acids comprising nucleotide sequences encoding: i) a first polypeptide comprising, in order from N-terminus to C-terminus: a TM domain, a calmodulin-binding polypeptide or a troponin I polypeptide, a LOV domain polypeptide (a LOV-domain light-activated polypeptide comprising an amino acid sequence having at least 80%, at least 85%, at least 90%, at least 95%, at least 98%, at least 99%, or 100% amino acid sequence identity to the amino acid sequence depicted in any one of FIG. 15A-15G), a proteolytically cleavable linker, and a transcription factor; and ii) a second polypeptide comprising, in order from N-terminus to C-terminus: a calmodulin polypeptide or a troponin C polypeptide, and a protease that cleaves the proteolytically cleavable linker; and B) a nucleic acid comprising: a) a nucleotide sequence encoding a fluorescent polypeptide; and b) a promoter that is responsive to the transcription factor, where the nucleotide sequence encoding the polypeptide is operably linked to the promoter. A suitable positive control can comprise one or more nucleic acids comprising nucleotide sequences encoding the FLARE components depicted in FIG. 25 and FIG. 26, and a nucleic acid comprising the nucleotide sequence depicted in FIG. 27. Those skilled in the art would be aware of other suitable positive controls.

Components of a subject kit can be in separate containers; or can be combined in a single container.

In addition to above-mentioned components, a subject kit can further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-141 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A nucleic acid system comprising: A) a first nucleic acid comprising, in order from 5′ to 3′: a) a nucleotide sequence encoding a light-activated, calcium-gated fusion polypeptide comprising, in order from amino terminus to carboxyl terminus: i) a transmembrane domain (or other tethering polypeptide); ii) a calmodulin-binding polypeptide or a troponin I polypeptide; iii) a LOV-domain light-activated polypeptide comprising an amino acid sequence having at least 80% amino acid sequence identity to the amino acid sequence depicted in any one of FIG. 15A-15G; and iv) a proteolytically cleavable linker; and b) an insertion site for a nucleic acid comprising a nucleotide sequence encoding a polypeptide of interest; and B) a second nucleic acid comprising a nucleotide sequence encoding a second fusion polypeptide comprising: i) a calcium-binding polypeptide selected from a calmodulin polypeptide and troponin C polypeptide; and ii) a protease that cleaves the proteolytically cleavable linker.

Aspect 2. A nucleic acid system comprising: a) a first nucleic acid comprising a nucleotide sequence encoding a light-activated, calcium-gated fusion polypeptide comprising, in order from amino terminus to carboxyl terminus: i) a transmembrane domain (or other tethering polypeptide); ii) a calmodulin-binding polypeptide or a troponin I polypeptide; iii) a LOV light-activated polypeptide comprising an amino acid sequence having at least 80% amino acid sequence identity to the amino acid sequence depicted in any one of FIG. 15A-15G; iv) a proteolytically cleavable linker; and v) a polypeptide of interest; and b) a second nucleic acid comprising a nucleotide sequence encoding a second fusion polypeptide comprising: i) a calcium-binding polypeptide selected from a calmodulin polypeptide and troponin C polypeptide; and ii) a protease that cleaves the proteolytically cleavable linker.

Aspect 3. The nucleic acid system of aspect 1, wherein the insertion site is a multiple cloning site.

Aspect 4. The nucleic acid system of any one of aspects 1-3, wherein the light-activated, calcium-gated fusion polypeptide comprises a calmodulin-binding polypeptide.

Aspect 5. The nucleic acid system of aspect 4, wherein the calmodulin-binding polypeptide comprises an amino acid sequence having at least 80% amino acid sequence identity to KRRWKKNFIAVSAANRFKKISSSGAL (SEQ ID NO://) or FNARRKLKGAILTTMLATRNFS (SEQ ID NO://).

Aspect 6. The nucleic acid system of aspect 4, wherein the calmodulin-binding polypeptide comprises an A14F substitution relative to the amino acid sequence KRRWKKNFIAVSAANRFKKISSSGAL.

Aspect 7. The nucleic acid system of aspect 5, wherein the calmodulin-binding polypeptide comprises T13F and K8A amino acid substitutions relative to the amino acid sequence FNARRKLKGAILTTMLATRNFS.

Aspect 8. The nucleic acid system of any one of aspects 1-3, wherein the light-activated, calcium-gated fusion polypeptide comprises a troponin I polypeptide.

Aspect 9. The nucleic acid system of aspect 8, wherein the troponin I polypeptide comprises an amino acid sequence having at least 80% amino acid sequence identity to the amino acid sequence depicted in FIG. 19A or FIG. 19B.

Aspect 10. The nucleic acid system of any one of aspects 1-9, wherein the LOV-domain light-activated polypeptide comprises one or more amino acid substitutions selected from L2R, N12S, A28V, H117R, and I130V substitutions relative to the amino acid sequence depicted in FIG. 15B.

Aspect 11. The nucleic acid system of any one of aspects 1-9, wherein the LOV domain light-activated polypeptide comprises L2R, N12S, I130V, A28V, and H117R substitutions relative to the amino acid sequence depicted in FIG. 15B.

Aspect 12. The nucleic acid system of any one of aspects 1-11, wherein the proteolytically cleavable linker comprises an amino acid sequence cleaved by a viral protease, a mammalian protease, or a recombinant protease.

Aspect 13. The nucleic acid system of any one of aspects 1-7 and 10-12, wherein the second fusion polypeptide comprises a calmodulin polypeptide.

Aspect 14. The nucleic acid system of aspect 13, wherein the calmodulin polypeptide comprises an amino acid sequence having at least 80% amino acid sequence identity to the amino acid sequence depicted in FIG. 16A or FIG. 16B.

Aspect 15. The nucleic acid system of aspect 14, wherein the calmodulin polypeptide comprises F19L and V35G substitutions relative to the amino acid sequence depicted in FIG. 16A.

Aspect 16. The nucleic acid system of any one of aspects 1-3 and 8-13, wherein the second fusion polypeptide comprises a troponin C polypeptide.

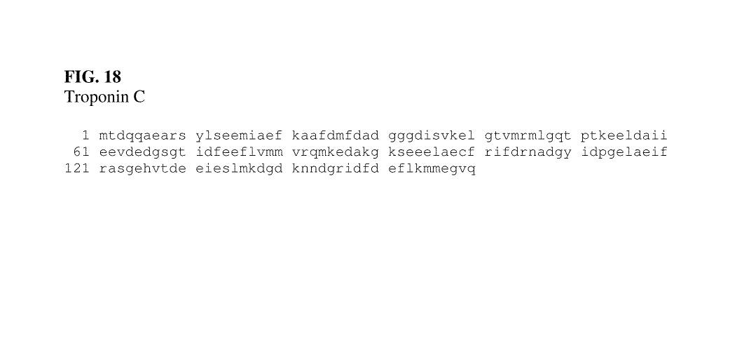

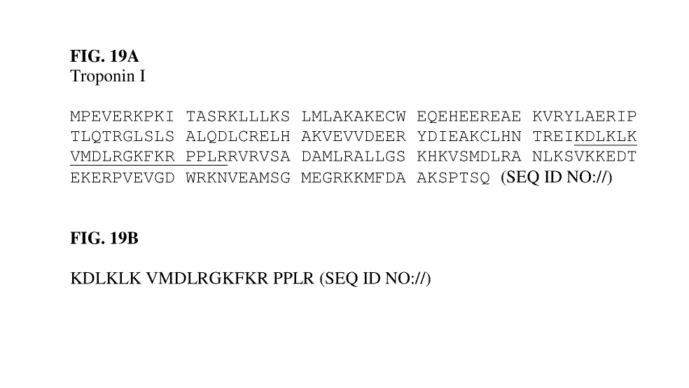

Aspect 17. The nucleic acid system of aspect 16, wherein the troponin C polypeptide comprises an amino acid sequence having at least 80% amino acid sequence identity to the amino acid sequence depicted in FIG. 18.

Aspect 18. The nucleic acid system of any one of aspects 1-17, wherein the protease is a viral protease, a mammalian protease, or a recombinant protease.

Aspect 19. The nucleic acid system of any one of aspects 1-18, wherein the first nucleic acid is present in a first expression vector, and the second nucleic acid is present in a second expression vector.

Aspect 20. The nucleic acid system of aspect 19, wherein the first expression vector and the second expression vector are recombinant viral vectors.

Aspect 21. The nucleic acid system of aspect 20, wherein the recombinant viral vector is a lentiviral vector, a retroviral vector, an adeno-associated viral vector, an adenoviral vector, or a herpes simplex virus vector.

Aspect 22. The nucleic acid system of any one of aspects 1-21, wherein the first and/or the second nucleic acid comprises a nucleotide sequence encoding a linker that is interposed between the transmembrane domain and the calmodulin-binding polypeptide or the troponin I polypeptide, between the calmodulin-binding polypeptide or the troponin I polypeptide and the LOV domain polypeptide, between the LOV domain polypeptide and the proteolytically cleavable linker, between the proteolytically cleavable linker and the polypeptide of interest, or between the calmodulin polypeptide or the troponin C polypeptide and the protease.

Aspect 23. The nucleic acid system of any one of aspects 2-21, wherein the polypeptide of interest is a reporter polypeptide, a light-activated polypeptide, a transcription factor, a toxin, a calcium sensor, a recombinase, an antibiotic resistance factor, a DREADD, an RNA-guided endonuclease, a drug-resistance factor, a kinase, a peroxidase, or an antibody.

Aspect 24. The nucleic acid system of aspect 23, wherein the polypeptide of interest is a reporter polypeptide selected from a fluorescent polypeptide, an enzyme that produces a colored product, an enzyme that produces a luminescent product, and an enzyme that produces a fluorescent product.

Aspect 25. The nucleic acid system of aspect 23, wherein the polypeptide of interest is a transcriptional activator or a transcriptional repressor.

Aspect 26. The nucleic acid system of aspect 23, wherein the polypeptide of interest is an antibiotic resistance factor.

Aspect 27. The nucleic acid system of aspect 23, wherein the polypeptide of interest is an RNA-guided endonuclease selected from a Cas9 polypeptide, a C2C2 polypeptide, or a Cpf1 polypeptide.

Aspect 28. A genetically modified host cell, wherein the host cell is genetically modified with the nucleic acid system of any one of aspects 1-27.

Aspect 29. The genetically modified host cell of aspect 28, wherein the cell is in vitro.

Aspect 30. The genetically modified host cell of aspect 28 or aspect 29, wherein the cell is a mammalian cell.

Aspect 31. The genetically modified host cell of any one of aspects 28-30, wherein the first and/or the second nucleic acid is stably integrated into the genome of the host cell.

Aspect 32. A nucleic acid comprising: a) a nucleotide sequence encoding a light-activated, calcium-gated fusion polypeptide comprising, in order from amino terminus to carboxyl terminus: i) a transmembrane domain; ii) a calmodulin-binding polypeptide or a troponin I polypeptide; iii) a LOV-domain light-activated polypeptide comprising an amino acid sequence having at least 80% amino acid sequence identity to the amino acid sequence depicted in any one of FIG. 15A-15G; and iv) a proteolytically cleavable linker; and b) an insertion site for a nucleic acid comprising a nucleotide sequence encoding a polypeptide of interest.

Aspect 33. A recombinant expression vector comprising the nucleic acid of aspect 32.

Aspect 34. A genetically modified host cell, wherein the host cell is genetically modified with the nucleic acid of aspect 32 or the recombinant expression vector of aspect 33.

Aspect 35. A nucleic acid comprising a nucleotide sequence encoding a light-activated, calcium-gated fusion polypeptide comprising, in order from amino terminus to carboxyl terminus: i) a transmembrane domain; ii) a calmodulin-binding polypeptide or a troponin I polypeptide; iii) a LOV light-activated polypeptide comprising an amino acid sequence having at least 80% amino acid sequence identity to the amino acid sequence depicted in any one of FIG. 15A-15G; iv) a proteolytically cleavable linker; and v) a gene product of interest.

Aspect 36. A recombinant expression vector comprising the nucleic acid of aspect 35.

Aspect 37. A genetically modified host cell, wherein the host cell is genetically modified with the nucleic acid of aspect 35 or the recombinant expression vector of aspect 36.

Aspect 38. A nucleic acid comprising a nucleotide sequence encoding a fusion polypeptide comprising: i) a calcium-binding polypeptide selected from a calmodulin polypeptide and troponin C polypeptide; and ii) a protease.

Aspect 39. A recombinant expression vector comprising the nucleic acid of aspect 38.

Aspect 40. A genetically modified host cell, wherein the host cell is genetically modified with the nucleic acid of aspect 38 or the recombinant expression vector of aspect 39.

Aspect 41. A kit comprising: a) the nucleic acid of aspect 33; and b) the genetically modified host cell of aspect 40.

Aspect 42. A light-activated, calcium-gated polypeptide comprising: i) a transmembrane domain; ii) a calmodulin-binding polypeptide or a troponin I polypeptide; iii) a LOV domain light-activated polypeptide comprising an amino acid sequence having at least 80% amino acid sequence identity to the amino acid sequence depicted in any one of FIG. 15A-15G; iv) a proteolytically cleavable linker; and v) a polypeptide of interest.

Aspect 43. A cell comprising the light-activated, calcium-gated polypeptide of aspect 42.

Aspect 44. The cell of aspect 43, wherein the cell is in vitro.

Aspect 45. The cell of aspect 43, wherein the cell is in vivo.

Aspect 46. A nucleic acid system comprising: a) a first nucleic acid comprising a nucleotide sequence encoding a light-activated, calcium-gated transcription control polypeptide comprising, in order from amino terminus to carboxyl terminus: i) a transmembrane domain; ii) a calmodulin-binding polypeptide or a troponin I polypeptide; iii) a LOV light-activated polypeptide comprising an amino acid sequence having at least 80% amino acid sequence identity to the amino acid sequence depicted in one of FIG. 15A-15G; iv) a proteolytically cleavable linker; and v) a transcription factor; and b) a second nucleic acid comprising a nucleotide sequence encoding a fusion polypeptide comprising: i) a calcium-binding polypeptide selected from a calmodulin polypeptide and troponin C polypeptide; and ii) a protease that cleaves the proteolytically cleavable linker.

Aspect 47. The nucleic acid system of aspect 46, wherein the calcium-binding polypeptide is calmodulin.

Aspect 48. The nucleic acid system of aspect 46 or aspect 47, wherein the first nucleic acid is a first recombinant expression vector, and the second nucleic acid is a second recombinant expression vector.

Aspect 49. The nucleic acid system of any one of aspects 46-48, comprising a third nucleic acid comprising a nucleotide sequence encoding a target gene product, wherein the target gene product-encoding nucleotide sequence is operably linked to a promoter that is activated by the transcription factor.

Aspect 50. The nucleic acid system of aspect 49, wherein the target gene product is a reporter polypeptide.

Aspect 51. The nucleic acid system of aspect 49, wherein the third nucleic acid is a third expression vector.

Aspect 52. The nucleic acid system of aspect 49 or aspect 50, wherein the third nucleic acid comprises a nucleotide sequence encoding a second light-responsive polypeptide, wherein the light-responsive polypeptide-encoding nucleotide sequence is operably linked to a promoter, wherein the second light activated polypeptide is activated by light of a wavelength that is different from the wavelength of light that activates the light-responsive polypeptide in the light-activated, calcium-gated transcription control polypeptide.

Aspect 53. A nucleic acid comprising: a) a first nucleotide sequence encoding the light-activated, calcium-gated transcription control polypeptide comprising: i) a transmembrane domain; ii) a calmodulin-binding polypeptide or a troponin I polypeptide; iii) a LOV domain light-activated polypeptide comprising an amino acid sequence having at least 80% amino acid sequence identity to the amino acid sequence depicted in any one of FIG. 15A-15G; iv) a proteolytically cleavable linker; and v) a transcription factor; and b) a second nucleotide sequence encoding a fusion polypeptide comprising: i) a calmodulin polypeptide or a troponin C polypeptide; and ii) a protease that cleaves the proteolytically cleavable linker.

Aspect 54. The nucleic acid of aspect 53, comprising an internal ribosome entry site between the first nucleotide sequence and the second nucleotide sequence.

Aspect 55. The nucleic acid of aspect 53, wherein the first nucleotide sequence is operably linked to a first promoter, and wherein the second nucleotide sequence is operably linked to a second promoter.

Aspect 56. A recombinant expression vector comprising the nucleic acid of any one of aspects 53-55.

Aspect 57. A nucleic acid comprising: a) a nucleotide sequence encoding a transmembrane domain; b) a nucleotide sequence encoding a polypeptide that binds a calcium-responsive polypeptide; c) a LOV domain light-activated polypeptide comprising an amino acid sequence having at least 80% amino acid sequence identity to the amino acid sequence depicted in any one of FIG. 15A-15G; d) a nucleotide sequence encoding a proteolytically cleavable linker; and e) an insertion site that provides for insertion of a nucleic acid of interest.

Aspect 58. The nucleic acid of aspect 57, wherein the insertion site is within 10 nucleotides of the 3′ end of the nucleotide sequence encoding the proteolytically cleavable linker.

Aspect 59. The nucleic acid of aspect 57, wherein the insertion site comprises one or more restriction endonuclease recognition sites.

Aspect 60. A recombinant expression vector comprising the nucleic acid of any one of aspects 57-59.

Aspect 61. The recombinant expression vector of aspect 60, wherein the recombinant expression vector is a recombinant lentiviral vector, a recombinant adeno-associated virus vector, or a recombinant retroviral vector.

Aspect 62. A light-activated, calcium-gated transcription control fusion polypeptide comprising, in order from amino terminus to carboxyl terminus: a) a transmembrane domain; b) a calmodulin-binding polypeptide or a troponin I polypeptide; c) a LOV domain light-activated polypeptide comprising an amino acid sequence having at least 80% amino acid sequence identity to the amino acid sequence depicted in any one of FIG. 15A-15G; d) a proteolytically cleavable linker; and e) a transcription factor, wherein the light-activated polypeptide undergoes a reversible conformational change when exposed to light of an activating wavelength, and wherein the conformational change exposes the proteolytically cleavable linker to a protease.

Aspect 63. The light-activated, calcium-gated transcription control polypeptide of aspect 62, comprising a calmodulin-binding polypeptide.

Aspect 64. The light-activated, calcium-gated transcription control polypeptide of aspect 62, wherein the calmodulin-binding polypeptide comprises an amino acid sequence having at least 80% amino acid sequence identity to KRRWKKNFIAVSAANRFKKISSSGAL (SEQ ID NO://) or FNARRKLKGAILTTMLATRNFS (SEQ ID NO://).

Aspect 65. The light-activated, calcium-gated transcription control polypeptide of aspect 64, wherein the calmodulin-binding polypeptide comprises an A14F substitution relative to the amino acid sequence KRRWKKNFIAVSAANRFKKISSSGAL (SEQ ID NO://).

Aspect 66. The light-activated, calcium-gated transcription control polypeptide of aspect 64, wherein the calmodulin-binding polypeptide comprises T13F and K8A amino acid substitutions relative to the amino acid sequence FNARRKLKGAILTTMLATRNFS.

Aspect 67. The light-activated, calcium-gated transcription control polypeptide of any one of aspects 62-66, wherein the light-activated polypeptide comprises an amino acid sequence having at least 90% amino acid sequence identity to the amino acid sequence depicted in any one of FIG. 15A-15G.

Aspect 68. The light-activated, calcium-gated transcription control polypeptide of any one of aspects 62-66, wherein the light-activated polypeptide comprises an amino acid sequence having at least 80% amino acid sequence identity to the amino acid sequence depicted in any one of FIG. 15A-15D and comprises L2R, N12S, I130V, A28V, and H117R substitutions relative to the amino acid sequence depicted in FIG. 15B.

Aspect 69. The light-activated, calcium-gated transcription control polypeptide of any one of aspects 62-68, wherein the proteolytically cleavable linker is cleavable by a protease that is not naturally produced by a mammalian cell.

Aspect 70. The light-activated, calcium-gated transcription control polypeptide of any one of aspects 62-69, wherein the proteolytically cleavable linker is cleavable by a viral protease.

Aspect 71. The light-activated, calcium-gated transcription control polypeptide of aspect 70, wherein the viral protease is a tobacco etch virus (TEV) protease.

Aspect 72. The light-activated, calcium-gated transcription control polypeptide of aspect 71, wherein the proteolytically cleavable linker comprises an amino acid sequence selected from ENLYFQS, ENLYFQY, ENLYFQL, ENLYFQW, ENLYFQM, ENLYFQH, ENLYFQN, ENLYFQA, and ENLYFQQ.

Aspect 73. The light-activated, calcium-gated transcription control polypeptide of aspect 62, comprising a troponin I polypeptide.

Aspect 74. The light-activated, calcium-gated transcription control polypeptide of aspect 73, wherein the troponin I polypeptide comprises an amino acid sequence having at least 80% amino acid sequence identity to the amino acid sequence depicted in FIG. 19A or FIG. 19B.

Aspect 75. A polypeptide system comprising: a) the light-activated, calcium-gated transcription control fusion polypeptide of any one of aspects 62-74; and b) a second fusion polypeptide comprising: i) a calmodulin polypeptide or a troponin C polypeptide; and ii) a protease that cleaves the proteolytically cleavable linker.

Aspect 76. The system of aspect 75, wherein the light-activated, calcium-gated transcription control fusion polypeptide comprises a calmodulin-binding polypeptide, and wherein the second fusion polypeptide comprises a calmodulin polypeptide.

Aspect 77. The system of aspect 75, wherein the light-activated, calcium-gated transcription control fusion polypeptide comprises a troponin I polypeptide, and wherein the second fusion polypeptide comprises a troponin C polypeptide.

Aspect 78. The system of aspect 76, wherein the calmodulin polypeptide comprises an amino acid sequence having at least 80% amino acid sequence identity to the amino acid sequence depicted in FIG. 16A or FIG. 16B.

Aspect 79. The system of aspect 77 or aspect 78, wherein the calmodulin polypeptide comprises F19L and V35G substitutions relative to the amino acid sequence depicted in FIG. 16A.

Aspect 80. The system of aspect 76, wherein the calmodulin-binding polypeptide comprises an amino acid sequence having at least 80% amino acid sequence identity to KRRWKKNFIAVSAANRFKKISSSGAL (SEQ ID NO://) or FNARRKLKGAILTTMLATRNFS (SEQ ID NO://).

Aspect 81. The system of aspect 80, wherein the calmodulin-binding polypeptide comprises an A14F substitution relative to the amino acid sequence KRRWKKNFIAVSAANRFKKISSSGAL.

Aspect 82. The system of aspect 80, wherein the calmodulin-binding polypeptide comprises T13F and K8A amino acid substitutions relative to the amino acid FNARRKLKGAILTTMLATRNFS.

Aspect 83. The system of aspect 77, wherein the troponin C polypeptide comprises an amino acid sequence having at least 80% amino acid sequence identity to the amino acid sequence depicted in FIG. 18.

Aspect 84. The system of aspect 77, wherein the troponin I polypeptide comprises an amino acid sequence having at least 80% amino acid sequence identity to the amino acid sequence depicted in FIG. 19A or FIG. 19B.

Aspect 85. The system of any one of aspects 75-84, wherein the LOV-domain light-activated polypeptide comprises one or more amino acid substitutions selected from L2R, N12S, A28V, H117R, and I130V substitutions relative to the amino acid sequence depicted in FIG. 15B.

Aspect 86. The system of any one of aspects 75-85, wherein the LOV domain light-activated polypeptide comprises L2R, N12S, I130V, A28V, and H117R substitutions relative to the amino acid sequence depicted in FIG. 15B.

Aspect 87. The system of any one of aspects 75-86, wherein the protease is not naturally produced by a mammalian cell.

Aspect 88. The system of aspect 87, wherein the protease is a viral protease.

Aspect 89. The system of aspect 88, wherein the viral protease is a tobacco etch virus (TEV) protease.

Aspect 90. The system of any one of aspects 75-86, wherein the protease is naturally produced by a mammalian cell.

Aspect 91. A mammalian cell comprising the system of any one of aspects 75-90.

Aspect 92. The mammalian cell of aspect 91, wherein the cell is a neuron.

Aspect 93. The mammalian cell of aspect 91 or aspect 92, wherein the cell is a human cell.

Aspect 94. The mammalian cell of any one of aspects 91-93, wherein the cell is in vitro.

Aspect 95. The mammalian cell of any one of aspects 91-93, wherein the cell is in vivo.

Aspect 96. The mammalian cell of any one of aspects 91-95, comprising a reporter nucleic acid comprising: i) a promoter that is activated by the transcription factor; and ii) a nucleotide sequence encoding a target gene product, wherein the nucleotide sequence is operably linked to the promoter.

Aspect 97. The mammalian cell of aspect 96, wherein the target gene product is a nucleic acid.

Aspect 98. The mammalian cell of aspect 97, wherein the nucleic acid is an inhibitory RNA, a ribozyme, or a microRNA.

Aspect 99. The mammalian cell of aspect 97, wherein the nucleic acid is a guide RNA that binds a target nucleotide sequence and an RNA-guided endonuclease.

Aspect 100. The mammalian cell of aspect 96, wherein the target gene product is a polypeptide.

Aspect 101. The mammalian cell of aspect 100, wherein the target gene product is a reporter, a light-activated polypeptide, a toxin, a DREADD, a kinase, an RNA-guided endonuclease, a transcription factor, an antibiotic resistance factor, a calcium sensor, a peroxidase, or an antibody.

Aspect 102. The mammalian cell of aspect 100, wherein the target gene product is a reporter gene product.

Aspect 103. The mammalian cell of aspect 102, wherein the reporter gene product is an enzyme.

Aspect 104. The mammalian cell of aspect 102, wherein the reporter gene product is a fluorescent polypeptide.

Aspect 105. The mammalian cell of any one of aspects 96-104, comprising a heterologous nucleic acid comprising: i) a promoter; and ii) a nucleotide sequence encoding a heterologous light-activated polypeptide, wherein the nucleotide sequence is operably linked to the promoter, and wherein the heterologous light activated polypeptide is activated by light of a wavelength that is different from the wavelength of light that activates the light-responsive polypeptide in the system.

Aspect 106. The mammalian cell of aspect 105, wherein the promoter is activated by the transcription factor present in the system.

Aspect 107. A genetically modified non-human organism that comprises, integrated into the genome of one or more cells of the organism, the nucleic acid system of any one of aspects 1-27 or 46-52, or the nucleic acid of any one of aspects 32, 35, 38, 53-55, and 57-59.

Aspect 108. The genetically modified non-human organism of aspect 107, wherein the organism is a mammal.

Aspect 109. The genetically modified non-human organism of aspect 108, wherein the mammal is a rodent.

Aspect 110. A method for detecting a change in the intracellular calcium concentration in a cell in response to a stimulus, the method comprising: exposing the cell to the stimulus; and substantially simultaneously exposing the cell to light of an activating wavelength; wherein the cell is genetically modified with the nucleic acid system of any one of aspects 46-52, wherein an increase in a product of the reporter gene, compared to a control level of the reporter gene product, indicates that exposure to the stimulus increases the intracellular calcium concentration in the cell.

Aspect 111. The method of aspect 110, wherein the stimulus is a ligand, a drug, a toxin, a neurotransmitter, contact with a second cell, heat, or hypoxia.

Aspect 112. The method of aspect 110 or aspect 111, wherein the reporter gene product is an enzyme that acts on a substrate to produce a detectable product.

Aspect 113. The method of aspect 110 or aspect 111, wherein the reporter gene product is a fluorescent protein.

Aspect 114. The method of any one of aspects 110-113, wherein the cell is in vitro.

Aspect 115. The method of any one of aspects 110-113, wherein the cell is in vivo.

Aspect 116. The method of any one of aspects 110-115, wherein the cell is a human cell.

Aspect 117. The method of any one of aspects 110-115, wherein the cell is a non-human animal cell.

Aspect 118. The method of any one of aspects 110-117, wherein a change in the intracellular calcium concentration is detected over a period of time of at least 1 minute.

Aspect 119. The method of any one of aspects 110-118, further comprising:

c) when the level of reporter gene product indicates that the intracellular calcium concentration is greater than 100 nM, modulating an activity of the cell.

Aspect 120. The method of aspect 119, wherein said modulating comprises inducing production of an effector polypeptide in the cell.

Aspect 121, The method of aspect 121, wherein the effector polypeptide is a hyperpolarizing opsin, a depolarizing opsin, a transcription factor, a recombinase, an RNA-guided endonuclease, a kinase, a DREADD, or a toxin.

Aspect 122. A method of modulating an activity of a cell, the method comprising: exposing the cell to light of an activating wavelength; and substantially simultaneously exposing the cell a second stimulus; wherein the cell is genetically modified with the nucleic acid system of any one of aspects 1-27, and wherein said exposing induces production of the polypeptide of interest, wherein the polypeptide of interest modulates an activity of the cell.

Aspect 123. The method of aspect 122, wherein the cell is in vitro.

Aspect 124. The method of aspect 122, wherein the cell is in vivo.

Aspect 125. The method of any one of aspects 122-124, wherein the cell is a human cell.

Aspect 126. The method of any one of aspects 122-124, wherein the cell is a non-human animal cell.

Aspect 126. A light-activated polypeptide comprising an amino acid sequence having at least 80% amino acid sequence identity to the amino acid depicted in FIG. 15B and comprises L2R, N12S, I130V, A28V, and H117R substitutions relative to the amino acid sequence depicted in FIG. 15B.

Aspect 128. A nucleic acid comprising a nucleotide sequence encoding the light-activated polypeptide of aspect 127.

Aspect 129. The nucleic acid of aspect 127, wherein the nucleotide sequence is operably linked to a promoter.

Aspect 130. The nucleic acid of aspect 129, wherein the promoter is an inducible promoter.

Aspect 131. A recombinant expression vector comprising the nucleic acid of any one of aspects 128-130.

Aspect 132. A recombinant cell comprising the nucleic acid of any one of aspects 128-130 or the recombinant expression vector of aspect 131.

Aspect 133. A nucleic acid comprising a nucleotide sequence encoding the light-activated, calcium-gated transcription control polypeptide of any one of aspects 62-74.

Aspect 134. The nucleic acid of aspect 133, wherein the nucleotide sequence is operably linked to a promoter.

Aspect 135. The nucleic acid of aspect 134, wherein the promoter is a cell type-specific promoter.

Aspect 136. The nucleic acid of aspect 134, wherein the promoter is a constitutively active promoter.

Aspect 137. The nucleic acid of aspect 134, wherein the promoter is a regulatable promoter.

Aspect 138. A recombinant expression vector comprising the nucleic acid of any one of aspects 133-137.

Aspect 139. A host cell genetically modified with the nucleic acid of any one of aspects 133-137 or the recombinant expression vector of aspect 138.

Aspect 140. The host cell of aspect 139, wherein the host cell is a mammalian cell.

Aspect 141. The host cell of aspect 139 or aspect 141, wherein the nucleic acid or the recombinant expression vector is stably integrated into the genome of the host cell.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: FLARE Systems and Methods of Using the Systems

A light and calcium gated transcription factor (TF) system was designed. A schematic depiction of an example of such a system shown in FIG. 1A. In the basal state, the TF is tethered to the cell's plasma membrane, unable to activate transcription of the reporter gene located in the cell's nucleus. Upon exposure to both blue light and high calcium, however, the TF is cleaved from the membrane and translocates to the nucleus because (1) the protease recognition site is unblocked by the light-sensitive LOV domain, and (2) the protease is recruited to its recognition site via a calcium-regulated intermolecular interaction between calmodulin (CaM) and a CaM binding peptide. Importantly, high calcium alone is not sufficient to give TF release because the protease site remains blocked, and light alone is not sufficient because the protease is far away, and its affinity for its recognition site is too low to afford cleavage in the absence of induced proximity. Also key to this design is that both calcium sensing and light sensing are fully reversible, such that sequential rather than coincident inputs (such as high calcium followed by light) are unable to trigger TF release.

This tool is referred to herein as FLARE, for Fast Light and Activity Reporter giving Expression. First, a proximity-dependent protease cleavage system was engineered to increase the signal-to-noise ratio (S/N). Second, a LOV domain for light gating was introduced. Directed evolution was performed to “customize” LOV for caging the TEV protease cleavage site specifically; this modified LOV is referred to herein as “eLOV”. Evolved LOV (eLOV) with 5 mutations gave more than 10-fold improved light gating in HEK cells. These variant components were further modified to improve membrane targeting and S/N. The FLARE tool gave a light/dark S/N>120 and a high/low calcium S/N of 10 in living neurons, and enabled functional re-activation of selected neurons via FLARE-driven channelrhodopsin expression.

### Materials and Methods

Cloning.

All of the constructs for testing in HEK cells and cultured neurons were cloned into an adeno-associated virus (AAV) viral vector. All the constructs for yeast display were cloned into pCTCON2 vector. CaM was amplified from GCaMP5 asLOV2 was synthesized through overlap polymerase chain reaction (PCR).

Expression and Purification of Tobacco Etch Virus (TEV) Protease.

MBP-TEV(S219V) fusion construct in pET21b vector was made and transformed into homemade BL21-CodonPlus(DE3)-RIPL competent cells. MBP (maltose binding protein) fusion helps solubilize TEV protease and increase the expression yield. Transformed BL21 cells were inoculated in 50 mL LB culture with 100 mg/L Ampicillin and grew in a shaker at 37° C. and 220 revolutions per minute (RPM). Ten ml of the overnight culture was transferred to 1 L Luria Broth (LB) with 100 mg/L Ampicillin and grew at 37° C. until OD600 reaches 0.6. IPTG was added to the culture to a final concentration of 1 mM and the culture was kept at RT shaker at 220 RPM for 12 hrs before harvesting. BL21 cell pellet was lysed in ice cold RIPA buffer (Thermo Fisher Scientific) supplemented with 1 mM dithiotreitol (DTT) (Sigma-Aldrich, freshly made) and spun down at 10,000 RPM for 15 min at 4° C. The supernatant was incubated with 1 mL Ni-NTA beads at 4° C. for 10 min and then loaded to a column. The beads were washed with 10 mL washing buffer (30 mM imidazole, 50 mM Tris, 300 mM NaCl, 1 mM DTT, pH=7.8) and eluted with 10 mL elution buffer (200 mM imidazole, 50 mM Tris, 300 mM NaCl, 1 mM DTT, pH=7.8). The eluent (from 5×1L) was combined and concentrated with a 15 mL 10,000 Da cutoff centrifugal unit (Millipore) to OD₂₈₀˜70. LOV-TEVcs required very high concentrations of TEV protease to get sufficient cleavage in dark, because the TEVsite used has low Kcat and it was caged in dark. The whole purification process should be performed at 4° C. and under reducing conditions; TEV protease was not stable under oxidizing conditions. Gel electrophoresis was performed to check the purity of the TEV protease. However, the quality of TEV protease varied from batch to batch.

Yeast Strains, Transformation, and Cell Culture.

Aga2p-HA-LOV-FLAG yeast was generated by transformation of the yeast display plasmid pCTCON2 (Chao, G., Lau, W. L., Hackel, B. J., Sazinsky, S. L., Lippow, S. M., and Wittrup, K. D. (2006) Isolating and engineering human antibodies using yeast surface display. Nat. Protoc. 1, 755-68) into the Saccharomyces cerevisiae strain EBY100, as described previously. Lam, S. S., Martell, J. D., Kamer, K. J., Deerinck, T. J., Ellisman, M. H., Mootha, V. K., and Ting, A. Y. (2014) Directed evolution of APEX2 for electron microscopy and proximity labeling. Nat. Methods 12, 51-54. Transformed cells containing the Trp1 gene were selected on synthetic dextrose plus casein amino acid (SDCAA) plates. Yeast cell culture and induction of pCTCON2 construct expression were performed as described previously. Lam et al. (2014) infra.

Generation of Error Prone PCR Libraries for Yeast Selection.

Libraries of LOV mutants were generated using error-prone PCR. In brief, 100 ng of the template gene was amplified for 20 rounds with 0.4 μM forward and reverse primers, 2 mM MgCl₂, 5 units of Taq polymerase (NEB), and 2 μM each of the mutagenic nucleotide analogs 8-oxo-2′-deoxyguanosine-5′tri-phosphate (8-oxo-dGTP) and 2′-deoxy-p-nucleoside-5′-triphosphate (dPTP). The PCR product was then gel-purified and re-amplified for another 30 cycles under normal PCR conditions with Taq polymerase. The error-prone PCR product was electroporated along with BamHI-NheI linearized pCTCON2 vector (10 μg insert: 1 μg vector) backbone into electrocompetent S. cerevisiae EBY100 cells. Electroporation was performed using a Bio-Rad Gene pulser XCell. Transformation efficiency was 3.6×10⁷. DNA sequencing of 12 distinct colonies showed a range of 0 to 2 nucleotides changed per clone. The electroporated cultures were rescued in 100 mL of SDCAA media supplemented with 50 units/mL penicillin and 50 g/mL streptomycin for 1 day at 30° C.

Yeast Display Selection.

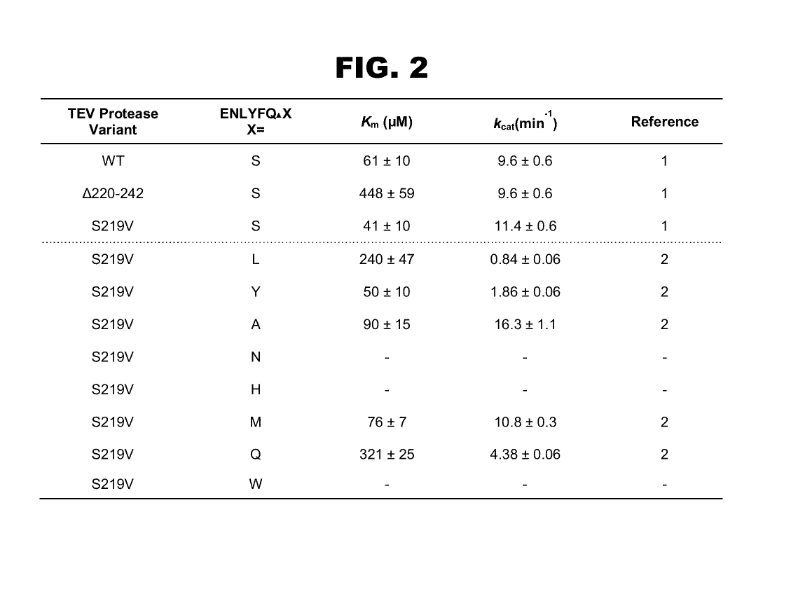

Yeast cells display a library of LOV mutants were induced by growing yeast in 1:9 SDCAA:SGCAA media overnight. For the 1^{st} round of selection, 1 mL of overnight yeast cell culture (OD₆₀₀˜15) were spun down in a microfuge Eppendorf tube at 5000×g for two minutes; for the following selections, 0.5 mL were spun down. Yeast cells were washed with PBSB (sterile phosphate buffer saline solution supplemented with 0.1% BSA) twice. To remove residue liquid on the Eppendorf tube wall, the pellet was spun down at 5000×g for 30 seconds and the remaining liquid was removed by gentle pipetting. The yeast cells were kept in dark for 5 minutes before TEV protease (˜30 μM, 100 μL) was added under red light. For cleavage in light, yeast cells were exposed to a daylight lamp (T5 Circline Fluorescent Lamp, 25 W, 6500K, 480 nm, 530 nm, 590 nm) in a rotator for 1 h; for cleavage in dark, yeast cells were wrapped up in alumina foil and placed in a rotator for 3 hrs. Yeast cells were spun down and washed with PBSB (room temperature) twice and then labeled with primary antibodies: mouse-anti-flag (1:200, Sigma) and rabbit-anti-HA (1:200, Rockland) and secondary antibodies: anti-mouse-647 (1:200, Life Technology) and anti-rabbit-PE. The labeled yeast cells were resuspended in PBSB to 5×10⁷ cells/mL and sorted by FACS. Six rounds of negative and positive selections were performed. Gates were drawn as shown in FIG. 2 to collect the following % of cells: 1^{st} round (negative selection), top 0.5% (2.8×10⁵ cells); 2^{nd} round (negative selection), top 25% (1×10⁶ cells), the second round of negative selection is more generous because a large portion of the yeast population is false negative; 3^{rd} round (positive selection), the bottom 3.5% (1.2×10⁵ cells); 4^{th} round (positive selection), bottom 9.3% (5.0×10⁵ cells); 5^{th} round (negative selection), top 1.35% (1.2×10⁵ cells); 6^{th} round (positive selection), bottom 3.1% (3.7×10⁵ cells).

Fluorescence Activated Cell Sorting (FACS) Analysis.

Induced yeast cells (0.25 mL of overnight culture at OD₆₀₀˜15) were spun down at 5000×g for two minutes. Yeast cells were washed with PBSB twice and treated with TEV protease (˜30 μM, 100 μL) in dark for 3 hrs and in light for 1 hr. Yeast cells were labeled with primary antibodies: mouse-anti-FLAG (1:200, Sigma) and rabbit-anti-HA (1:200, Rockland) and secondary antibodies: anti-mouse-647 (1:200, Life Technology) and anti-rabbit-phycoerythrin (PE) before FACS analysis.

HEK293T Cell Culture and Transfection.

HEK293T cells from ATCC (passage number<20) were cultured as a monolayer in complete growth media, DMEM (Gibco) supplemented with 10% FBS (Sigma), at 37° C. under 5% CO₂. For large field microscopic experiment (10× objective), cells were grown in 48 well plate that were pretreated with 50 μg/mL fibronectin (Millipore) for at least 10 min at 37° C. before cell plating. For high resolution fluorescence experiment (40× objective), cells were grown on a 7×7 mm glass cover slips in 48 well plate that were pretreated with human fibronectin. Cells were transfected at 60-90% confluence with 1 mg/mL PEI Max solution (pH=7.3). For imaging experiment in the 48 well plate, a mix of DNA (15 ng of UAS-citrine reporter construct, 15 ng of TEV protease construct, 50-100 ng of the transcription factor construct) were incubated with 0.8 μL PEI Max in 10 μL serum free DMEM media for 15 min at RT. DMEM media supplemented with 10% FBS (100 μL) was mixed with the DNA-PEI Max solution and added to the HEK293T cells in 48 well plates and incubate for 18 hours before stimulation.

HEK293T Cell Stimulation, Imaging and Analysis of the Data for the Calcium Dependent Protease Cleavage.

HEK293T cells were stimulated 18 hours post transfection. For high Ca²⁺ conditions, 100 μL ionomycin and CaCl₂ in complete growth media were added gently to the top of the media in a 48-well plate to a final concentration of 2 μM and 5 μM respectively. For low Ca²⁺ conditions, 100 μL complete growth media was added. Five minutes later, the solution in the 48-well plates was replaced with 200 μL fresh complete growth media. After stimulation, HEK293T cells were incubated for 12-18 hrs before fixation with 4% paraformaldehyde in PBS. HEK293T cells were permeabilized by incubation with cold methanol at −20° C. for 5 min, followed by immunostaining against mouse-anti-V5 (1:2000 dilution, Life Technology) and rabbit-anti-HA (1:1000 dilution, Rockland) and anti-mouse-alexafluoro568 (1:1000 dilution, Life Technology) and anti-rabbit-alexafluoro647 (1:1000 dilution, Life Technology) in 2% BSA PBS solution. HEK293T cells directly plated on the 48-well plate were imaged with 10× air objective in the Zeiss LSM510 confocal microscope. Eight to ten fields of view were acquired for each condition. A mask was defined according to the immunofluorescence of the V5 (protease expression) and mean intensity of citrine within the mask was calculated as Intensity 1. A second mask was drawn in the area outside of V5 immunofluorescence and mean intensity of citrine within this mask was calculated as Intensity 2, attributed as background fluorescence due to autofluorescence of untransfected cells or plates. Intensity 1 was subtracted by intensity 2 for each image to get the corrected mean intensity of citrine, reporter gene expression. The average value of the corrected mean intensity of citrine was calculated across 8-10 fields of view for each condition. Error bar was defined as the SEM, STD/Sqrt(# of the fields of view), for the corrected mean intensity of citrine across 8-10 fields of view.

HEK293T Cell Stimulation, Imaging and Analysis of the Data for the Light and Calcium Dependent Protease Cleavage.

HEK293T cells were kept in dark after transfection and the following processes should be performed in a dark room with red light illumination. HEK293T cells were stimulated 18 hours post transfection. High and low Ca²⁺ conditions were induced right before blue light irradiation. For high Ca²⁺ conditions, 100 μL ionomycin (Sigma-aldrich) and CaCl₂ in complete growth media were added gently to the top of the media in a 48-well plate to a final concentration of 2 μM and 5 μM respectively. For low Ca²⁺ conditions, 100 μL complete growth media was added. For light stimulation, HEK293T cells in 48-well plate was placed on top of a custom-built light box with 467 nm blue light at 60 mW/cm² intensity and 33% duty cycles. For the dark condition, HEK293T cells were kept in dark by wrapping the plates in alumina foil. After stimulation, HEK293T cells were kept in dark for 5 more minutes before the solution in the well were replaced with 200 μL fresh complete growth media. HEK293T cells were incubated for additional 12-18 hours before fixation with 4% paraformaldehyde in PBS. The rest of the procedures are the same as that for calcium dependent protease cleavage, see above.

HEK293T Cell Imaging for the Comparison of the Original and Evolved LOV Domain.

HEK293T cells were cultured on coverslips pretreated with human fibronectin. For the evolved LOV conditions, HEK293T cells were transfected with a mix of DNA constructs P16 (50-100 ng/well), P7 (15 ng/well), P9 (15 ng/well) in 10 μL DMEM and 0.8 μL PEI max. For the original LOV, HEK293T cells were transfected with a mix of DNA constructs P11 (50-100 ng/well), P7 (15 ng/well), P9 (15 ng/well) in 10 μL DMEM and 0.8 μL PEI max. HEK293T cells were stimulated 18 post transfection under four conditions as above, light+high calcium, light+low calcium, dark+high calcum, dark+low calcium. HEK293T cells were fixed and immunostained as above. HEK293T cells were then imaged on an imaging dish with 40× oil objective in the Zeiss LSM510 confocal microscope.

AAV Virus Supernatant Production.

HEK293T cells were transfected at 60-90% confluence. For each well in the 6-well plate, 0.35 μg viral DNA, 0.29 μg AAV1, 0.29 μg AAV2, 0.7 μg DF6 were incubated with 80 μL serum free DMEM for 15 min. Two mL DMEM supplemented with FBS were mixed with the PEI Max solution. Media was removed from the HEK293T cells in the 6-well plate right before the PEI Max solution was added. HEK293T cells were incubated for 48 hrs and the supernatant was collected and filtered through a 0.45 μm syringe filter (VWR). AAV virus was aliquoted into 0.5 mL, flash frozen in liquid nitrogen and stored at −80° C.

Concentrated AAV Virus Production.

Concentrated AAV virus was prepared as described previously. Konermann, S., Brigham, M. D., Trevino, A. E., Hsu, P. D., Heidenreich, M., Cong, L., Platt, R. J., Scott, D. a, Church, G. M., and Zhang, F. (2013) Optical control of mammalian endogenous transcription and epigenetic states. Nature 500, 472-6. Briefly, two T150 flasks of HEK293T cells under the passage of 10 were transfected at 80% confluence. For each T150 flask, 5.2 μg vector of interest plasmid, 4.35 μg of both AAV1 and AAV2 serotype plasmid, 10.4 μg pDF6 plasmid (adenovirus helper genes) were incubated with 130 μL PEI in 500 μL serum free DMEM media at RT for 10 min. The media in the T150 flask was aspirated and replaced with 30 mL of complete growth media added to the DNA mix. HEK293T cells were incubated for 48 hours at 37° C. and then the cell pellet were collected by centrifugation at 800×g for 10 min. The pellet was resuspended in 20 mL tris buffer containing 150 mM NaCl, 20 mM Tris, pH=8.0. Freshly made 10% sodium deoxycholate (Sigma-aldrich) in H₂O was added to the resuspended cells to a final concentration of 0.5% and benzonase nuclease (Sigma-aldrich) was added to a final concentration of 50 units per mL. The solution was incubated at 37° C. for 1 hour and then centrifuged at 3000×g for 15 min to remove the cellular debris. The supernatant was then loaded using a peristaltic pump (Gilson MP4) at 1 mL/min flow rate to a HiTrap heparin column (GE healthcare Life Sciences) that was pre-equilibrated with 10 mL 150 mM NaCl, 20 mM Tris, pH=8.0 solution. The column was washed with 20 mL 100 mM NaCl, 20 mM Tris, pH=8.0 using peristaltic pump, followed by washing with 1 mL 200 mM NaCl, 20 mM Tris, pH=8.0 and 1 mL 300 mM NaCl, 20 mM Tris, pH=8.0 using a 5 mL syringe. The virus was eluted using 5 mL syringes with 1.5 mL 400 mM NaCl, 20 mM Tris, pH=8.0; 3.0 mL 450 mM NaCl, 20 mM Tris, pH=8.0 and 1.5 mL 500 mM NaCl, 20 mM Tris, pH=8.0. The eluted virus was concentrated down using Amicon ultra 15 mL centrifugal units with a 100,000 molecular weight cut off at 2000×g for 2 min to a volume of 500 μL. One mL sterile DPBS was added to the filter unit and centrifuged to a final volume of ˜200 μL. The concentrated AAV virus was aliquoted at 10 μL to precoated eppendorf tubes and stored at −80° C.

AAV Virus Titration by Quantitative PCR (qPCR).

AAV virus (2 μL) was incubated with 1 μL DNAseI (NEB) in a final volume of 40 μL at 37° C. for 30 min and then deactivated at 75° C. for 15 min. Five μL of the DNAse treated solution was incubated with 1 μL proteinase K (Thermo Fisher Scientific) at a total volume of 20 μL at 50° C. for 30 min and proteinase K was deactivated at 98° C. for 10 min. Two μL sample from the proteinase K reaction was used for qPCR reactions following sybergreen protocol in qPCR (Applied Biosystems), along with the standard samples prepared from linearized AAV DNA plasmid. AAV virus titer was quantified by dividing the dilution factors 1:20×1:4×2=1:40 and multiply 2 for the single stranded genome as compared to the standard AAV DNA plasmid.

Rat Cortical Neuron Culture.

Cortical neurons were harvested from rat embryos euthanized at embryonic day 18 and plated in 24-well plates. At DIV4, 500 μL complete neurobasal media (neurobasal supplemented with 1×B27, Glutamax and Penstrep) with 5-Fluorodexoyuridine was added to each well, replacing 30% of the media in the well. Subsequently, around 30% of the media were replaced with fresh complete neurobasal media every three days.

Cortical Neuron Culture Transduction and Stimulation with Media Change.

A mixture of AAV virus supernatant (50 μL of each AAV virus) was added to the neurons at DIV10-15 and incubated for two days before 30% of the solution in the well was replaced with fresh complete neurobasal media. Neurons were kept in dark and the following procedures were performed in a dark room with red light illumination. Six days post-transduction, neurons were stimulated and high Ca²⁺ condition was induced right before the light irradiation. For high Ca²⁺, 90% of the media in the well was replaced with fresh neurobasal media. For low Ca²⁺, neurons were left at basal levels without perturbations. For light stimulations, neurons in a 24-well plate were placed on top of the custom-built light box and irradiated by 467 nm blue light at 60 mW/cm² and 33% duty cycles. After stimulation, neurons were incubated for 16-24 hrs before fixation with paraformaldehyde fixative (4% paraformaldehyde, 60 mM PIPES, 25 mM HEPES, 10 mM EGTA, 2 mM MgCl₂, 0.12 M sucrose, pH=7.3).

Immunostain of Fixed Neurons and Imaging.

Fixed neurons were permeabilized by incubation with cold methanol at −20° C. for 5 min and blocked with 2% BSA in PBS at RT for 1 hr. Neurons were immunostained against mouse-anti-V5 (1:2000 dilution, Invitrogen) and rabbit-anti-VP16 (1:2000 dilution, Abcam), followed by anti-mouse-alexafluoro488 (1:1000 dilution) and anti-rabbit-alexafluoro647 (1:1000 dilution) in 2% BSA solution in PBS. Neurons directly plated on the 48-well plate were imaged with 10× air objective in the Zeiss LSM510 confocal microscope and neurons plated on glass cover slips were imaged with 40× oil objective in the Zeiss LSM510 confocal microscope. Eight to ten fields of view were collected for each condition.

Analysis of the Neuronal Imaging Data.

For each field of view, a mask was created in the areas where there was anti-V5 immunofluorescence and mean fluorescence intensity of mCherry (reporter gene) was calculated within the mask as the uncorrected mCherry intensity. A second mask was created in areas where there was no anti-V5 immunofluorescence and mean mCherry intensity was calculated within the mask as the background mCherry intensity. mCherry intensity was the subtraction of uncorrected mCherry intensity by the background mCherry intensity for each field of view. Mean reporter gene fluorescence intensity is calculated across 8-10 fields of view for each stimulation condition. Error bar is SEM.

Field Stimulation of Neurons Infected with GCaMP5.

Neurons were infected with 50 μL GCaMP5 virus and 30% of the media was replaced with fresh complete neurobasal media at day two post-transduction. At day 6 post-transduction, field stimulation was performed. Master 8 from AMPI was used to induce trains of electric stimuli; Stimulator isolator unit (Warner Instrument, SIU-102b) was used to provide constant current output ranging from 10-50 mA. Platinum iridium alloy (70:30) wire from Alfa-Aesar was folded into a pair of rectangles and placed right above the neurons on the edge of the well to act as electrodes. A time-lapse recording of GCaMP5 fluorescence was acquired with 10× air objective in the Zeiss LSM510 confocal microscope when field stimulation was delivered. 40 mA is the minimum current required to get robust GCaMP5 activation. To achieve reliable neuronal activation, 48 or 50 mA was applied for field stimulation. To optimize the duration of the stimuli, 0.1, 0.2, 0.5, 1 and 5 millisecond were tried, a minimum of 1 millisecond is required. 1 millisecond and 5 millisecond did not make a difference. To minimize the damage to neurons, 1-millisecond pulse was used. GCaMP5 activation with 5 pulses of 1-millisecond 20 Hz stimulation is better than 1 pulse of 5-millisecond stimulation at 48 mA.

Field Stimulation of Neurons Transduced with FLARE AAV Viruses.

Neurons were transduced with FLARE supernatant AAV virus containing P24, P26 and P27. Six days post-transduction, neurons were either irradiated with light (467 nm, 60 mW/cm², 10% duty cycles: 500 msec/5 sec) or kept in dark when field stimulation was performed. Neurons were activated by field stimulation (3 second trains consisting of 32 1-millisecond 48 mA stimulation at 20 Hz) for 4, 8, 15 minutes.

Reactivation of Chrimson.

Cultured neurons were transduced with FLARE AAV viruses and GCaMP5 lentivirus at DIV13 and stimulated at DIV19 with light (467 nm, 60 mW/cm², 10% duty cycles: 500 msec/5 sec) and field stimulation (3 second trains consisting of 32 1-millisecond 48 mA stimulation at 20 Hz for 15 minutes). 18 hours later, live neurons were imaged with 10× air objective in the confocal microscope. Chrimson was activated by 568 nm laser (800 msec, 60 mW/cm²) from the microscopic objective every 5 second and GCaMP5 fluorescence was recorded.

Virus Infusion.

Adult wild-type male C57BL/6 mice ˜8 weeks old (Jackson Laboratory, Bar Harbor, Me.) were used for all experiments. All procedures were preformed in accordance with the guidelines from NIH and with approval from the MIT Committee on Animal Care (CAC). All surgeries were conducted under aseptic conditions using a digital small animal stereotaxic instrument (David Kopf Instruments, Tujunga, Calif.). Mice were anaesthetized with isoflurane (5% for induction, 1.5-2.0% after) in the stereotaxic frame for the entire surgery and their body temperature was maintained using a heating pad. The motor cortex was targeted using the following coordinates from bregma: +1.78 mm AP, 1.5 mm ML, and -1.75 mm DV. The 4 AAV viruses encoding the reporter were injected bilaterally using 10 μL microsyringe with a beveled 33 gauge microinjection needle (nanofil; WPI, Sarasota, Fla.). 1000 nL of the viral suspensions at a rate of 150 nL/min was infused using a microsyringe pump (UMP3; WPI, Sarasota, Fla.) and its controller (Micro4; WPI, Sarasota, Fla.). After each injection the needle was raised 100 μm for an additional 10 minutes to allow for viral diffusion at the injection site and then slowly withdrawn. In one hemisphere an optic fiber (300 μm core, 0.37 NA) (Thorlabs, Newton, N.J., USA) held in a 1.25 mm ferrule (Precision Fiber Products, Milpitas, Calif., USA) was implanted 0.5 mm above the injection site. The optic fiber was held in place using a layer of adhesive cement (C&B metabond; Parkell, Edgewood, N.Y.) followed by a layer of cranioplastic cement (Ortho-Jet; Lang, Wheeling, Ill., USA).

Stimulation in Animals.

Light stimulation was preformed seven days following viral injection. The optic fiber implants were connected to a 473-nm diode-pumped solid state (DPSS) laser (OEM Laser Systems, Draper, Utah, USA). A Master-8 pulse stimulator (A.M.P.I., Jerusalem, Israel) was used to deliver 0.5 mW of 473-nm light 2 second pulses every 4 second, for 30 minutes. To induce seizures, 15 minutes prior to stimulation mice received an intraperitoneal injection of kainic acid 10 mg/kg in saline (Sigma-Aldrich, St. Louis, Mo., USA). For anesthetized experiments, the mice received isoflurane anesthesia (5% for induction, 2-2.5% after) 15 minutes prior to receiving stimulation and remained under anesthesia for an additional 30 minutes following light administration.

Perfusion.

Animals were sacrificed 24 hrs after receiving stimulation by being deeply anesthetized with sodium pentobarbital (200 mg/kg; I.P.) and transcardially perfused with 10 mL of Ringer's solution followed by 10 mL of cold 4% PFA dissolved in 1×PBS. The excised brains were held in a 4% PFA solution for at least 24 hours before being transferred to a 30% sucrose solution in 1×PBS for. The brains were then sectioned into 50 μm slices using a sliding microtome (HM420; Thermo Fischer Scientific, Waltham, Mass., USA) before being mounted on glass microscope slides, and cover-slipped using PVA mounting medium with DABCO (Sigma-Aldrich, St. Louis, Mo., USA).

Confocal Microscopy of Brain Slides.

Fluorescent images were obtained using a confocal laser scanning microscope (Olympus FV1000, Olympus, Center Valley, Pa., USA) with FluoView software (Olympus, Center Valley, Pa., USA) under a 10×/0.40 NA dry objective or a 40×/1.30 NA oil immersion objective.

### Results

### Engineering the Calcium Response

In the FLARE design, high calcium is sensed by calmodulin (CaM), which binds to its effector peptide (CaMbp), bringing a fused protease into proximity of its cleavage site. In order for this design to work, the affinity between CaM and CaMbp in the high calcium state must be much higher than the affinity between protease and cleavage site. Furthermore, the latter affinity is capped by typical expression levels of tool components in neurons, which can exceed 150 μM (Huber, D., Gutnisky, D. a., Peron, S., O'Connor, D. H., Wiegert, J. S., Tian, L., Oertner, T. G., Looger, L. L., and Svoboda, K. (2012) Multiple dynamic representations in the motor cortex during sensorimotor learning. Nature 484, 473-478). In other words, even at high FLARE component expression levels approaching 150 μM, the protease and its cleavage site must not significantly interact so long as calcium levels are low.

The TANGO system developed to visualize GPCR activation (Barnea, G., Strapps, W., Herrada, G., Berman, Y., Ong, J., Kloss, B., Axel, R., and Lee, K. J. (2008) The genetic design of signaling cascades to record receptor activation. Proc. Natl. Acad. Sci. U.S.A. 105, 64-9; and Inagaki, H. K., Ben-Tabou De-Leon, S., Wong, A. M., Jagadish, S., Ishimoto, H., Barnea, G., Kitamoto, T., Axel, R., and Anderson, D. J. (2012) Visualizing neuromodulation in vivo: TANGO-mapping of dopamine signaling reveals appetite control of sugar sensing. Cell 148, 583-595) has a similar design. Hence this was used as a starting point for the FLARE design. The TEV (Tobacco Etch Virus) protease used in TANGO is orthogonal in neurons—it does not recognize and cleave any endogenous neuronal proteins, which minimizes its toxicity—and there are numerous known peptide cleavage substrates (TEVcs). To incorporate Tango into FLARE, TEV protease was fused to CaM (a F19L/V35G engineered mutant (Palmer, A. E., Giacomello, M., Kortemme, T., Hires, S. A., Lev-Ram, V., Baker, D., and Tsien, R. Y. (2006) Ca2+ Indicators Based on Computationally Redesigned Calmodulin-Peptide Pairs. Chem. Biol. 13, 521-530) that does not bind to endogenous CaM effectors), and the Tango TEVcs (ENLYFQ_{̂}L; SEQ ID NO://) was sandwiched between a plasma membrane anchor (the transmembrane helix from CD4 (Feinberg, E. H., VanHoven, M. K., Bendesky, A., Wang, G., Fetter, R. D., Shen, K., and Bargmann, C. I. (2008) GFP Reconstitution Across Synaptic Partners (GRASP) Defines Cell Contacts and Synapses in Living Nervous Systems. Neuron 57, 353-363)), CaM binding peptide M13 (with a A13F “bump” mutation that complements the “hole” mutations in CaM (Palmer, A. E., Giacomello, M., Kortemme, T., Hires, S. A., Lev-Ram, V., Baker, D., and Tsien, R. Y. (2006) Ca2+ Indicators Based on Computationally Redesigned Calmodulin-Peptide Pairs. Chem. Biol. 13, 521-530)), and the Gal4 transcription factor, as shown in FIG. 1B. Constructs were transfected into HEK cells, along with a UAS-GFP plasmid whose expression is driven by nuclear-localized Gal4. Comparing GFP expression in untreated HEK cells to those bathed in high calcium for 5 minutes, no significant difference was observed (FIG. 1C, 4^{th} set of columns).

FIG. 1 depicts the FLARE design and optimization of calcium response. (FIG. 1A) FLARE components in the dark, low Ca⁺² state (left) and in the light-exposed, high Ca⁺² state (right). The LOV domain undergoes a reversible conformational change upon blue light exposure that allows steric access to an adjoining peptide (Wu, Y. I., Frey, D., Lungu, O. I., Jaehrig, A., Schlichting, I., Kuhlman, B., and Hahn, K. M. (2009) A genetically encoded photoactivatable Rac controls the motility of living cells. Nature 461, 104-108; and Strickland, D., Yao, X., Gawlak, G., Rosen, M. K., Gardner, K. H., and Sosnick, T. R. (2010) Rationally improving LOV domain-based photoswitches. Nat. Methods 7, 623-6), in this case, a protease recognition sequence. On the left, the transcription factor is tethered to the plasma membrane, sequestered from the cell nucleus. On the right, the coincidence of neuronal activity (which leads to rises in cytosolic calcium) and blue light causes the LOV domain to “uncage” the protease cleavage site, and brings the protease (TEV) into proximity of its cleavage site, via the intermolecular calmodulin-calmodulin binding peptide interaction. Consequently, the transcription factor is irreversibly cleaved from the plasma membrane, translocates to the nucleus, and activates transcription of the reporter gene of interest (FP, fluorescent protein). (FIG. 1B) Summary of constructs tested to optimize calcium response. Note that none of these contain the light-sensitive LOV domain, which is introduced later. For testing in HEK cells, Gal4 was used as the transcription factor and the transmembrane domain of CD4 to target it to the plasma membrane. Three different calmodulin (CaM) binding peptides (CaMbp), two different TEV cleavage sites (TEVcs), and two different forms of TEV protease (wild-type and truncated) were tested. (FIG. 1C) Results from testing 12 construct combinations under low and high calcium conditions in HEK cells. Gal4 drove expression of GFP, whose intensity was quantified across >2000 cells from 8-10 fields of view per condition. To elevate cytosolic calcium, HEK were treated with 5 mM CaCl₂ in the presence of 2 μM ionomycin for 5 minutes; cells were then returned to regular media and GFP was imaged 12 hours later. S/N ratios at top quantify GFP mean intensities under high versus low calcium. Error bars represent standard error of the mean.

In the TANGO system, TEV protease has a Kₘ of 240 μM for its TEVcs (Barnea, G., Strapps, W., Herrada, G., Berman, Y., Ong, J., Kloss, B., Axel, R., and Lee, K. J. (2008) The genetic design of signaling cascades to record receptor activation. Proc. Natl. Acad. Sci. U.S.A 105, 64-9; and Kapust, R. B., Tözsér, J., Copeland, T. D., and Waugh, D. S. (2002) The P1′ specificity of tobacco etch virus protease. Biochem. Biophys. Res. Commun. 294, 949-955). The expression levels of the FLARE tool components in HEK may approach or exceed this value, leading to significant TEV-mediated TEVcs cleavage even in the basal state (without CaM-CaMbp interaction). Efforts were made to weaken the affinity between TEV and TEVcs while maintaining high catalytic activity in the context of induced proximity. At the same time, ways of minimizing affinity between CaM and CaMbp in the low calcium state were explored, should this contribute to background as well.

Previous literature has shown that a truncated form of TEV missing its 23 C-terminal residues has unchanged k_{cat} for cleavage of a specific TEVcs but 7-fold higher Kₘ (450 μM instead of 61 μM for full-length TEV acting on the same TEVcs (Kapust, R. B., Tözsór, J., Fox, J. D., Anderson, D. E., Cherry, S., Copeland, T. D., and Waugh, D. S. (2001) Tobacco etch virus protease: mechanism of autolysis and rational design of stable mutants with wild-type catalytic proficiency. Protein Eng. 14, 993-1000); see FIG. 2 for summary of TEV/TEVcs kinetic constants). TEVΔ220-242 was tested in the context of FLARE. To further engineer the CaM-CaMbp interaction, two additional CaMbp peptides derived from CaMKII which are reported to have reduced CaM affinity in the low calcium state was also tested (Bayley, P. M., Findlay, W. A., and Martin, S. R. (1996) Target recognition by calmodulin: dissecting the kinetics and affinity of interaction using short peptide sequences. Protein Sci. 5, 1215-28; Evans, T. I. A., and Shea, M. A. (2009) Energetics of calmodulin domain interactions with the calmodulin binding domain of CaMKII. Proteins 76, 47-61; and Gao, X. J., Riabinina, O., Li, J., Potter, C. J., Clandinin, T. R., and Luo, L. (2015) A transcriptional reporter of intracellular Ca2+ in Drosophila. Nat. Neurosci. 18, 917-925). All 12 permutations are summarized in FIGS. 1B-1C (full length and truncated TEV×three CaMbp sequences×two TEVcs sequences). As expected, truncated TEV reduced background signal overall, giving less GFP expression in the basal state. Of the three CaMbps tested, M2 gave the lowest background.

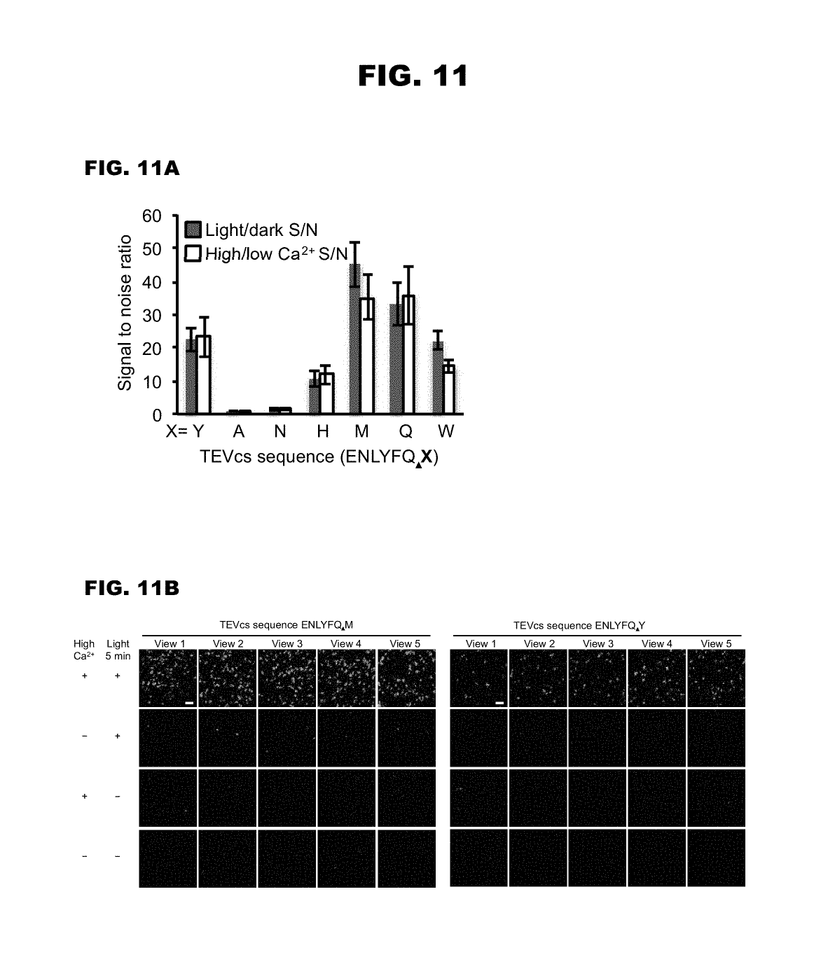

FIG. 2 shows a summary table of published TEV protease catalytic constants. The S219V mutation in TEV prevents TEV autolysis at position 218. X=N, H, and W do not have published characterization but were included as TEV cleavage site (TEVcs) variants in our screen (FIG. 11). Reference 1: Kapust, R. B., Tözsér, J., Fox, J. D., Anderson, D. E., Cherry, S., Copeland, T. D., and Waugh, D. S. (2001) Tobacco etch virus protease: mechanism of autolysis and rational design of stable mutants with wild-type catalytic proficiency. Protein Eng. 14, 993-1000; and Reference 2: Kapust, R. B., Tözsér, J., Copeland, T. D., and Waugh, D. S. (2002) The P1′ specificity of tobacco etch virus protease. Biochem. Biophys. Res. Commun. 294, 949-955.

The use of two TEVcs sequences in the screen—a lower affinity one derived from TANGO (Kₘ 240 μM and k_{cat} 0.84 min⁻¹) and a higher affinity one (Kₘ 50 μM and k_{cat} 1.9 min⁻¹) (Kapust, R. B., Tözsér, J., Copeland, T. D., and Waugh, D. S. (2002) The P1′ specificity of tobacco etch virus protease. Biochem. Biophys. Res. Commun. 294, 949-955) allowed for the comparison of the designs in two activity regimes. It was decided to move ahead with both TEVcs sequences, knowing that after addition of light gating to the system, background signal would be lessened overall, because the time window for possible accumulation of calcium-independent background signal would be greatly reduced. In such a context, the higher k_{cat} of the higher affinity TEVcs could be beneficial.

### Insertion of LOV Domain for Light Gating

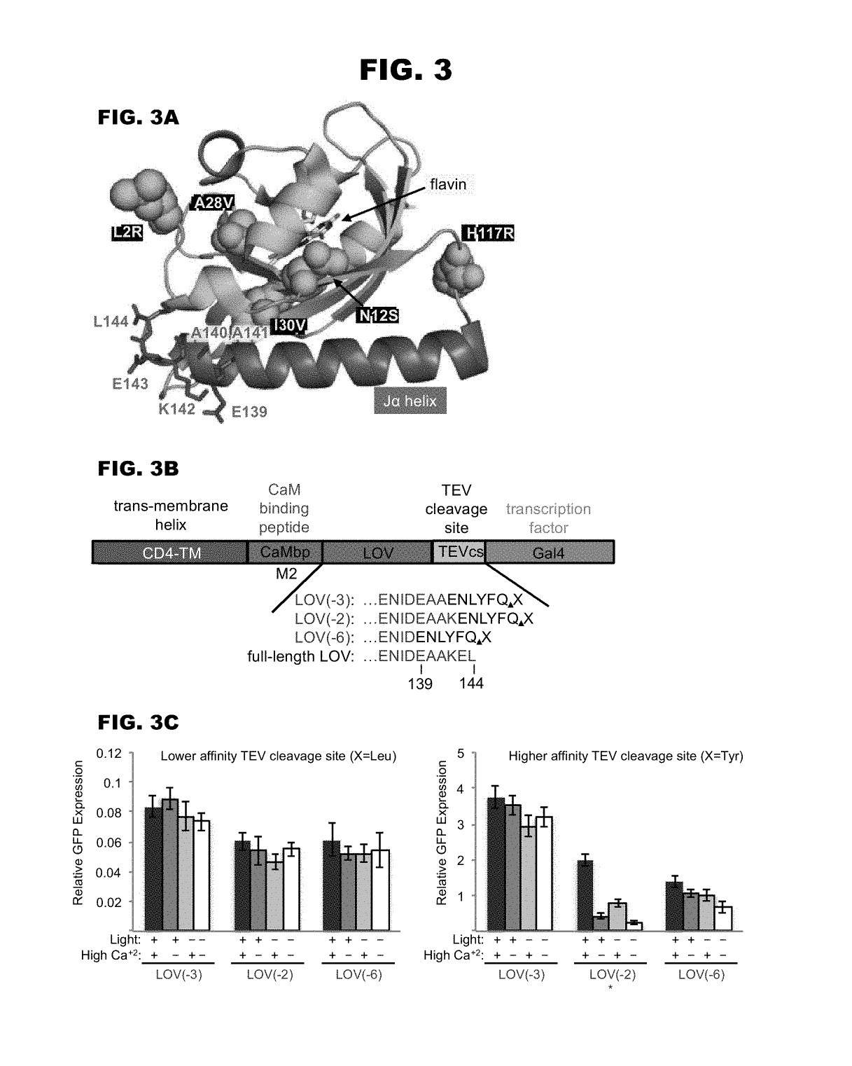

The LOV domain was selected for light gating of FLARE because it has been used in vivo (Hayashi-Takagi, A., Yagishita, S., Nakamura, M., Shirai, F., Wu, Y. I., Loshbaugh, A. L., Kuhlman, B., Hahn, K. M., and Kasai, H. (2015) Labelling and optical erasure of synaptic memory traces in the motor cortex. Nature advance on, 333-8), is reversible (Pudasaini, A., El-Arab, K. K., and Zoltowski, B. D. (2015) LOV-based optogenetic devices: light-driven modules to impart photoregulated control of cellular signaling. Front. Mol. Biosci. 2, 18), and does not require addition of exogenous cofactors as the Phy-PIF system does (Levskaya, A., Weiner, O. D., Lim, W. A., and Voigt, C. A. (2009) Spatiotemporal control of cell signalling using a light-switchable protein interaction. Nature 461, 997-1001). LOV2 from Avena sativa has been engineered for superior light/dark S/N (Wu, Y. I., Frey, D., Lungu, O. I., Jaehrig, A., Schlichting, I., Kuhlman, B., and Hahn, K. M. (2009) A genetically encoded photoactivatable Rac controls the motility of living cells. Nature 461, 104-108; and Lungu, O. I., Hallett, R. a., Choi, E. J., Aiken, M. J., Hahn, K. M., and Kuhlman, B. (2012) Designing Photoswitchable Peptides Using the AsLOV2 Domain. Chem. Biol. 19, 507-517) and is 16 kD with a flavin cofactor that becomes covalently attached via Cys48 upon blue light irradiation. This leads to a rapid (<1 sec) conformational change of the C-terminal Jα helix, which alters steric accessibility of any adjoined peptide (Konold, P. E., Mathes, T., Weiβenborn, J., Groot, M. L., Hegemann, P., and Kennis, J. T. M. (2016) Unfolding of the C-Terminal Jα Helix in the LOV2 Photoreceptor Domain Observed by Time-Resolved Vibrational Spectroscopy. J. Phys. Chem. Lett. 3472-3476) (FIG. 3A). The ability of LOV2 to photocage both TEVcs sequences (lower affinity ENLYFQL (SEQ ID NO://) and higher affinity ENLYFQY (SEQ ID NO://)) was tested by fusing them to LOV2's C-terminus. To increase the odds of beneficial communication between LOV2's flavin core and TEVcs, constructs were created in which up to 6 amino acids of Jα were “bitten back” to bring the TEVcs sequence closer to the LOV2 core (FIG. 3B). A total of 6 constructs were tested in HEK cells, under 4 conditions (±light and ±high calcium) (FIG. 3C). The best construct, LOV(−2) fused to the higher affinity TEVcs, gave a light/dark S/N of only 2. Background signal (GFP expression in the dark state) was considerable for all LOV2 fusion constructs.

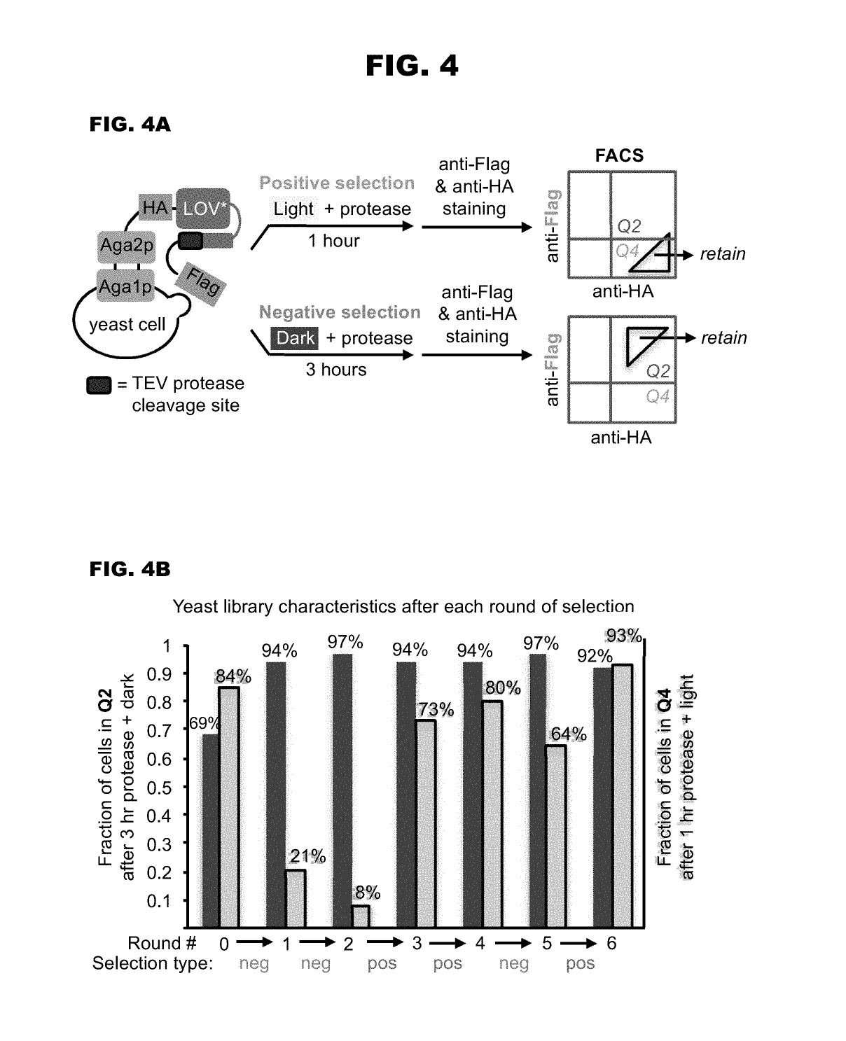

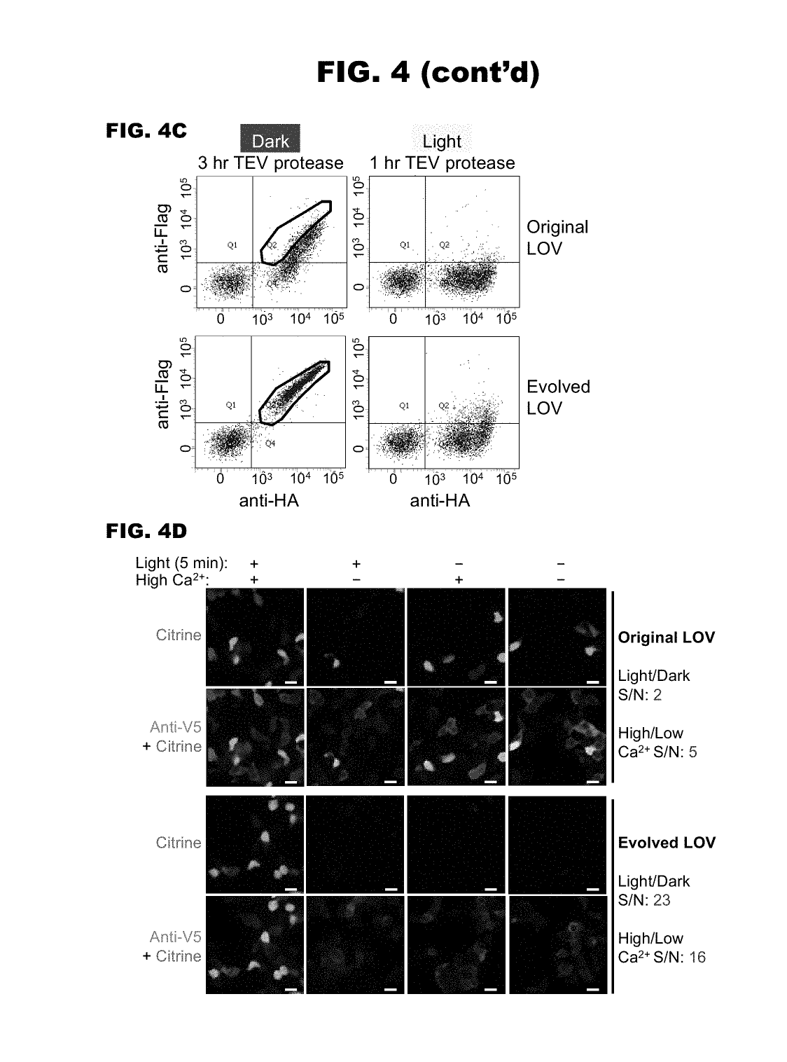

FIG. 3 shows the insertion of LOV domain to provide light gating. (FIG. 3A) Crystal structure of asLOV2 in the dark state (PDB:2V1A; Halavaty, A. S., and Moffat, K. (2007) N- and C-terminal flanking regions modulate light-induced signal transduction in the LOV2 domain of the blue light sensor phototropin 1 from Avena sativa. Biochemistry 46, 14001-14009). The C-terminal Jα helix dissociates from the LOV2 core upon blue light irradiation. The residues shown as dark sticks at the C-terminal end of Jα were targeted for replacement by the TEV cleavage site (“biting back”). The five mutations found in the evolved LOV domain (FIG. 4) are rendered in space-filling mode. (FIG. 3B) Summary of LOV2-TEVcs (TEV cleavage site, X=Y or L) fusion constructs tested. (FIG. 3C) Results from testing six LOV2-TEVcs fusion constructs in HEK cells. Each construct was tested under 4 conditions and GFP expression was quantified as in FIG. 1C. To elevate cytosolic calcium, HEK were treated with 5 mM CaCl₂ in the presence of 2 μM ionomycin for 5 minutes as in FIG. 1C. Light treatment was 5 minutes of 467 nm blue light at 60 mW/cm², 33% duty cycle. A star marks the fusion construct with the best performance in this assay (LOV(−2) fused to higher affinity TEVcs). Error bars represent standard error of the mean.

For FLARE to be a useful tool for neuroscience and other fields, it is imperative to minimize dark state leak. FLARE will be expressed in cells for days or even weeks prior to the experiment of interest. During this time, the cells may experience many calcium rises, but negligible TF release is required. Subsequently, a short period of light irradiation permits TF release, if calcium is also elevated. The large difference in duration between the dark period (days to weeks) and the light exposure period (minutes) necessitates a very large light/dark S/N for FLARE. It was found that this was not possible to achieve with the published LOV2 (Strickland, D., Yao, X., Gawlak, G., Rosen, M. K., Gardner, K. H., and Sosnick, T. R. (2010) Rationally improving LOV domain-based photoswitches. Nat. Methods 7, 623-6), whose caging efficiency varies greatly with the specific peptide sequence to which it is fused.

### Directed Evolution of LOV Domain to Improve Light Gating

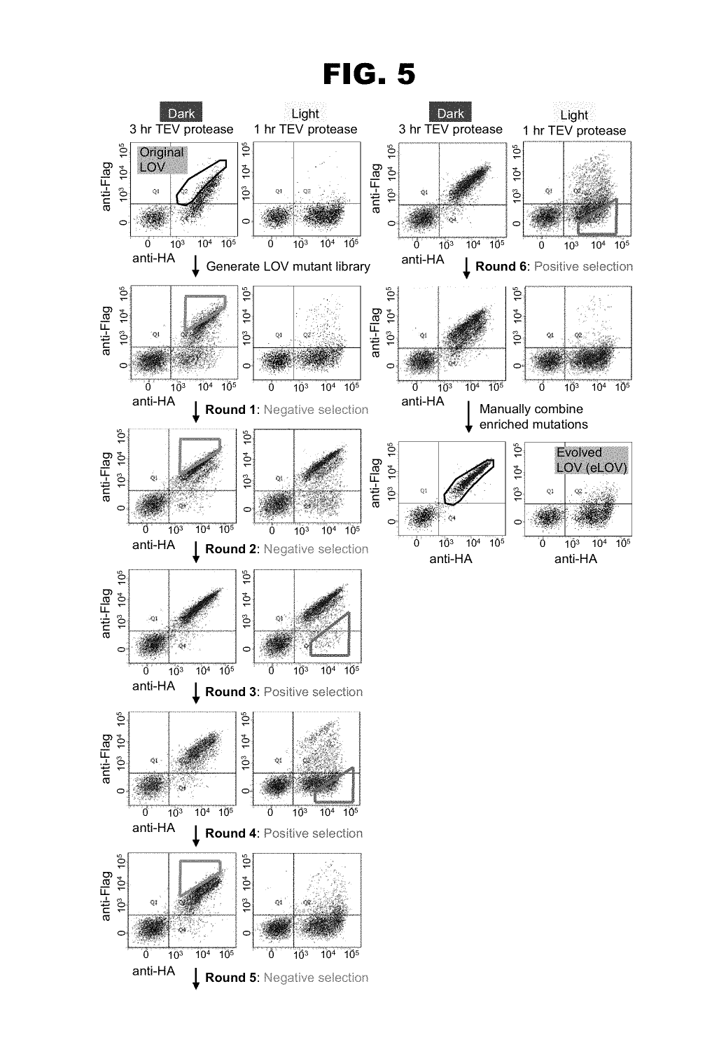

Directed evolution was used to improve the light caging efficiency of LOV for the TEVcs sequence in particular. It was reasoned that specific mutations in LOV2 might enhance the interactions between LOV2 and C-terminally fused TEVcs, leading to better steric protection and minimal cleavage by TEV protease in the dark state. To implement the evolution (FIG. 4A), LOV2 was mutagenized by error prone PCR, fused it to the TEVcs (higher affinity sequence ENLYFQY, because this gave the best results in FIG. 3C) and displayed the library on the yeast cell surface via fusion to the Aga2p mating protein. To perform positive selections for efficient TEVcs cleavage in the presence of blue light, the yeast library was incubated with purified TEV protease for 1 hour under a light source. After staining with antibody-fluorophore conjugates, fluorescence activated cell sorting (FACS) was used to enrich yeast cells displaying low anti-Flag/anti-HA fluorescence intensity ratios, indicative of TEVcs cleavage. Negative selections for resistance to TEVcs cleavage in the dark were implemented by incubating the yeast library with purified TEV protease in the dark for 3 hours, then using FACS to enrich cells with high anti-Flag/anti-HA fluorescence intensity ratios, indicative of intact TEVcs. Six rounds of alternating positive and negative selections were performed (FIG. 5). These served to gradually enrich the population of yeast displaying LOV mutants with both high TEVcs cleavage in the light state (yellow bars, FIG. 4B) and low TEVcs cleavage in the dark state (grey bars, FIG. 4B).

FIG. 4 shows the directed evolution of LOV domain to provide improved light gating in FLARE. (FIG. 4A) Selection scheme. A >10⁷ library of LOV variants was displayed on the yeast surface as a fusion to Aga2p protein. The TEV cleavage site ENLYFQY (SEQ ID NO://) (higher affinity) was fused to LOV's C-terminal end, and HA and Flag are flanking epitope tags. The positive selection enriches mutants with low Flag staining (i.e., high TEVcs cleavage) after protease treatment in the light. The negative selection enriches mutants with high Flag staining (i.e., low TEVcs cleavage) after prolonged protease treatment in the dark. (FIG. 4B) Graph summarizing yeast library characteristics after each round of selection. Accompanying FACS plots in FIG. 5. Dark bars indicate the fraction of yeast cells in quadrant Q2 (out of all cells in Q2+Q4) after 3 hours of TEV protease incubation in the dark (left y-axis). Quadrants are defined in FIG. 4A. Light bars indicate the fraction of yeast cells in Q4 (out of all cells in Q2+Q4) after 1 hour of TEV protease incubation in blue light (right y-axis). (FIG. 4C) FACS analysis of original LOV2 (Strickland, D., Yao, X., Gawlak, G., Rosen, M. K., Gardner, K. H., and Sosnick, T. R. (2010) Rationally improving LOV domain-based photoswitches. Nat. Methods 7, 623-6) (top) and our evolved eLOV (bottom) on yeast. Evolved LOV displays superior protection of TEVcs against TEV cleavage in the dark state (left). (FIG. 4D) Comparison of original LOV2 (Strickland, D., et al. (2010) infra) (top) and the evolved eLOV (bottom) in HEK cells, in the context of FLARE. Constructs were CD4-TM:CaMbp(M2):(e)LOV:TEVcs(high affinity):Gal4 and CaM-TEV(truncated). Gal4 drives expression of the fluorescent protein Citrine. High calcium (5 minutes) and light conditions were the same as those in FIG. 3C. Anti-V5 staining detects expression of CaM-TEV. S/N ratios on right are based on mean Citrine intensities across >500 cells from 10 fields of view per condition. Scale bars, 20 μm.

FIG. 5 shows library progression during directed evolution of LOV domain. This figure is related to FIG. 4. Re-amplified yeast cultures following each round of selection were compared under identical conditions. The original LOV2 and final eLOV are also shown for comparison. To evaluate dark state leak, yeast were treated with ˜30 μM wild-type TEV protease in the dark for 3 hours, then stained with anti-Flag and anti-HA antibodies as in FIG. 4C. To evaluate TEVcs accessibility in the light state, yeast were treated with ˜30 μM TEV protease under a broad wavelength light source for 1 hour, then stained with antibodies. The polygons indicate the FACS sorting gates used in the type of selection as indicated beneath each plot.

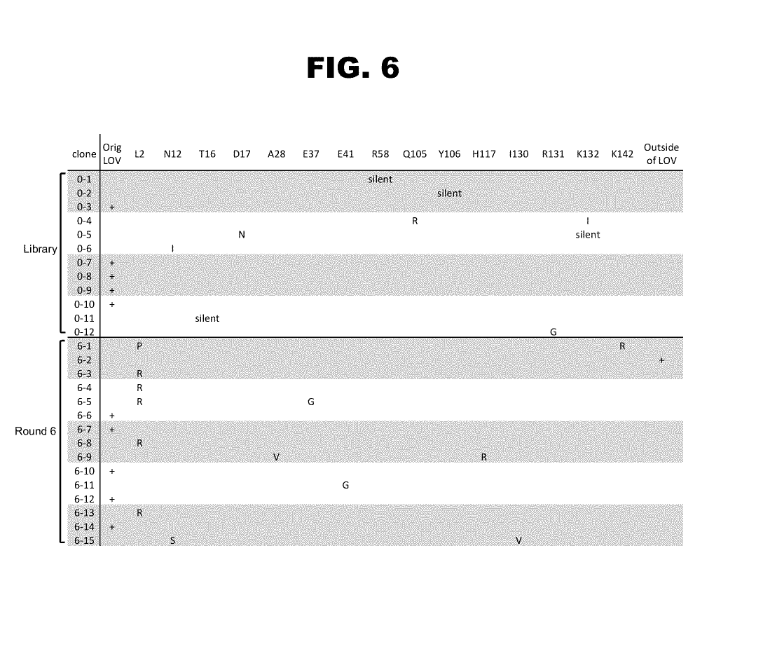

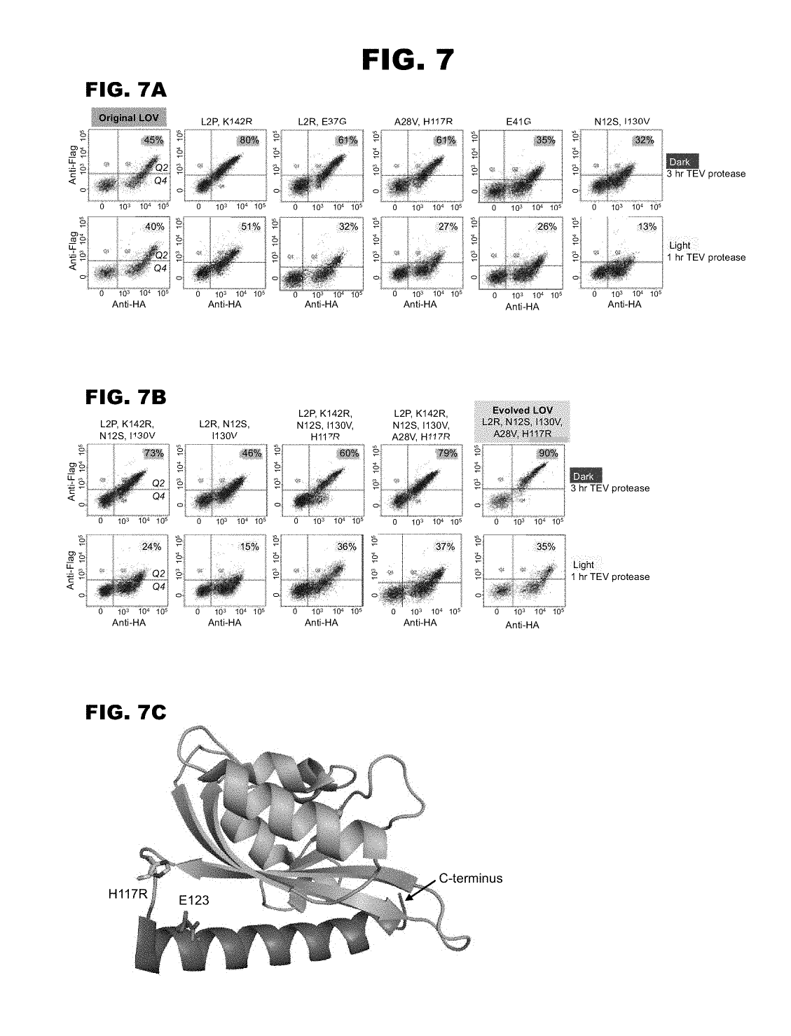

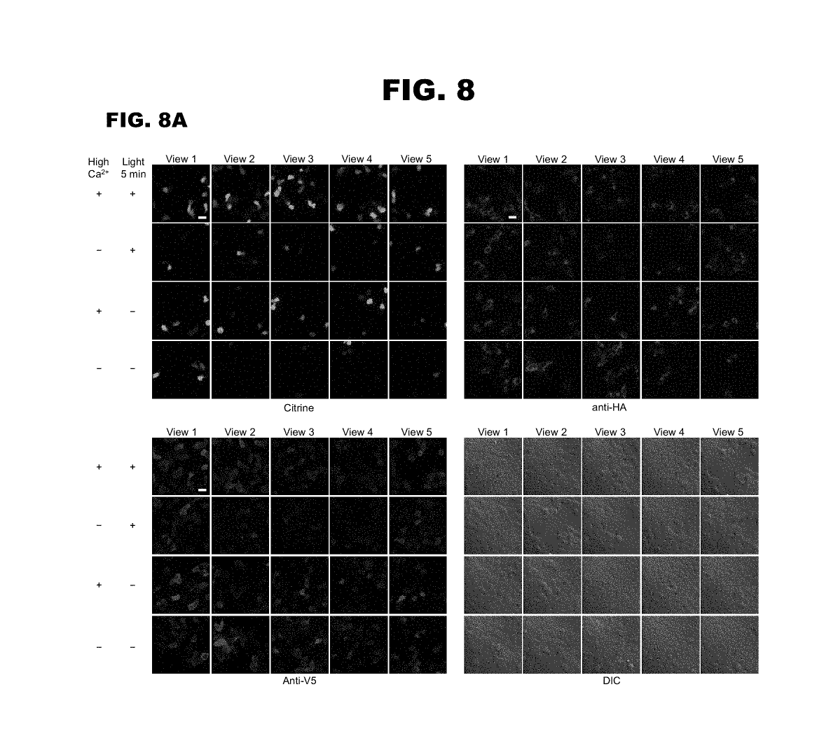

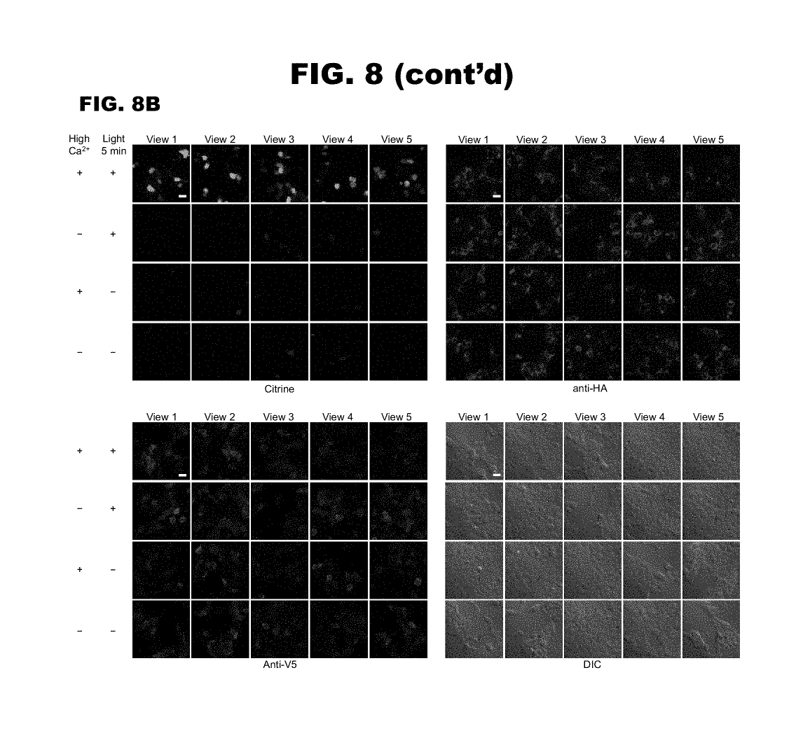

Sequencing of enriched clones from round 6 (FIG. 6) highlighted five mutants of interest, three of which showed superior performance to original LOV2 on the yeast surface (FIG. 7). Mutations present in these clones were manually combined into a single LOV gene to give “eLOV” for evolved LOV. On the yeast surface (FIG. 4C) and in HEK mammalian cells (FIG. 4D and FIG. 8), eLOV was clearly superior to the original LOV for light gating of the TEVcs, especially in the dark state, where GFP expression resulting from TEVcs cleavage was now minimal. The quantified light/dark S/N in HEK was 23, in contrast to 2 for the original LOV2. As anticipated, the introduction of light gating also improved the calcium response of the tool—by reducing the time window for possible accumulation of background signal. The same modules (truncated TEV, M2 CaMbp) that gave a high/low Ca²⁺ S/N of only 2 in HEK (FIG. 1C) now gave a S/N of 16 with eLOV incorporated (S/N of 5 with original LOV incorporated) (FIG. 4D).

FIG. 6 shows the sequencing analysis of yeast clones from LOV directed evolution experiment. 12 clones were sequenced from the original LOV library, and 15 clones from the final round of selection (round 6). Mutations with respect to the original LOV2 (Strickland, D., Yao, X., Gawlak, G., Rosen, M. K., Gardner, K. H., and Sosnick, T. R. (2010) Rationally improving LOV domain-based photoswitches. Nat. Methods 7, 623-6) are shown. Some clones were the original LOV2 (first column), some contained silent mutations, and one had a mutation outside the LOV2 gene.

FIG. 7 shows the FACS analysis of specific LOV mutants. (FIG. 7A) Analysis of five LOV mutants enriched after 6 rounds of selection. Original LOV2 is shown for comparison. Each clone is evaluated for dark state protection and light state cleavage as in FIG. 3C and FIG. 5. Numbers in top right of each graph give the percentage of yeast in quadrant Q2 (out of total yeast in Q2+Q4). (FIG. 7B) Five designed LOV mutants based on manual combination of mutations in (FIG. 7A). Clones were evaluated on yeast as in (FIG. 7A). (FIG. 7C) LOV2 structure (PDB:2V1A; Halavaty, A. S., and Moffat, K. (2007) N- and C-terminal flanking regions modulate light-induced signal transduction in the LOV2 domain of the blue light sensor phototropin 1 from Avena sativa. Biochemistry 46, 14001-14009) highlighting proximity between H117 in the LOV core and E123 in the Jα helix. eLOV has a H117R mutation, which may interact with E123 to help stabilize eLOV in the dark state, leading to improved caging.

FIG. 8 Same as FIG. 4D, but with additional fields of view, and immunofluorescence staining of the transcription factor component (anti-HA) as well. (FIG. 8A) is original LOV2 and (FIG. 8B) is eLOV. DIC, Differential Interference Contrast image. Scale bars, 20 μm.

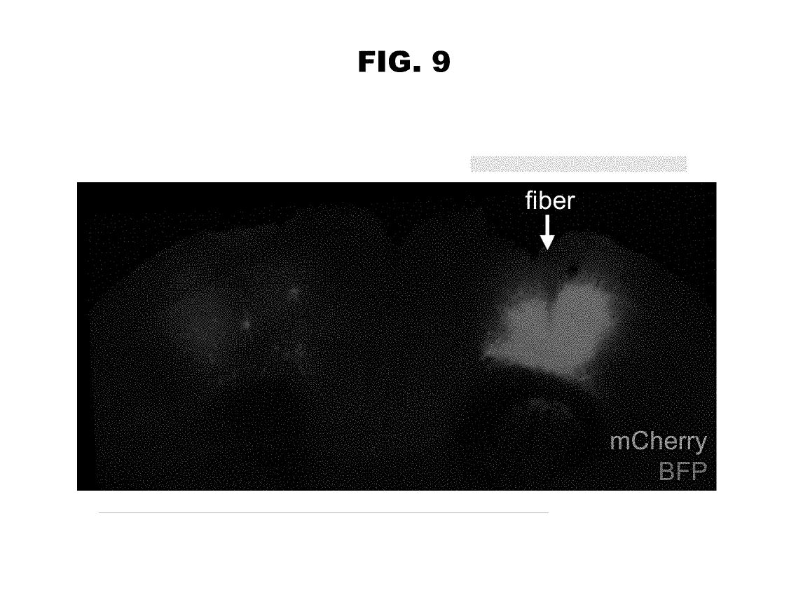

To test whether eLOV could provide sufficient light gating and suppress dark state leak even in in vivo applications, eLOV-containing FLARE components were introduced by AAV transduction into both hemispheres of adult mice. After 7 days of expression, mice were injected with kainate to induce seizure (and maximally activate neurons throughout the cortex), and 473 nm light was delivered by implanted optical fiber into one hemisphere only, for 30 minutes. Twenty four hours later, the mice were sacrificed and imaged. FIG. 9 shows robust mCherry expression (resulting from TEVcs cleavage, transcription factor release, and transcription and translation of mCherry) in the right, light-exposed hemisphere only. The left hemisphere has minimal mCherry expression, indicating that eLOV cages TEVcs tightly over the 7 day expression window, and during the 30 minute kainate seizure period, preventing protease cleavage and transcription factor release. This result was not possible to achieve with earlier tool generations that utilized the original LOV2 domain.

FIG. 9 shows the testing of light gating by eLOV in the in vivo mouse brain. Adult mice were injected in both hemispheres with AAVs encoding FLARE components: CD4-TM:CaMbp(M2):eLOV:TEVcs(ENLYFQY):tTA, CaM-TEV(full length), TET-mCherry, and BFP (as a viral expression marker). An optical fiber was surgically implanted into the right hemisphere only. 7 days later, mice were injected intraperitoneally with kainate to induce seizure, and 5 minutes later, blue light was applied to the right hemisphere only via the fiber (30 minutes of 467 nm light at 0.5 mW, 50% duty cycle). The following day, mice were sacrificed and sections were imaged by confocal microscopy. mCherry indicates activation of FLARE.

The 5 mutations in eLOV enriched via directed evolution are highlighted in FIG. 3A. For example, Leu2, located in a flexible loop, is mutated to Arg in eLOV. Perhaps this permits it to form a salt bridge with the Glu sidechain in TEVcs (ENLYFQY), leading to tighter dark state caging. H117 is located in the loop that connects the Jα helix to the rest of the LOV domain. H117R in eLOV could potentially stabilize Jα in the dark state by forming a salt bridge to E123 (FIG. 7C).

### Further Improvements to FLARE and Testing in Neurons

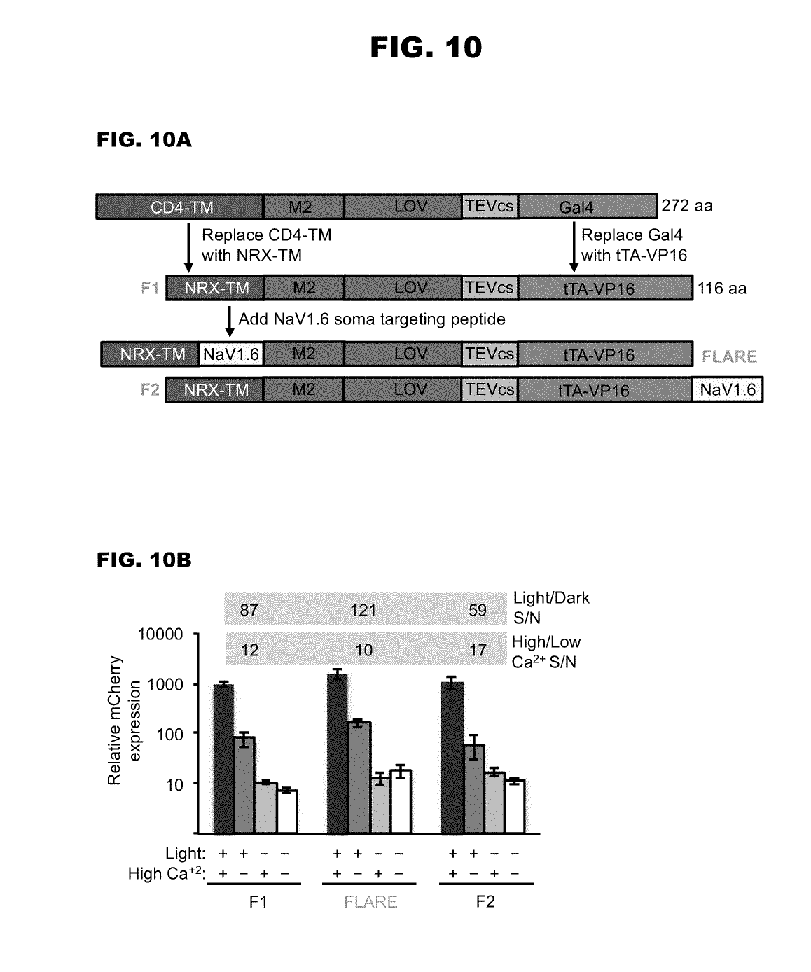

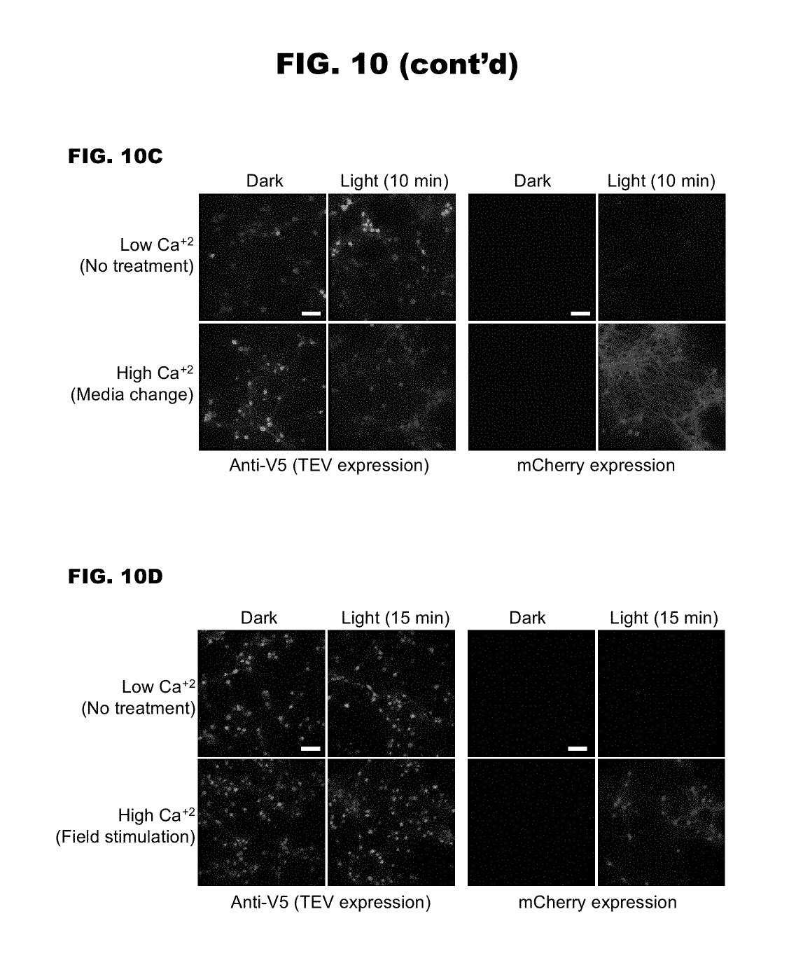

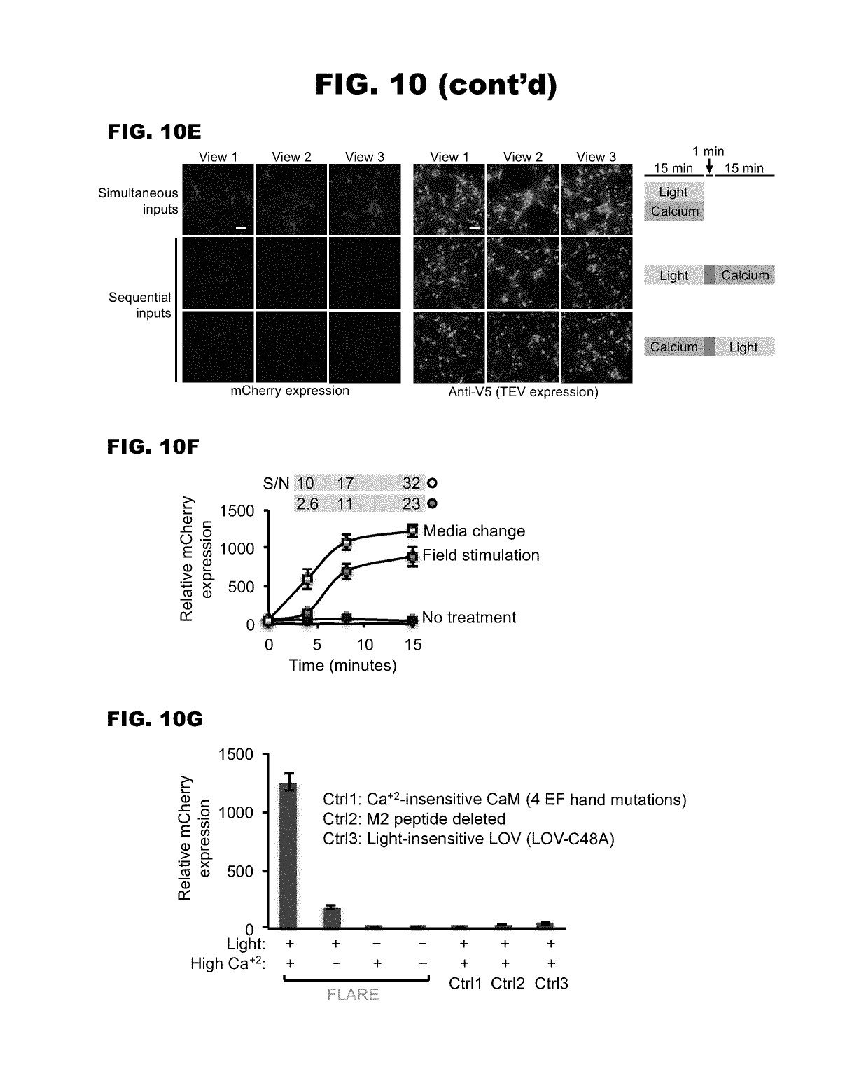

Though encouraged by the results in HEK cells, neurons present a considerably greater challenge. Natural calcium rises in neurons are not like the sustained 5-minute long >1 μM CaCl₂ rises that were artificially induced with ionomycin in HEK cells. Cell surface proteins that traffic well in HEK frequently fail to do so in neurons. To address these and other challenges in transitioning FLARE from HEK to neurons, a number of changes and improvements were made to the tool, as follows (FIG. 10A).

FIG. 10 shows FLARE optimization and testing in neurons. (FIG. 10A) Summary of sequential improvements and changes to FLARE. F1 and F2 are earlier versions of the tool. (FIG. 10B) Comparison of tool versions in neurons. tTA transcription factor drives expression of mCherry. To elevate cytosolic calcium, half of the culture medium was replaced with fresh neurobasal media (of identical composition), and mixed by gentle pipetting. Calcium elevation under these conditions was confirmed by GCaMP5 imaging. Low calcium samples were not treated. Light stimulation was for 10 minutes using 467 nm light at 60 mW/cm², 33% duty cycle. Mean mCherry intensities were quantified across >400 cells from 10 fields of view per condition, and presented on a log scale. (FIG. 10C) Confocal imaging of FLARE in rat cortical neurons at DIV20. Constructs were introduced by AAV viral transduction at DIV13. Calcium and light conditions were identical to those in (FIG. 10B). 18 hours after treatment, neurons were fixed, stained with anti-V5 antibody (to visualize CaM-TEV expression), and imaged. (FIG. 10D) Confocal imaging of FLARE after field stimulation. Neurons were transduced with AAVs at DIV10 and imaged at DIV17. Field stimulation parameters were 3-second trains consisting of 32 1-millisecond 50 mA pulses at 20 Hz for a total of 15 minutes. Light was applied for 15 minutes at 467 nm, 60 mW/cm², 10% duty cycle. Neurons were fixed, stained, and imaged 18 hours later. (FIG. 10E) Comparison of FLARE response with simultaneous (top) versus sequential (middle and bottom) light/calcium inputs. DIV10 cortical neurons expressing FLARE components were activated by field stimulation and blue light (same conditions as in (FIG. 10D). In the case of sequential inputs, a 1 minute pause separated the two inputs. Three separate fields of view shown per condition. (FIG. 10F) FLARE sensitivity. DIV18 neurons expressing FLARE were untreated, or activated with field stimulation (same parameters as in (FIG. 10D) or media change (90% of culture medium exchanged) for 4, 8, or 15 minutes with simultaneous application of blue light (467 nm, 60 mW/cm², 10% duty cycle). S/N values reflect mean mCherry intensity ratios with versus without neuronal activity, from >800 cells across 10 fields of view per condition. (FIG. 10G) Control experiments to probe FLARE mechanism. Conditions were the same as in (FIG. 10B). Control constructs contained mutations in calcium-binding, CaM-binding, and light sensitive regions, as described. All scale bars, 100 μm.

First, to further improve the calcium response, testing of TEVcs sequences was expanded. The P1′ position, which was previously varied between L (lower affinity) and Y (higher affinity), was also mutated to A, N, H, M, Q, and W. A striking improvement in both calcium S/N and light/dark S/N with P1′=M was observed (FIG. 11), mainly due to higher GFP signal in the +light+high Ca²⁺ state. This is consistent with previous literature showing that P1′=M gives 6-fold faster k_{cat} for TEV cleavage in addition to a slightly higher Kₘ, compared to P1′=Y (Kapust, R. B., Tözsér, J., Copeland, T. D., and Waugh, D. S. (2002) The P1′ specificity of tobacco etch virus protease. Biochem. Biophys. Res. Commun. 294, 949-955) (FIG. 2).

FIG. 11 shows the screening of alternative TEV cleavage site (TEVcs) sequences in HEK cells. (FIG. 11A) Summary of results. The following constructs were introduced by PEI max transfection into HEK cells: CD4-TM:CaMbp(M2):eLOV:TEVcs:Gal4, CaM-TEV(truncated), and UAS-Citrine. The specific TEVcs sequence varied at the P1′ position as shown. High calcium (5 minutes) and light conditions were the same as those in FIG. 2C. S/N ratios were based on mean Citrine intensities across >2000 cells from 10 fields of view per condition. Error bars represent standard error of the mean. (FIG. 11B) Fluorescence images for the X=M and X=Y constructs in (FIG. 11A). Citrine channels are shown at 10× magnification, 5 fields of view per condition. Scale bars, 100 μm.

Second, to reduce the size of the largest FLARE component, necessary for packaging into AAVs, the CD4 transmembrane domain was replaced with a Neurexin-3b-derived transmembrane domain, which is 2 times smaller. Third, to maximize FLARE sensitivity, Gal4 was replaced with the tTA-VP16 transcription factor, which has subnanomolar DNA binding affinity and a stronger transcriptional activation domain (Orth, P., Schnappinger, D., Hillen, W., Saenger, W., and Hinrichs, W. (2000) Structural basis of gene regulation by the tetracycline inducible Tet repressor-operator system. Nat. Struct. Biol. 7, 215-219).

Fourth, to facilitate the translocation of cleaved transcription factor from the plasma membrane to the nucleus, a soma targeting sequence was inserted (Garrido, J. J., Giraud, P., Carlier, E., Fernandes, F., Moussif, A., Fache, M.-P., Debanne, D., and Dargent, B. (2003) A targeting motif involved in sodium channel clustering at the axonal initial segment. Sci. (New York, N.Y.) 300, 2091-2094). FIG. 10B shows that these modifications all contributed to improved FLARE performance in neuron culture.

FIGS. 10C-10D show imaging of a FLARE tool in cultured rat neurons at DIV17. The tTA TF drives expression of TRE-mCherry in the nucleus. Light stimulation was 10 or 15 minutes using 467 nm blue light at 60 mW/cm² and 10-33% duty cycle. To elevate intracellular calcium, field stimulation was used (FIG. 10D), or half of the culture media was replaced with fresh media of the same composition (FIG. 4C); GCaMP5 imaging showed that this treatment produced calcium transients for 10 minutes or more. Neurons were allowed, 18 hours after calcium and light stimulation, to transcribe and translate mCherry. FIGS. 10C-10D show that mCherry expression was robust only in one of four conditions in each experiment, when neurons were subjected to both light and activity. There is some detectable background signal in the light exposed/non-stimulated cells (>10 fold less than with stimulation) but this may reflect basal calcium activity, as these neurons were not repressed/silenced. mCherry expression was barely detectable in all dark state conditions, attesting to the effectiveness of eLOV in caging TEVcs from protease cleavage over the entire 7 day expression window (light/dark S/N 121 and 17, respectively, in FIGS. 10C-10D).

An essential control is to test whether FLARE generates transcription only upon coincident detection of light and activity inputs; sequential inputs, even if closely spaced, must not produce transcription. Alternative designs, for example using split TEV (Wehr, M. C., Laage, R., Bolz, U., Fischer, T. M., Grünewald, S., Scheek, S., Bach, A., Nave, K.-A., and Rossner, M. J. (2006) Monitoring regulated protein-protein interactions using split TEV. Nat. Methods 3, 985-93; and Gray, D. C., Mahrus, S., and Wells, J. A. (2010) Activation of specific apoptotic caspases with an engineered small-molecule-activated protease. Cell 142, 637-646) that reconstitutes in the presence of high calcium, give rise to the concern that sequential, rather than coincident, inputs could also activate transcription. This is because split TEV reconstitution may be irreversible or slowly reversible, such that functional protease accumulates (and persists) in activated neurons outside of the light window. FIG. 10E shows that the FLARE design is highly specific for simultaneous light and calcium inputs, and sequential inputs (light followed by high calcium, or high calcium followed by light) do not produce any mCherry expression.

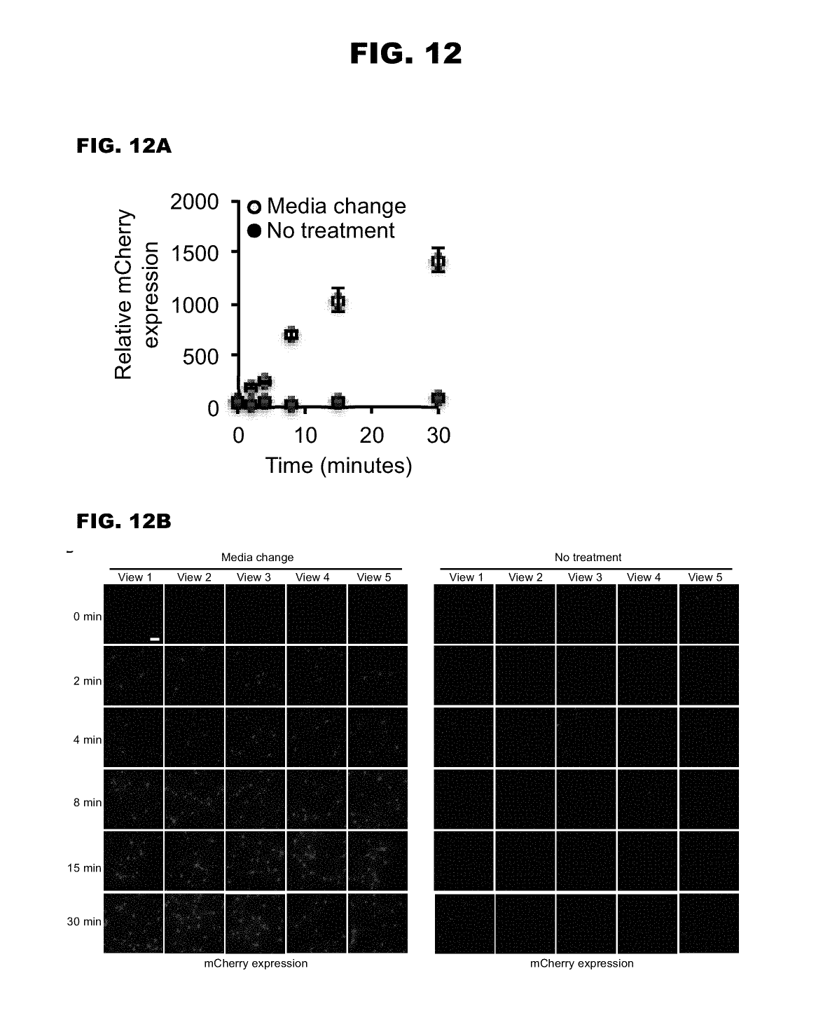

To characterize the sensitivity, or temporal resolution, of FLARE, light was delivered to neurons for various lengths of time, coincident with two forms of activity stimulation (field stimulation or media change) (FIG. 10F and FIG. 12). Media change produced a robust signal in just 4 minutes, while field stimulation gave a S/N of 11 after 8 minutes.

FIG. 12 is the same as FIG. 10F, but with additional time points and accompanying fluorescence images. (FIG. 12A) Summary graph of FLARE response as a function of stimulation time. 90% of the culture media was replaced one time (at t=0), and then blue light (473 nm LED, 60 mW/cm², 10% duty cycle) was applied for 2-30 minutes, as indicated. Error bars represent standard error of the mean (FIG. 12B) Fluorescence images for datapoints in (FIG. 12A). For each condition, 5 fields of view are shown. Scale bars, 100 μm.

Finally, to test if FLARE works by the mechanism that was designed, imaging was performed in neurons using FLARE components with targeted mutations. FIG. 10G shows that mutation of the calcium-binding EF hands of the calmodulin domain, or deletion of the M2 peptide from the TF component of FLARE, or mutation of eLOV to remove the cysteine that crosslinks with flavin (C48A) all abolished mCherry expression in the +light+activity condition. Together, these controls suggest that calcium and light-sensing by FLARE operate in the manner that was designed.

### Example 2: FLARE Activity in Neurons

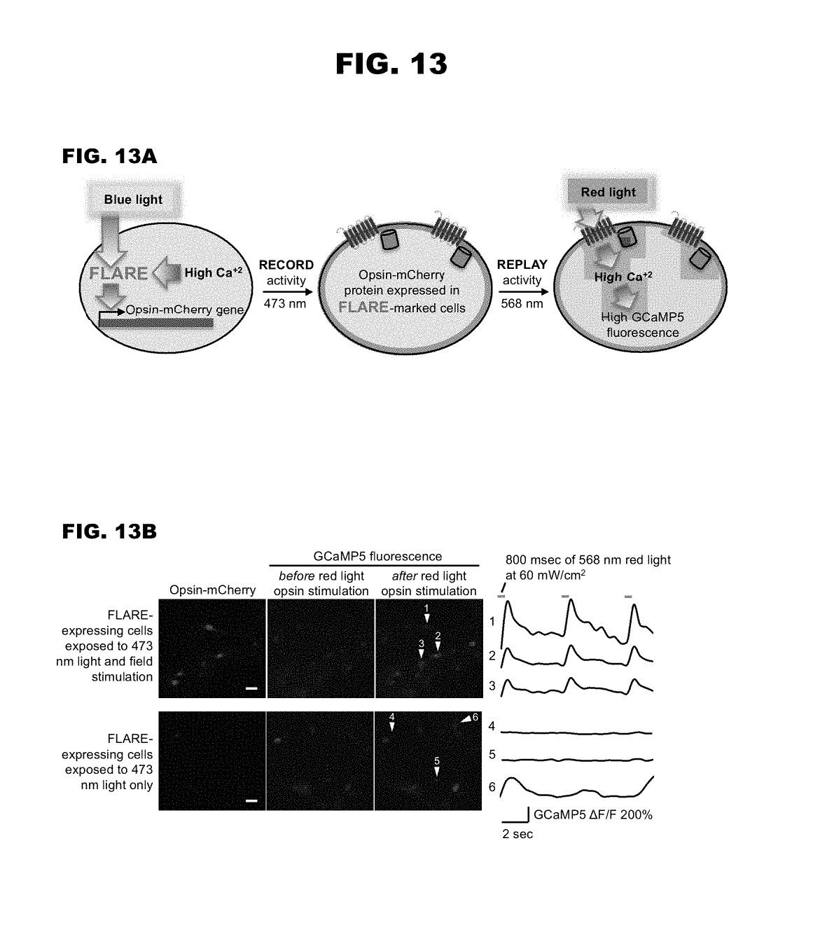

Having characterized the properties of FLARE in neuron culture, it was tested whether FLARE could be used not only to mark neurons active during defined time windows, but to manipulate them (FIG. 13A). Thus, instead of driving mCherry expression, FLARE was used to drive expression of a light-activated ion channel, Chrimson-mCherry (Chrimson from Chlamydomonas noctigama is a red light-activated channelrhodopsin (Klapoetke et al. (2014) Nat. Methods 11, 338-46)). With only a 15-minute blue light plus field stimulation time window, would opsin expression levels be sufficient to enable functional reactivation of FLARE-marked neurons? FIG. 13B shows imaging of these neurons 18 hours after blue light exposure. Opsin-mCherry expression can be seen in stimulated neurons (top row) but not in untreated neurons (bottom row). Recording of GCaMP5 fluorescence in response to pulses of opsin-activating red light shows that FLARE-marked cells can indeed be re-activated to give calcium transients. In the negative control (neurons not subjected to field stimulation), GCaMP5 fluorescence either does not rise, or rises periodically in a manner uncorrelated with the red light pulses.

FIG. 13 shows functional reactivation of neurons marked by FLARE. (FIG. 13A) Scheme. The coincidence of blue light and high calcium activate FLARE, resulting in expression of opsin-mCherry in subsets of neurons. To re-activate FLARE-marked neurons, red light is applied to stimulate opsin, resulting in cytosolic calcium rises, which can be read out with the GCaMP5 fluorescent calcium indicator (Akerboom, et al. (2012) J. Neurosci. Off. J. Soc. Neurosci. 32, 13819-13840). (FIG. 13B) Imaging results from experiment performed as in (FIG. 13A). Cultured neurons were transduced with FLARE AAV viruses (including the reporter gene TET-Chrimson-mCherry) and GCaMP5 lentivirus at DIV13. At DIV19, neurons were treated with blue light (467 nm at 60 mW/cm², 10% duty cycle) and field stimulation (15 minutes of 3-second long trains, each consisting of 32 1-millisecond 48 mA pulses at 20 Hz) for 15 minutes total. 18 hours later, at DIV20, GCaMP5 fluorescence timecourses were recorded (for the 6 indicated cells) while stimulating the Chrimson channelrhodopsin with pulses of red 568 nm light as indicated. The bottom image set shows a negative control in which field stimulation was withheld at DIV13, but blue light was applied. Scale bars, 50 μm.

### Example 3

A second FLARE tool was modified and designed for use with other calcium induced protein interactions. In the basal state, the TF is tethered to the cell's plasma membrane, unable to activate transcription of the reporter gene located in the cell's nucleus. Upon exposure to both light and high calcium, however, the TF is cleaved from the membrane and translocates to the nucleus because (1) the protease recognition site is unblocked by the light-sensitive eLOV domain, and (2) the protease is recruited to its recognition site via a calcium-regulated intermolecular interaction between troponin C (TnC) and a TnC binding peptide (e.g., TnI(95-139)). Importantly, high calcium alone is not sufficient to give TF release because the protease site remains blocked, and light alone is not sufficient because the protease is far away, and its affinity for its recognition site is too low to afford cleavage in the absence of induced proximity. Also key to this design is that both calcium sensing and light sensing are fully reversible, such that sequential rather than coincident inputs (such as high calcium followed by light) are unable to trigger TF release.

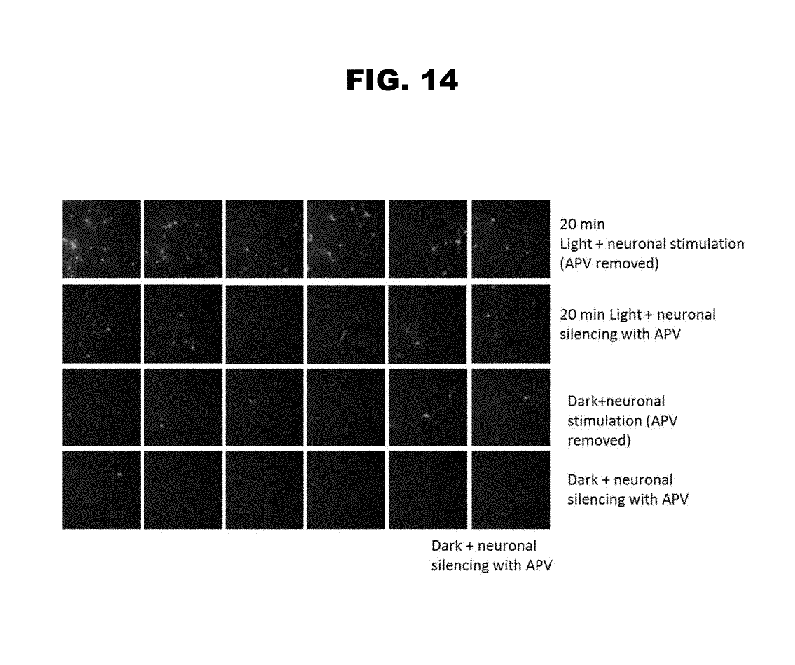

In this second FLARE tool, the transmembrane component includes CD4-TnI(95-139)-eLOV-TEVcs(ENLYFQY)-tTA, the protease component includes TnC(2 mutations)-TEVfl and the reporter gene includes TET-EYFP. As shown in FIG. 14, 20 min of light exposure together with neuronal stimulation enhanced the expression of the reporter gene. Neuronal stimulation was achieved by the removal of the selective NMDA receptor antagonist 2-amino-5-phosphonopentanoic acid (APV). Removal of APV from neurons is known to increase neuronal Ca²⁺. Enhanced expression of the reporter gene is not evident with 20 min of light exposure together with neuronal silencing using APV, dark conditions together with neuronal stimulation by removal of APV, or dark conditions together with neuronal silencing using APV.

### Example 4: Use of FLARE In Vivo

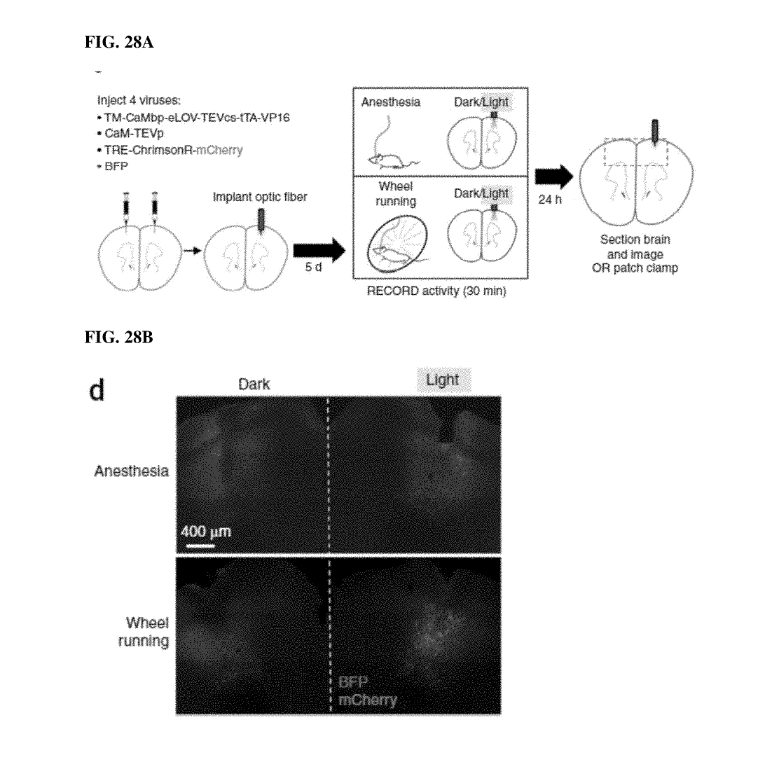

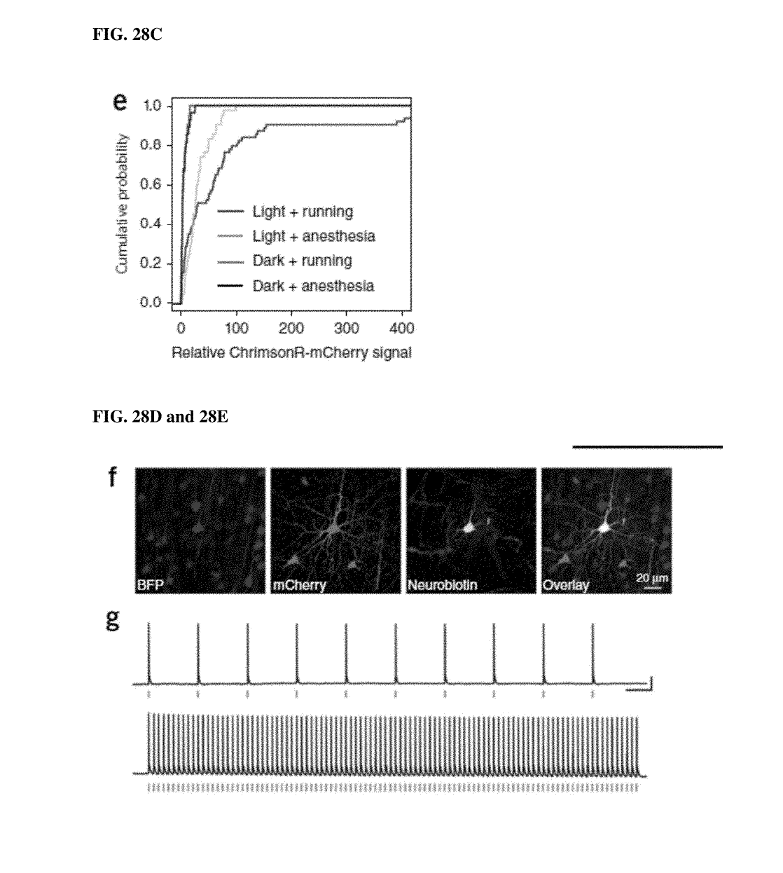

To test the function of FLARE in vivo recombinant AAV viruses comprising a nucleotide sequence encoding FLARE components (as described in Example 2) were injected into the motor cortex of adult mice. Blue light was delivered via an implanted optical fiber; and the mice were stimulated via wheel running (single 30-minute session) or were anesthetized. 24 hours later, mice were perfused and imaged for ChrimsonR-mCherry expression to quantify FLARE activation. FIG. 28A. As depicted in FIGS. 28B and 28C, FLARE is minimally activated in the absence of blue light. A small but statistically significant (P=0.013) increase in mCherry intensity was observed in animals that were running during the blue light period compared to animals that were inactive. FIG. 28C. To see if FLARE could drive sufficient levels of ChrimsonR expression for functional manipulation, whole-cell patch-clamp recordings from mChaerry-positive neurons n the motor cortex of light/running animals were performed. As shown in FIGS. 28D and 28E, robuts red-light-induced action potentials were observed. These results suggest that FLARE is gated by light and elevated calcium in the in vivo context.

FIG. 28A-28E. Functional testing of FLARE in vivo. FIG. 28A: Scheme for testing FLARE in the mouse brain. Concentrated AAV viruses encoding FLARE components (in addition to blue fluorescent protein (BFP), an infection maker), were injected into the motor cortex of adult mice (both left and right hemispheres). After 5 days of expression, blue light was delivered to the right hemisphere via implanted optical fiber (single 30-min session of 473-nm light at 0.5 mW, 50% duty cycle (2 s light every 4 s)), while mice were running on an exercise wheel or were anesthetized. 24 hours later, mice were perfused for imaging analysis. FIG. 28B. Two representative brain sections from experiments in FIG. 28A, for anesthetized mouse (top) and wheel running mouse (bottom. Right hemisphere was illuminated for 30 min., whereas left hemisphere was kept in the dark. Activated FLARE drives expression of mCherry. BFP is an AAV infection marker. FIG. 28C. Quantitation of brain imaging data. For each brain hemisphere with BFP signal above background, the total ChrimsonR-mCherry fluorescence intensity across seven consecutive brain sections around the virus injection site were quantified. 21-63 brain sections were analyzed from 3-9 mice per condition. Ligh+running animals have significantly higher mCherry expression than light+anesthetized animals (Kolmogorov-Smirnov Test, P=0.013). FIG. 28D. Whole-cell patch-clamp electrophysiology was used to record from ChrimsonR-mCherry-expressing neurons in the mouse brain 24 h after light+running stimulation. Neurobiotin was injected into the patched neuron. FIG. 28E. Sample traces showing action potentials elicited in response to 5-ms pulses of 589-nm light delivered at 1 Hz (upper panel) or 10 Hz (lower panel). Scale bars=20 mV, 500 mx. Experiments in FIG. 28B-28G have each been performed once.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A nucleic acid system comprising:
A) a first nucleic acid comprising, in order from 5′ to 3′:
a) a nucleotide sequence encoding a light-activated, calcium-gated fusion polypeptide comprising, in order from amino terminus to carboxyl terminus:
i) a transmembrane domain;
ii) a calmodulin-binding polypeptide or a troponin I polypeptide;
iii) a LOV-domain light-activated polypeptide comprising an amino acid sequence having at least 80% amino acid sequence identity to an amino acid sequence selected from the group consisting of SEQ ID NOS:36-40 and SEQ ID NOS:150-152; and
iv) a proteolytically cleavable linker; and
b) an insertion site for a nucleic acid comprising a nucleotide sequence encoding a polypeptide of interest; and
B) a second nucleic acid comprising a nucleotide sequence encoding a second fusion polypeptide comprising:
i) a calcium-binding polypeptide selected from a calmodulin polypeptide and troponin C polypeptide; and
ii) a protease that cleaves the proteolytically cleavable linker.

2. A nucleic acid system comprising:
a) a first nucleic acid comprising a nucleotide sequence encoding a light-activated, calcium-gated fusion polypeptide comprising, in order from amino terminus to carboxyl terminus:
i) a transmembrane domain;
ii) a calmodulin-binding polypeptide or a troponin I polypeptide;
iii) a LOV light-activated polypeptide comprising an amino acid sequence having at least 80% amino acid sequence identity to an amino acid sequence selected from the group consisting of SEQ ID NOS:36-40 and SEQ ID NOS:150-152;
iv) a proteolytically cleavable linker; and
v) a polypeptide of interest; and
b) a second nucleic acid comprising a nucleotide sequence encoding a second fusion polypeptide comprising:
i) a calcium-binding polypeptide selected from a calmodulin polypeptide and troponin C polypeptide; and
ii) a protease that cleaves the proteolytically cleavable linker.

3. The nucleic acid system of claim 1, wherein the insertion site is a multiple cloning site.

4. The nucleic acid system of claim 2, wherein the light-activated, calcium-gated fusion polypeptide comprises a calmodulin-binding polypeptide.

5. The nucleic acid system of claim 4, wherein the calmodulin-binding polypeptide comprises an amino acid sequence having at least 80% amino acid sequence identity to KRRWKKNFIAVSAANRFKKISSSGAL (SEQ ID NO:22) or FNARRKLKGAILTTMLATRNFS (SEQ ID NO:148).

6. The nucleic acid system of claim 4, wherein the calmodulin-binding polypeptide comprises an A14F substitution relative to the amino acid sequence KRRWKKNFIAVSAANRFKKISSSGAL (SEQ ID NO:22).

7. The nucleic acid system of claim 5, wherein the calmodulin-binding polypeptide comprises T13F and K8A amino acid substitutions relative to the amino acid sequence FNARRKLKGAILTTMLATRNFS (SEQ ID NO:148).

8. The nucleic acid system of claim 2, wherein the light-activated, calcium-gated fusion polypeptide comprises a troponin I polypeptide.

9. The nucleic acid system of claim 8, wherein the troponin I polypeptide comprises an amino acid sequence having at least 80% amino acid sequence identity to an amino acid sequence selected from the group consisting of SEQ ID NO:30 and SEQ ID NO:31.

10. The nucleic acid system of claim 2, wherein the LOV-domain light-activated polypeptide comprises one or more amino acid substitutions selected from L2R, N12S, A28V, H117R, and I130V substitutions relative to the amino acid sequence of SEQ ID NO:37.

11. The nucleic acid system of claim 2, wherein the LOV domain light-activated polypeptide comprises L2R, N12S, I130V, A28V, and H117R substitutions relative to the amino acid sequence of SEQ ID NO:37.

12. The nucleic acid system of claim 2, wherein the proteolytically cleavable linker comprises an amino acid sequence cleaved by a viral protease, a mammalian protease, or a recombinant protease.

13. The nucleic acid system of claim 2, wherein the second fusion polypeptide comprises a calmodulin polypeptide.

14. The nucleic acid system of claim 13, wherein the calmodulin polypeptide comprises an amino acid sequence having at least 80% amino acid sequence identity to an amino acid sequence selected from the group consisting of SEQ ID NO:28 and SEQ ID NO:29.

15. The nucleic acid system of 14, wherein the calmodulin polypeptide comprises F19L and V35G substitutions relative to the amino acid sequence of SEQ ID NO:28.

16. The nucleic acid system of claim 2, wherein the second fusion polypeptide comprises a troponin C polypeptide.

17. The nucleic acid system of claim 16, wherein the troponin C polypeptide comprises an amino acid sequence having at least 80% amino acid sequence identity to the amino acid sequence of SEQ ID NO:34.

18. The nucleic acid system of claim 2, wherein the protease is a viral protease, a mammalian protease, or a recombinant protease.

19. The nucleic acid system of claim 2, wherein the first nucleic acid is present in a first expression vector, and the second nucleic acid is present in a second expression vector.

20-21. (canceled)

22. The nucleic acid system of claim 2, wherein the first and/or the second nucleic acid comprises a nucleotide sequence encoding a linker that is interposed between the transmembrane domain and the calmodulin-binding polypeptide or the troponin I polypeptide, between the calmodulin-binding polypeptide or the troponin I polypeptide and the LOV domain polypeptide, between the LOV domain polypeptide and the proteolytically cleavable linker, between the proteolytically cleavable linker and the polypeptide of interest, or between the calmodulin polypeptide or the troponin C polypeptide and the protease.

23. The nucleic acid system of claim 2, wherein the polypeptide of interest is a reporter polypeptide, a light-activated polypeptide, a transcription factor, a toxin, a calcium sensor, a recombinase, an antibiotic resistance factor, a DREADD, an RNA-guided endonuclease, a kinase, a peroxidase, a synaptic marker, or an antibody.

24. The nucleic acid system of claim 23, wherein the polypeptide of interest is a reporter polypeptide selected from a fluorescent polypeptide, an enzyme that produces a colored product, an enzyme that produces a luminescent product, and an enzyme that produces a fluorescent product.

25. The nucleic acid system of claim 23, wherein the polypeptide of interest is a transcriptional activator or a transcriptional repressor.

26. The nucleic acid system of claim 23, wherein the polypeptide of interest is an antibiotic resistance factor.

27. The nucleic acid system of claim 23, wherein the polypeptide of interest is an RNA-guided endonuclease selected from a Cas9 polypeptide, a C2C2 polypeptide, or a Cpf1 polypeptide.

28. A genetically modified host cell, wherein the host cell is genetically modified with the nucleic acid system of claim 2.

29-45. (canceled)

46. A nucleic acid system comprising:
a) a first nucleic acid comprising a nucleotide sequence encoding a light-activated, calcium-gated transcription control polypeptide comprising, in order from amino terminus to carboxyl terminus:
i) a transmembrane domain;
ii) a calmodulin-binding polypeptide or a troponin I polypeptide;
iii) a LOV light-activated polypeptide comprising an amino acid sequence having at least 80% amino acid sequence identity to an amino acid sequence selected from the group consisting of SEQ ID NOS:36-40 and SEQ ID NOS:150-152;
iv) a proteolytically cleavable linker; and
v) a transcription factor; and
b) a second nucleic acid comprising a nucleotide sequence encoding a fusion polypeptide comprising:
i) a calcium-binding polypeptide selected from a calmodulin polypeptide and troponin C polypeptide; and
ii) a protease that cleaves the proteolytically cleavable linker.

47-48. (canceled)

49. The nucleic acid system of claim 46, comprising a third nucleic acid comprising a nucleotide sequence encoding a target gene product, wherein the target gene product-encoding nucleotide sequence is operably linked to a promoter that is activated by the transcription factor.

50. The nucleic acid system of claim 49, wherein the target gene product is a reporter polypeptide.

51. The nucleic acid system of claim 49, wherein the third nucleic acid is a third expression vector.

52. The nucleic acid system of claim 49, wherein the third nucleic acid comprises a nucleotide sequence encoding a second light-responsive polypeptide, wherein the light-responsive polypeptide-encoding nucleotide sequence is operably linked to a promoter, wherein the second light activated polypeptide is activated by light of a wavelength that is different from the wavelength of light that activates the light-responsive polypeptide in the light-activated, calcium-gated transcription control polypeptide.

53-61. (canceled)

62. A light-activated, calcium-gated transcription control fusion polypeptide comprising, in order from amino terminus to carboxyl terminus:
a) a transmembrane domain;
b) a calmodulin-binding polypeptide or a troponin I polypeptide;
c) a LOV domain light-activated polypeptide comprising an amino acid sequence having at least 80% amino acid sequence identity to an amino acid sequence selected from the group consisting of SEQ ID NOS:36-40 and SEQ ID NOS: 150-152;
d) a proteolytically cleavable linker; and
e) a transcription factor,
wherein the light-activated polypeptide undergoes a reversible conformational change when exposed to light of an activating wavelength, and wherein the conformational change exposes the proteolytically cleavable linker to a protease.

63-74. (canceled)

75. A polypeptide system comprising
a) the light-activated, calcium-gated transcription control fusion polypeptide of claim 62; and
b) a second fusion polypeptide comprising:
i) a calmodulin polypeptide or a troponin C polypeptide; and
ii) a protease that cleaves the proteolytically cleavable linker.

76. The system of claim 75, wherein the light-activated, calcium-gated transcription control fusion polypeptide comprises a calmodulin-binding polypeptide, and wherein the second fusion polypeptide comprises a calmodulin polypeptide.

77. The system of claim 75, wherein the light-activated, calcium-gated transcription control fusion polypeptide comprises a troponin I polypeptide, and wherein the second fusion polypeptide comprises a troponin C polypeptide.

78. The system of claim 76, wherein the calmodulin polypeptide comprises an amino acid sequence having at least 80% amino acid sequence identity to an amino acid sequence selected from the group consisting of SEQ ID NO:28 and SEQ ID NO:29.

79. The system of claim 77, wherein the calmodulin polypeptide comprises F19L and V35G substitutions relative to the amino acid sequence of SEQ ID NO:28.

80. The system of claim 76, wherein the calmodulin-binding polypeptide comprises an amino acid sequence having at least 80% amino acid sequence identity to KRRWKKNFIAVSAANRFKKISSSGAL (SEQ ID NO:22) or FNARRKLKGAILTTMLATRNFS (SEQ ID NO:148).

81. The system of claim 80, wherein the calmodulin-binding polypeptide comprises an A14F substitution relative to the amino acid sequence KRRWKKNFIAVSAANRFKKISSSGAL (SEQ ID NO:22).

82. The system of claim 80, wherein the calmodulin-binding polypeptide comprises T13F and K8A amino acid substitutions relative to the amino acid sequence FNARRKLKGAILTTMLATRNFS (SEQ ID NO:148).

83. The system of claim 77, wherein the troponin C polypeptide comprises an amino acid sequence having at least 80% amino acid sequence identity to the amino acid sequence of SEQ ID NO:34.

84. The system of claim 77, wherein the troponin I polypeptide comprises an amino acid sequence having at least 80% amino acid sequence identity to an amino acid sequence selected from the group consisting of SEQ ID NO:30 and SEQ ID NO:31.

85. The system of claim 75, wherein the LOV-domain light-activated polypeptide comprises one or more amino acid substitutions selected from L2R, N12S, A28V, H117R, and I130V substitutions relative to the amino acid sequence of SEQ ID NO:37.

86. The system of claim 75, wherein the LOV domain light-activated polypeptide comprises L2R, N12S, I130V, A28V, and H117R substitutions relative to the amino acid sequence of SEQ ID NO:37.

87-90. (canceled)

91. A mammalian cell comprising the system of claim 75.

92. The mammalian cell of claim 91, wherein the cell is a neuron.

93. The mammalian cell of claim 91, wherein the cell is a human cell.

94-106. (canceled)

107. A genetically modified non-human organism that comprises, integrated into the genome of one or more cells of the organism, the nucleic acid system of claim 2.

108-109. (canceled)

110. A method for detecting a change in the intracellular calcium concentration in a cell in response to a stimulus, the method comprising:
a) exposing the cell to the stimulus; and
b) substantially simultaneously exposing the cell to light of an activating wavelength;
wherein the cell is genetically modified with the nucleic acid system of claim 46,
wherein an increase in a product of the reporter gene, compared to a control level of the reporter gene product, indicates that exposure to the stimulus increases the intracellular calcium concentration in the cell.

111. The method of claim 110, wherein the stimulus is a ligand, a drug, a toxin, a neurotransmitter, contact with a second cell, heat, or hypoxia.

112. The method of claim 110, wherein the reporter gene product is a fluorescent protein or an enzyme that acts on a substrate to produce a detectable product.

113-118. (canceled)

119. The method of claim 110, further comprising:
c) when the level of reporter gene product indicates that the intracellular calcium concentration is greater than 100 nM, modulating an activity of the cell.

120. The method of claim 119, wherein said modulating comprises inducing production of an effector polypeptide in the cell.

121. The method of claim 120, wherein the effector polypeptide is a hyperpolarizing opsin, a depolarizing opsin, a transcription factor, a recombinase, an RNA-guided endonuclease, a kinase, a DREADD, or a toxin.

122. A method of modulating an activity of a cell, the method comprising:
a) exposing the cell to light of an activating wavelength; and
b) substantially simultaneously exposing the cell to a second stimulus;
wherein the cell is genetically modified with the nucleic acid system of claim 2, and
wherein said exposing induces production of the polypeptide of interest, wherein the polypeptide of interest modulates an activity of the cell.

123-141. (canceled)
